(19) United States
(12) Patent Application Publication
(10) Pub. No.: US 2009/0192138 A1
(43) Pub. Date: Jul. 30, 2009

(54) COMPOUNDS

(76) Inventors: Daniel Kaspar Baeschlin, Arlesheim (CH); David Edward Clark, Essex (GB); Stephen John Dunsdon, Essex (GB); Garry Fenton, Essex (GB); Amanda Fillmore, Essex (GB); Neil Victor Harris, Essex (GB); Christopher Higgs, Essex (GB); Christopher Antony Hurley, Essex (GB); Sussie Lerche Krintel, Essex (GB); Robert Edward Mackenzie, Essex (GB); Nils Ostermann, Binzen (DE); Finton Sirockin, St. Louis (FR); Jonathan Mark Sutton, Essex (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/158,872

(22) PCT Filed: Dec. 20, 2006
(86) PCT No.: PCT/EP06/70029

§ 371 Date: Sep. 17, 2008

Related U.S. Application Data
(60) Provisional application No. 60/753,382, filed on Dec. 23, 2005.

(51) Int. Cl. A61K 31/397; C07D 487/02; A61K 31/496; A61K 31/519; C07D 243/08; A61K 31/551; C07D 413/14; A61K 31/5377; C07D 403/14; A61K 31/501; A61P 3/00; A61P 9/00; A61P 25/00; A61P 1/00; A61P 35/00

(52) U.S. Cl. 514/210.21; 544/280; 514/255.05; 514/265.1; 540/575; 514/218; 544/117; 514/234.2; 544/238; 514/252.02

(57) ABSTRACT

The invention provides novel deazaxanthine and deazahypoxanthine compounds. The compounds may be useful in the therapy of diseases and conditions in which dipeptidylpeptidase-IV (DPP-IV) is implicated.

### FIELD OF THE INVENTION

The present invention relates to compounds and their use in therapy.

### BACKGROUND TO THE INVENTION

Dipeptidylpeptidase-IV (DPP-IV) is a serine protease which cleaves N-terminal dipeptides from a peptide chain containing, in general, a proline residue in the penultimate position. DPP-IV is widely expressed in mammalian tissue as a type II integral membrane protein. The protease is expressed on the surface of differentiated epithelial cells of the intestine, liver, kidney proximal tubules, prostate, corpus luteum, and on leukocyte subsets such as lymphocytes and macrophages. A soluble form of the enzyme is found in serum that has structure and function identical to the membrane-bound form of the enzyme but lacks the hydrophobic transmembrane domain.

DPP-IV has many physiologically relevant substrates including chemokines (e.g. eotaxin and macrophage-derived chemokine), neuropeptides (e.g. neuropeptide Y and substance P), vasoactive peptides, and incretins (e.g. GLP-1 and GIP). GLP-1 (glucagon-like peptide-1) is a hormone produced in the L cells of the distal small intestine in response to ingested nutrients. GLP-1 receptor binding on various tissues stimulates insulin gene expression, biosynthesis and glucose-dependent insulin secretion, inhibits glucagon secretion, promotes satiety, slows gastric emptying and promotes growth of pancreatic beta cells.

Although the biological role of DPP-IV in mammalian systems has not been completely established, it is believed to play an important role in neuropeptide metabolism, T-cell activation, attachment of cancer cells to the endothelium and the entry of HIV into lymphoid cells. It has also been discovered that DPP-IV is responsible for inactivating glucagon-like peptide-1 (GLP-1). Since GLP-1 is a major stimulator of pancreatic insulin secretion and has direct beneficial effects on glucose disposal, DPP-IV inhibition appears to represent an attractive approach for treating, for example, non-insulin-dependent diabetes mellitus (NIDDM).

DPP-IV has also been shown to play a part in the immune response. Expressed by T-CD4+ lymphocytes, where it is synonymous with the antigen CD26, DPP-IV plays an important part in the mechanism of transplant rejection (Transplantation 1997, 63 (10), 1495-500). By allowing more selective suppression of the immune response, inhibition of DPP-IV accordingly represents an extremely promising approach in the prevention of transplant rejection in transplant patients.

Inhibitors of DPP-IV are described inter alia in WO-A-02/068420, WO-A-04/018468, WO-A-04/111051, EP-A-1338595, WO-A-03/104229, WO-A-04/050656, WO-A-04/048379, WO-A-04/096806, WO-A-05/021550, WO-A-04/108730, WO-A-03/004496, WO-A-03/024965 and WO-A-04/033455.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

A first aspect of the invention is a compound of formula (I):

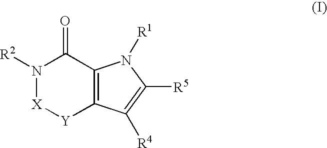

wherein
X is —CH═ and Y is ═N—; or X is —C(O)— and Y is —N(R³)—;
R¹, R² and R³ are independently each hydrogen, —W-hydrocarbyl or —W-heterocyclyl, any of which is optionally substituted, particularly on the hydrocarbyl or heterocyclyl part, with 1, 2, 3, 4 or 5 R¹²; wherein the or each W is independently a bond or a linker having from 1 to 8 in-chain atoms and selected from, for example, —CH₂—, —O—, —C(O)—, —S(O)ₘ—, —NR^{a}, carbocyclylene (e.g. cyclopropylene), heterocyclylene; C₁, C₂, C₃, C₄, C₅ or C₆ alkyl; and chemically appropriate combinations thereof; and wherein the or each R^{a} is independently hydrogen, hydroxy or hydrocarbyl optionally interrupted by an —O— or —NH— linkage;
R⁴ is hydrogen or an electron withdrawing group, for example —CF₃, —CN, —C(O)OR⁸, —C(O)NR⁸R⁹, —S(O)ₘR⁸ or —CH₂OR¹⁰;
R⁵ is a group of formula (i):

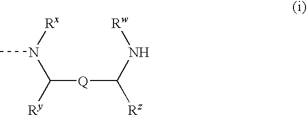

wherein
Q is a bond or alkylene comprising 1, 2 or 3 in-chain carbon atoms optionally substituted with 1, 2, 3, 4 or 5 R¹²; and
R^{w}, R^{x}, R^{y} and R^{z} are each independently hydrogen or C₁₋₆ alkyl optionally substituted with 1, 2, 3, 4 or 5 R¹²;
or two of R^{w}, R^{x}, R^{y} and R^{z} taken together form an alkylene bridge comprising 1, 2, 3, 4, 5 or 6 in-chain carbon atoms, the bridge optionally substituted with 1, 2, 3, 4 or 5 R¹²; and the other two are each hydrogen or C₁₋₆ alkyl optionally substituted with 1, 2, 3, 4 or 5 R¹²;
R⁸ and R⁹ are independently each hydrogen or C₁₋₆ alkyl optionally substituted with 1, 2, 3, 4 or 5 R¹²; or R⁸ and R⁹ taken together with the nitrogen atom to which they are attached form heterocyclyl optionally substituted with 1, 2, 3, 4 or 5 R¹²;
R¹⁰ is C₁₋₆ alkyl, C₂₋₆ alkenyl or C₂₋₆ alkynyl, any of which is optionally substituted with 1, 2, 3, 4 or 5 substituents selected from R¹¹ and R¹²;
R¹¹ is aryl or heteroaryl, either of which is optionally substituted with 1, 2, 3, 4 or 5 R¹²;
each R¹² is independently selected from:
   (i) functional moieties selected from hydroxy, halogen, amino and —CN;
   (ii) alkyl having 1, 2, 3, 4, 5 or 6 carbon atoms and optionally substituted with 1, 2, 3, 4 or 5 halogens;
   (iii) alkyl having 1, 2, 3, 4, 5 or 6 carbon atoms and optionally substituted with one or two of said functional moieties (i);
   (iv) alkoxy having 1, 2, 3, 4, 5 or 6 carbon atoms and optionally substituted with 1, 2, 3, 4 or 5 halogens; and
   (v) alkoxy having 1, 2, 3, 4, 5 or 6 carbon atoms and optionally substituted with one or two of said functional moieties (i);
or two R¹² attached to the same carbon atom form oxo (i.e. together with the attached carbon atom form carbonyl); and
m is 0, 1 or 2;
or a pharmaceutically acceptable salt or prodrug thereof.

Included in the invention are compounds in which, particularly when R⁵ is other than homopiperazinyl optionally substituted with 1, 2, 3, 4 or 5 R¹², at least two of the following provisos apply:
(i) R¹ is selected from C₁₋₆ alkyl, C₂₋₆ alkenyl and C₂₋₆ alkynyl, any of which is optionally substituted with 1, 2, 3, 4 or 5 substituents selected from R¹², carbocyclyl and heterocyclyl; or R¹ is carbocyclyl or heterocyclyl, either of which is optionally substituted with 1, 2, 3, 4 or 5 R¹²;
(ii) R² is —W-hydrocarbyl or —W-heterocyclyl, either of which is optionally substituted with 1, 2, 3, 4 or 5 R¹², wherein W is a linker; and
(iii) R⁴ is cyano;
and pharmaceutically acceptable salts and prodrugs thereof.

A second aspect of the invention is a compound of the invention for therapeutic use.

Another aspect of the invention is a pharmaceutical formulation comprising a compound of the invention and, optionally, a pharmaceutically acceptable diluent or carrier.

A further aspect of the invention is a product comprising a compound of the invention and a therapeutic agent; as a combined preparation for simultaneous, separate or sequential use in therapy.

Another aspect of the invention is the use of a compound of the invention for the manufacture of a medicament for the treatment or prevention of a disease or condition selected from non-insulin-dependent diabetes mellitus, arthritis, obesity, allograft transplantation, calcitonin-osteoporosis, heart failure, impaired glucose metabolism or impaired glucose tolerance, neurodegenerative diseases, cardiovascular or renal diseases, and neurodegenerative or cognitive disorders.

Another aspect of the invention is the use of a compound of the invention for the manufacture of a medicament for producing a sedative or anxiolytic effect, attenuating post-surgical catabolic changes or hormonal responses to stress, reducing mortality and morbidity after myocardial infarction, modulating hyperlipidemia or associated conditions, or lowering VLDL, LDL or Lp(a) levels.

Another aspect of the invention is a method of treating or preventing a disease or condition in a patient, which comprises administering a therapeutically effective amount of a compound of the invention.

The compounds of the invention can exist in different forms, such as free acids, free bases, esters and other prodrugs, salts and tautomers, for example, and the disclosure includes all variant forms of the compounds.

It will be understood that the invention specifically includes variants of individual or exemplary compounds or compound classes in which one or more moieties have been replaced by alternatives described in this application.

The extent of protection includes counterfeit or fraudulent products which contain or purport to contain a compound of the invention irrespective of whether they do in fact contain such a compound and irrespective of whether any such compound is contained in a therapeutically effective amount. Included in the scope of protection therefore are packages which include a description or instructions which indicate that the package contains a species or pharmaceutical formulation of the invention and a product which is or comprises, or purports to be or comprise, such a formulation or species.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Throughout the description and claims of this specification, the words “comprise” and “contain” and variations of the words, for example “comprising” and “comprises”, mean “including but not limited to”, and are not intended to (and do not) exclude other moieties, additives, components, integers or steps.

Further aspects and embodiments of the disclosure are set forth in the following description and claims.

### DESCRIPTION OF VARIOUS EMBODIMENTS

The following terms and abbreviations are used in this specification:

### Hydrocarbyl

The term “hydrocarbyl” as used herein includes reference to a moiety consisting exclusively of hydrogen and carbon atoms; such a moiety may comprise an aliphatic and/or an aromatic moiety. Cyclohydrocarbyl therefore includes saturated or unsaturated cyclic hydrocarbyl groups. The moiety may comprise 1, 2, 3, 4, 5, 6, 7, 8, 9, 10, 11, 12, 13, 14, 15 or 16 carbon atoms. Example of hydrocarbyl groups include C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. phenyl) or by cycloalkyl; cycloalkyl (e.g. cyclopropyl, cyclobutyl, cyclopentyl or cyclohexyl); aryl (e.g. phenyl, naphthyl or fluorenyl) and the like.

### Carbocyclyl

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered ring or ring system and, in particular, a 5- or 6-membered ring, which may be saturated or unsaturated. A carbocyclic moiety is, for example, selected from cyclopropyl, cyclobutyl, cyclopentyl, cyclohexyl, norbornyl, bicyclo[2.2.2]octyl, phenyl, naphthyl, fluorenyl, azulenyl, indenyl, anthryl and the like.

### Heterocyclyl

The term “heterocyclyl” as used herein includes reference to a saturated (e.g. heterocycloalkyl) or unsaturated (e.g. heteroaryl) heterocyclic ring moiety having from 4, 5, 6, 7, 8, 9, 10, 11, 12, 13, 14, 15 or 16 ring atoms, at least one of which is selected from nitrogen, oxygen, phosphorus and sulphur. This term includes reference to groups such as pyrazolyl, piperidinyl, pyrrolidinyl, morpholinyl, oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl (e.g. piperidin-1-yl), piperazinyl (e.g. piperazin-1-yl), pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, and the like.

### Alkyl

The terms “alkyl” and “C₁₋₆ alkyl” as used herein include reference to a straight or branched chain alkyl moiety having 1, 2, 3, 4, 5 or 6 carbon atoms. These terms include reference to groups such as methyl, ethyl, propyl (n-propyl or isopropyl), butyl (n-butyl, sec-butyl or tert-butyl), pentyl, hexyl and the like. In one class of embodiments alkyl has 1, 2, 3 or 4 carbon atoms.

### Alkenyl

The terms “alkenyl” and “C₂₋₆ alkenyl” as used herein include reference to a straight or branched chain alkyl moiety having 2, 3, 4, 5 or 6 carbon atoms and having, in addition, at least one double bond, of either E or Z stereochemistry where applicable. These terms include reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

### Alkynyl

The terms “alkynyl” and “C₂₋₆ alkynyl” as used herein include reference to a straight or branched chain alkyl moiety having 2, 3, 4, 5 or 6 carbon atoms and having, in addition, at least one triple bond. These terms include reference to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like.

### Alkoxy

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments alkoxy has 1, 2, 3 or 4 carbon atoms. These terms include reference to groups such as methoxy, ethoxy, propoxy, isopropoxy, butoxy, tert-butoxy, pentoxy, hexoxy and the like.

### Cycloalkyl

The term “cycloalkyl” as used herein includes reference to an alicyclic moiety having 3, 4, 5 or 6 carbon atoms. The group may be a polycyclic ring system. More often cycloalkyl groups are monocyclic. This term includes reference to groups such as cyclopropyl, cyclobutyl, cyclopentyl, cyclohexyl and the like.

### Aryl

The term “aryl” as used herein includes reference to an aromatic ring system comprising 6, 7, 8, 9, 10, 11, 12, 13, 14, 15 or 16 ring carbon atoms. The group is often phenyl but may be a polycyclic ring system, having two or more rings, at least one of which is aromatic. This term includes reference to groups such as phenyl, naphthyl, fluorenyl and the like.

### Heterocycloalkyl

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

### Heteroaryl

The term “heteroaryl” as used herein includes reference to an aromatic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic but is more often monocyclic. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

### Halogen

The term “halogen” as used herein refers to F, Cl, Br or I. In a particular class of embodiments halogen is F or Cl, of which F is more common.

It will be appreciated that linear organic moieties mentioned herein may comprise, for example, 1, 2, 3, 4, 5, 6, 7 or 8 carbon atoms, while cyclic moieties may comprise single rings having 4, 5, 6, 7 or 8 (e.g. 5, 6 or 7) ring atoms or may comprise fused rings of which each ring has 4, 5, 6, 7 or 8 (e.g. 5, 6 or 7) ring atoms.

### Substituted

The term “substituted” as used herein in reference to a moiety or group means that one or more hydrogen atoms in the respective moiety, especially up to 5, more especially 1, 2 or 3 of the hydrogen atoms are replaced independently of each other by the corresponding number of the described substituents . Where the substituent is halo, particularly fluoro, any number of hydrogens may in principle be replaced.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

The term “pharmaceutically acceptable” as used herein refers to compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The term “electron withdrawing group” as used herein refers to any atom or group which has an electronegativity greater than that of a hydrogen atom (i.e. as defined on the Pauling scale). Examples of electron withdrawing groups include halo (e.g. bromo, fluoro, chloro and iodo); nitro, carboxy (including esterified carboxy), C₂₋₆ alkenyl, C₂₋₆ alkynyl, formyl, carboxyamido, sulfonyl, aryl, quaternary ammonium, haloalkyl (e.g. trifluoromethyl), cyano and the like. Exemplary are functional groups, for example cyano, nitro, carboxy, formyl, sulfonyl, and quaternary ammonium; also exemplary are C₁-C₂ haloalkyl, notably trifluoromethyl.

### Compounds

For the avoidance of doubt, compounds of formula (I) in which X is —CH═ and Y is ═N— have the following structure:

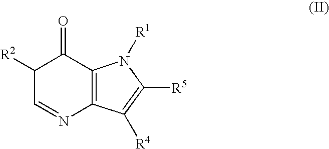

Compounds in which X is —C(O)— and Y is —N(R³)— have the following structure:

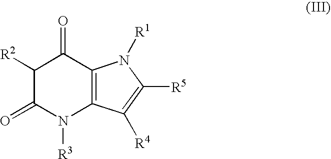

Embodiments of compounds of the invention are described below. It will be appreciated that the features specified in each embodiment may be combined with other specified features, to provide further embodiments.

### R¹

In one embodiment of the invention, R¹ is hydrogen.

In another embodiment, R¹ is —W-hydrocarbyl, wherein W is as previously defined and more particularly is selected from a bond, —(CH₂)ₙ—, —(CH₂)ₙ—O—(CH₂)ₖ—, —(CH₂)ₙ—C(O)—(CH₂)ₖ—, —(CH₂)ₙ—C(O)O—, —(CH₂)ₙ—OC(O)—, —(CH₂)ₙ—C(O)NR^{a}—, —(CH₂)ₙ—NR^{a}—, —(CH₂)ₙ—NR^{a}C(O)—, —(CH₂)ₙ—NR^{a}C(O)O—, and —(CH₂)ₙ—S(O)_{M}—, wherein k and n are independently each 0, 1, 2, 3, 4, 5 or 6; and hydrocarbyl is, for example, aryl, in particular phenyl, optionally substituted with 1, 2, 3, 4 or 5 R¹².

In a further embodiment, R¹ is —W-heterocyclyl, wherein W is selected from a bond, —(CH₂)ₙ—, —(CH₂)ₙ—O—, —(CH₂)ₙ—C(O)—, —(CH₂)ₙ—C(O)O—, —(CH₂)ₙ—O—(CH₂)ₖ—, —(CH₂)ₙ—C(O)—(CH₂)ₖ—, —(CH₂)ₙ—OC(O)—, —(CH₂)ₙ—C(O)NR^{a}—, —(CH₂)ₙ—NR^{a}—, —(CH₂)ₙ—NR^{a}C(O)—, —(CH₂)ₙ—NR^{a}C(O)O—, and —(CH₂)ₙ—S(O)_{M}—, wherein k and n are independently each 0, 1, 2, 3, 4, 5 or 6; and heterocyclyl is, for example, heteroaryl, in particular pyridinyl or thienyl, and is optionally substituted with 1, 2, 3, 4 or 5 R¹².

Typically, W is —(CH₂)ₙ—, particularly —CH₂—, or is —(CH₂)ₙ—O—, particularly —CH₂—O— or CH₂CH₂O—.

In a further embodiment, R¹ is C₁₋₆ alkyl, for example C₁, C₂, C₃ or C₄ alkyl (e.g. methyl, ethyl, propyl, isopropyl, n-butyl, sec-butyl or tert-butyl), any of which is optionally substituted with 1, 2, 3, 4 or 5 R¹²; wherein the or each R¹² is, for example, C₁₋₆ alkoxy, hydroxy or halogen (e.g. chlorine or fluorine). Alkoxy may be unsubstituted or substituted, for example by 1, 2, 3, 4 or 5 halogens, e.g. selected from F and Cl. Substituted and unsubstituted alkoxyalkyl having 2, 3, 4 or 5 carbon atoms may be mentioned as R¹ groups. Exemplary R¹ groups include linear alkyl and linear alkoxyalkyl, for example in either case having a chain length of up to 6 atoms, e.g. straight chain alkoxyalkyl having 2, 3 or 4 carbon atoms. In a particular embodiment, R¹ is methyl, ethyl, propyl, butyl or 2-methoxyethyl.

In a further embodiment, R¹ is C₂₋₆ alkenyl optionally substituted with 1, 2, 3, 4 or 5 R¹². For example, R¹ may be C₂, C₃, C₄, C₅ or C₆ alkenyl (e.g. ethenyl, 2-propenyl, 1-butenyl, 2-butenyl, 3-butenyl, 3-methyl-but-2-enyl, 1-pentenyl, 2-pentenyl, 3-pentenyl, 1-hexenyl, 2-hexenyl or 3-hexenyl), any of which is optionally substituted with 1, 2, 3, 4 or 5 R¹², wherein the or each R¹² is, for example, C₁₋₆ alkoxy, hydroxy or halogen (e.g. chlorine or fluorine). In a particular embodiment, R¹ is 3-methyl-buten-2-yl.

In a further embodiment, R¹ is C₂₋₆ alkynyl, for example C₂, C₃, C₄, C₅ or C₆ alkynyl (e.g. ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl or 3-hexynyl), any of which is optionally substituted with 1, 2, 3, 4 or 5 R¹², wherein the or each R¹² is, for example, C₁₋₆ alkoxy, hydroxy or halogen (e.g. chlorine or fluorine). In a particular embodiment, R¹ is but-2-ynyl.

In a further embodiment, R¹ is —(CH₂)ₙ—R⁶, wherein n is 0, 1, 2, 3, 4, 5 or 6, and R⁶ is carbocyclyl (e.g. cycloalkyl or aryl) or heterocyclyl (e.g. heterocycloalkyl or heteroaryl), either of which is optionally substituted with 1, 2, 3, 4 or 5 R¹²; wherein the or each R¹² is selected from, for example, hydroxy; halogen (e.g. chlorine or fluorine); C₁, C₂, C₃ or C₄ alkyl (e.g. methyl, ethyl, propyl, isopropyl, n-butyl, sec-butyl or tert-butyl) optionally substituted with 1, 2 or 3 hydroxy or 1, 2, 3, or 3 or more halogen (e.g. chlorine or fluorine); and C₁, C₂, C₃ or C₄ alkoxy (e.g. methoxy, ethoxy, propoxy, isopropoxy, butoxy, tert-butoxy), optionally substituted with 1, 2, 3 or more halogen (e.g. fluorine or chlorine) atoms.

In a further embodiment, R¹ is —(CH₂)ₙ-aryl, wherein n is 0, 1 or 2, and aryl is phenyl, naphthyl or fluorenyl, any of which is optionally substituted with 1, 2, 3, 4 or 5 R¹². When aryl is phenyl, it is preferably substituted at any of the 2-, 3-, 4- and 5-positions with a substituent selected from halogen (e.g. fluorine or chlorine), hydroxy, cyano, methoxy, ethoxy, methyl, trifluoromethyl and ethyl.

In a further embodiment, R¹ is benzyl optionally substituted with 1, 2 or 3 R¹², wherein the or each R¹² is selected from hydroxy, halogen (e.g. chlorine or fluorine); C₁, C₂, C₃ or C₄ alkyl (e.g. methyl, ethyl, propyl, isopropyl, n-butyl, sec-butyl or tert-butyl) optionally substituted with 1, 2 or 3 hydroxy or halogen (e.g. chlorine or fluorine); and C₁, C₂, C₃ or C₄ alkoxy (e.g. methoxy, ethoxy, propoxy, isopropoxy, butoxy, tert-butoxy), optionally substituted with 1, 2 or 3 halogen (e.g. fluorine or chlorine) atoms. Exemplary substituents are halogen. The phenyl part of the benzyl group is preferably substituted at any of the 2-, 3-, 4- and 5-positions with a substituent selected from, for example, halogen (e.g. fluorine or chlorine), hydroxy, cyano, methoxy, ethoxy, methyl, trifluoromethyl and ethyl. In a particular embodiment, R¹ is 2-chlorobenzyl. In another embodiment, R¹ is 2-chloro-5-fluoromethylbenzyl. In another embodiment, R¹ is 3-methyl-buten-2-yl, but-2-ynyl,2-fluorobenzyl or unsubstituted benzyl. Of particular mention are compounds in which R¹ is unsubstituted benzyl.

In a further embodiment, R¹ is —(CH₂)ₙ-cycloalkyl, wherein n is 0, 1 or 2, and cycloalkyl is cyclopropyl, cyclobutyl, cyclopentyl or cyclohexyl, any of which is optionally substituted with 1, 2, 3, 4 or 5 R¹². When cycloalkyl is cyclopropyl, it is preferably substituted at either of the 2- and 3-positions with a substituent selected from halogen (e.g. fluorine or chlorine), hydroxy, cyano, methoxy, ethoxy, methyl, trifluoromethyl and ethyl. In a particular embodiment, R¹ is cyclopropylmethyl, 2-methylcyclopropylmethyl, cyclopropylethyl, or cyclobutylmethyl.

In a further embodiment, R¹ is —(CH₂)ₙ-heterocycloalkyl, wherein n is 0, 1 or 2, and heterocycloalkyl is azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl, any of which is optionally substituted with 1, 2, 3, 4 or 5 R¹². Of mention are compounds in which the heterocycloalkyl portion is unsubstituted. In a particular embodiment, R¹ is tetrahydrofuranylmethyl, for example tetrahydrofuran-2-ylmethyl.

In a further embodiment, R¹ is —(CH₂)ₙ-heteroaryl, wherein n is 0, 1 or 2 and heteroaryl is pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl or pteridinyl, any of which is optionally substituted with 1, 2, 3, 4 or 5 R¹², wherein the or each R¹² is selected from cyano, trifluoromethyl, hydroxy, halogen (e.g. chlorine or fluorine); C₁, C₂, C₃ or C₄ alkyl (e.g. methyl, ethyl, propyl, isopropyl, n-butyl, sec-butyl or tert-butyl) optionally substituted with 1, 2, 3 or more hydroxy or halogen (e.g. chlorine or fluorine); and C₁, C₂, C₃ or C₄ alkoxy (e.g. methoxy, ethoxy, propoxy, isopropoxy, butoxy, tert-butoxy), optionally substituted with 1, 2, 3 or more halogen (e.g. fluorine or chlorine) atoms. Of mention are compounds in which the heteroaryl portion is unsubstituted. In a particular embodiment, R¹ is thiazolylmethyl, furanylmethyl or oxazolylmethyl.

In a further embodiment of the invention, R¹ is a group selected from:

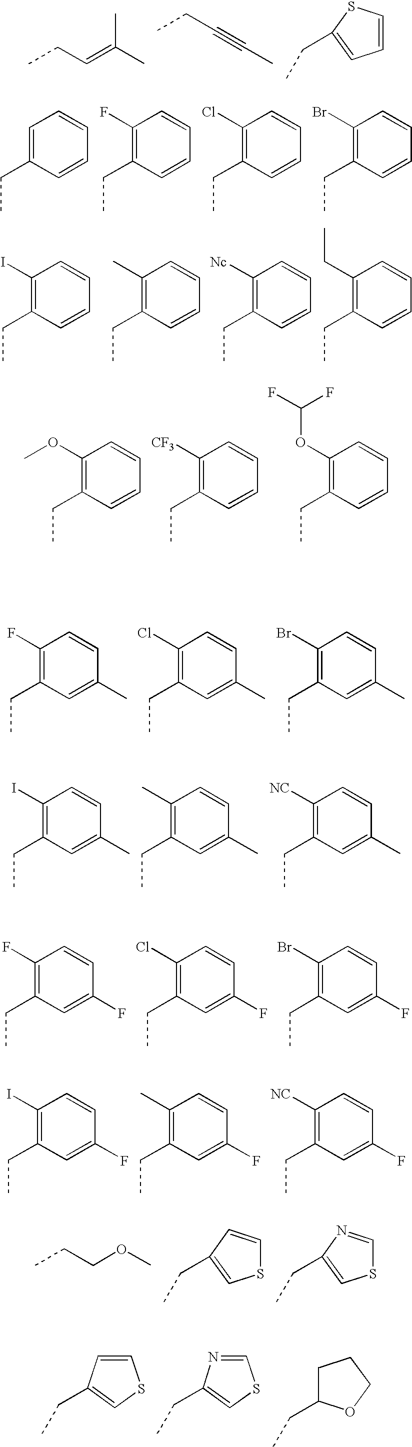

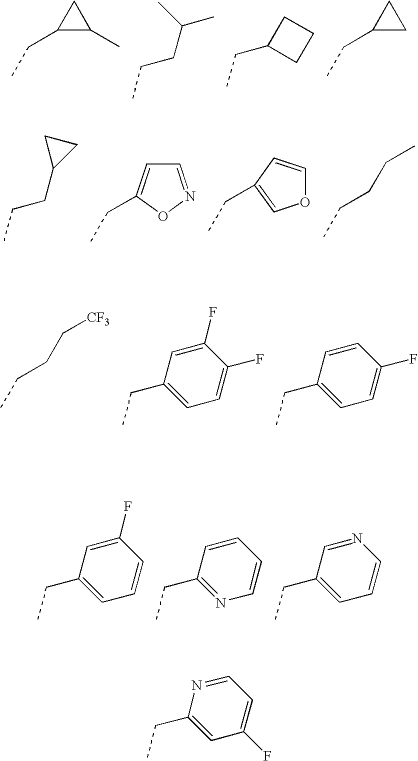

Often , R¹ is 3-methyl-buten-2-yl, but-2-ynyl,2-fluorobenzyl or unsubstituted benzyl.

### R²

In one embodiment of the invention, R² is hydrogen.

In further embodiment, R² is —W-hydrocarbyl, wherein W is as defined previously and more particularly is selected from a bond, —(CH₂)ₙ—, —(CH₂)ₙ—O—(CH₂)ₖ—, —(CH₂)ₙ—C(O)—(CH₂)ₖ—, —(CH₂)ₙ—C(O)O—, —(CH₂)ₙ—OC(O)—, —(CH₂)ₙ—C(O)NR^{a}, —(CH₂)ₙ—NR^{a}, —(CH₂)ₙ—S(O)ₘ—NR^{a}(CH₂)ₖ, —(CH₂)ₙ—NR^{a}C(O)—, —(CH₂)ₙ—NR^{a}C(O)O—, —(CH₂)ₙ—NR^{a}C(O)—NR^{a}—(CH₂)ₖ and —(CH₂)ₙ—S(O)ₘ—, wherein k and n are independently each 0, 1, 2, 3, 4, 5 or 6; and hydrocarbyl is, for example, C₁, C₂, C₃ or C₄ alkyl (e.g. methyl, ethyl, propyl, isopropyl, n-butyl, sec-butyl or tert-butyl), cycloalkyl or aryl, in particular methyl, ethyl, cyclohexyl, phenyl or naphthyl, any of which is optionally substituted with 1, 2, 3, 4 or 5 R¹². Also of mention are compounds in which W is a linker comprising a carbocyclylene or heterocyclylene linkage.

In a further embodiment, R² is C₁₋₆ alkyl, for example C₁, C₂, C₃ or C₄ alkyl (e.g. methyl, ethyl, propyl, isopropyl, n-butyl, sec-butyl or tert-butyl), any of which is optionally substituted with 1, 2, 3, 4 or 5 R¹²; wherein the or each R¹² is, for example, C₁₋₆ alkoxy, hydroxy or halogen (e.g. chlorine or fluorine). Alkoxy may be unsubstituted or substituted, for example by 1, 2, 3, 4 or 5 halogens, e.g. selected from F and Cl. Substituted and unsubstituted alkoxyalkyl having 2, 3, 4 or 5 carbon atoms may be mentioned as R² groups. Exemplary R² groups include linear alkyl and linear alkoxyalkyl, for example in either case having a chain length of up to 6 atoms, e.g. straight chain alkoxyalkyl having 2, 3 or 4 carbon atoms.

In a further embodiment, R² is C₂₋₆ alkenyl, for example C₂, C₃, C₄, C₅ or C₆ alkenyl (e.g. ethenyl, 2-propenyl, 1-butenyl, 2-butenyl, 3-butenyl, 3-methyl-but-2-enyl, 1-pentenyl, 2-pentenyl, 3-pentenyl, 1-hexenyl, 2-hexenyl or 3-hexenyl), any of which is optionally substituted with 1, 2, 3, 4 or 5 R¹², wherein the or each R¹² is, for example, C₁₋₆ alkoxy, hydroxy or halogen (e.g. chlorine or fluorine). In a particular embodiment, R² is 3-methyl-buten-2-yl.

In a further embodiment, R² is C₂₋₆ alkynyl, for example C₂, C₃, C₄, C₅ or C₆ alkynyl (e.g. ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl or 3-hexynyl), any of which is optionally substituted with 1, 2, 3, 4 or 5 R¹², wherein the or each R¹² is, for example, C₁₋₆ alkoxy, hydroxy or halogen (e.g. chlorine or fluorine). In a particular embodiment, R² is but-2-ynyl.

In a further embodiment, R² is —W-heterocyclyl, wherein W is selected from a bond, —(CH₂)ₙ—, —(CH₂)ₙ—O—(CH₂)ₖ—, —(CH₂)ₙ—C(O)—(CH₂)ₖ—, —(CH₂)ₙ—C(O)O—, —(CH₂)ₙ—OC(O)—, —(CH₂)ₙ—C(O)NR^{a}—, —(CH₂)ₙ—NR^{a}—, —(CH₂)ₙ—S(O)ₘ—NR^{a}(CH₂)ₖ, —(CH₂)ₙ—NR^{a}C(O)—, —(CH₂)ₙ—NR^{a}C(O)O—, —(CH₂)ₙ—NR^{a}C(O)—NR^{a}—(CH₂)ₖ and —(CH₂)ₙ—S(O)ₘ—, wherein k and n are independently each 0, 1, 2, 3, 4, 5 or 6 and R^{a} is selected from hydrogen, hydroxy, hydrocarbyl optionally substituted with 1, 2, 3, 4 or 5 R¹⁰; and heterocyclyl optionally substituted with 1, 2, 3, 4 or 5 R¹⁰; and heterocyclyl is, for example, heterocycloalkyl or heteroaryl, in particular piperidin-1-yl, thiophen-1-yl, thiophen-2-yl, benzo[b]thiophenyl, pyridin-1-yl, pyridin-2-yl, pyridin-3-yl, pyrazin-2-yl or quinolin-4-yl, any of which is optionally substituted with 1, 2, 3, 4 or 5 R¹². In one class of compounds, R² is quinolinyl or isoquinolinyl, e.g. isoquinolin-1-yl. Also of mention are compounds in which W is a linker comprising a carbocyclylene or heterocyclylene linkage.

Typically, W is —(CH₂)ₙ—, e.g. —CH₂—, or is —(CH₂)ₙ—C(O)—(CH₂)ₘ—, e.g. —CH₂—C(O)—.

In a further embodiment, R² is —CH₂C(O)-hydrocarbyl, —CH₂C(O)O-hydrocarbyl, —CH₂C(O)-heterocyclyl or —CH₂-heterocyclyl; wherein hydrocarbyl is in particular C₁, C₂, C₃ or C₄ alkyl (e.g. methyl, ethyl, propyl, isopropyl, n-butyl, sec-butyl or tert-butyl), cycloalkyl (e.g. cyclohexyl) or aryl (e.g. phenyl or naphthyl); and heterocyclyl is in particular heterocycloalkyl (e.g. piperidin-1-yl) or heteroaryl (e.g. thiophen-1-yl, thiophen-2-yl, benzo[b]thiophenyl, pyridin-1-yl, pyridin-2-yl, pyridin-3-yl, pyrazin-2-yl or quinolin-4-yl); and wherein the group is optionally substituted with 1, 2, 3, 4 or 5 R¹².

In a further embodiment, R² is —(CH₂)ₙ—R⁷, —(CH₂)ₙ—OR⁷, —(CH₂)ₙ—C(O)R⁷, —(CH₂)ₙ—NR^{a}C(O)R⁷, —(CH₂)ₙ—NR^{a}S(O)ₘR⁷, —(CH₂)ₙ—S(O)ₘNR^{a}R7 or —(CH₂)ₙ—S(O)ₘR⁷, wherein n is 0, 1, 2, 3, 4, 5 or 6, and R⁷ is carbocyclyl (e.g. aryl) or heterocyclyl (e.g. heteroaryl), either of which is optionally substituted with 1, 2, 3, 4 or 5 R¹²; wherein the or each R¹² is in particular selected from, for example, cyano, trifluoromethyl, hydroxy; halogen (e.g. chlorine or fluorine); C₁, C₂, C₃ or C₄ alkyl (e.g. methyl, ethyl, propyl, isopropyl, n-butyl, sec-butyl or tert-butyl) optionally substituted with 1, 2 or 3 hydroxy or with 1, 2, 3 or more halogen (e.g. chlorine or fluorine); and C₁, C₂, C₃ or C₄ alkoxy (e.g. methoxy, ethoxy, propoxy, isopropoxy, butoxy, tert-butoxy), optionally substituted with 1, 2 or 3 or more halogen (e.g. fluorine or chlorine) atoms. Also, particularly when R⁷ is heterocyclyl, two R¹² attached to the same carbon atom taken together may form oxo. Particular R¹² groups are selected from methoxy, ethoxy, methyl, ethyl and halogen, wherein any of methoxy, ethoxy, methyl and ethyl is optionally substituted by one or more halogens, e.g. to form CF₃. In one embodiment, R⁷ is phenyl, naphthyl, thiophen-1-yl, thiophen-2-yl, benzo[b]thiophenyl, pyridin-1-yl, pyridin-2-yl, pyridin-3-yl, pyrazin-2-yl or quinolin-4-yl, any of which is optionally substituted with 1, 2, 3, 4 or 5 R¹². It is typically preferable that n is 1 or 2.

In a further embodiment, R² is —(CH₂)ₙ—C(O)-aryl, wherein n is 0, 1 or 2 (particularly 1), and aryl is phenyl or naphthyl, either of which is optionally substituted with 1, 2 or 3 R¹². When aryl is phenyl, it may be unsubstituted or substituted, for example at any of the 2-, 3- and 4-positions with a substituent selected from, for example, halogen (e.g. fluorine or chlorine), hydroxy, cyano, methoxy, ethoxy, trifluoromethyl, methyl and ethyl. In a particular embodiment, R² is 2-oxo-2-phenyl-ethyl or 2-oxo-2-(3-methoxyphenyl)-ethyl.

In a further embodiment, R² is —(CH₂)ₙ-heteroaryl, wherein n is 0, 1 or 2 (particularly 1), and heteroaryl is for example a mono- or bicyclic ring containing at least one heteroatom, for example containing one or more nitrogens. Exemplary heteroaryl groups are 6-membered rings and heteroaryl analogues of naphthyl, i.e. groups corresponding to naphthyl in which at least one carbon has been replaced by a heteroatom, e.g. nitrogen; quinolinyl and isoquinolinyl may be mentioned. Particular heteroaryl moieties are thiophen-1-yl, thiophen-2-yl, benzo[b]thiophenyl, isoquinolin-1-yl, phthalazin-6-yl, pyridin-1-yl, pyridin-2-yl, pyridin-3-yl, pyrazin-2-yl, quinazolin-2-yl quinoxalin-6-yl or quinolin-4-yl, any of which is optionally substituted with 1, 2, 3, 4 or 5 R¹², wherein the or each R¹² is in particular selected from cyano, trifluoromethyl, hydroxy, halogen (e.g. chlorine or fluorine); C₁, C₂, C₃ or C₄ alkyl (e.g. methyl, ethyl, propyl, isopropyl, n-butyl, sec-butyl or tert-butyl) optionally substituted with 1, 2 or 3 hydroxy or with 1, 2, 3 or more halogen (e.g. chlorine or fluorine); and C₁, C₂, C₃ or C₄ alkoxy (e.g. methoxy, ethoxy, propoxy, isopropoxy, butoxy, tert-butoxy), optionally substituted with 1, 2, 3 or more halogen (e.g. fluorine or chlorine) atoms. In a particular embodiment, R² is isoquinolin-1-ylmethyl.

Often, R² is 2-oxo-2-phenyl-ethyl, isoquinolin-1-ylmethyl or 2-oxo-2-(3-methoxyphenyl)-ethyl.

In a further embodiment, R² is a group selected from:

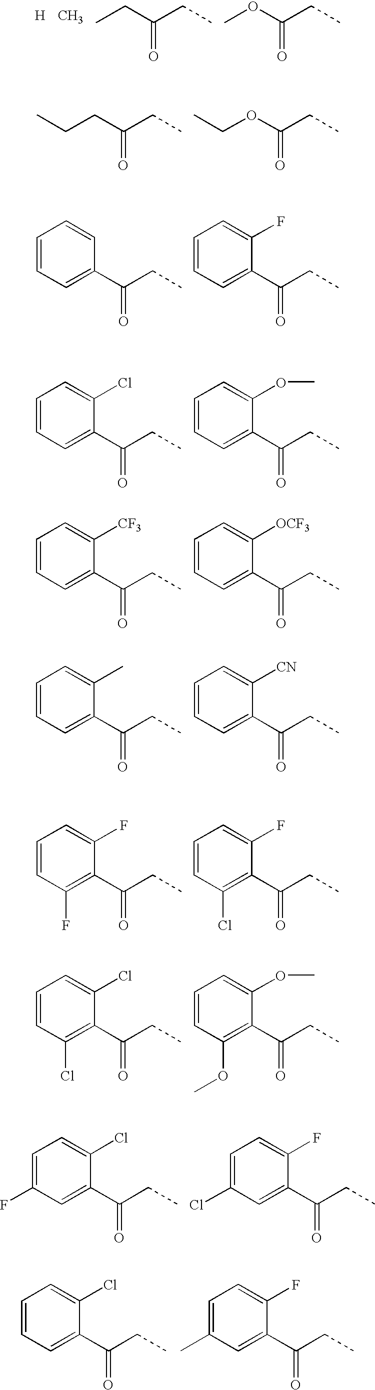

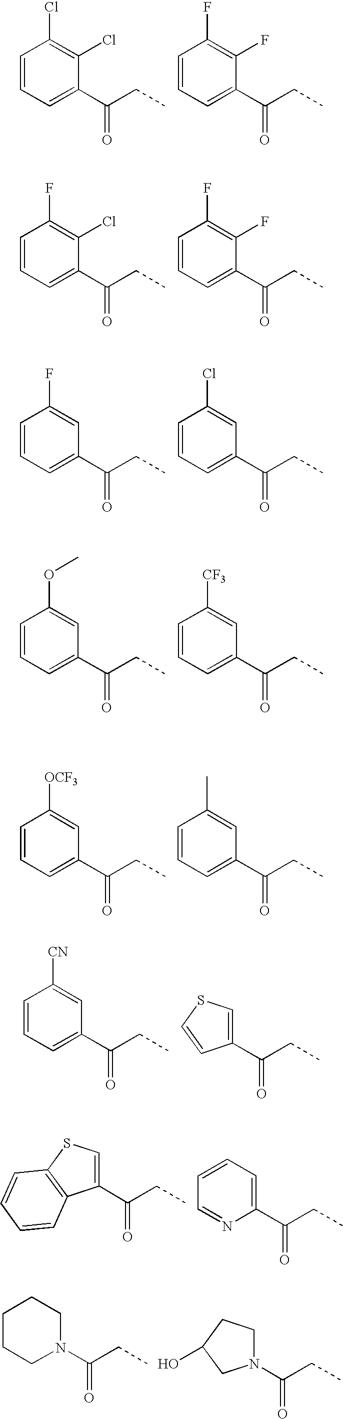

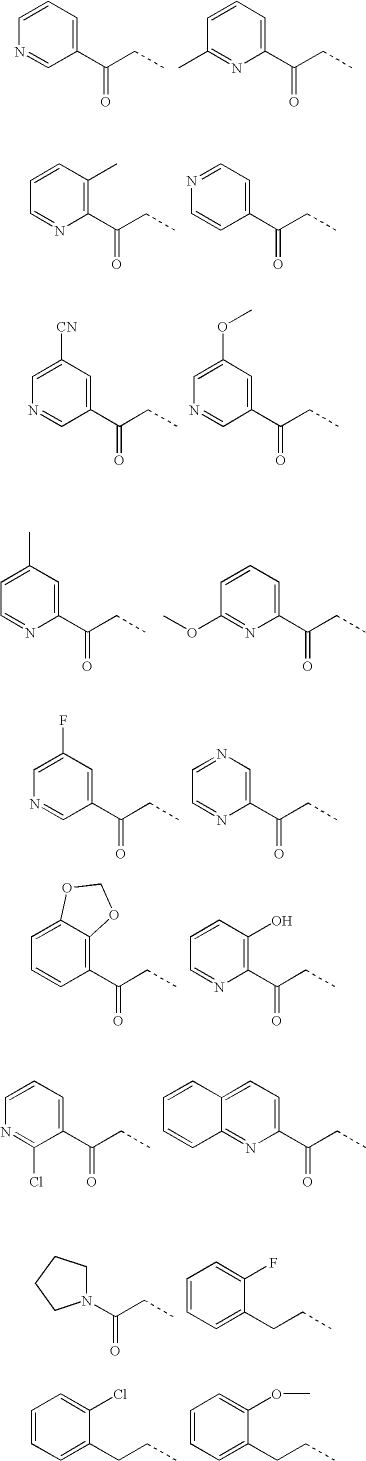

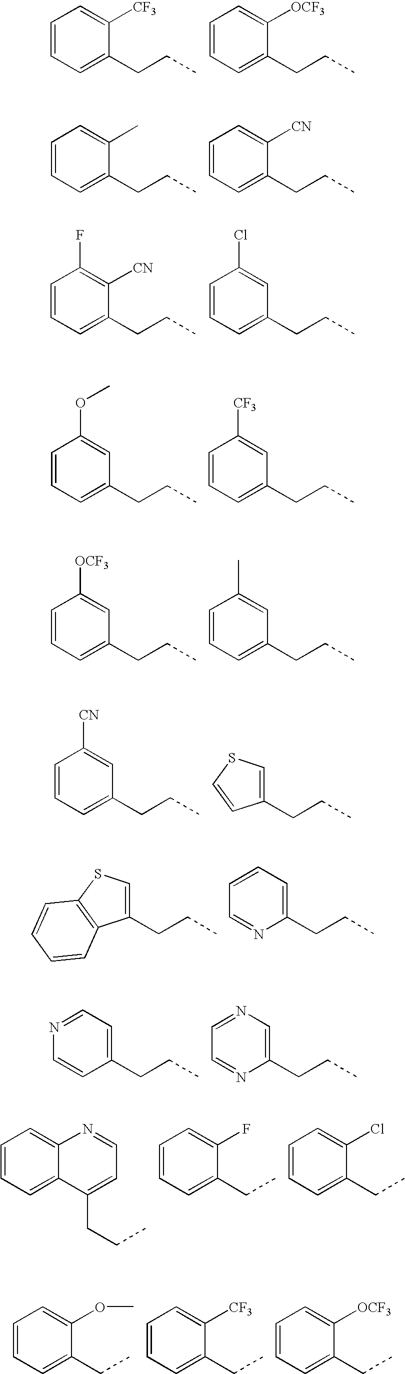

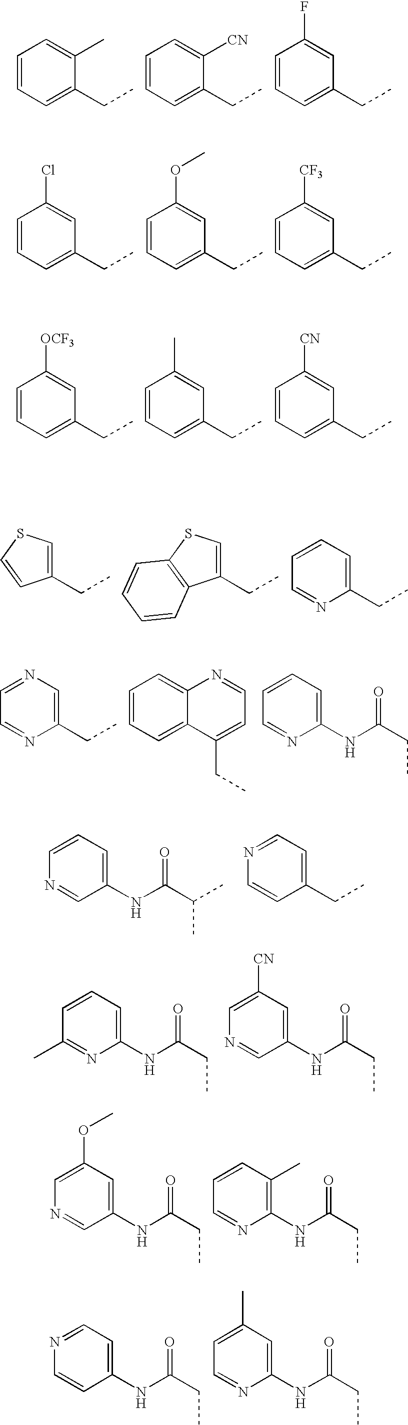

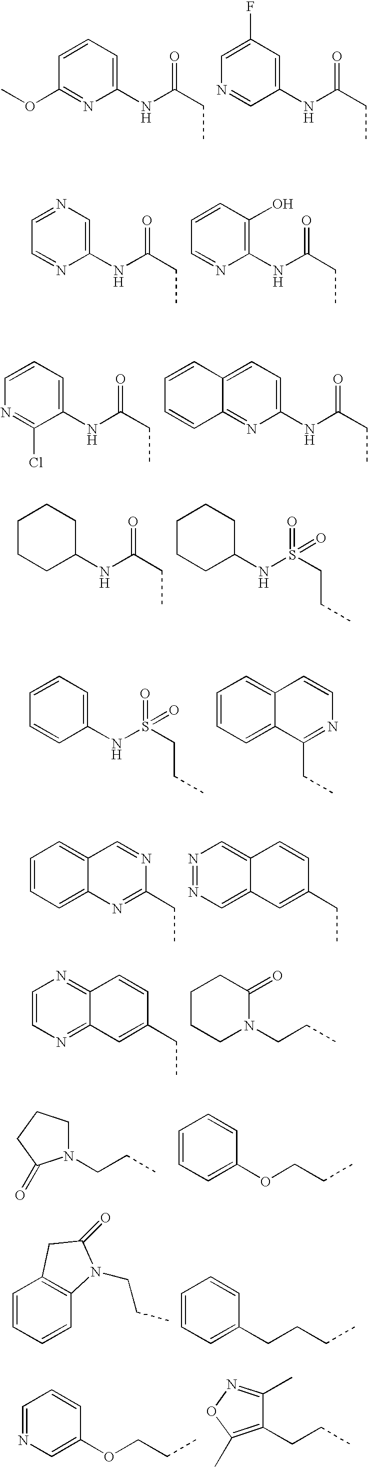

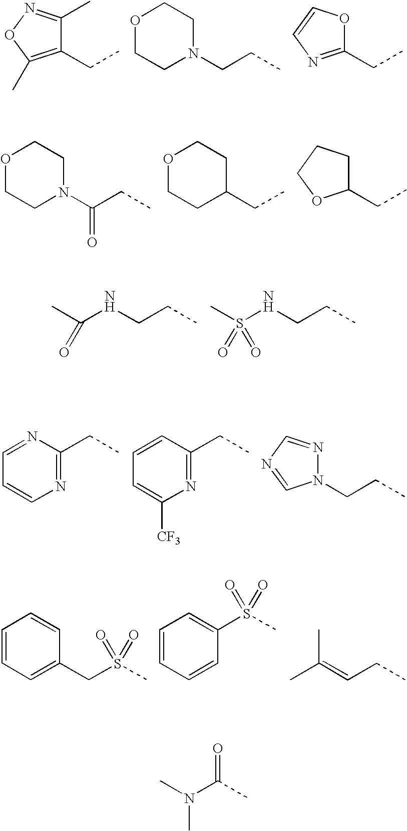

### R³

In one embodiment of the invention, R³ is any group described above in relation to R¹ or R².

In another embodiment, R³ is hydrogen.

In a further embodiment, R³ is C₁₋₆ alkyl, C₁₋₆ alkenyl, C₁₋₆ alkynyl, cycloalkyl, heterocycloalkyl, aryl, heteroaryl, —(CH₂)ₙ-cycloalkyl, —(CH₂)ₙ-aryl, —(CH₂)ₙ-heterocycloalkyl or —(CH₂)ₙ-heteroaryl, any of which is optionally substituted with 1, 2, 3, 4 or 5 R¹², wherein the or each R¹² is, for example, hydroxy or halogen (e.g. chlorine or fluorine).

In a further embodiment, R³ is hydrogen or C₁₋₆ alkyl.

In a further embodiment, R³ is hydrogen or methyl.

### R⁴

In one embodiment of the invention, R⁴ is hydrogen or an electron withdrawing group, e.g. —CF₃, —CN, —C(O)OR⁸, —C(O)NR⁸R⁹ or —S(O)ₘR⁸; wherein R⁸ and R⁹ are independently each hydrogen or C₁, C₂, C₃ or C₄ alkyl (e.g. methyl, ethyl, propyl, isopropyl, n-butyl, sec-butyl or tert-butyl) optionally substituted with 1, 2 or 3 hydroxy or halogen (e.g. chlorine or fluorine); or R⁸ and R⁹, taken together with the nitrogen atom to which they are attached, form heterocyclyl (including heterocycloalkyl, for example azetidinyl, pyrrolidinyl, piperidinyl, piperazinyl or morpholinyl) optionally substituted with 1, 2 or 3 hydroxy or halogen (e.g. fluorine or chlorine) atoms. In one class of compounds, R⁴ is not hydrogen but is an electron withdrawing group such as —CN, for example.

In a further embodiment, R⁴ is hydrogen, or more usually —CN, —C(O)OR⁸, —C(O)NR⁸R⁹, wherein R⁸ and R⁹ are, in particular, each independently hydrogen or C₁, C₂, C₃ or C₄ alkyl (e.g. methyl).

In a further embodiment, R⁴ is —CH₂OR¹⁰, wherein R¹⁰ is C₁, C₂, C₃ or C₄ alkyl (e.g. methyl, ethyl, propyl, isopropyl, n-butyl, sec-butyl or tert-butyl) optionally substituted with 1, 2 or 3 hydroxy or halogen (e.g. chlorine or fluorine); or R¹⁰ is —(CH₂)ₙ-aryl, for example phenyl or benzyl.

In a further embodiment, R⁴ is cyano.

In a further embodiment, R⁴ is —C(O)OR⁸. In certain compounds, R⁸ is hydrogen or C₁, C₂, C₃ or C₄ alkyl (e.g. methyl).

In a further embodiment, R⁴ is —C(O)NR⁸R⁹. In certain compounds, R⁸ and R⁹ are each independently hydrogen or C₁, C₂, C₃ or C₄ alkyl (e.g. methyl). In other compounds, R⁸ and R⁹ are taken together with the nitrogen atom to which they are attached to form heterocyclyl (e.g. heterocycloalkyl) optionally substituted with 1, 2, 3, 4 or 5 R¹². In particular, R⁸ and R⁹ may be taken together with the nitrogen atom to which they are attached to form morpholinyl, piperidinyl or pyrrolidinyl, any of which is optionally substituted with 1, 2, 3, 4 or 5 R¹².

In a further embodiment, R⁴ is —C(O)R⁸ or —S(O)ₘR⁸. In certain compounds, R⁸ is C₁₋₆ alkyl (e.g. C₁, C₂, C₃ or C₄ alkyl) or carbocyclyl (e.g. cycloalkyl or aryl), either of which is optionally substituted with 1, 2, 3, 4 or 5 R¹². Of mention are compounds in which m is 0 or 2, e.g. 0.

In a further embodiment, R⁴ is —S(O)ₘNR⁸R⁹. In certain compounds, R⁸ and R⁹ are each independently hydrogen or C₁, C₂, C₃ or C₄ alkyl (e.g. methyl). In other compounds, R⁸ and R⁹ are taken together with the nitrogen atom to which they are attached to form heterocyclyl (e.g. heterocycloalkyl) optionally substituted with 1, 2, 3, 4 or 5 R¹². In particular, R⁸ and R⁹ may be taken together with the nitrogen atom to which they are attached to form morpholinyl or pyrimidinyl, either of which is optionally substituted with 1, 2, 3, 4 or 5 R¹². By way of example, R⁴ may be —S(O)₂N(CH₃)₂.

### R⁵

R⁵ is a group of formula (i):

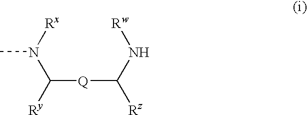

wherein
Q is a bond or alkylene comprising 1, 2 or 3 in-chain carbon atoms optionally substituted with 1, 2, 3, 4 or 5 R¹²; and
R^{w}, R^{x}, R^{y} and R^{z} are each independently hydrogen or C₁₋₆ alkyl optionally substituted with 1, 2, 3, 4 or 5 R¹²;
or two of R^{w}, R^{x}, R^{y} and R^{z} taken together form an alkylene bridge comprising 1, 2, 3, 4, 5 or 6 in-chain carbon atoms, the bridge optionally substituted with 1, 2, 3, 4 or 5 R¹²; and the other two are each hydrogen or C₁₋₆ alkyl optionally substituted with 1, 2, 3, 4 or 5 R¹²,

In one embodiment of the invention, Q is a bond, i.e. R⁵ is of formula (ii):

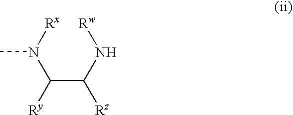

In another embodiment of the invention, Q is alkylene comprising 1, 2 or 3 in-chain carbon atoms optionally substituted with 1, 2, 3 or 4 R¹². More usually, Q is methylene optionally substituted with 1 or 2 R¹²; or ethylene optionally substituted with 1, 2, 3 or 4 R¹². In a particular embodiment, Q is methylene.

In a further embodiment, R^{w} and R^{x} together form —CH₂—, —(CH₂)₂—, —(CH₂)₃— or —(CH₂)—; and R^{y} and R^{z} are each hydrogen. Often, R^{w} and R^{x} together form —(CH₂)₂— or —(CH₂)₃—. In a class of compounds, therefore, R^{w} and R^{x} form a substituted or unsubstituted ethylene or propylene bridge. In these embodiments, Q is usually a bond; methylene optionally substituted with 1 or 2 R¹²; or ethylene optionally substituted with 1, 2, 3 or 4 R¹². In particular, Q may be a bond.

In another embodiment, R^{x} and R^{z} together form —CH₂—, —(CH₂)₂—, —(CH₂)₃— or —(CH₂)₄—; and R^{w} and R^{z} are each hydrogen. Often, R^{x} and R^{z} together form —(CH₂)₂— or —(CH₂)₃—. In a class of compounds, therefore, R^{w} and R^{z} form a substituted or unsubstituted propylene or butylene bridge. In these embodiments, Q is usually a bond; methylene optionally substituted with 1 or 2 R¹²; or ethylene optionally substituted with 1, 2, 3 or 4 R¹². In particular, Q may be a bond.

In a further embodiment, R^{y} and R^{z} together form —(CH₂)₃—, —(CH₂)₄— or —(CH₂)₅—; and R^{x} and R^{w} are each hydrogen. Often, R^{y} and R^{z} together form —(CH₂)₃— or —(CH₂)₄—. In a class of compounds, therefore, R^{y} and R^{z} form a substituted or unsubstituted propylene bridge. In these embodiments, Q is usually a bond; methylene optionally substituted with 1 or 2 R¹²; or ethylene optionally substituted with 1, 2, 3 or 4 R¹². In particular, Q may be a bond.

In a further embodiment, R^{x} and R^{w} taken together form an alkylene bridge comprising 2, 3, 4, 5 or 6 in-chain carbon atoms, the bridge optionally substituted with 1, 2, 3, 4 or 5 R¹²; and R^{y} and R^{z} are each hydrogen or C₁₋₆ alkyl optionally substituted with 1, 2, 3, 4 or 5 R¹²,

In a further embodiment, R^{x} and R^{w} taken together form an alkylene bridge comprising 2 or 3 in-chain carbon atoms, the bridge optionally substituted with 1, 2, 3, 4 or 5 R¹²; and R^{y} and R^{z} are each hydrogen or C₁₋₆ alkyl optionally substituted with 1, 2, 3, 4 or 5 R¹².

In a further embodiment, R^{x} and R^{w} taken together form an alkylene bridge comprising 3, 4, 5 or 6 in-chain carbon atoms, the bridge optionally substituted with 1, 2, 3, 4 or 5 R¹²; and R^{y} and R^{z} are each hydrogen or C₁₋₆ alkyl optionally substituted with 1, 2, 3, 4 or 5 R¹².

In a further embodiment, R^{x} and R^{w} taken together form an alkylene bridge comprising 3 in-chain carbon atoms, the bridge optionally substituted with 1, 2, 3, 4 or 5 R¹²; and R^{y} and R^{z} are each hydrogen or C₁₋₆ alkyl optionally substituted with 1, 2, 3, 4 or 5 R¹².

In a further embodiment, R⁵ is homopiperazinyl optionally substituted with 1, 2, 3, 4 or 5 R¹².

In a further embodiment, R⁵ is a group selected from:

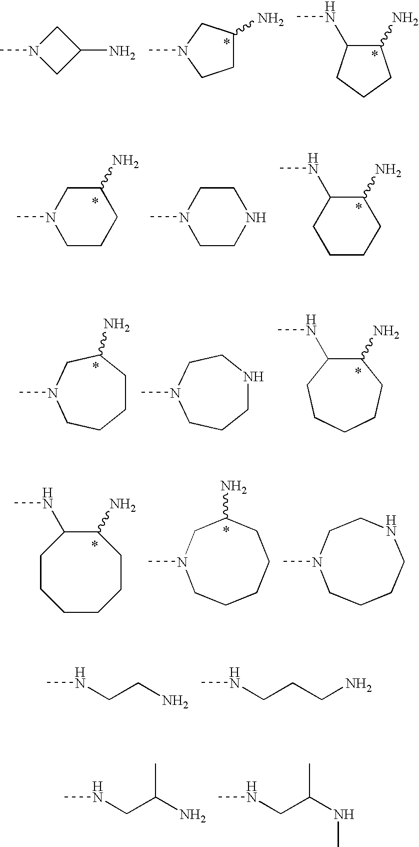

wherein the symbol * signifies a chiral centre of (S)- or (R)-configuration.

In a further embodiment, R⁵ is a group of formula (iii) or formula (iv):

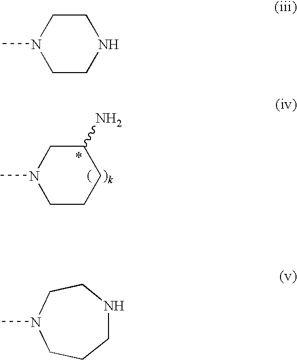

wherein the symbol * signifies a chiral centre of (S)- or (R)-configuration, and k is 0, 1 or 2, in particular 1.

Particular embodiments of the present invention include compounds of formulae (IV), (V), (VI), (VII), (VIII) and (IX), and pharmaceutically acceptable salts and prodrugs thereof:

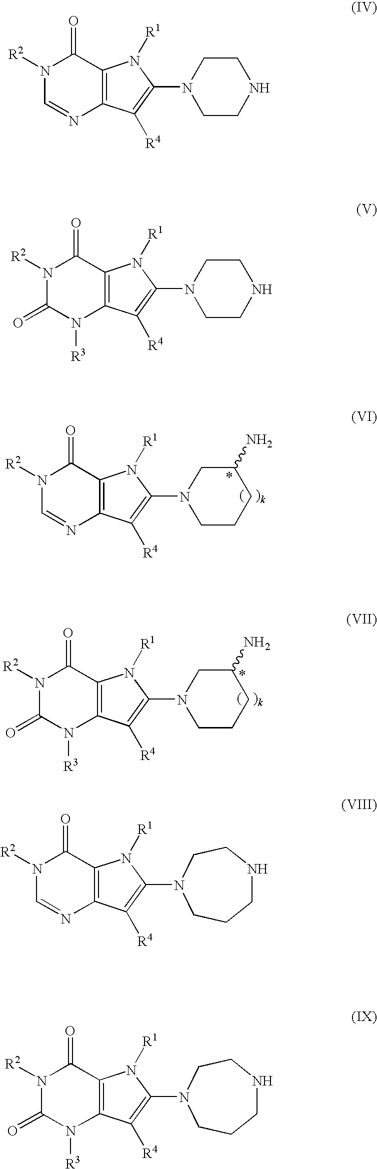

wherein the symbol * designates a chiral centre of (S)- or (R)-configuration.

### R¹²

For the avoidance of doubt, where a group is substituted with more than one R¹², each R¹² is independently selected from the range of substituents specified. The same applies to compounds of the invention comprising more than one R¹² substituent; each R¹² is selected independently of any other R¹² substituent present in the compound. As previously indicated, where R¹² is halo, particularly fluoro, any number of hydrogens may in principle be replaced. Also, when two R¹² are attached to the same carbon atom, they may together form oxo.

Of mention are compounds in which at least two of the following provisos apply:
(i) R¹ is selected from C₁₋₆ alkyl, C₂₋₆ alkenyl and C₂₋₆ alkynyl, any of which is optionally substituted with 1, 2, 3, 4 or 5 substituents selected from R¹², carbocyclyl and heterocyclyl; or R¹ is carbocyclyl or heterocyclyl, either of which is optionally substituted with 1, 2, 3, 4 or 5 R¹²;
(ii) R² is —W-hydrocarbyl or —W-heterocyclyl, either of which is optionally substituted with 1, 2, 3, 4 or 5 R¹², wherein W is a linker; and
(iii) R⁴ is cyano;
and pharmaceutically acceptable salts and prodrugs thereof.

Of particular mention are compounds in which at least two of said provisos apply and in which R⁵ is other than homopiperazinyl optionally substituted with 1, 2, 3, 4 or 5 R¹².

In compounds in which proviso (i) applies, R¹ is selected from C₁₋₆ alkyl, C₂₋₆ alkenyl and C₂₋₆ alkynyl, any of which is optionally substituted with 1, 2, 3, 4 or 5 substituents selected from R¹², carbocyclyl and heterocyclyl; or R¹ is carbocyclyl or heterocyclyl, either of which is optionally substituted with 1, 2, 3, 4 or 5 R¹².

In one embodiment, R¹ is C₁₋₆ alkyl, for example C₁, C₂, C₃ or C₄ alkyl (e.g. methyl, ethyl, propyl, isopropyl, n-butyl, sec-butyl or tert-butyl), any of which is optionally substituted with 1, 2, 3, 4 or 5 R¹²; wherein the or each R¹² is, for example, C₁₋₆ alkoxy, hydroxy or halogen (e.g. chlorine or fluorine). Alkoxy may be unsubstituted or substituted, for example by 1, 2, 3, 4 or 5 halogens, e.g. selected from F and Cl. Substituted and unsubstituted alkoxyalkyl may be mentioned as R¹ groups. Exemplary R¹ groups include linear alkyl and linear alkoxyalkyl, for example in either case having chain length of up to 6 atoms, e.g. straight chain alkoxyalkyl in which the total number of oxygen and carbon atoms is 3, 4 or 5. In a particular embodiment, R¹ is 2-methoxyethyl.

In another embodiment, R¹ is C₂₋₆ alkenyl, for example C₂, C₃, C₄, C₅ or C₆ alkenyl (e.g. ethenyl, 2-propenyl, 1-butenyl, 2-butenyl, 3-butenyl, 3-methyl-but-2-enyl, 1-pentenyl, 2-pentenyl, 3-pentenyl, 1-hexenyl, 2-hexenyl or 3-hexenyl), any of which is optionally substituted with 1, 2, 3, 4 or 5 R¹², wherein the or each R¹² is, for example, hydroxy or halogen (e.g. chlorine or fluorine). In a particular embodiment, R¹ is 3-methyl-buten-2-yl.

In further embodiments, R¹ is C₂₋₆ alkynyl, for example C₂, C₃, C₄, C₅ or C₆ alkynyl (e.g. ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl or 3-hexynyl), any of which is optionally substituted with 1, 2, 3, 4 or 5 R¹², wherein the or each R¹² is, for example, hydroxy or halogen (e.g. chlorine or fluorine). In a particular embodiment, R¹ is but-2-ynyl.

In further embodiments, R¹ is —(CH₂)ₙ-aryl, wherein n is 0, 1, 2 or 3, and aryl is phenyl, naphthyl or fluorenyl. When R¹ is aryl (e.g. phenyl), it may be substituted at any of the 2-, 3- and 4-positions with a substituent selected from halogen (e.g. fluorine or chlorine), hydroxy, cyano, methoxy, ethoxy, methyl, trifluoromethyl and ethyl.

In further embodiments, R¹ is benzyl.

In further embodiments, R¹ is —(CH₂)ₙ-cycloalkyl, wherein n is 0, 1 or 2, and cycloalkyl is cyclopropyl, cyclobutyl, cyclopentyl or cyclohexyl, When R¹ is cycloalkyl (e.g. cyclopropyl), it may be substituted at either of the 2- and 3-positions with a substituent selected from halogen (e.g. fluorine or chlorine), hydroxy, cyano, methoxy, ethoxy, methyl, trifluoromethyl and ethyl. In a particular embodiment, R¹ is cyclopropylmethyl, cyclopropylethyl, or cyclobutylmethyl. Of particular mention are compounds in which R¹ is cyclopropylmethyl.

In further embodiments, R¹ is —(CH₂)ₙ-heterocycloalkyl, wherein n is 0, 1 or 2, and heterocycloalkyl is azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl. In a particular embodiment, R¹ is tetrahydrofuranylmethyl, for example tetrahydrofuran-2-ylmethyl.

In further embodiments, R¹ is —(CH₂)ₙ-heteroaryl, wherein n is 0, 1 or 2 and heteroaryl is pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl or pteridinyl. Of particular mention are compounds in which the heteroaryl portion is unsubstituted. In a particular embodiment, R¹ is thiazolylmethyl, furanylmethyl or oxazolylmethyl.

In a further class of embodiments, R¹ is selected from (i) benzyl-type and/or (ii) alkenyl/alkynyl-type groups.

Particularly when proviso (i) applies, R¹ may be, for example, a group of formula (vi), (vii) or (viii):

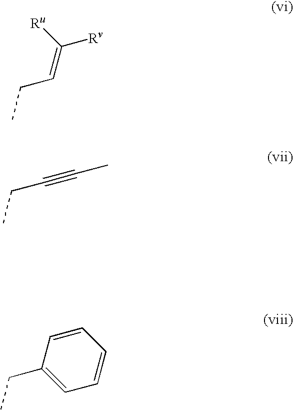

wherein R^{u} and R^{v} are each independently selected from hydrogen and R¹², or taken together with the carbon atom to which they are attached form cyclopropyl.

With regard to formula (vi), R^{u} and R^{v} may be, for example, independently each selected from hydrogen, halogen (e.g. fluorine, chlorine or bromine), hydroxy, cyano, C₁₋₆ alkyl optionally substituted with 1, 2, 3, 4 or 5 substituents selected from hydrogen, halogen (e.g. fluorine, chlorine or bromine), hydroxy and cyano. In certain compounds, R^{u} and R^{v} are independently each selected from hydrogen, fluorine, chlorine and methyl. In particular compounds, R^{u} and R^{v} are the same and are each fluorine, chlorine or methyl. In further compounds, one of R^{u} and R^{v} is methyl, and the other is selected from fluorine, chlorine and methyl. Exemplary R¹ groups include 3-methyl-buten-2-yl, 3,3-difluoroprop-2-en-1-yl, 3,3-dichloroprop-2-en-1-yl, 3-fluoroprop-2-en-1-yl and 3-chloroprop-2-en-1-yl.

In compounds in which proviso (ii) applies, R² is —W-hydrocarbyl or —W-heterocyclyl, either of which is optionally substituted with 1, 2, 3, 4 or 5 R¹², wherein W is a linker as defined in formula (I).

In one embodiment, R² is —W-hydrocarbyl, wherein W is a linker and more particularly is selected from —(CH₂)ₙ—, —(CH₂)ₙ—O—(CH₂)ₖ—, —(CH₂)ₙ—C(O)—(CH₂)ₖ—, —(CH₂)ₙ—C(O)O—, —(CH₂)ₙ—OC(O)—, —(CH₂)ₙ—C(O)NR^{a}—, —(CH₂)ₙ—NR^{a}—, —(CH₂)ₙ—S(O)ₘ—NR^{a}(CH₂)ₖ, —(CH₂)ₙ—NR^{a}C(O)—, —(CH₂)ₙ—NR^{a}C(O)O—, —(CH₂)ₙ—NR^{a}C(O)—NR^{a}—(CH₂)ₖ and —(CH₂)ₙ—S(O)ₘ—, wherein k and n are independently each 0, 1, 2, 3, 4, 5 or 6; and hydrocarbyl is, for example, C₁, C₂, C₃ or C₄ alkyl (e.g. methyl, ethyl, propyl, isopropyl, n-butyl, sec-butyl or tert-butyl), cycloalkyl or aryl, in particular methyl, ethyl, cyclohexyl, phenyl or naphthyl, any of which is optionally substituted with 1, 2, 3, 4 or 5 R¹².

In a further embodiment, R² is C₂₋₆ alkyl, for example C₂, C₃ or C₄ alkyl (e.g. ethyl, propyl, isopropyl, n-butyl, sec-butyl or tert-butyl), any of which is optionally substituted with 1, 2, 3, 4 or 5 R¹²; wherein the or each R¹² is, for example, C₁₋₆ alkoxy, hydroxy or halogen (e.g. chlorine or fluorine). Alkoxy may be unsubstituted or substituted, for example by 1, 2, 3, 4 or 5 halogens, e.g. selected from F and Cl. Substituted and unsubstituted alkoxyalkyl having 2, 3, 4 or 5 carbon atoms may be mentioned as R² groups. Exemplary R² groups include linear alkyl and linear alkoxyalkyl, for example in either case having a chain length of up to 6 atoms, e.g. straight chain alkoxyalkyl having 2, 3 or 4 carbon atoms.

In a further embodiment, R² is C₂₋₆ alkenyl, for example C₂, C₃, C₄, C₅ or C₆ alkenyl (e.g. ethenyl, 2-propenyl, 1-butenyl, 2-butenyl, 3-butenyl, 3-methyl-but-2-enyl, 1-pentenyl, 2-pentenyl, 3-pentenyl, 1-hexenyl, 2-hexenyl or 3-hexenyl), any of which is optionally substituted with 1, 2, 3, 4 or 5 R¹², wherein the or each R¹² is, for example, C₁₋₆ alkoxy, hydroxy or halogen (e.g. chlorine or fluorine). In a particular embodiment, R² is 3-methyl-buten-2-yl.

In a further embodiment, R² is C₂₋₆ alkynyl, for example C₂, C₃, C₄, C₅ or C₆ alkynyl (e.g. ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl or 3-hexynyl), any of which is optionally substituted with 1, 2, 3, 4 or 5 R¹², wherein the or each R¹² is, for example, C₁₋₆ alkoxy, hydroxy or halogen (e.g. chlorine or fluorine). In a particular embodiment, R² is but-2-ynyl.

In a further embodiment, R² is —W-heterocyclyl, wherein W is a linker and more particularly is selected from —(CH₂)ₙ—, —(CH₂)ₙ—O—(CH₂)ₖ—, —(CH₂)ₙ—C(O)—(CH₂)ₖ—, —(CH₂)ₙ—C(O)O—, —(CH₂)ₙ—OC(O)—, —(CH₂)ₙ—C(O)NR^{a}—, —(CH₂)ₙ—NR^{a}—, —(CH₂)ₙ—S(O)ₘ—NR^{a}(CH₂)ₖ, —(CH₂)ₙ—NR^{a}C(O)—, —(CH₂)ₙ—NR^{a}C(O)O—, —(CH₂)ₙ—NR^{a}C(O)—NR^{a}—(CH₂)ₖ and —(CH₂)ₙ—S(O)ₘ—, wherein k and n are independently each 0, 1, 2, 3, 4, 5 or 6 and R^{a} is selected from hydrogen, hydroxy, hydrocarbyl optionally substituted with 1, 2, 3, 4 or 5 R¹⁰; and heterocyclyl optionally substituted with 1, 2, 3, 4 or 5 R¹⁰; and heterocyclyl is, for example, heterocycloalkyl or heteroaryl, in particular piperidin-1-yl, thiophen-1-yl, thiophen-2-yl, benzo[b]thiophenyl, pyridin-1-yl, pyridin-2-yl, pyridin-3-yl, pyrazin-2-yl or quinolin-4-yl, any of which is optionally substituted with 1, 2, 3, 4 or 5 R¹².

Typically, W is —(CH₂)ₙ—, e.g. —CH₂—, or is —(CH₂)ₙ—C(O)—(CH₂)ₘ—, e.g. —CH₂—C(O)—.

In a further embodiment, R² is —CH₂C(O)-hydrocarbyl, —CH₂C(O)O-hydrocarbyl, —CH₂C(O)-heterocyclyl or —CH₂-heterocyclyl; wherein hydrocarbyl is in particular C₁, C₂, C₃ or C₄ alkyl (e.g. methyl, ethyl, propyl, isopropyl, n-butyl, sec-butyl or tert-butyl), cycloalkyl (e.g. cyclohexyl) or aryl (e.g. phenyl or naphthyl); and heterocyclyl is in particular heterocycloalkyl (e.g. piperidin-1-yl) or heteroaryl (e.g. thiophen-1-yl, thiophen-2-yl, benzo[b]thiophenyl, pyridin-1-yl, pyridin-2-yl, pyridin-3-yl, pyrazin-2-yl or quinolin-4-yl); and wherein the group is optionally substituted with 1, 2, 3, 4 or 5 R¹².

In a further embodiment, R² is —(CH₂)ₙ—C(O)-aryl, wherein n is 0, 1 or 2 (particularly 1), and aryl is phenyl or naphthyl, either of which is optionally substituted with 1, 2 or 3 R¹². When aryl is phenyl, it may be unsubstituted or substituted, for example at any of the 2-, 3- and 4-positions with a substituent selected from, for example, halogen (e.g. fluorine or chlorine), hydroxy, cyano, methoxy, ethoxy, trifluoromethyl, methyl and ethyl. In a particular embodiment, R² is 2-oxo-2-phenyl-ethyl or 2-oxo-2-(3-methoxyphenyl)-ethyl.

In a further embodiment, R² is —(CH₂)ₙ-heteroaryl, wherein n is 1 or 2 (particularly 1), and heteroaryl is for example a mono- or bicyclic ring containing at least one heteroatom, for example containing one or more nitrogens. Exemplary heteroaryl groups are 6-membered rings and heteroaryl analogues of naphthyl, i.e. groups corresponding to naphthyl in which at least one carbon has been replaced by a heteroatom, e.g. nitrogen; quinolinyl may be mentioned. Particular heteroaryl moieties are thiophen-1-yl, thiophen-2-yl, benzo[b]thiophenyl, isoquinolin-1-yl, phthalazin-6-yl, pyridin-1-yl, pyridin-2-yl, pyridin-3-yl, pyrazin-2-yl, quinazolin-2-yl quinoxalin-6-yl or quinolin-4-yl, any of which is optionally substituted with 1, 2, 3, 4 or 5 R¹², wherein the or each R¹² is in particular selected from cyano, trifluoromethyl, hydroxy, halogen (e.g. chlorine or fluorine); C₁, C₂, C₃ or C₄ alkyl (e.g. methyl, ethyl, propyl, isopropyl, n-butyl, sec-butyl or tert-butyl) optionally substituted with 1, 2 or 3 hydroxy or with 1, 2, 3 or more halogen (e.g. chlorine or fluorine); and C₁, C₂, C₃ or C₄ alkoxy (e.g. methoxy, ethoxy, propoxy, isopropoxy, butoxy, tert-butoxy), optionally substituted with 1, 2, 3 or more halogen (e.g. fluorine or chlorine) atoms. In a particular embodiment, R² is isoquinolin-1-ylmethyl.

Often, R² is 2-oxo-2-phenyl-ethyl, isoquinolin-1-ylmethyl or 2-oxo-2-(3-methoxyphenyl)-ethyl.

Particularly when proviso (ii) applies, R² may be, for example, a group of formula (ix):

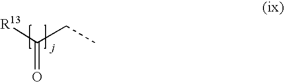

wherein
R¹³ is hydrocarbyl or heterocyclyl, either of which is optionally substituted with 1, 2, 3, 4 or 5 R¹²; and
j is 0 or 1.

In one embodiment, R¹³ is carbocyclyl or heterocyclyl, either of which is optionally substituted with 1, 2, 3, 4 or 5 R¹².

In another embodiment, R¹³ is aryl or heteroaryl, either of which is optionally substituted with 1, 2, 3, 4 or 5 R¹². Aryl and heteroaryl may have, for example, from 6 to 13 ring-members, e.g. from 6 to 12 ring members. Aryl and heteroaryl are often mono- or bi-cyclic, for example a 6-membered ring or a bicyclic ring comprising two interfused 6-membered rings. Structures containing, for example, 5-membered rings as well as or in addition to 6-membered rings are not excluded.

In further embodiments, R¹³ is aryl, in particular phenyl, naphthyl (for example naphth-1-yl) or fluorenyl, any of which is optionally substituted with 1, 2, 3, 4 or 5 R¹², e.g. with a single R¹² In one sub-class of compounds, aryl is phenyl which is unsubstituted or is substituted at any of the 2-, 3- and 4-positions (e.g. substituted solely at two or, more often, one of these positions, the 3-position in any event being exemplary); exemplary substituents in the case of said sub-class of compounds (and otherwise) are selected from halogen (e.g. fluorine or chlorine), hydroxy, cyano, methoxy, trifluoromethoxy, ethoxy, methyl, trifluoromethyl and ethyl, of which methoxy may be mentioned in particular.

In further embodiments, R¹³ is heteroaryl, for example 6-membered rings and quinolinyl or another heteroaryl analogue of naphthyl. In particular, R¹³ may be thiophen-1-yl, thiophen-2-yl, benzo[b]thiophenyl, pyridin-1-yl, pyridin-2-yl, pyridin-3-yl, pyrazin-2-yl or quinolinyl, particularly quinolin-4-yl, any of which is optionally substituted with 1, 2, 3, 4 or 5 R¹², e.g. with a single R¹². Exemplary substituents are selected from halogen (e.g. fluorine or chlorine), hydroxy, cyano, methoxy, trifluoromethoxy, ethoxy, methyl, trifluoromethyl and ethyl, for example halogen.

In one class of embodiments, R¹³ is selected from (i) phenyl or substituted phenyl (e.g. 3-substituted phenyl such as 3-methoxyphenyl, for example) and/or (ii) substituted or unsubstituted quinolinyl, for example 4-quinolinyl. Also to be mentioned are naphthyl and its heteroaryl analogues, i.e. groups corresponding to naphthyl in which at least one carbon has been replaced by a heteroatom, e.g. nitrogen; these groups may be substituted or unsubstituted.

In certain embodiments, j is 0; in other embodiments j is 1.

In one embodiment, provisos (i) and (ii) apply. Of particular mention are compounds of this type in which R¹ is a group of formula (vi), (vii) or (viii); and R² is a group of formula (ix).

In another embodiment, provisos (i) and (iii) apply. Of particular mention are compounds of this type in which R¹ is a group of formula (vi), (vii) or (viii).

In a further embodiment, provisos (ii) and (iii) apply. Of particular mention are compounds of this type in which R² is a group of formula (ix).

In a further embodiment, provisos (i), (ii) and (iii) apply. Of particular mention are compounds of this type in which R¹ is a group of formula (vi), (vii) or (viii); and R² is a group of formula (ix).

In one embodiment, the compound is of the formula (X), (XI) or (XII):

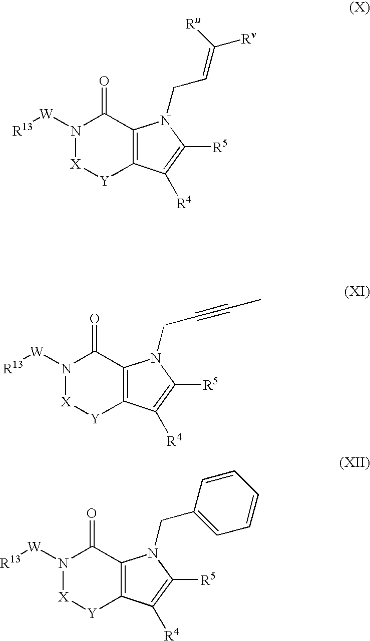

wherein
R^{u} and R^{v} are each independently selected from hydrogen and R¹², or taken together with the carbon atom to which they are attached form cyclopropyl;
R¹³ is hydrocarbyl or heterocyclyl, either of which is optionally substituted with 1, 2, 3, 4 or 5 R¹²; and
W is a linker;
   or, in each case, a pharmaceutically acceptable salt or prodrug thereof.

In a further embodiment, the compound is of the formula (XIII), (XIV) or (XV):

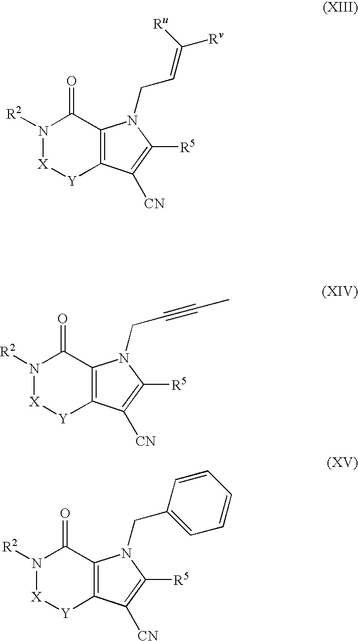

or, in each case, a pharmaceutically acceptable salt or prodrug thereof.

In a further embodiment, the compound is of the formula (XVI), (XVII) or (XVIII):

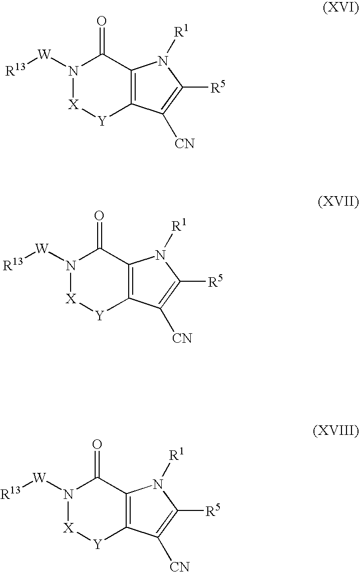

wherein
R¹³ is hydrocarbyl or heterocyclyl, either of which is optionally substituted with 1, 2, 3, 4 or 5 R¹²; and
W is a linker;
   or, in each case, a pharmaceutically acceptable salt or prodrug thereof.

Of further mention are compounds subject to at least two said provisos in which R⁵ is a group of formula (iii), (iv) or (v):

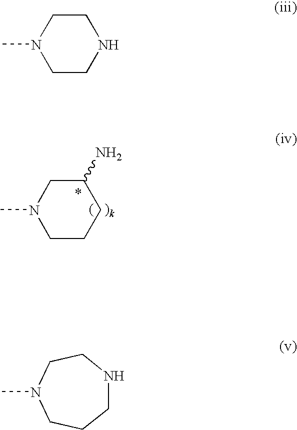

and pharmaceutically acceptable salts and prodrugs thereof.

Of particular mention are compounds of any of formulae (X) to (XVIII) or pharmaceutically acceptable salts or prodrugs thereof in which R⁵ is a group of formula (iii), (iv) or (v).

In one embodiment, the compound is of the formula (XIX), (XX) or (XXI):

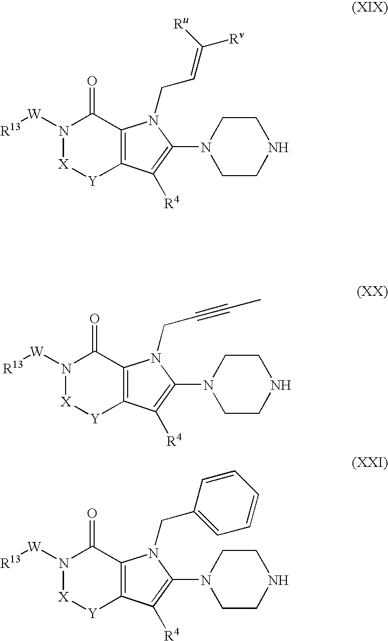

wherein
R^{u} and R^{v} are each independently selected from hydrogen and R¹², or taken together with the carbon atom to which they are attached form cyclopropyl;
R¹³ is hydrocarbyl or heterocyclyl, either of which is optionally substituted with 1, 2, 3, 4 or 5 R¹²; and
W is a linker;
   or, in each case, a pharmaceutically acceptable salt or prodrug thereof.

In another embodiment, the compound is of the formula (XXII), (XXIII) or (XXIV):

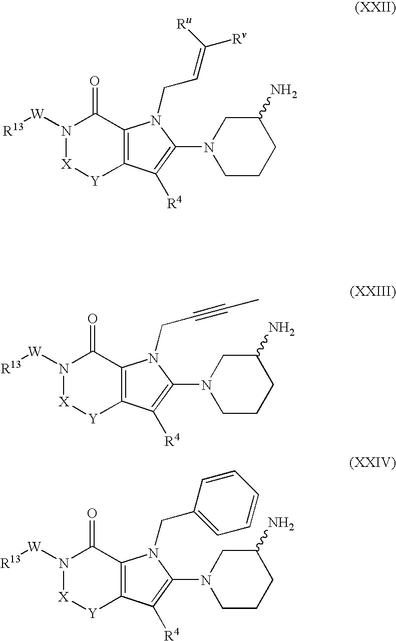

wherein
R^{u} and R^{v} are each independently selected from hydrogen and R¹², or taken together with the carbon atom to which they are attached form cyclopropyl;
R¹³ is hydrocarbyl or heterocyclyl, either of which is optionally substituted with 1, 2, 3, 4 or 5 R¹²; and
W is a linker;
   or, in each case, a pharmaceutically acceptable salt or prodrug thereof.

In a further embodiment, the compound is of the formula (XXV), (XXVI) or (XXVII):

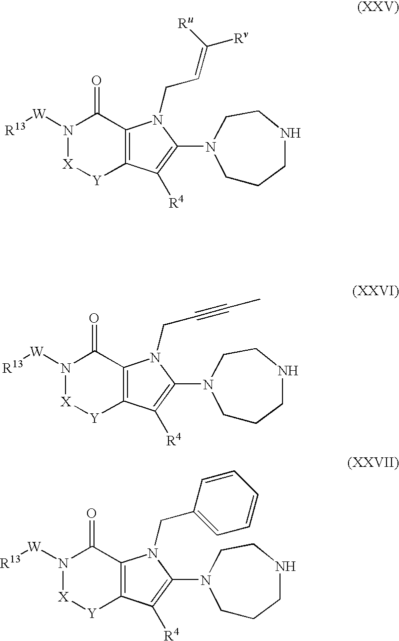

wherein
R^{u} and R^{v} are each independently selected from hydrogen and R¹², or taken together with the carbon atom to which they are attached form cyclopropyl;
R¹³ is hydrocarbyl or heterocyclyl, either of which is optionally substituted with 1, 2, 3, 4 or 5 R¹²; and
W is a linker;
   or, in each case, a pharmaceutically acceptable salt or prodrug thereof.

In a further embodiment, the compound is of the formula (XXVIII), (XXIX) or (XXX):

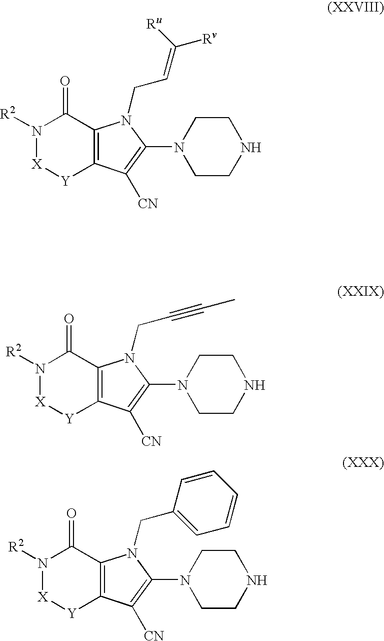

or, in each case, a pharmaceutically acceptable salt or prodrug thereof.

In a further embodiment, the compound is of the formula (XXXI), (XXXII) or (XXXIII):

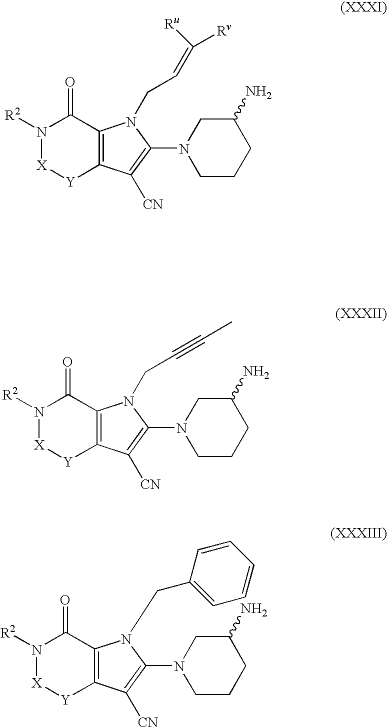

wherein R^{u} and R^{v} are each independently selected from hydrogen and R¹², or taken together with the carbon atom to which they are attached form cyclopropyl;
   or, in each case, a pharmaceutically acceptable salt or prodrug thereof.

In a further embodiment, the compound is of the formula (XXXIV), (XXXV) or (XXXVI):

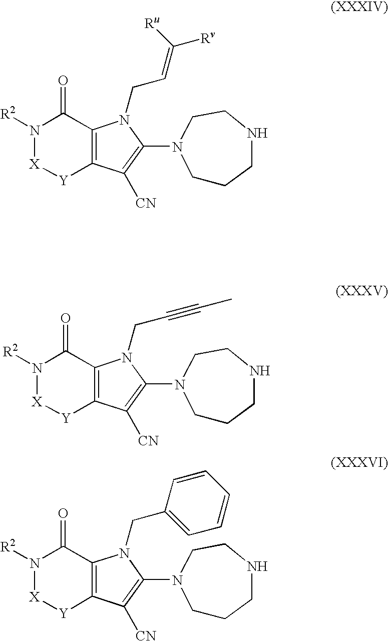

R^{u} and R^{v} are each independently selected from hydrogen and R¹², or taken together with the carbon atom to which they are attached form cyclopropyl;
   or, in each case, a pharmaceutically acceptable salt or prodrug thereof.

Also of mention are compounds subject to two or more of said provisos in which X is —CH═ and Y is ═N—.

Also of mention are compounds subject to two or more of said provisos in which X is —C(O)— and Y is —N(R³)—. In this case, Y is typically hydrogen or methyl.

Other embodiments of the invention include compounds of formula (XXXVII), (XXXVIII) and (XXXIX):

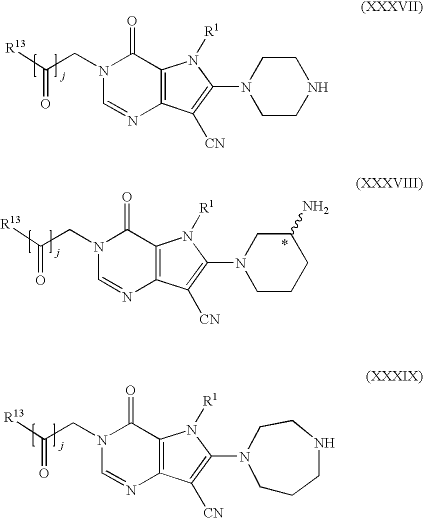

wherein
R¹³ is aryl or heteroaryl, either of which is optionally substituted with 1, 2, 3, 4 or 5 R¹²; and
j is 0 or 1.

Examples of compounds of the invention include the following compounds. It will of course be appreciated that, where appropriate, each compound may be in the form of the free compound, an acid or base addition salt, or a prodrug. Thus, for example, where a particular salt form is mentioned, it will be appreciated that the compound in question may exist in another form, for example in the form of the free compound or in the form of another salt.

### A1

5-But-2-ynyl-4-oxo-3-(2-oxo-2-phenyl-ethyl)-6-piperazin-1-yl-4,5-dihydro-3H-pyrrolo[3,2-d]pyrimidine-7-carbonitrile;

### A2

5-But-2-ynyl-4-oxo-6-piperazin-1-yl-3-quinolin-4-ylmethyl-4,5-dihydro-3H-pyrrolo[3,2-d]pyrimidine-7-carbonitrile;

### B1

6-((R)-3-Amino-piperidin-1-yl)-5-but-2-ynyl-4-oxo-3-(2-oxo-2-phenyl-ethyl)-4,5-dihydro-3H-pyrrolo[3,2-d]pyrimidine-7-carbonitrile;

### C1

5-But-2-ynyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C2

3-[2-(3-Methoxy-phenyl)-2-oxo-ethyl]-5-(3-methyl-but-2-enyl)-4-oxo-6-piperazin-1-yl-4,5-dihydro-3H-pyrrolo[3,2-d]pyrimidine-7-carbonitrile;

### C3

5-(2-Chloro-benzyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C4

5-(2-Chloro-5-fluoro-benzyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C5

5-(2-Methoxy-ethyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C6

5-Benzyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C7

3-[2-(3-Methoxy-phenyl)-2-oxo-ethyl]-5-methyl-4-oxo-6-piperazin-1-yl-4,5-dihydro-3H-pyrrolo[3,2-d]pyrimidine-7-carbonitrile;

### C8

3-[2-(3-Methoxy-phenyl)-2-oxo-ethyl]-5-(3-methyl-butyl)-4-oxo-6-piperazin-1-yl-4,5-dihydro-3H-pyrrolo[3,2-d]pyrimidine-7-carbonitrile;

### C9

5-Cyclopropylmethyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C10

5-Cyclobutylmethyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C11

3-[2-(3-Methoxy-phenyl)-2-oxo-ethyl]-4-oxo-6-piperazin-1-yl-5-(tetrahydro-furan-2-ylmethyl)-4,5-dihydro-3H-pyrrolo[3,2-d]pyrimidine-7-carbonitrile;

### C12

6-[1,4]Diazepan-1-yl-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### C13 (1S,2S)

3-[2-(3-Methoxy-phenyl)-2-oxo-ethyl]-5-((1S,2S-2-methyl-cyclopropyl methyl)-4-oxo-6-piperazin-1-yl-4,5-dihydro-3H-pyrrolo[3,2-d]pyrimidine-7-carbonitrile;

### C13 (1R,2R)

3-[2-(3-methoxy-phenyl)-2-oxo-ethyl]-5-((1R,2R-2-methyl-cyclopropylmethyl)-4-oxo-6-piperazin-1-yl-4,5-dihydro-3H-pyrrolo[3,2-d]pyrimidine-7-carbonitrile;

### C14

3-[2-(3-Methoxy-phenyl)-2-oxo-ethyl]-4-oxo-6-piperazin-1-yl-5-thiazol-4-ylmethyl-4,5-dihydro-3H-pyrrolo[3,2-d]pyrimidine-7-carbonitrile;

### C15

5-(2-Cyclopropyl-ethyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C16

5-Furan-3-ylmethyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### C17

6-(2-Amino-ethylamino)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### C18

5-But-2-yn-1-yl-3-(isoquinolin-1-ylmethyl)-4-oxo-6-piperazin-1-yl-4,5-dihydro-3H-pyrrolo[3,2-d]pyrimidine-7-carbonitrile;

### C19

5-But-2-yn-1-yl-6-(1,4-diazepan-1-yl)-3-(isoquinolin-1-ylmethyl)-4-oxo-4,5-dihydro-3H-pyrrolo[3,2-d]pyrimidine-7-carbonitrile dihydrochloride;

### C20

5-But-2-yn-1-yl-6-(1,4-diazepan-1-yl)-3-(2-(3-(methyloxy)phenyl)-2-oxoethyl)-4-oxo-4,5-dihydro-3H-pyrrolo[3,2-d]pyrimidine-7-carbonitrile hydrochloride;

### C21

5-But-2-yn-1-yl-6-(1,4-diazepan-1-yl)-3-methyl-4-oxo-4,5-dihydro-3H-pyrrolo[3,2-d]pyrimidine-7-carbonitrile hydrochloride;

### C22

5-But-2-yn-1-yl-3-methyl-4-oxo-6-piperazin-1-yl-4,5-dihydro-3H-pyrrolo[3,2-d]pyrimidine-7-carbonitrile hydrochloride;

### C23

6-(1,4-Diazepan-1-yl)-3-(isoquinolin-1-ylmethyl)-5-(3-methylbut-2-en-1-yl)-4-oxo-4,5-dihydro-3H-pyrrolo[3,2-d]pyrimidine-7-carbonitrile;

### C24

3-(Isoquinolin-1-ylmethyl)-5-(3-methylbut-2-en-1-yl)-4-oxo-6-piperazin-1-yl-4,5-dihydro-3H-pyrrolo[3,2-d]pyrimidine-7-carbonitrile;

### C25

6-(1,4-Diazepan-1-yl)-5-(3,3-dichloroprop-2-en-1-yl)-3-(isoquinolin-1-ylmethyl)-4-oxo-4,5-dihydro-3H-pyrrolo[3,2-d]pyrimidine-7-carbonitrile;

### C26

3-((3-Cyanopyridin-2-yl)methyl)-6-(1,4-diazepan-1-yl)-5-(3,3-dichloroprop-2-en-1-yl)-4-oxo-4,5-dihydro-3H-pyrrolo[3,2-d]pyrimidine-7-carbonitrile;

### D1

6-((R)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### D2

6-((R)-3-Amino-piperidin-1-yl)-5-but-2-ynyl-3-[2-(3-methoxy-phenyl)-2-oxo-ethyl]-4-oxo-4,5-dihydro-3H-pyrrolo[3,2-d]pyrimidine-7-carbonitrile;

### D3

6-((R)-3-Amino-piperidin-1-yl)-3-[2-(3-methoxy-phenyl)-2-oxo-ethyl]-5-methyl-4-oxo-4,5-dihydro-3H-pyrrolo[3,2-d]pyrimidine-7-carbonitrile;

### D4

6-((R)-3-Amino-piperidin-1-yl)-3-[2-(3-methoxy-phenyl)-2-oxo-ethyl]-5-(3-methyl-butyl)-4-oxo-4,5-dihydro-3H-pyrrolo[3,2-d]pyrimidine-7-carbonitrile;

### D5

6-((R)-3-Amino-piperidin-1-yl)-5-cyclopropylmethyl-3-[2-(3-methoxy-phenyl)-2-oxo-ethyl]-4-oxo-4,5-dihydro-3H-pyrrolo[3,2-d]pyrimidine-7-carbonitrile;

### D6

6-((R)-3-Amino-piperidin-1-yl)-5-cyclobutylmethyl-3-[2-(3-methoxy-phenyl)-2-oxo-ethyl]-4-oxo-4,5-dihydro-3H-pyrrolo[3,2-d]pyrimidine-7-carbonitrile;

### D7

6-(3-Amino-pyrrolidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### D8

6-((R)-3-Amino-piperidin-1-yl)-3-[2-(3-methoxy-phenyl)-2-oxo-ethyl]-4-oxo-5-thiazol-4-ylmethyl-4,5-dihydro-3H-pyrrolo[3,2-d]pyrimidine-7-carbonitrile;

### D9

6-((S)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### D10 (1S,2S)

6-((R)-3-Amino-piperidin-1-yl)-3-[2-(3-methoxy-phenyl)-2-oxo-ethyl]-5-((1S,2S)-2-methyl-cyclopropylmethyl)-4-oxo-4,5-dihydro-3H-pyrrolo[3,2-d]pyrimidine-7-carbonitrile;

### D10 (1R,2R)

6-((R)-3-Amino-piperidin-1-yl)-3-[2-(3-methoxy-phenyl)-2-oxo-ethyl]-5-((1R,2R)-2-methyl-cyclopropylmethyl)-4-oxo-4,5-dihydro-3H-pyrrolo[3,2-d]pyrimidine-7-carbonitrile;

### D11

6-((R)-3-Amino-piperidin-1-yl)-3,5-bis-(3-methyl-but-2-enyl)-4-oxo-4,5-dihydro-3H-pyrrolo[3,2-d]pyrimidine-7-carbonitrile;

### D12

6-((R)-3-Amino-piperidin-1-yl)-3-[2-(3-methoxy-phenyl)-2-oxo-ethyl]-5-(2-methyl-thiazol-4-ylmethyl)-4-oxo-4,5-dihydro-3H-pyrrolo[3,2-d]pyrimidine-7-carbonitrile;

### D13

6-((R)-3-Amino-piperidin-1-yl)-5-(2-cyclopropyl-ethyl)-3-[2-(3-methoxy-phenyl)-2-oxo-ethyl]-4-oxo-4,5-dihydro-3H-pyrrolo[3,2-d]pyrimidine-7-carbonitrile;

### D14

6-((R)-3-Amino-piperidin-1-yl)-5-isoxazol-5-ylmethyl-3-[2-(3-methoxy-phenyl)-2-oxo-ethyl]-4-oxo-4,5-dihydro-3H-pyrrolo[3,2-d]pyrimidine-7-carbonitrile;

### D15

6-((S)-3-Amino-piperidin-1-yl)-5-benzyl-3-[2-(3-methoxy-phenyl)-2-oxo-ethyl]-4-oxo-4,5-dihydro-3H-pyrrolo[3,2-d]pyrimidine-7-carbonitrile;

### D16

6-((R)-3-Amino-piperidin-1-yl)-5-furan-3-ylmethyl-3-[2-(3-methoxy-phenyl)-2-oxo-ethyl]-4-oxo-4,5-dihydro-3H-pyrrolo[3,2-d]pyrimidine-7-carbonitrile;

### D17

6-((R)-3-Amino-piperidin-1-yl)-5-benzyl-3-[2-(3-methoxy-phenyl)-2-oxo-ethyl]-4-oxo-4,5-dihydro-3H-pyrrolo[3,2-d]pyrimidine-7-carbonitrile;

### D18

6-((R)-3-Amino-piperidin-1-yl)-3-[2-(3-methoxy-phenyl)-2-oxo-ethyl]-4-oxo-5-thiophen-2-ylmethyl-4,5-dihydro-3H-pyrrolo[3,2-d]pyrimidine-7-carbonitrile;

### D19

6-((S)-3-Amino-piperidin-1-yl)-3-[2-(3-methoxy-phenyl)-2-oxo-ethyl]-4-oxo-5-thiophen-2-ylmethyl-4,5-dihydro-3H-pyrrolo[3,2-d]pyrimidine-7-carbonitrile;

### D20

6-((R)-3-Amino-piperidin-1-yl)-3-[2-(3-methoxy-phenyl)-2-oxo-ethyl]-4-oxo-5-thiophen-3-ylmethyl-4,5-dihydro-3H-pyrrolo[3,2-d]pyrimidine-7-carbonitrile;

### D21

6-((S)-3-Amino-piperidin-1-yl)-3-[2-(3-methoxy-phenyl)-2-oxo-ethyl]-4-oxo-5-thiophen-3-ylmethyl-4,5-dihydro-3H-pyrrolo[3,2-d]pyrimidine-7-carbonitrile;

### D22

6-((S)-3-Amino-piperidin-1-yl)-5-(3-methyl-but-2-enyl)-4-oxo-3-quinolin-4-ylmethyl-4,5-dihydro-3H-pyrrolo[3,2-d]pyrimidine-7-carbonitrile;

### D23

6-((R)-3-Amino-piperidin-1-yl)-5-(3-methyl-but-2-enyl)-4-oxo-3-quinolin-4-ylmethyl-4,5-dihydro-3H-pyrrolo[3,2-d]pyrimidine-7-carbonitrile;

### D24

6-(3-Amino-azetidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### D25

6-((S)-3-Amino-piperidin-1-yl)-5-(3-methyl-but-2-enyl)-4-oxo-3-quinolin-6-ylmethyl-4,5-dihydro-3H-pyrrolo[3,2-d]pyrimidine-7-carbonitrile;

### D26

6-((S)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile;

### D27

6-((S)-3-Amino-piperidin-1-yl)-5-butyl-3-[2-(3-methoxy-phenyl)-2-oxo-ethyl]-4-oxo-4,5-dihydro-3H-pyrrolo[3,2-d]pyrimidine-7-carbonitrile;

### D28

6-((R)-3-Amino-piperidin-1-yl)-5-butyl-3-[2-(3-methoxy-phenyl)-2-oxo-ethyl]-4-oxo-4,5-dihydro-3H-pyrrolo[3,2-d]pyrimidine-7-carbonitrile;

### D29

6-((R)-3-Amino-piperidin-1-yl)-3-[2-(3-methoxy-phenyl)-2-oxo-ethyl]-4-oxo-5-(4,4,4-trifluoro-butyl)-4,5-dihydro-3H-pyrrolo[3,2-d]pyrimidine-7-carbonitrile;

### D30

6-((S)-3-Amino-piperidin-1-yl)-5-(3,4-difluoro-benzyl)-3-[2-(3-methoxy-phenyl)-2-oxo-ethyl]-4-oxo-4,5-dihydro-3H-pyrrolo[3,2-d]pyrimidine-7-carbonitrile;

### D31

6-((S)-3-Amino-piperidin-1-yl)-3-[2-(3-methoxy-phenyl)-2-oxo-ethyl]-4-oxo-5-(2,4,5-trifluoro-benzyl)-4,5-dihydro-3H-pyrrolo[3,2-d]pyrimidine-7-carbonitrile;

### D32

6-((R)-3-Amino-piperidin-1-yl)-5-(3,4-difluoro-benzyl)-3-[2-(3-methoxy-phenyl)-2-oxo-ethyl]-4-oxo-4,5-dihydro-3H-pyrrolo[3,2-d]pyrimidine-7-carbonitrile;

### D33

6-((S)-3-Amino-piperidin-1-yl)-3-isoquinolin-1-ylmethyl-5-(3-methyl-but-2-enyl)-4-oxo-4,5-dihydro-3H-pyrrolo[3,2-d]pyrimidine-7-carbonitrile;

### D34

6-((R)-3-Amino-piperidin-1-yl)-3-[2-(3-methoxy-phenyl)-2-oxo-ethyl]-4-oxo-5-(4,4,4-trifluoro-butyl)-4,5-dihydro-3H-pyrrolo[3,2-d]pyrimidine-7-carbonitrile;

### D35

6-((S)-3-Amino-piperidin-1-yl)-5-(3,4-difluoro-benzyl)-3-[2-(3-methoxy-phenyl)-2-oxo-ethyl]-4-oxo-4,5-dihydro-3H-pyrrolo[3,2-d]pyrimidine-7-carbonitrile;

### D36

6-((S)-3-Amino-piperidin-1-yl)-5-(4-fluoro-benzyl)-3-[2-(3-methoxy-phenyl)-2-oxo-ethyl]-4-oxo-4,5-dihydro-3H-pyrrolo[3,2-d]pyrimidine-7-carbonitrile;

### D37

6-((R)-3-Amino-piperidin-1-yl)-5-(4-fluoro-benzyl)-3-[2-(3-methoxy-phenyl)-2-oxo-ethyl]-4-oxo-4,5-dihydro-3H-pyrrolo[3,2-d]pyrimidine-7-carbonitrile;

### D38

6-((S)-3-Amino-piperidin-1-yl)-5-benzyl-4-oxo-3-quinolin-4-ylmethyl-4,5-dihydro-3H-pyrrolo[3,2-d]pyrimidine-7-carbonitrile;

### D39

6-((S)-3-Amino-piperidin-1-yl)-5-benzyl-4-oxo-3-quinolin-4-ylmethyl-4,5-dihydro-3H-pyrrolo[3,2-d]pyrimidine-7-carbonitrile hydrochloride;

### D40

6-((S)-3-Amino-piperidin-1-yl)-5-benzyl-3-[2-(3-methoxy-phenyl)-2-oxo-ethyl]-4-oxo-4,5-dihydro-3H-pyrrolo[3,2-d]pyrimidine-7-carbonitrile hydrochloride;

### D41

6-((S)-3-Aminopiperidin-1-yl)-5-((3-fluorophenyl)methyl)-3-(2-(3-(methyloxy)phenyl)-2-oxoethyl)-4-oxo-4,5-dihydro-3H-pyrrolo[3,2-d]pyrimidine-7-carbonitrile;

### D42

6-((S)-3-Aminopiperidin-1-yl)-3-(2-(3-(methyloxy)phenyl)-2-oxoethyl)-4-oxo-5-(pyridin-3-ylmethyl)-4,5-dihydro-3H-pyrrolo[3,2-d]pyrimidine-7-carbonitrile;

### D43

6-((S)-3-Aminopiperidin-1-yl)-5-but-2-yn-1-yl-3-methyl-4-oxo-4,5-dihydro-3H-pyrrolo[3,2-d]pyrimidine-7-carbonitrile;

### D44

6-((R)-3-Aminopiperidin-1-yl)-5-but-2-yn-1-yl-3-(isoquinolin-1-ylmethyl)-4-oxo-4,5-dihydro-3H-pyrrolo[3,2-d]pyrimidine-7-carbonitrile;

### D45

6-((S)-3-Aminopiperidin-1-yl)-3-methyl-5-(3-methylbut-2-en-1-yl)-4-oxo-4,5-dihydro-3H-pyrrolo[3,2-d]pyrimidine-7-carbonitrile;

### D46

6-((S)-3-Aminopiperidin-1-yl)-5-(3,3-dichloroprop-2-en-1-yl)-3-methyl-4-oxo-4,5-dihydro-3H-pyrrolo[3,2-d]pyrimidine-7-carbonitrile;

### D47

6-((S)-3-Aminopiperidin-1-yl)-3-((3-cyanopyridin-2-yl)methyl)-5-(3,3-dichloroprop-2-en-1-yl)-4-oxo-4,5-dihydro-3H-pyrrolo[3,2-d]pyrimidine-7-carbonitrile;

### D48

6-((S)-3-Aminopiperidin-1-yl)-5-(3,3-dichloroprop-2-en-1-yl)-3-(2-(3-(methyloxy)phenyl)-2-oxoethyl)-4-oxo-4,5-dihydro-3H-pyrrolo[3,2-d]pyrimidine-7-carbonitrile;

### D49

6-((S)-3-Aminopiperidin-1-yl)-5-(3,3-dichloroprop-2-en-1-yl)-3-(isoquinolin-1-ylmethyl)-4-oxo-4,5-dihydro-3H-pyrrolo[3,2-d]pyrimidine-7-carbonitrile;

### D50

6-((S)-3-Aminopiperidin-1-yl)-5-buta-2,3-dien-1-yl-3-(2-(3-(methyloxy)phenyl)-2-oxoethyl)-4-oxo-4,5-dihydro-3H-pyrrolo[3,2-d]pyrimidine-7-carbonitrile;

### D51

6-((S)-3-aminopiperidin-1-yl)-5-buta-2,3-dien-1-yl-3-(isoquinolin-1-ylmethyl)-4-oxo-4,5-dihydro-3H-pyrrolo[3,2-d]pyrimidine-7-carbonitrile;

### D52

6-((S)-3-aminopiperidin-1-yl)-5-((E)-3-chloroprop-2-en-1-yl)-3-(2-(3-(methyloxy)phenyl)-2-oxoethyl)-4-oxo-4,5-dihydro-3H-pyrrolo[3,2-d]pyrimidine-7-carbonitrile; or
6-((S)-3-aminopiperidin-1-yl)-5-((Z)-3-chloroprop-2-en-1-yl)-3-(2-(3-(methyloxy)phenyl)-2-oxoethyl)-4-oxo-4,5-dihydro-3H-pyrrolo[3,2-d]pyrimidine-7-carbonitrile;

### D53

6-((S)-3-aminopiperidin-1-yl)-5-((E)-3-chlorobut-2-en-1-yl)-3-(2-(3-(methyloxy)phenyl)-2-oxoethyl)-4-oxo-4,5-dihydro-3H-pyrrolo[3,2-d]pyrimidine-7-carbonitrile; and/or
6-((S)-3-aminopiperidin-1-yl)-5-((Z)-3-chlorobut-2-en-1-yl)-3-(2-(3-(methyloxy)phenyl)-2-oxoethyl)-4-oxo-4,5-dihydro-3H-pyrrolo[3,2-d]pyrimidine-7-carbonitrile;

### D54

6-((S)-3-aminopiperidin-1-yl)-5-((E)-3-chloroprop-2-en-1-yl)-3-(isoquinolin-1-ylmethyl)-4-oxo-4,5-dihydro-3H-pyrrolo[3,2-d]pyrimidine-7-carbonitrile; and/or
6-((S)-3-aminopiperidin-1-yl)-5-((Z)-3-chloroprop-2-en-1-yl)-3-(isoquinolin-1-ylmethyl)-4-oxo-4,5-dihydro-3H-pyrrolo[3,2-d]pyrimidine-7-carbonitrile;

### D55

6-((S)-3-aminopiperidin-1-yl)-5-((E)-3-chlorobut-2-en-1-yl)-3-(isoquinolin-1-ylmethyl)-4-oxo-4,5-dihydro-3H-pyrrolo[3,2-d]pyrimidine-7-carbonitrile; and/or
6-((S)-3-aminopiperidin-1-yl)-5-((Z)-3-chlorobut-2-en-1-yl)-3-(isoquinolin-1-ylmethyl)-4-oxo-4,5-dihydro-3H-pyrrolo[3,2-d]pyrimidine-7-carbonitrile;

### D56

6-((S)-3-Aminopiperidin-1-yl)-5-but-2-yn-1-yl-3-(2-(3-(methyloxy)phenyl)-2-oxoethyl)-4-oxo-4,5-dihydro-3H-pyrrolo[3,2-d]pyrimidine-7-carbonitrile;

### D57

6-[(3S)-3-Aminopiperidin-1-yl]-5-(3,3-difluoroprop-2-en-1-yl)-3-[2-(3-methoxyphenyl)-2-oxoethyl]-4-oxo-4,5-dihydro-3H-pyrrolo[3,2-d]pyrimidine-7-carbonitrile;

### D58

6-[(3S)-3-Aminopiperidin-1-yl]-5-(2-cyclopropylidene-ethyl)-3-(isoquinolin-1-ylmethyl)-4-oxo-4,5-dihydro-3H-pyrrolo[3,2-d]pyrimidine-7-carbonitrile;

### E1

5-(2-Chloro-phenyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile;

### E2

6-((S)-3-Aminopiperidin-1-yl)-3-(2-(3-(methyloxy)phenyl)-2-oxoethyl)-4-oxo-5-phenyl-4,5-dihydro-3H-pyrrolo[3,2-d]pyrimidine-7-carbonitrile;

### E3

6-((R)-3-Aminopiperidin-1-yl)-3-(2-(3-(methyloxy)phenyl)-2-oxoethyl)-4-oxo-5-phenyl-4,5-dihydro-3H-pyrrolo[3,2-d]pyrimidine-7-carbonitrile;

### E4

6-((S)-3-Aminopiperidin-1-yl)-3-(isoquinolin-1-ylmethyl)-4-oxo-5-phenyl-4,5-dihydro-3H-pyrrolo[3,2-d]pyrimidine-7-carbonitrile hydrochloride;

### E5

6-((S)-3-Aminopiperidin-1-yl)-3-methyl-4-oxo-5-phenyl-4,5-dihydro-3H-pyrrolo[3,2-d]pyrimidine-7-carbonitrile hydrochloride;

### E6

6-((S)-3-Aminopiperidin-1-yl)-5-(4-fluorophenyl)-3-(isoquinolin-1-ylmethyl)-4-oxo-4,5-dihydro-3H-pyrrolo[3,2-d]pyrimidine-7-carbonitrile hydrochloride;

### E7

6-((S)-3-Aminopiperidin-1-yl)-5-(4-fluorophenyl)-3-methyl-4-oxo-4,5-dihydro-3H-pyrrolo[3,2-d]pyrimidine-7-carbonitrile hydrochloride;

### E8

6-((S)-3-Aminopiperidin-1-yl)-5-(3,4-difluorophenyl)-3-(isoquinolin-1-ylmethyl)-4-oxo-4,5-dihydro-3H-pyrrolo[3,2-d]pyrimidine-7-carbonitrile hydrochloride;

### E9

6-((S)-3-Aminopiperidin-1-yl)-5-(3,4-difluorophenyl)-3-methyl-4-oxo-4,5-dihydro-3H-pyrrolo[3,2-d]pyrimidine-7-carbonitrile hydrochloride;

### E10

6-((S)-3-Aminopiperidin-1-yl)-5-(3-fluorophenyl)-3-(isoquinolin-1-ylmethyl)-4-oxo-4,5-dihydro-3H-pyrrolo[3,2-d]pyrimidine-7-carbonitrile hydrochloride;

### E11

6-((S)-3-Aminopiperidin-1-yl)-5-(3-fluorophenyl)-3-methyl-4-oxo-4,5-dihydro-3H-pyrrolo[3,2-d]pyrimidine-7-carbonitrile hydrochloride;

### E12

6-((S)-3-Aminopiperidin-1-yl)-5-(4-chlorophenyl)-3-(isoquinolin-1-ylmethyl)-4-oxo-4,5-dihydro-3H-pyrrolo[3,2-d]pyrimidine-7-carbonitrile hydrochloride;

### E13

6-((S)-3-Aminopiperidin-1-yl)-5-(4-chlorophenyl)-3-methyl-4-oxo-4,5-dihydro-3H-pyrrolo[3,2-d]pyrimidine-7-carbonitrile hydrochloride;

### E14

6-((S)-3-Aminopiperidin-1-yl)-5-(2,4-difluorophenyl)-3-(isoquinolin-1-ylmethyl)-4-oxo-4,5-dihydro-3H-pyrrolo[3,2-d]pyrimidine-7-carbonitrile hydrochloride;

### E15

6-((S)-3-Aminopiperidin-1-yl)-5-(2,4-difluorophenyl)-3-methyl-4-oxo-4,5-dihydro-3H-pyrrolo[3,2-d]pyrimidine-7-carbonitrile hydrochloride;

### E16

6-((S)-3-Aminopiperidin-1-yl)-5-(2-chloro-4-fluorophenyl)-3-(isoquinolin-1-ylmethyl)-4-oxo-4,5-dihydro-3H-pyrrolo[3,2-d]pyrimidine-7-carbonitrile hydrochloride;

### E17

6-((S)-3-Aminopiperidin-1-yl)-3-(2-(3-(methyloxy)phenyl)-2-oxoethyl)-4-oxo-5-pyridin-2-yl-4,5-dihydro-3H-pyrrolo[3,2-d]pyrimidine-7-carbonitrile hydrochloride;

### F1

6-((R)-3-Aminopiperidin-1-yl)-5-benzyl-3-[2-(3-methoxyphenyl)-2-oxoethyl]-1-methyl-2,4-dioxo-2,3,4,5-tetrahydro-1H-pyrrolo[3,2-d]pyrimidine-7-carbonitrile;

### F2

6-((S)-3-Aminopiperidin-1-yl)-5-benzyl-3-[2-(3-methoxyphenyl)-2-oxoethyl]-1-methyl-2,4-dioxo-2,3,4,5-tetrahydro-1H-pyrrolo[3,2-d]pyrimidine-7-carbonitrile;

### F3

6-((S)-3-Aminopiperidin-1-yl)-1,3-dimethyl-2,4-dioxo-5-(phenylmethyl)-2,3,4,5-tetrahydro-1H-pyrrolo[3,2-d]pyrimidine-7-carbonitrile;

### F4

6-((S)-3-Aminopiperidin-1-yl)-3-(isoquinolin-1-ylmethyl)-1-methyl-2,4-dioxo-5-(phenylmethyl)-2,3,4,5-tetrahydro-1H-pyrrolo[3,2-d]pyrimidine-7-carbonitrile;

### F5

6-((S)-3-Aminopiperidin-1-yl)-1-methyl-2,4-dioxo-5-(phenylmethyl)-2,3,4,5-tetrahydro-1H-pyrrolo[3,2-d]pyrimidine-7-carbonitrile hydrochloride;

### F6

1-methyl-2,4-dioxo-5-(phenylmethyl)-6-piperazin-1-yl-2,3,4,5-tetrahydro-1H-pyrrolo[3,2-d]pyrimidine-7-carbonitrile hydrochloride;

### G1

5-But-2-ynyl-3-[2-(3-methoxy-phenyl)-2-oxo-ethyl]-4-oxo-6-piperazin-1-yl-4,5-dihydro-3H-pyrrolo[3,2-d]pyrimidine-7-carboxylic acid methyl ester;

### G2

3-[2-(3-methoxy-phenyl)-2-oxo-ethyl]-5-(3-methyl-but-2-enyl)-4-oxo-6-piperazin-1-yl-4,5-dihydro-3H-pyrrolo[3,2-d]pyrimidine-7-carboxylic acid methyl ester;

### H1

6-((R)-3-amino-piperidin-1-yl)-5-but-2-ynyl-3-[2-(3-methoxy-phenyl)-2-oxo-ethyl]-4-oxo-4,5-dihydro-3H-pyrrolo[3,2-d]pyrimidine-7-carboxylic acid methyl ester;

### H2

6-((R)-3-amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid methyl ester;

### I1

5-But-2-ynyl-3-[2-(3-methoxy-phenyl)-2-oxo-ethyl]-6-piperazin-1-yl-3,5-dihydro-pyrrolo[3,2-d]pyrimidin-4-one;

### J1

6-((R)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid amide;

### J2

6-((R)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid methylamide;

### J3

6-((R)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid dimethylamide;

### J4

6-((S)-3-Aminopiperidin-1-yl)-5-but-2-yn-1-yl-N,N-dimethyl-3-(2-(3-(methyloxy)phenyl)-2-oxoethyl)-4-oxo-4,5-dihydro-3H-pyrrolo[3,2-d]pyrimidine-7-carboxamide;

### K1

6-((R)-3-Amino-piperidin-1-yl)-3-[2-(3-methoxy-phenyl)-2-oxo-ethyl]-5-(3-methyl-but-2-enyl)-3,5-dihydro-pyrrolo[3,2-d]pyrimidin-4-one;

### L1

6-((S)-3-Aminopiperidin-1-yl)-3-(2-(3-(methyloxy)phenyl)-2-oxoethyl)-4-oxo-5-(pyridin-2-ylmethyl)-4,5-dihydro-3H-pyrrolo[3,2-d]pyrimidine-7-carbonitrile dihydrochloride;

### M1

3-(Isoquinolin-1-ylmethyl)-4-oxo-5-(phenylmethyl)-6-piperazin-1-yl-4,5-dihydro-3H-pyrrolo[3,2-d]pyrimidine-7-carbonitrile dihydrochloride;

### M2

6-(1,4-Diazepan-1-yl)-3-(isoquinolin-1-ylmethyl)-4-oxo-5-(phenylmethyl)-4,5-dihydro-3H-pyrrolo[3,2-d]pyrimidine-7-carbonitrile dihydrochloride;

### M3

6-((S)-Aminopiperidin-1-yl)-4-oxo-5-(phenylmethyl)-3-(quinolin-6-ylmethyl)-4,5-dihydro-3H-pyrrolo[3,2-d]pyrimidine-7-carbonitrile;

### M4

6-((S)-3-Aminopiperidin-1-yl)-3-(isoquinolin-1-ylmethyl)-4-oxo-5-(phenylmethyl)-4,5-dihydro-3H-pyrrolo[3,2-d]pyrimidine-7-carbonitrile;

### M5

6-((S)-3-Aminopiperidin-1-yl)-4-oxo-5-(phenylmethyl)-4,5-dihydro-3H-pyrrolo[3,2-d]pyrimidine-7-carbonitrile hydrochloride;

### M6

6-(1,4-Diazepan-1-yl)-3-methyl-4-oxo-5-(phenylmethyl)-4,5-dihydro-3H-pyrrolo[3,2-d]pyrimidine-7-carbonitrile hydrochloride;

### M7

3-Methyl-4-oxo-5-(phenylmethyl)-6-piperazin-1-yl-4,5-dihydro-3H-pyrrolo[3,2-d]pyrimidine-7-carbonitrile hydrochloride;

### M8

6-((S)-3-Aminopiperidin-1-yl)-4-oxo-5-(phenylmethyl)-3-(2,2,2-trifluoroethyl)-4,5-dihydro-3H-pyrrolo[3,2-d]pyrimidine-7-carbonitrile hydrochloride;

### N1

6-((S)-3-Aminopiperidin-1-yl)-4-oxo-5-(phenylmethyl)-3-(quinolin-8-ylmethyl)-4,5-dihydro-3H-pyrrolo[3,2-d]pyrimidine-7-carbonitrile hydrochloride;

### N2

6-((S)-3-Aminopiperidin-1-yl)-4-oxo-3-((3-phenylisoxazol-5-yl)methyl)-5-(phenylmethyl)-4,5-dihydro-3H-pyrrolo[3,2-d]pyrimidine-7-carbonitrile hydrochloride;

### N3

6-((S)-3-Aminopiperidin-1-yl)-4-oxo-5-(phenylmethyl)-3-(2-(phenyloxy)ethyl)-4,5-dihydro-3H-pyrrolo[3,2-d]pyrimidine-7-carbonitrile hydrochloride;

### N4

6-((S)-3-Aminopiperidin-1-yl)-3-((2-methyl-1,3-thiazol-4-yl)methyl)-4-oxo-5-(phenylmethyl)-4,5-dihydro-3H-pyrrolo[3,2-d]pyrimidine-7-carbonitrile hydrochloride;

### N5

6-((S)-3-Aminopiperidin-1-yl)-3-(2-morpholin-4-ylethyl)-4-oxo-5-(phenylmethyl)-4,5-dihydro-3H-pyrrolo[3,2-d]pyrimidine-7-carbonitrile dihydrochloride;

### N6

6-((S)-3-Aminopiperidin-1-yl)-3-(2,3-dihydro-1,4-benzodioxin-2-ylmethyl)-4-oxo-5-(phenylmethyl)-4,5-dihydro-3H-pyrrolo[3,2-d]pyrimidine-7-carbonitrile hydrochloride;

### N7

6-((S)-3-Aminopiperidin-1-yl)-3-((2-cyanophenyl)methyl)-4-oxo-5-(phenylmethyl)-4,5-dihydro-3H-pyrrolo[3,2-d]pyrimidine-7-carbonitrile hydrochloride;

### N8

6-((S)-3-Aminopiperidin-1-yl)-3-((5-methyl-1,3,4-oxadiazol-2-yl)methyl)-4-oxo-5-(phenylmethyl)-4,5-dihydro-3H-pyrrolo[3,2-d]pyrimidine-7-carbonitrile;

### N9

N-(2-(6-((S)-3-Aminopiperidin-1-yl)-7-cyano-4-oxo-5-(phenylmethyl)-4,5-dihydro-3H-pyrrolo[3,2-d]pyrimidin-3-yl)ethyl)benzenesulfonamide;

### N10

6-((S)-3-Aminopiperidin-1-yl)-3-(2-(3,5-dimethylisoxazol-4-yl)ethyl)-4-oxo-5-(phenylmethyl)-4,5-dihydro-3H-pyrrolo[3,2-d]pyrimidine-7-carbonitrile;

### N11

6-((S)-3-Aminopiperidin-1-yl)-3-(1,3-benzoxazol-2-ylmethyl)-4-oxo-5-(phenylmethyl)-4,5-dihydro-3H-pyrrolo[3,2-d]pyrimidine-7-carbonitrile;

### N12

6-((S)-3-Aminopiperidin-1-yl)-3-methyl-4-oxo-5-(phenylmethyl)-4,5-dihydro-3H-pyrrolo[3,2-d]pyrimidine-7-carbonitrile hydrochloride;

### N13

6-((S)-3-Aminopiperidin-1-yl)-4-oxo-5-(phenylmethyl)-3-(pyridin-2-ylmethyl)-4,5-dihydro-3H-pyrrolo[3,2-d]pyrimidine-7-carbonitrile hydrochloride;

### N14

6-((S)-3-Aminopiperidin-1-yl)-4-oxo-5-(phenylmethyl)-3-(pyridin-3-ylmethyl)-4,5-dihydro-3H-pyrrolo[3,2-d]pyrimidine-7-carbonitrile hydrochloride;

### N15

2-(6-((S)-3-Aminopiperidin-1-yl)-7-cyano-4-oxo-5-(phenylmethyl)-4,5-dihydro-3H-pyrrolo[3,2-d]pyrimidin-3-yl)-N,N-DMA hydrochloride;

### N16

6-((S)-3-Aminopiperidin-1-yl)-4-oxo-5-(phenylmethyl)-3-((5-phenyl-1,2,4-oxadiazol-3-yl)methyl)-4,5-dihydro-3H-pyrrolo[3,2-d]pyrimidine-7-carbonitrile hydrochloride;

### N17

2-(6-((S)-3-Aminopiperidin-1-yl)-7-cyano-4-oxo-5-(phenylmethyl)-4,5-dihydro-3H-pyrrolo[3,2-d]pyrimidin-3-yl)-N-phenylacetamide hydrochloride;

### N18

6-((S)-3-Aminopiperidin-1-yl)-3-(2-morpholin-4-yl-2-oxoethyl)-4-oxo-5-(phenylmethyl)-4,5-dihydro-3H-pyrrolo[3,2-d]pyrimidine-7-carbonitrile hydrochloride;

### N19

6-((S)-3-Aminopiperidin-1-yl)-4-oxo-5-(phenylmethyl)-3-((1-(phenylmethyl)-1H-imidazol-2-yl)methyl)-4,5-dihydro-3H-pyrrolo[3,2-d]pyrimidine-7-carbonitrile hydrochloride;

### N20

6-((S)-3-Aminopiperidin-1-yl)-3-(2-(ethyloxy)ethyl)-4-oxo-5-(phenylmethyl)-4,5-dihydro-3H-pyrrolo[3,2-d]pyrimidine-7-carbonitrile hydrochloride;

### N21

6-((S)-3-Aminopiperidin-1-yl)-4-oxo-5-(phenylmethyl)-3-((6-(trifluoromethyl)pyridin-3-yl)methyl)-4,5-dihydro-3H-pyrrolo[3,2-d]pyrimidine-7-carbonitrile hydrochloride;

### N22

6-((S)-3-Aminopiperidin-1-yl)-3-(imidazo[1,2-a]pyridin-3-ylmethyl)-4-oxo-5-(phenylmethyl)-4,5-dihydro-3H-pyrrolo[3,2-d]pyrimidine-7-carbonitrile hydrochloride;

### N23

6-((S)-3-Aminopiperidin-1-yl)-4-oxo-5-(phenylmethyl)-3-(tetrahydrofuran-2-ylmethyl)-4,5-dihydro-3H-pyrrolo[3,2-d]pyrimidine-7-carbonitrile hydrochloride;

### N24

6-((S)-3-Aminopiperidin-1-yl)-4-oxo-3-(2-phenylethyl)-5-(phenylmethyl)-4,5-dihydro-3H-pyrrolo[3,2-d]pyrimidine-7-carbonitrile hydrochloride;

### N25

6-((S)-3-Aminopiperidin-1-yl)-4-oxo-3-(3-phenylpropyl)-5-(phenylmethyl)-4,5-dihydro-3H-pyrrolo[3,2-d]pyrimidine-7-carbonitrile hydrochloride;

### N26

6-((S)-3-Aminopiperidin-1-yl)-7-cyano-N,N-dimethyl-4-oxo-5-(phenylmethyl)-4,5-dihydro-3H-pyrrolo[3,2-d]pyrimidine-3-carboxamide hydrochloride;

### N27

6-((S)-3-Aminopiperidin-1-yl)-4-oxo-5-(phenylmethyl)-3-(2-(1H-pyrazol-1-yl)ethyl)-4,5-dihydro-3H-pyrrolo[3,2-d]pyrimidine-7-carbonitrile hydrochloride;

### N28

6-((S)-3-Aminopiperidin-1-yl)-3-(2-hydroxyethyl)-4-oxo-5-(phenylmethyl)-4,5-dihydro-3H-pyrrolo[3,2-d]pyrimidine-7-carbonitrile hydrochloride;

### N29

6-((S)-3-Aminopiperidin-1-yl)-3-(cyclopropylmethyl)-4-oxo-5-(phenylmethyl)-4,5-dihydro-3H-pyrrolo[3,2-d]pyrimidine-7-carbonitrile hydrochloride;

### N30

6-((S)-3-Aminopiperidin-1-yl)-4-oxo-5-(phenylmethyl)-3-((2E)-3-phenylprop-2-en-1-yl)-4,5-dihydro-3H-pyrrolo[3,2-d]pyrimidine-7-carbonitrile hydrochloride;

### N31

6-((S)-3-Aminopiperidin-1-yl)-3-(2-cyclohexylethyl)-4-oxo-5-(phenylmethyl)-4,5-dihydro-3H-pyrrolo[3,2-d]pyrimidine-7-carbonitrile hydrochloride;

### N32

6-((S)-3-Aminopiperidin-1-yl)-3-(2,3-dihydro-1,4-benzodioxin-6-ylmethyl)-4-oxo-5-(phenylmethyl)-4,5-dihydro-3H-pyrrolo[3,2-d]pyrimidine-7-carbonitrile hydrochloride;

### N33

6-((S)-3-Aminopiperidin-1-yl)-3-((1,3-dimethyl-1H-pyrazol-5-yl)methyl)-4-oxo-5-(phenylmethyl)-4,5-dihydro-3H-pyrrolo[3,2-d]pyrimidine-7-carbonitrile hydrochloride;

### N34

6-((S)-3-Aminopiperidin-1-yl)-3-(2-(4-methyl-1,3-thiazol-5-yl)ethyl)-4-oxo-5-(phenylmethyl)-4,5-dihydro-3H-pyrrolo[3,2-d]pyrimidine-7-carbonitrile hydrochloride;

### N35

6-((S)-3-Aminopiperidin-1-yl)-3-((1-methyl-1H-benzimidazol-2-yl)methyl)-4-oxo-5-(phenylmethyl)-4,5-dihydro-3H-pyrrolo[3,2-d]pyrimidine-7-carbonitrile hydrochloride;

### N36

6-((S)-3-Aminopiperidin-1-yl)-4-oxo-5-(phenylmethyl)-3-(quinazolin-2-ylmethyl)-4,5-dihydro-3H-pyrrolo[3,2-d]pyrimidine-7-carbonitrile hydrochloride;

### O1

6-((S)-3-Aminopiperidin-1-yl)-N,N-dimethyl-3-(2-(3-(methyloxy)phenyl)-2-oxoethyl)-4-oxo-5-(phenylmethyl)-4,5-dihydro-3H-pyrrolo[3,2-d]pyrimidine-7-carboxamide hydrochloride;

### O2

6-((S)-3-Aminopiperidin-1-yl)-3-(2-(3-(methyloxy)phenyl)-2-oxoethyl)-7-(morpholin-4-ylcarbonyl)-5-(phenylmethyl)-3,5-dihydro-4H-pyrrolo[3,2-d]pyrimidin-4-one hydrochloride;

### O3

6-((S)-3-Aminopiperidin-1-yl)-5-but-2-yn-1-yl-3-(2-(3-(methyloxy)phenyl)-2-oxoethyl)-7-(morpholin-4-ylcarbonyl)-3,5-dihydro-4H-pyrrolo[3,2-d]pyrimidin-4-one hydrochloride;

### O4

6-((S)-3-Aminopiperidin-1-yl)-N,N-dimethyl-5-(3-methylbut-2-en-1-yl)-3-(2-(3-(methyloxy)phenyl)-2-oxoethyl)-4-oxo-4,5-dihydro-3H-pyrrolo[3,2-d]pyrimidine-7-carboxamide;

### O5

5-But-2-yn-1-yl-6-(1,4-diazepan-1-yl)-3-(isoquinolin-1-ylmethyl)-N,N-dimethyl-4-oxo-4,5-dihydro-3H-pyrrolo[3,2-d]pyrimidine-7-carboxamide hydrochloride;

### O6

5-But-2-yn-1-yl-6-(1,4-diazepan-1-yl)-3-(isoquinolin-1-ylmethyl)-7-(piperidin-1-ylcarbonyl)-3,5-dihydro-4H-pyrrolo[3,2-d]pyrimidin-4-one hydrochloride;

### O7

5-But-2-yn-1-yl-6-(1,4-diazepan-1-yl)-3-(isoquinolin-1-ylmethyl)-7-(pyrrolidin-1-ylcarbonyl)-3,5-dihydro-4H-pyrrolo[3,2-d]pyrimidin-4-one hydrochloride;

### O8

5-But-2-yn-1-yl-6-(1,4-diazepan-1-yl)-7-((4,4-difluoropiperidin-1-yl)carbonyl)-3-(isoquinolin-1-ylmethyl)-3,5-dihydro-4H-pyrrolo[3,2-d]pyrimidin-4-one hydrochloride;

### O9

6-((S)-3-Aminopiperidin-1-yl)-5-but-2-yn-1-yl-3-(isoquinolin-1-ylmethyl)-N,N-dimethyl-4-oxo-4,5-dihydro-3H-pyrrolo[3,2-d]pyrimidine-7-carboxamide hydrochloride;

### O10

6-((S)-3-Aminopiperidin-1-yl)-5-but-2-yn-1-yl-3-(isoquinolin-1-ylmethyl)-7-(morpholin-4-ylcarbonyl)-3,5-dihydro-4H-pyrrolo[3,2-d]pyrimidin-4-one hydrochloride;

### O11

6-((S)-3-Aminopiperidin-1-yl)-3-(isoquinolin-1-ylmethyl)-N,N-dimethyl-4-oxo-5-(phenylmethyl)-4,5-dihydro-3H-pyrrolo[3,2-d]pyrimidine-7-carboxamide hydrochloride;

### O12

6-((S)-3-Aminopiperidin-1-yl)-5-(3-methylbut-2-en-1-yl)-3-(2-(3-(methyloxy)phenyl)-2-oxoethyl)-7-(morpholin-4-ylcarbonyl)-3,5-dihydro-4H-pyrrolo[3,2-d]pyrimidin-4-one hydrochloride;

### O13

6-((S)-3-Aminopiperidin-1-yl)-3-(isoquinolin-1-ylmethyl)-7-(morpholin-4-ylcarbonyl)-5-(phenylmethyl)-3,5-dihydro-4H-pyrrolo[3,2-d]pyrimidin-4-one hydrochloride;

### P1

6-((S)-3-Aminopiperidin-1-yl)-3-(2-(3-(methyloxy)phenyl)-2-oxoethyl)-5-(phenylmethyl)-3,5-dihydro-4H-pyrrolo[3,2-d]pyrimidin-4-one;

### P2

6-((S)-3-Aminopiperidin-1-yl)-5-but-2-yn-1-yl-3-(2-(3-(methyloxy)phenyl)-2-oxoethyl)-3,5-dihydro-4H-pyrrolo[3,2-d]pyrimidin-4-one;

### P3

6-((S)-3-Aminopiperidin-1-yl)-5-(3-methylbut-2-en-1-yl)-3-(2-(3-(methyloxy)phenyl)-2-oxoethyl)-3,5-dihydro-4H-pyrrolo[3,2-d]pyrimidin-4-one;

### P4

6-((S)-3-Aminopiperidin-1-yl)-5-but-2-yn-1-yl-3-(isoquinolin-1-ylmethyl)-3,5-dihydro-4H-pyrrolo[3,2-d]pyrimidin-4-one;

### P5

6-((S)-3-Aminopiperidin-1-yl)-3-(isoquinolin-1-ylmethyl)-5-(phenylmethyl)-3,5-dihydro-4H-pyrrolo[3,2-d]pyrimidin-4-one;

### P6

5-But-2-yn-1-yl-6-(1,4-diazepan-1-yl)-3-(isoquinolin-1-ylmethyl)-3,5-dihydro-4H-pyrrolo[3,2-d]pyrimidin-4-one;

### Q1

6-((S)-3-Aminopiperidin-1-yl)-5-(3-methylbut-2-en-1-yl)-4-oxo-3-(quinazolin-2-ylmethyl)-4,5-dihydro-3H-pyrrolo[3,2-d]pyrimidine-7-carbonitrile;

### Q2

6-((S)-3-Aminopiperidin-1-yl)-5-but-2-yn-1-yl-4-oxo-3-(quinolin-4-ylmethyl)-4,5-dihydro-3H-pyrrolo[3,2-d]pyrimidine-7-carbonitrile;

### Q3

6-((S)-3-Aminopiperidin-1-yl)-5-but-2-yn-1-yl-3-(isoquinolin-1-ylmethyl)-4-oxo-4,5-dihydro-3H-pyrrolo[3,2-d]pyrimidine-7-carbonitrile;

### Q4

6-((S)-3-Aminopiperidin-1-yl)-5-but-2-yn-1-yl-4-oxo-4,5-dihydro-3H-pyrrolo[3,2-d]pyrimidine-7-carbonitrile;

### Q5

6-((S)-3-Aminopiperidin-1-yl)-5-((2-cyanophenyl)methyl)-3-methyl-4-oxo-4,5-dihydro-3H-pyrrolo[3,2-d]pyrimidine-7-carbonitrile hydrochloride;

### Q6

6-((S)-3-Aminopiperidin-1-yl)-5-((2-cyanophenyl)methyl)-3-(isoquinolin-1-ylmethyl)-4-oxo-4,5-dihydro-3H-pyrrolo[3,2-d]pyrimidine-7-carbonitrile hydrochloride;

### Q7

5-But-2-yn-1-yl-6-(1,4-diazepan-1-yl)-3-((4-methylquinazolin-2-yl)methyl)-4-oxo-4,5-dihydro-3H-pyrrolo[3,2-d]pyrimidine-7-carbonitrile;

### Q8

4-((5-But-2-yn-1-yl-7-cyano-6-(1,4-diazepan-1-yl)-4-oxo-4,5-dihydro-3H-pyrrolo[3,2-d]pyrimidin-3-yl)methyl)-quinoline-3-carbonitrile;

### Q9

5-But-2-yn-1-yl-3-((3-cyanopyridin-2-yl)methyl)-6-(1,4-diazepan-1-yl)-4-oxo-4,5-dihydro-3H-pyrrolo[3,2-d]pyrimidine-7-carbonitrile hydrochloride;

### Q10

5-But-2-yn-1-yl-6-(1,4-diazepan-1-yl)-4-oxo-3-(quinoxalin-2-ylmethyl)-4,5-dihydro-3H-pyrrolo[3,2-d]pyrimidine-7-carbonitrile hydrochloride;

### Q11

5-But-2-yn-1-yl-6-(1,4-diazepan-1-yl)-3-((4-methyl-3-oxidoquinazolin-2-yl)methyl)-4-oxo-4,5-dihydro-3H-pyrrolo[3,2-d]pyrimidine-7-carbonitrile hydrochloride;

### Q12

6-((S)-3-Aminopiperidin-1-yl)-5-(3-methylbut-2-en-1-yl)-3-((2-oxidoisoquinolin-1-yl)methyl)-4-oxo-4,5-dihydro-3H-pyrrolo[3,2-d]pyrimidine-7-carbonitrile;

### Q13

6-((S)-3-Aminopiperidin-1-yl)-5-(3-methylbut-2-en-1-yl)-3-((4-methylquinazolin-2-yl)methyl)-4-oxo-4,5-dihydro-3H-pyrrolo[3,2-d]pyrimidine-7-carbonitrile;

### Q14

6-((S)-3-Aminopiperidin-1-yl)-5-(3-methylbut-2-en-1-yl)-3-((4-methyl-3-oxidoquinazolin-2-yl)methyl)-4-oxo-4,5-dihydro-3H-pyrrolo[3,2-d]pyrimidine-7-carbonitrile;

### R1

6-((S)-3-Aminopiperidin-1-yl)-5-but-2-yn-1-yl-1,3-dimethyl-2,4-dioxo-2,3,4,5-tetrahydro-1H-pyrrolo[3,2-d]pyrimidine-7-carbonitrile;

### R2

6-((S)-3-Aminopiperidin-1-yl)-1,3-dimethyl-5-(3-methylbut-2-en-1-yl)-2,4-dioxo-2,3,4,5-tetrahydro-1H-pyrrolo[3,2-d]pyrimidine-7-carbonitrile;

### S1

1,3-Dimethyl-2,4-dioxo-5-(phenylmethyl)-6-piperazin-1-yl-2,3,4,5-tetrahydro-1H-pyrrolo[3,2-d]pyrimidine-7-carbonitrile hydrochloride;

### S2

1,3-Dimethyl-2,4-dioxo-5-(phenylmethyl)-6-piperazin-1-yl-2,3,4,5-tetrahydro-1H-pyrrolo[3,2-d]pyrimidine-7-carbonitrile hydrochloride;

### S3

3-(Isoquinolin-1-ylmethyl)-1-methyl-2,4-dioxo-5-(phenylmethyl)-6-piperazin-1-yl-2,3,4,5-tetrahydro-1H-pyrrolo[3,2-d]pyrimidine-7-carbonitrile hydrochloride;

### S4

6-(1,4-Diazepan-1-yl)-1,3-dimethyl-2,4-dioxo-5-(phenylmethyl)-2,3,4,5-tetrahydro-1H-pyrrolo[3,2-d]pyrimidine-7-carbonitrile hydrochloride;

### S5

6-(1,4-Diazepan-1-yl)-1-methyl-3-(2-(3-(methyloxy)phenyl)-2-oxoethyl)-2,4-dioxo-5-(phenylmethyl)-2,3,4,5-tetrahydro-1H-pyrrolo[3,2-d]pyrimidine-7-carbonitrile hydrochloride;

### S6

6-(1,4-Diazepan-1-yl)-3-(isoquinolin-1-ylmethyl)-1-methyl-2,4-dioxo-5-(phenylmethyl)-2,3,4,5-tetrahydro-1H-pyrrolo[3,2-d]pyrimidine-7-carbonitrile hydrochloride;

### T1

5-But-2-yn-1-yl-6-(1,4-diazepan-1-yl)-3-(isoquinolin-1-ylmethyl)-1-methyl-2,4-dioxo-2,3,4,5-tetrahydro-1H-pyrrolo[3,2-d]pyrimidine-7-carbonitrile hydrochloride;

### T2

6-((S)-3-Aminopiperidin-1-yl)-5-but-2-yn-1-yl-3-(isoquinolin-1-ylmethyl)-1-methyl-2,4-dioxo-2,3,4,5-tetrahydro-1H-pyrrolo[3,2-d]pyrimidine-7-carbonitrile hydrochloride;

### T3

6-((S)-3-Aminopiperidin-1-yl)-5-but-2-yn-1-yl-1-methyl-3-(2-(3-(methyloxy)phenyl)-2-oxoethyl)-2,4-dioxo-2,3,4,5-tetrahydro-1H-pyrrolo[3,2-d]pyrimidine-7-carbonitrile hydrochloride;

### U1

6-(1,4-Diazepan-1-yl)-3-(isoquinolin-1-ylmethyl)-1-methyl-5-(3-methylbut-2-en-1-yl)-2,4-dioxo-2,3,4,5-tetrahydro-1H-pyrrolo[3,2-d]pyrimidine-7-carbonitrile;

### U2

6-(1,4-Diazepan-1-yl)-1-methyl-5-(3-methylbut-2-en-1-yl)-3-(2-(3-(methyloxy)phenyl)-2-oxoethyl)-2,4-dioxo-2,3,4,5-tetrahydro-1H-pyrrolo[3,2-d]pyrimidine-7-carbonitrile;

### U3

6-((S)-3-Aminopiperidin-1-yl)-3-(isoquinolin-1-ylmethyl)-1-methyl-5-(3-methylbut-2-en-1-yl)-2,4-dioxo-2,3,4,5-tetrahydro-1H-pyrrolo[3,2-d]pyrimidine-7-carbonitrile;

### U4

6-((S)-3-Aminopiperidin-1-yl)-1-methyl-5-(3-methylbut-2-en-1-yl)-3-(2-(3-(methyloxy)phenyl)-2-oxoethyl)-2,4-dioxo-2,3,4,5-tetrahydro-1H-pyrrolo[3,2-d]pyrimidine-7-carbonitrile;

### U5

1-Methyl-5-(3-methylbut-2-en-1-yl)-3-(2-(3-(methyloxy)phenyl)-2-oxoethyl)-2,4-dioxo-6-piperazin-1-yl-2,3,4,5-tetrahydro-1H-pyrrolo[3,2-d]pyrimidine-7-carbonitrile;

### U6

6-((S)-3-Aminopiperidin-1-yl)-1-methyl-5-(3-methylbut-2-en-1-yl)-3-((4-methylquinazolin-2-yl)methyl)-2,4-dioxo-2,3,4,5-tetrahydro-1H-pyrrolo[3,2-d]pyrimidine-7-carbonitrile;

### U7

6-((S)-3-Aminopiperidin-1-yl)-1-methyl-5-(3-methylbut-2-en-1-yl)-3-((4-methyl-3-oxidoquinazolin-2-yl)methyl)-2,4-dioxo-2,3,4,5-tetrahydro-1H-pyrrolo[3,2-d]pyrimidine-7-carbonitrile;

### U8

6-((S)-3-Aminopiperidin-1-yl)-3-(cyanomethyl)-1-methyl-5-(3-methylbut-2-en-1-yl)-2,4-dioxo-2,3,4,5-tetrahydro-1H-pyrrolo[3,2-d]pyrimidine-7-carbonitrile;

### U9

6-((S)-3-Aminopiperidin-1-yl)-3-ethyl-1-methyl-5-(3-methylbut-2-en-1-yl)-2,4-dioxo-2,3,4,5-tetrahydro-1H-pyrrolo[3,2-d]pyrimidine-7-carbonitrile;

### U10

6-((S)-3-Aminopiperidin-1-yl)-3-((2-cyanophenyl)methyl)-1-methyl-5-(3-methylbut-2-en-1-yl)-2,4-dioxo-2,3,4,5-tetrahydro-1H-pyrrolo[3,2-d]pyrimidine-7-carbonitrile;

### U11

6-((S)-3-Aminopiperidin-1-yl)-1-methyl-5-(3-methylbut-2-en-1-yl)-2,4-dioxo-3-(pyridazin-3-ylmethyl)-2,3,4,5-tetrahydro-1H-pyrrolo[3,2-d]pyrimidine-7-carbonitrile;

### U12

6-((S)-3-Aminopiperidin-1-yl)-1-methyl-5-(3-methylbut-2-en-1-yl)-2,4-dioxo-3-(pyrimidin-4-ylmethyl)-2,3,4,5-tetrahydro-1H-pyrrolo[3,2-d]pyrimidine-7-carbonitrile;

### U13

6-((S)-3-Aminopiperidin-1-yl)-1-methyl-5-(3-methylbut-2-en-1-yl)-2,4-dioxo-3-(pyridin-2-ylmethyl)-2,3,4,5-tetrahydro-1H-pyrrolo[3,2-d]pyrimidine-7-carbonitrile;

### U14

6-((S)-3-Aminopiperidin-1-yl)-1-methyl-5-(3-methylbut-2-en-1-yl)-2,4-dioxo-3-(pyridin-4-ylmethyl)-2,3,4,5-tetrahydro-1H-pyrrolo[3,2-d]pyrimidine-7-carbonitrile;

### U15

6-((S)-3-Aminopiperidin-1-yl)-3-(2-(ethyloxy)ethyl)-1-methyl-5-(3-methylbut-2-en-1-yl)-2,4-dioxo-2,3,4,5-tetrahydro-1H-pyrrolo[3,2-d]pyrimidine-7-carbonitrile;

### U16

6-((S)-3-Aminopiperidin-1-yl)-1-methyl-5-(3-methylbut-2-en-1-yl)-3-([1,3]oxazolo[4,5-b]pyridin-2-ylmethyl)-2,4-dioxo-2,3,4,5-tetrahydro-1H-pyrrolo[3,2-d]pyrimidine-7-carbonitrile;

### U17

6-((S)-3-Aminopiperidin-1-yl)-1-methyl-5-(3-methylbut-2-en-1-yl)-3-((5-methylisoxazol-3-yl)methyl)-2,4-dioxo-2,3,4,5-tetrahydro-1H-pyrrolo[3,2-d]pyrimidine-7-carbonitrile;

### U18

6-((S)-3-Aminopiperidin-1-yl)-3-((3-cyanopyridin-2-yl)methyl)-1-methyl-5-(3-methylbut-2-en-1-yl)-2,4-dioxo-2,3,4,5-tetrahydro-1H-pyrrolo[3,2-d]pyrimidine-7-carbonitrile;

### U19

6-((S)-3-Aminopiperidin-1-yl)-1-methyl-5-(3-methylbut-2-en-1-yl)-2,4-dioxo-3-(quinoxalin-2-ylmethyl)-2,3,4,5-tetrahydro-1H-pyrrolo[3,2-d]pyrimidine-7-carbonitrile;

### U20

6-((S)-3-Aminopiperidin-1-yl)-1-methyl-5-(3-methylbut-2-en-1-yl)-2,4-dioxo-3-(pyrazin-2-ylmethyl)-2,3,4,5-tetrahydro-1H-pyrrolo[3,2-d]pyrimidine-7-carbonitrile;

### U21

6-(1,4-Diazepan-1-yl)-1-methyl-5-(3-methylbut-2-en-1-yl)-3-[(2-oxidoisoquinolin-1-yl)methyl]-2,4-dioxo-2,3,4,5-tetrahydro-1H-pyrrolo[3,2-d]pyrimidine-7-carbonitrile;

### U22

4-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}quinoline-3-carbonitrile;

### U23

6-(1,4-Diazepan-1-yl)-1-methyl-5-(3-methyl but-2-en-1-yl)-3-[(4-methylquinazolin-2-yl)methyl]-2,4-dioxo-2,3,4,5-tetrahydro-1H-pyrrolo[3,2-d]pyrimidine-7-carbonitrile;

### U24

6-(1,4-Diazepan-1-yl)-1-methyl-5-(3-methyl but-2-en-1-yl)-3-[(4-methyl-3-oxidoquinazolin-2-yl)methyl]-2,4-dioxo-2,3,4,5-tetrahydro-1H-pyrrolo[3,2-d]pyrimidine-7-carbonitrile;

### U25

6-(1,4-Diazepan-1-yl)-3-(imidazo[1,2-a]pyridin-2-ylmethyl)-1-methyl-5-(3-methylbut-2-en-1-yl)-2,4-dioxo-2,3,4,5-tetrahydro-1H-pyrrolo[3,2-d]pyrimidine-7-carbonitrile;

### U26

Methyl 2-{[7-cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}nicotinate;

### U27

2-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}-N-ethylnicotinamide;

### U28

2-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}nicotinamide;

### U29

3-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}isoquinoline-4-carbonitrile;

### U30

4-{[5-But-2-yn-1-yl-7-cyano-6-(1,4-diazepan-1-yl)-1-methyl-2,4-dioxo-1,2,4,5-tetrahydro-3H-pyrrolo[3,2-d]pyrimidin-3-yl]methyl}quinoline-3-carbonitrile;

### U31

3-{[5-But-2-yn-1-yl-7-cyano-6-(1,4-diazepan-1-yl)-1-methyl-2,4-dioxo-1,2,4,5-tetrahydro-3H-pyrrolo[3,2-d]pyrimidin-3-yl]methyl}isoquinoline-4-carbonitrile;

### V1

5-Benzyl-6-(1,4-diazepan-1-yl)-3-(isoquinolin-1-ylmethyl)-1-methyl-2,4-dioxo-2,3,4,5-tetrahydro-1H-pyrrolo[3,2-d]pyrimidine-7-carboxamide hydrochloride;

### V2

5-Benzyl-6-(1,4-diazepan-1-yl)-3-(isoquinolin-1-ylmethyl)-N,N,1-trimethyl-2,4-dioxo-2,3,4,5-tetrahydro-1H-pyrrolo[3,2-d]pyrimidine-7-carboxamide hydrochloride;

### V3

5-Benzyl-6-(1,4-diazepan-1-yl)-3-(isoquinolin-1-ylmethyl)-1-methyl-7-(morpholin-4-ylcarbonyl)-1H-pyrrolo[3,2-d]pyrimidine-2,4(3H,5H)-dione;

### V4

5-Benzyl-6-(1,4-diazepan-1-yl)-1,3-dimethyl-2,4-dioxo-2,3,4,5-tetrahydro-1H-pyrrolo[3,2-d]pyrimidine-7-carboxamide hydrochloride;

### V5

5-Benzyl-6-(1,4-diazepan-1-yl)-N,N,1,3-tetramethyl-2,4-dioxo-2,3,4,5-tetrahydro-1H-pyrrolo[3,2-d]pyrimidine-7-carboxamide hydrochloride;

### W1

6-(1,4-Diazepan-1-yl)-3-(isoquinolin-1-ylmethyl)-N,N,1-trimethyl-5-(3-methylbut-2-en-1-yl)-2,4-dioxo-2,3,4,5-tetrahydro-1H-pyrrolo[3,2-d]pyrimidine-7-carboxamide;

### W2

6-(1,4-Diazepan-1-yl)-3-(isoquinolin-1-ylmethyl)-1-methyl-5-(3-methylbut-2-en-1-yl)-2,4-dioxo-2,3,4,5-tetrahydro-1H-pyrrolo[3,2-d]pyrimidine-7-carboxamide;

### W3

6-(1,4-Diazepan-1-yl)-3-(isoquinolin-1-ylmethyl)-1-methyl-5-(3-methylbut-2-en-1-yl)-7-(morpholin-4-ylcarbonyl)-1H-pyrrolo[3,2-d]pyrimidine-2,4(3H,5H)-dione;

### W4

6-(1,4-Diazepan-1-yl)-3-(isoquinolin-1-ylmethyl)-1-methyl-5-(3-methylbut-2-en-1-yl)-7-(piperidin-1-ylcarbonyl)-1H-pyrrolo[3,2-d]pyrimidine-2,4(3H,5H)-dione;

### W5

5-But-2-yn-1-yl-6-(1,4-diazepan-1-yl)-3-(isoquinolin-1-ylmethyl)-N,N,1-trimethyl-2,4-dioxo-2,3,4,5-tetrahydro-1H-pyrrolo[3,2-d]pyrimidine-7-carboxamide;

### X1

6-(1,4-Diazepan-1-yl)-3-(isoquinolin-1-ylmethyl)-5-(3-methylbut-2-en-1-yl)-3,5-dihydro-4H-pyrrolo[3,2-d]pyrimidin-4-one;

### Y1

5-But-2-yn-1-yl-6-(1,4-diazepan-1-yl)-N,N,1,3-tetramethyl-2,4-dioxo-2,3,4,5-tetrahydro-1H-pyrrolo[3,2-d]pyrimidine-7-carboxamide; and

### Z1

6-[(3S)-3-Aminopiperidin-1-yl]-5-benzyl-1,3-dimethyl-7-(methylthio)-1H-pyrrolo[3,2-d]pyrimidine-2,4(3H,5H)-dione;
   the corresponding structures of which are shown below, respectively (ordered left to right):

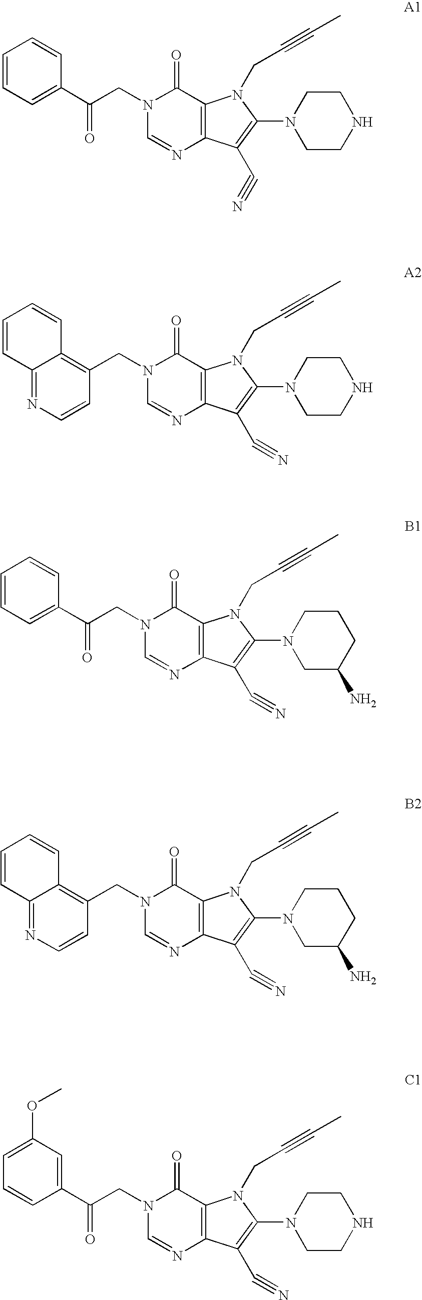

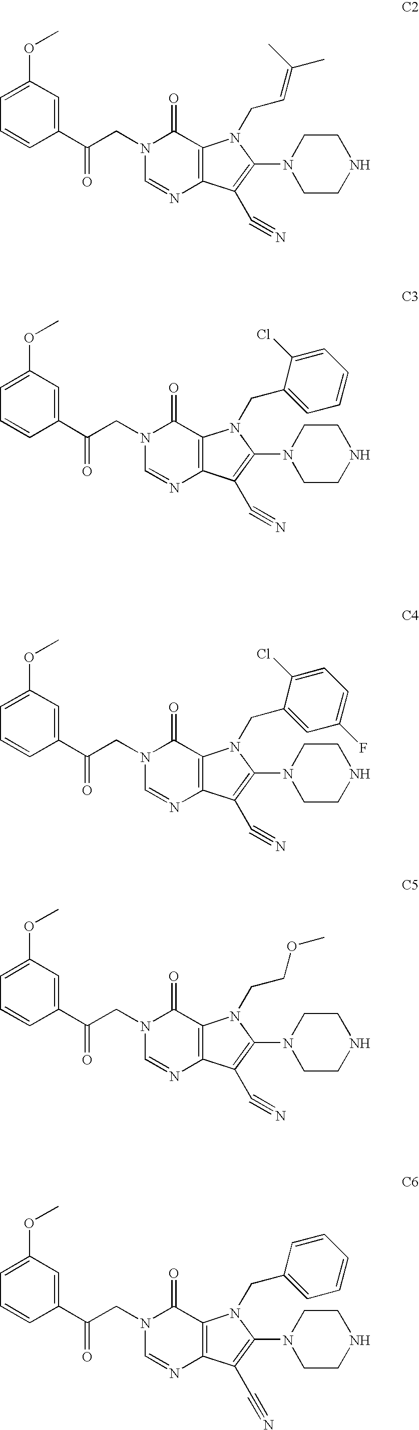

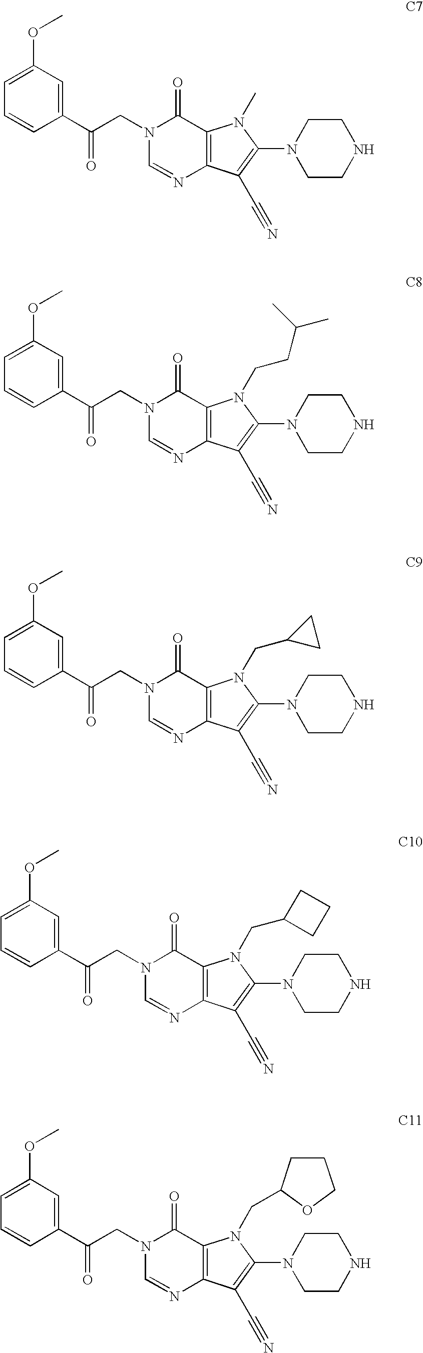

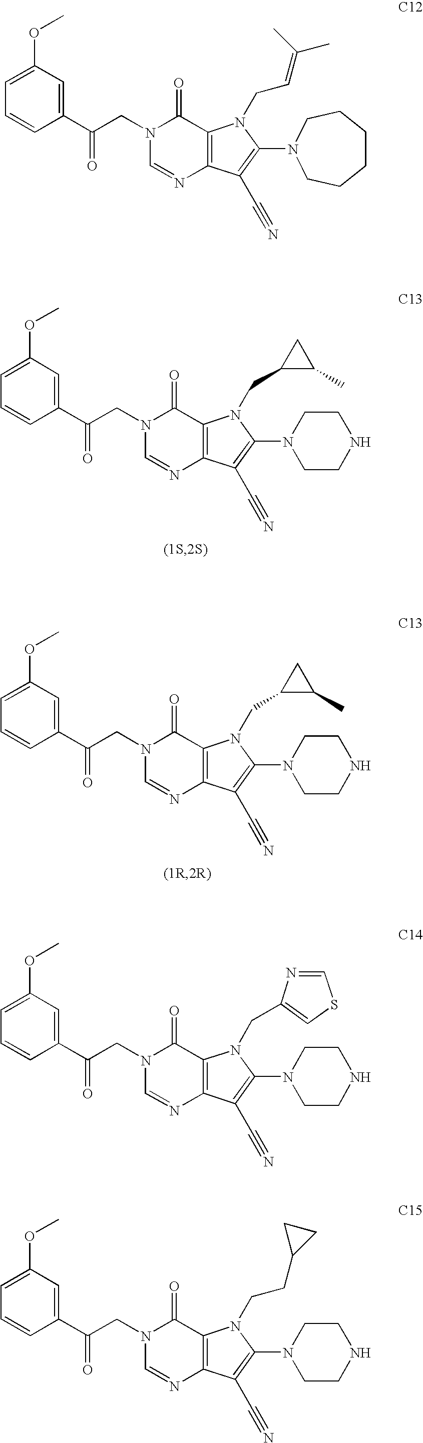

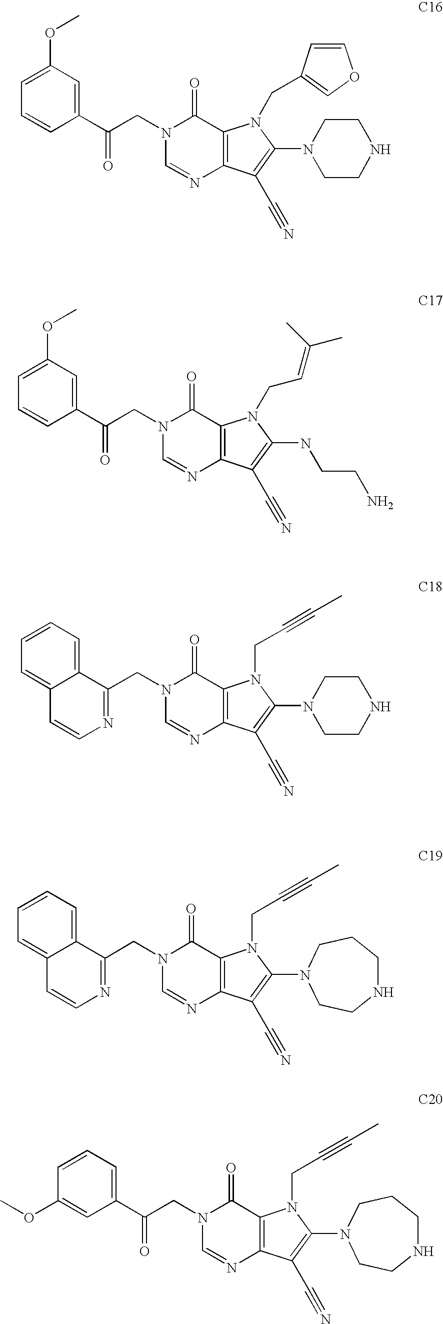

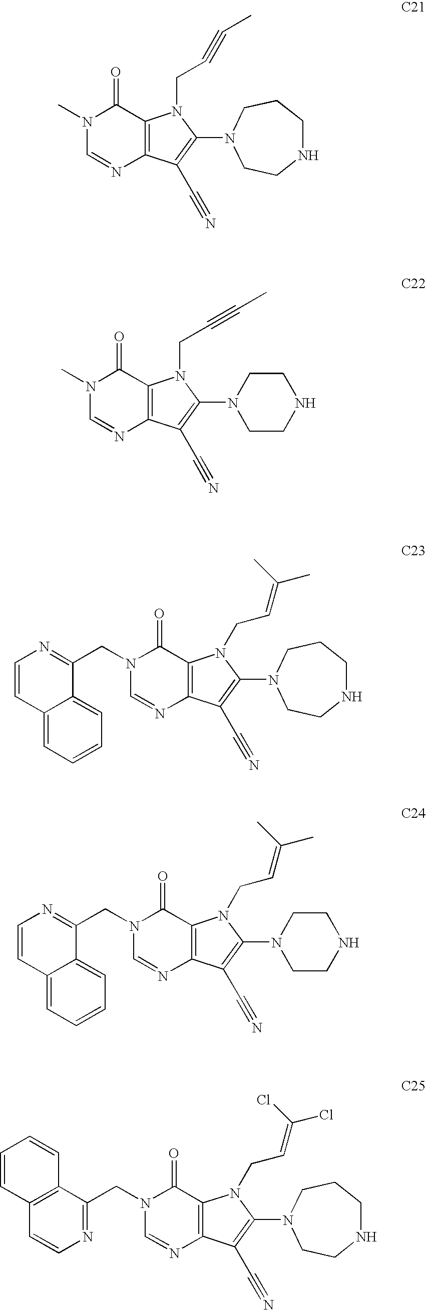

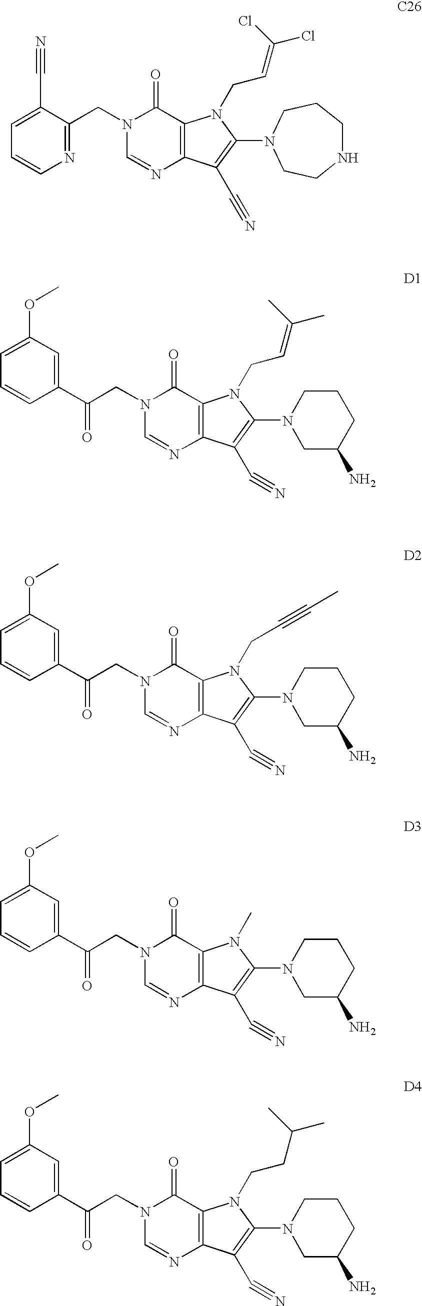

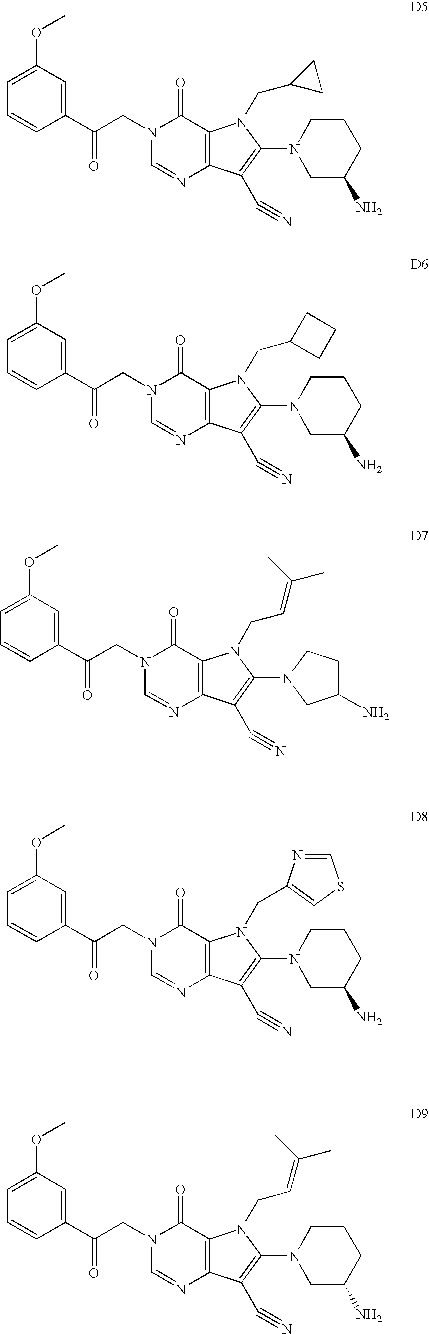

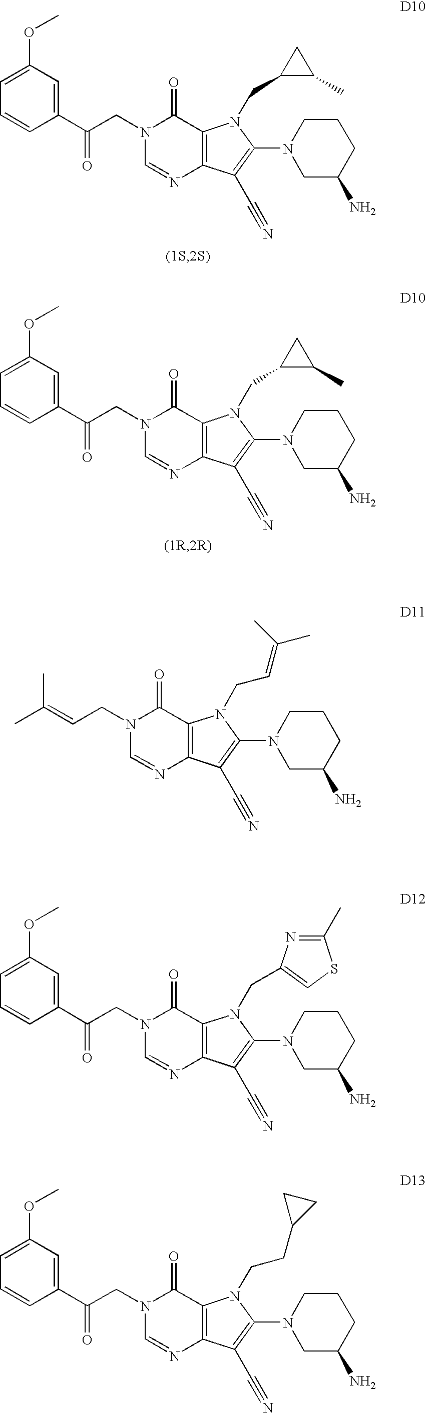

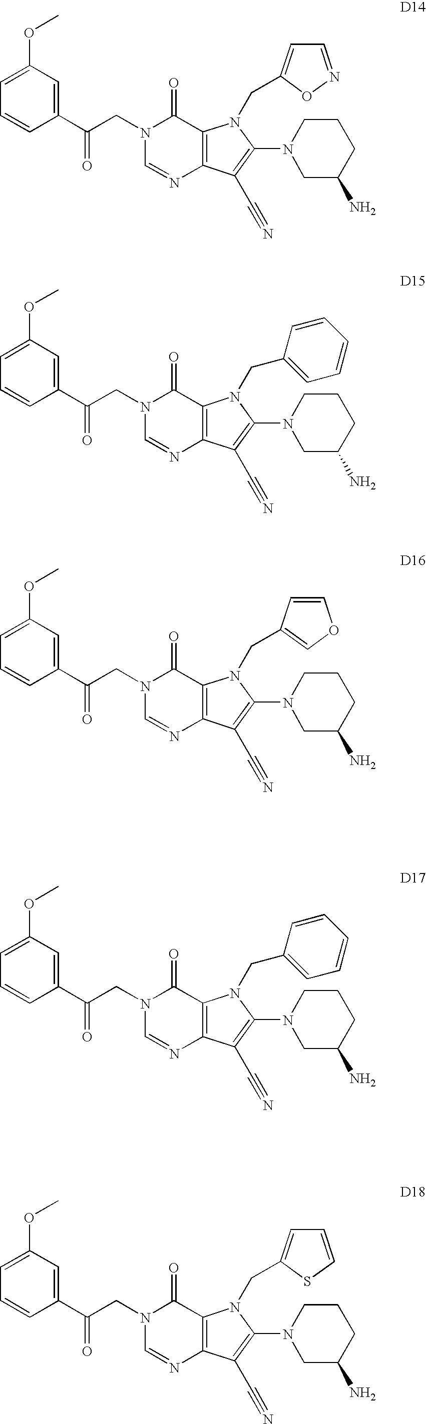

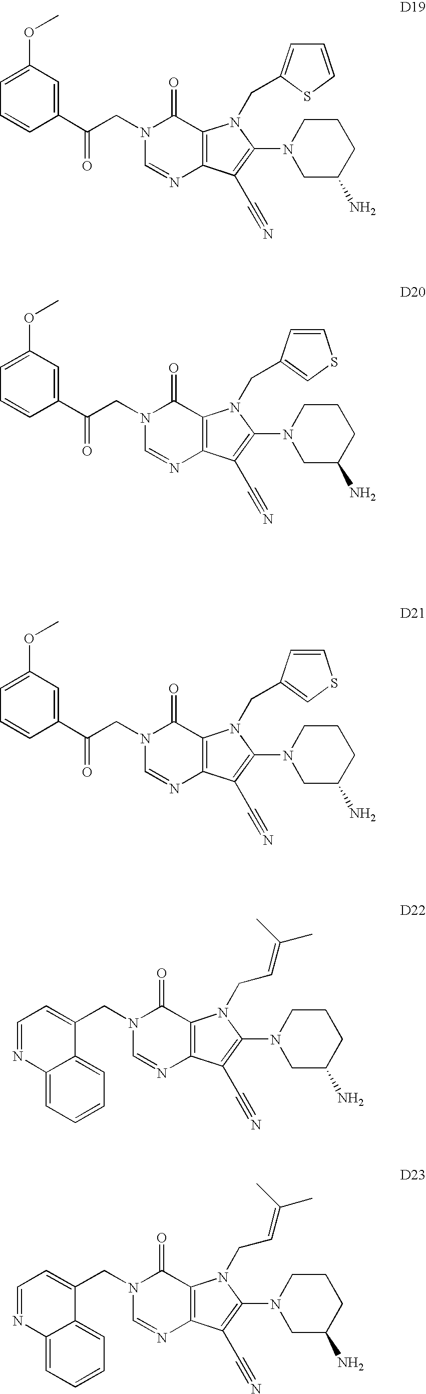

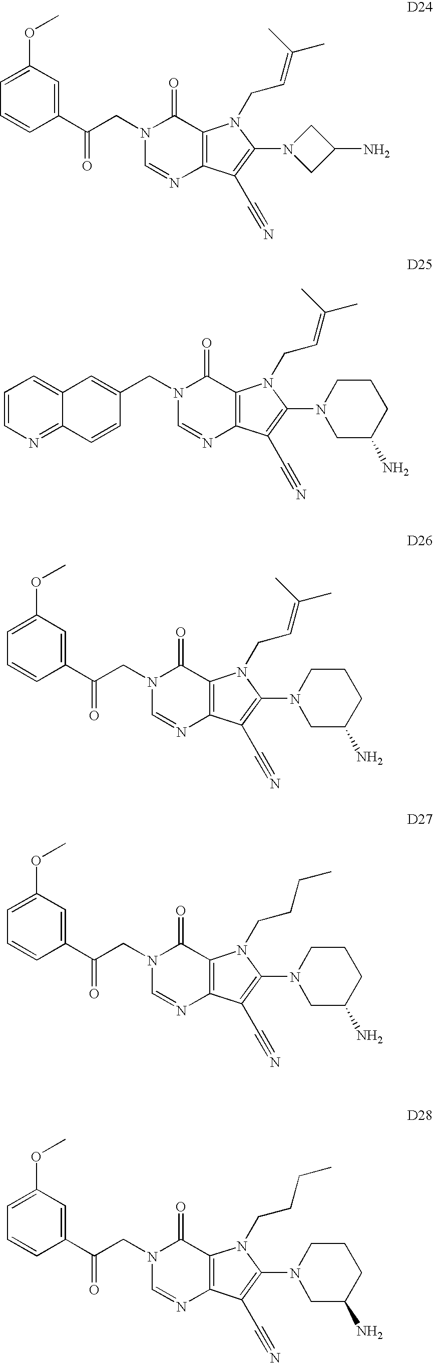

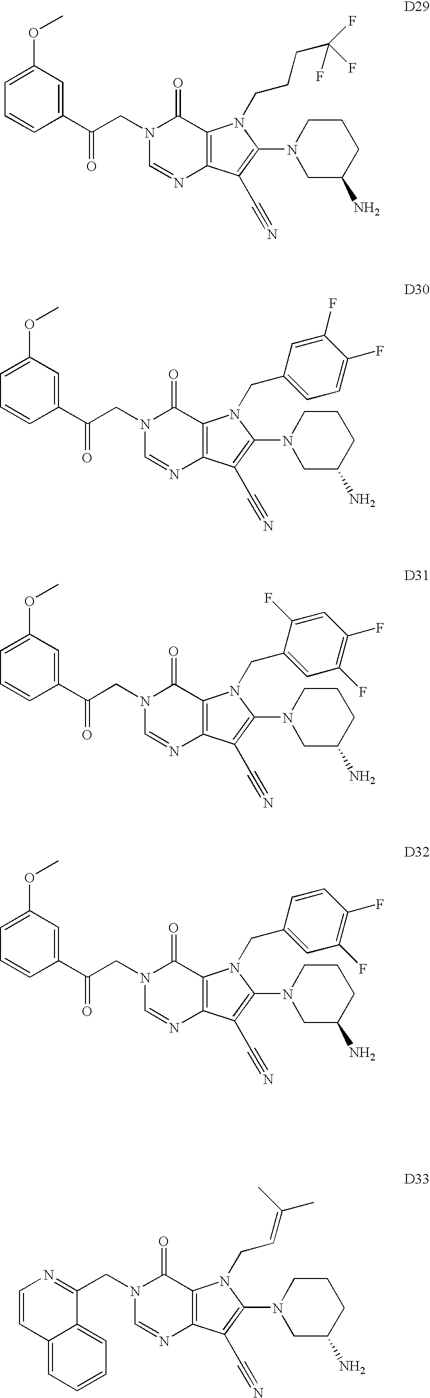

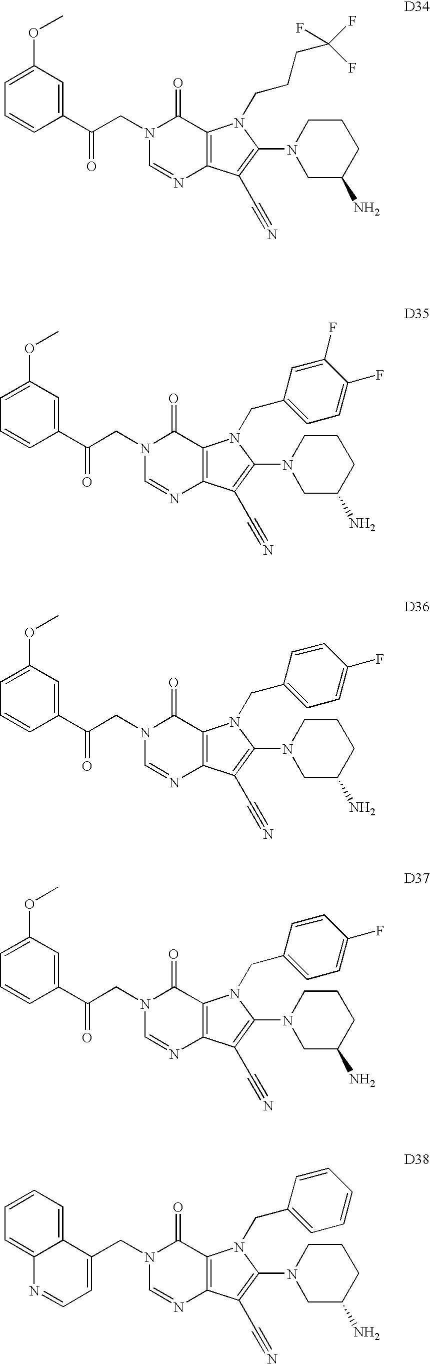

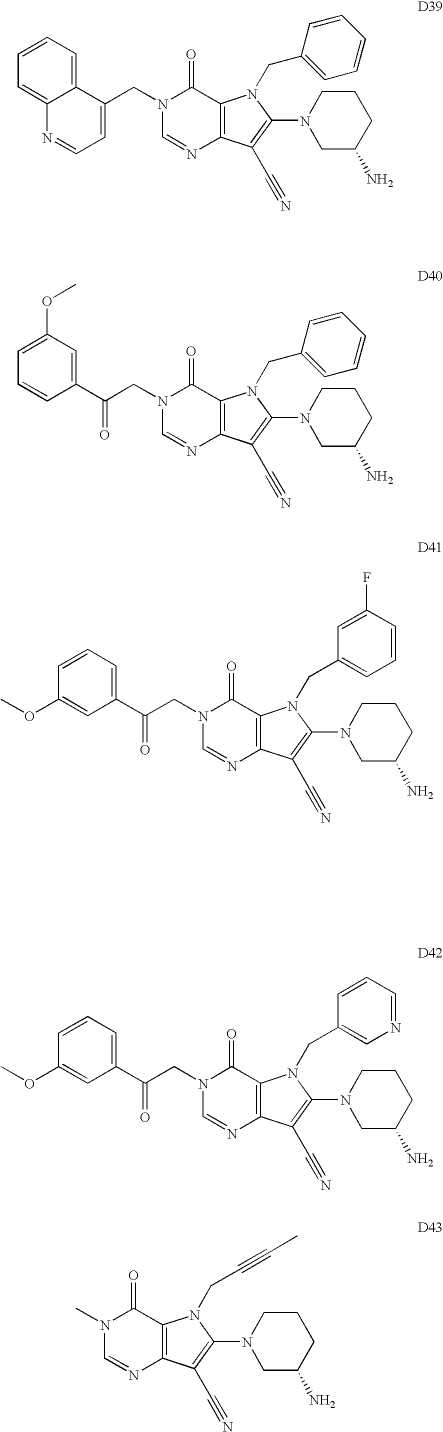

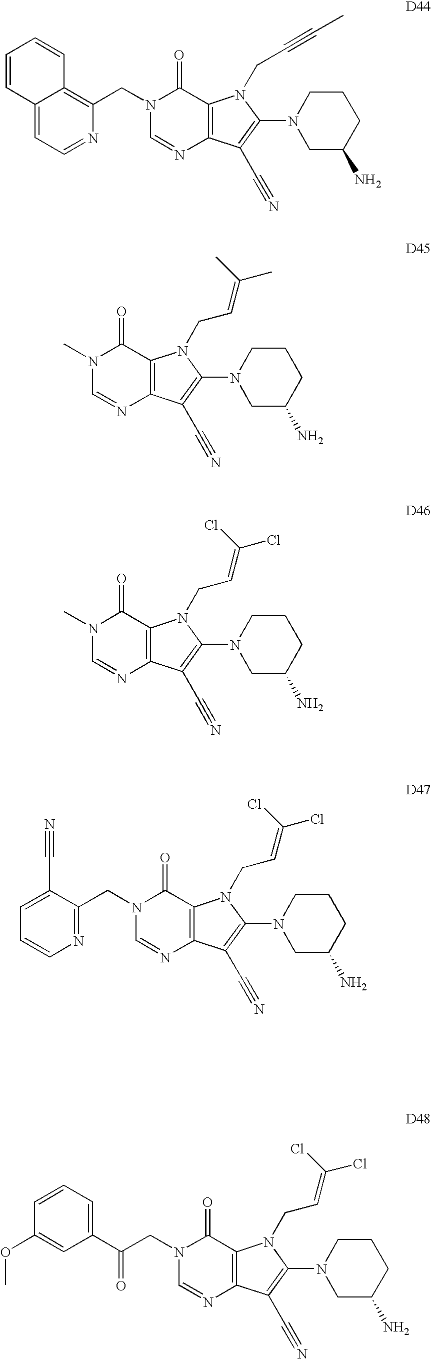

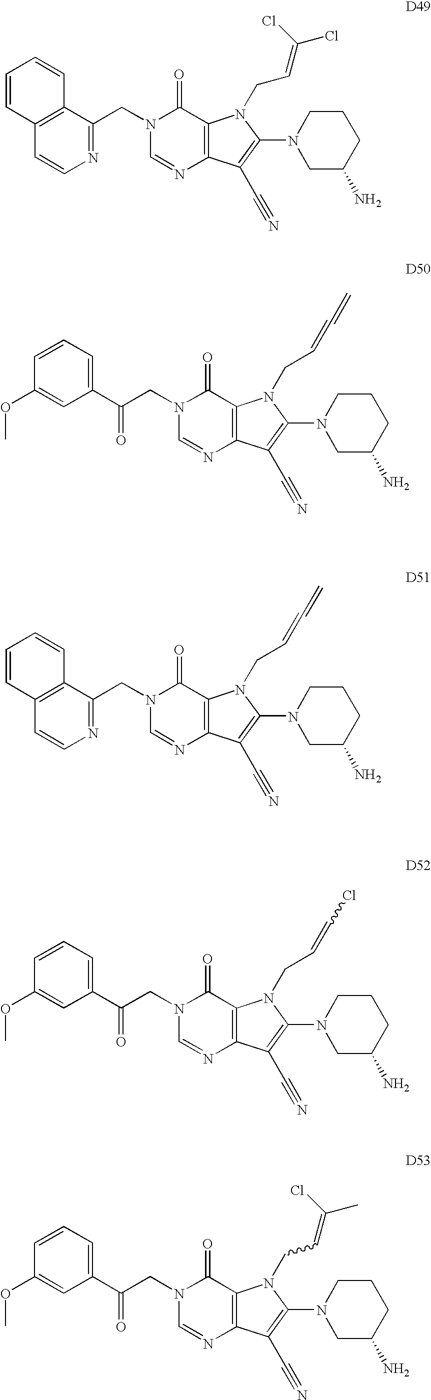

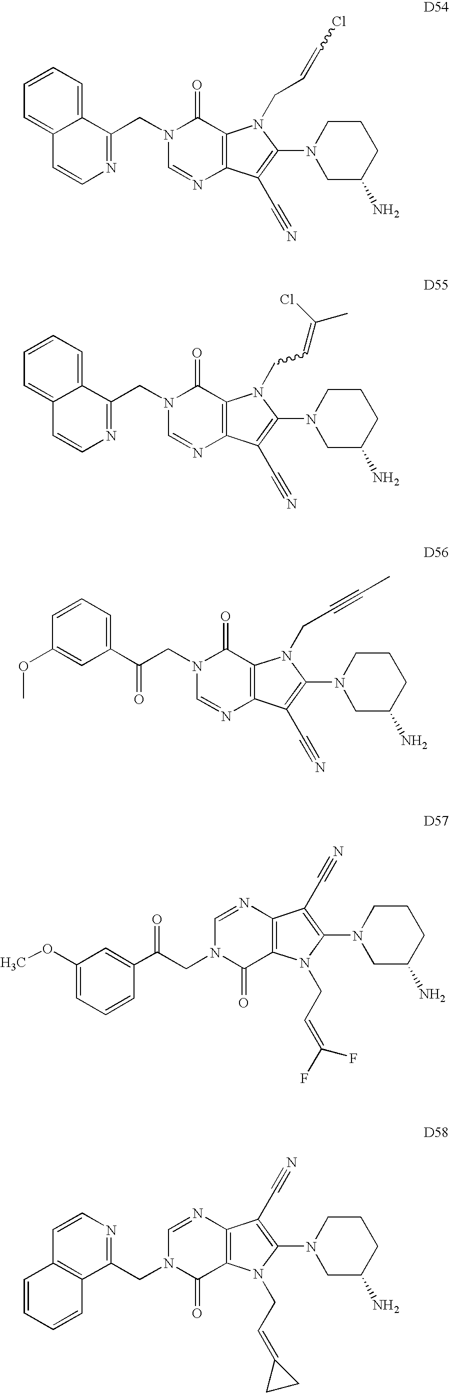

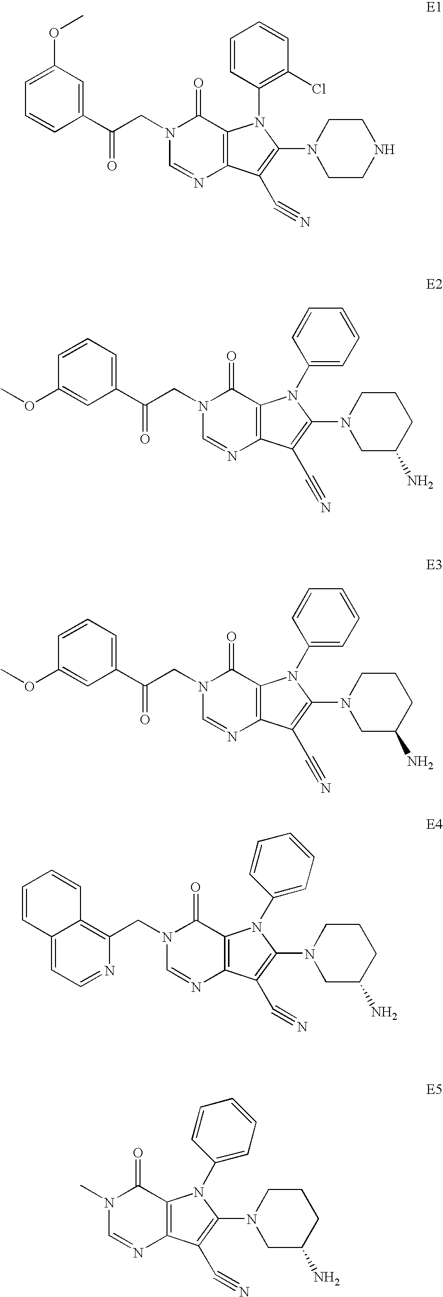

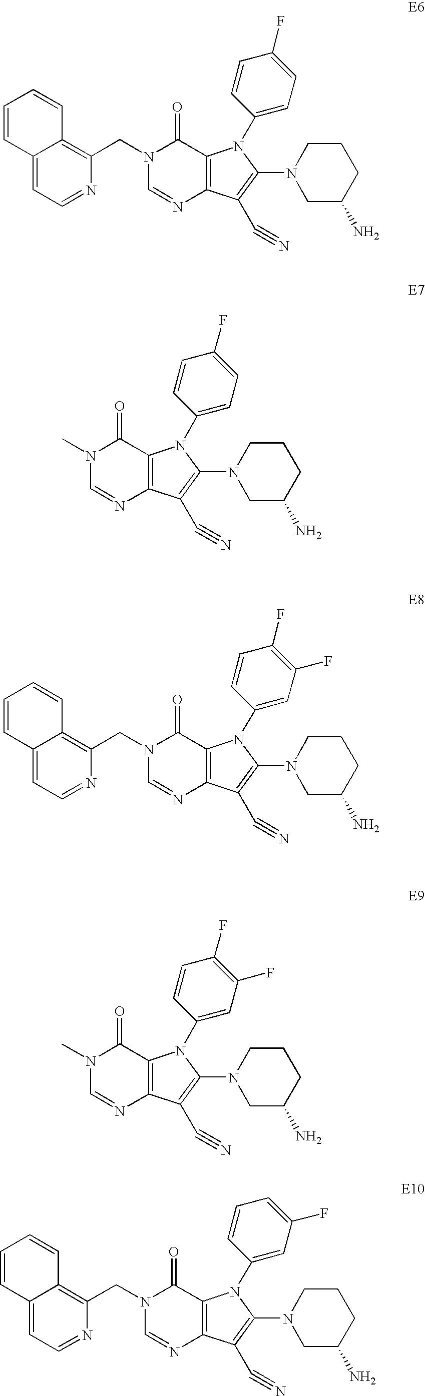

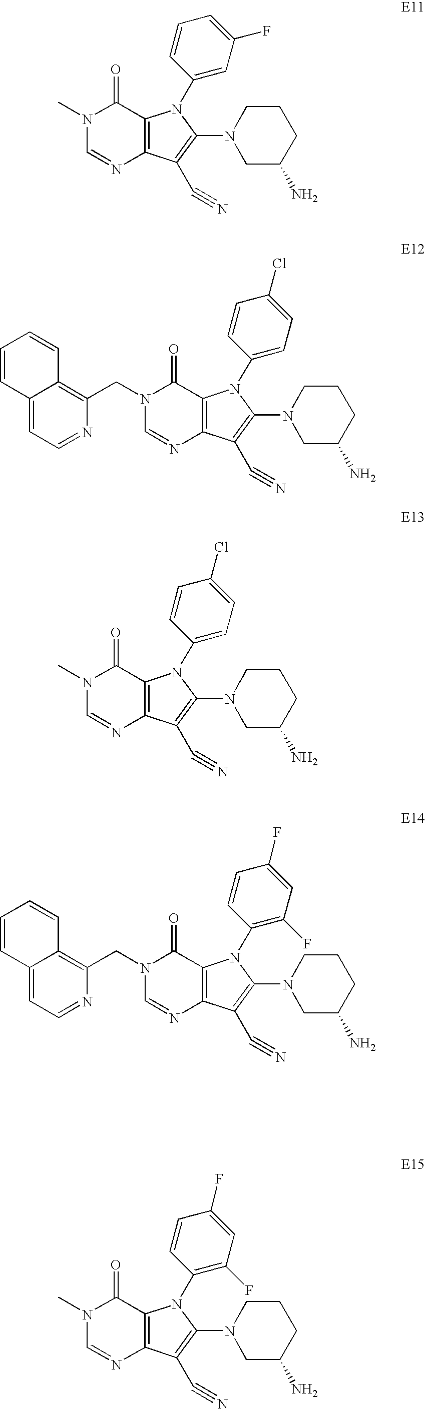

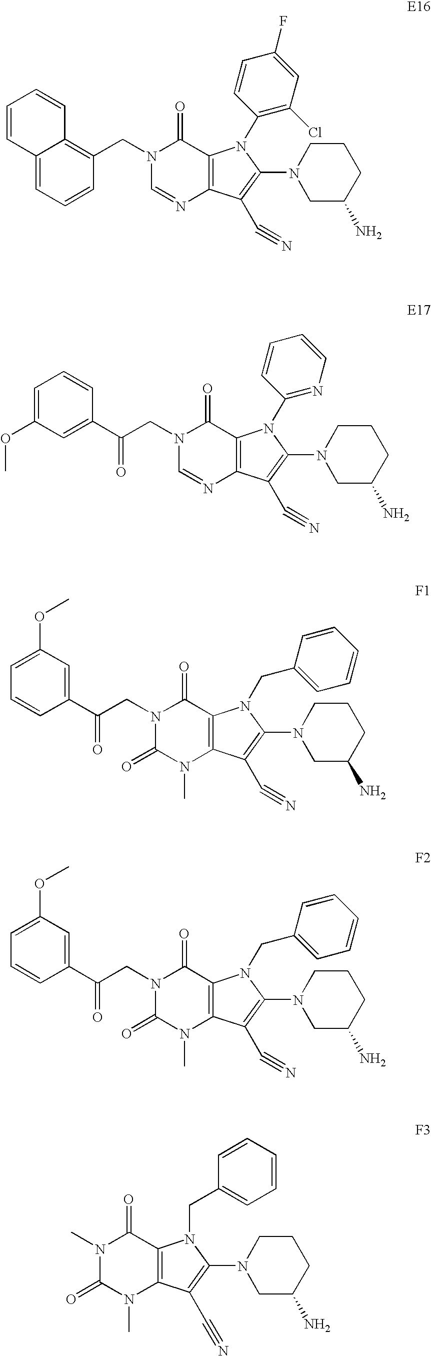

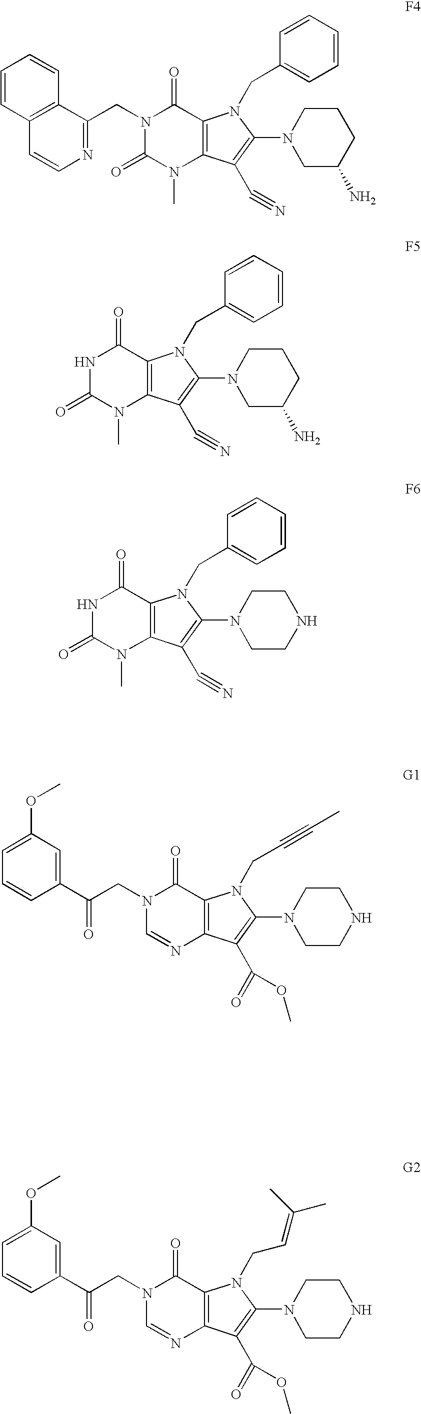

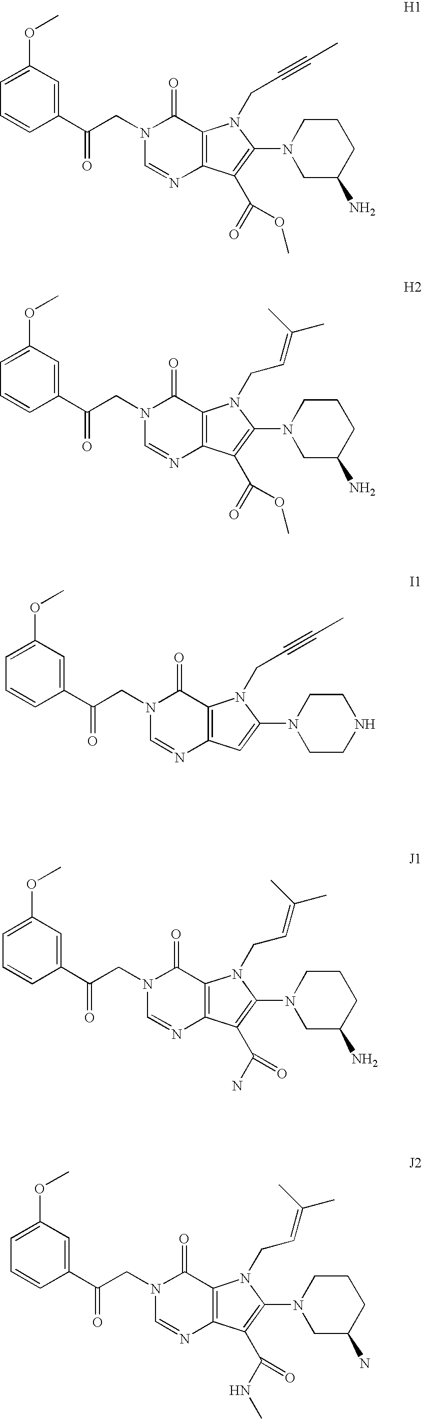

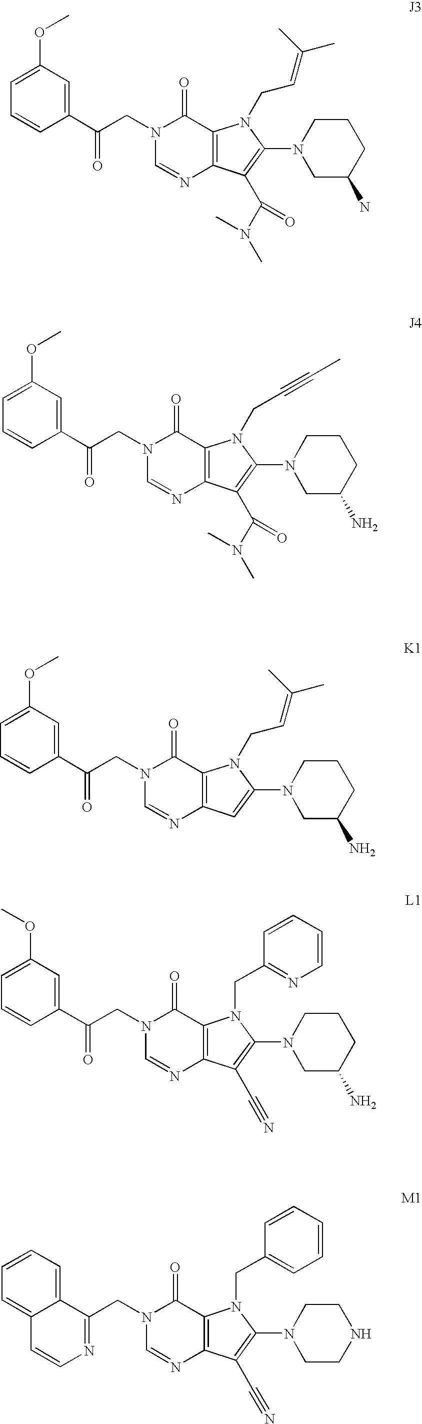

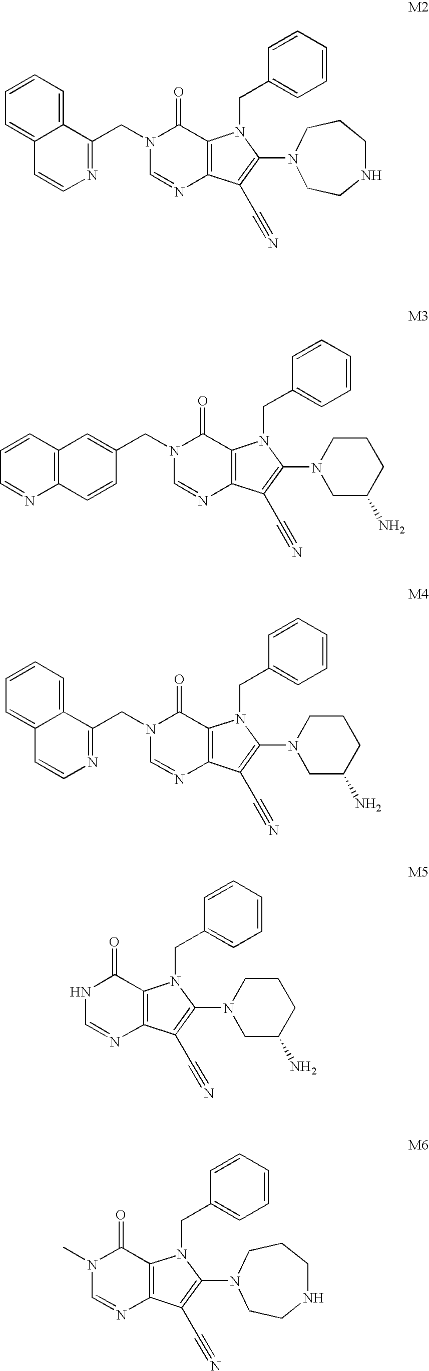

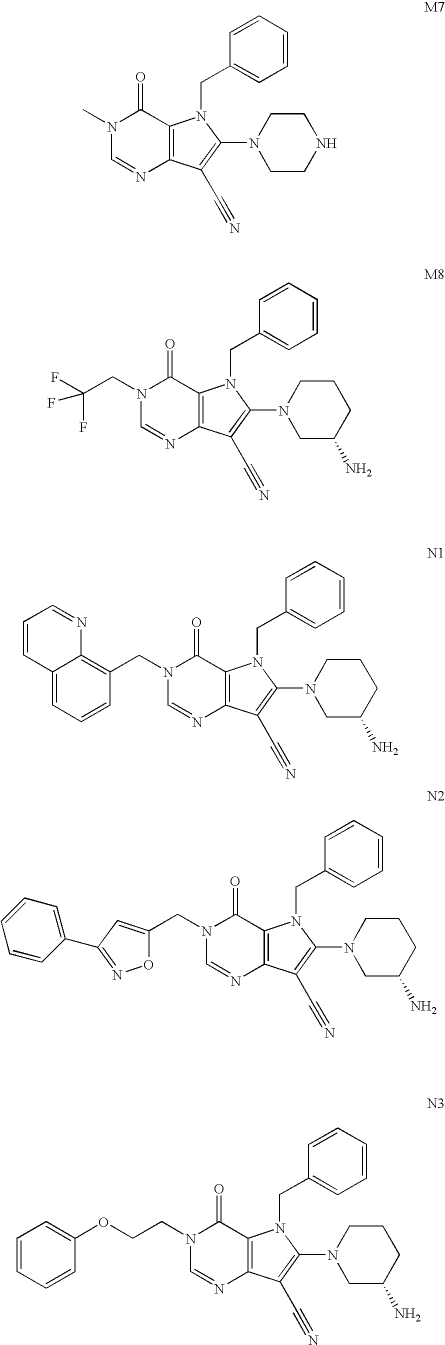

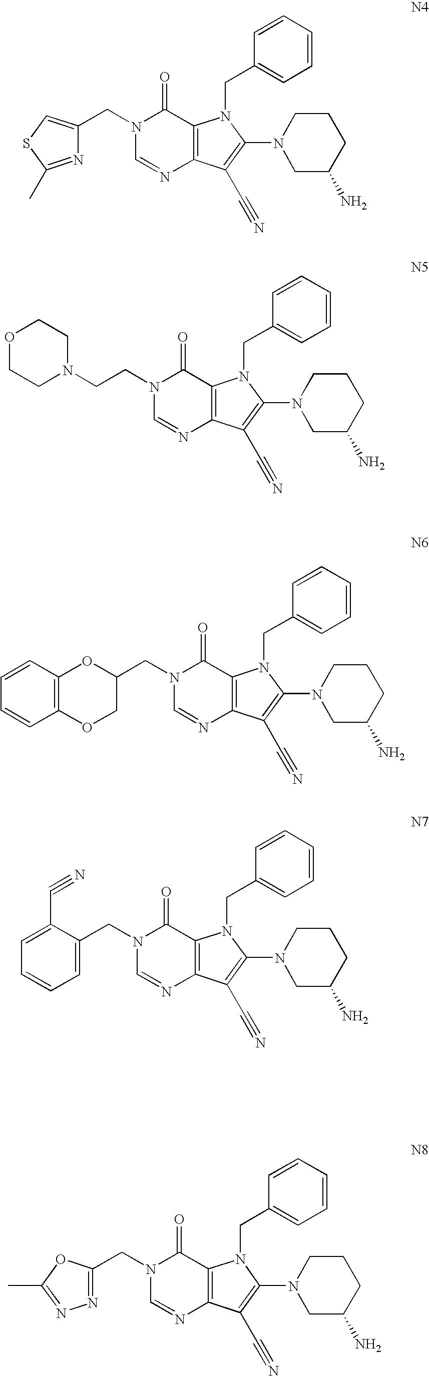

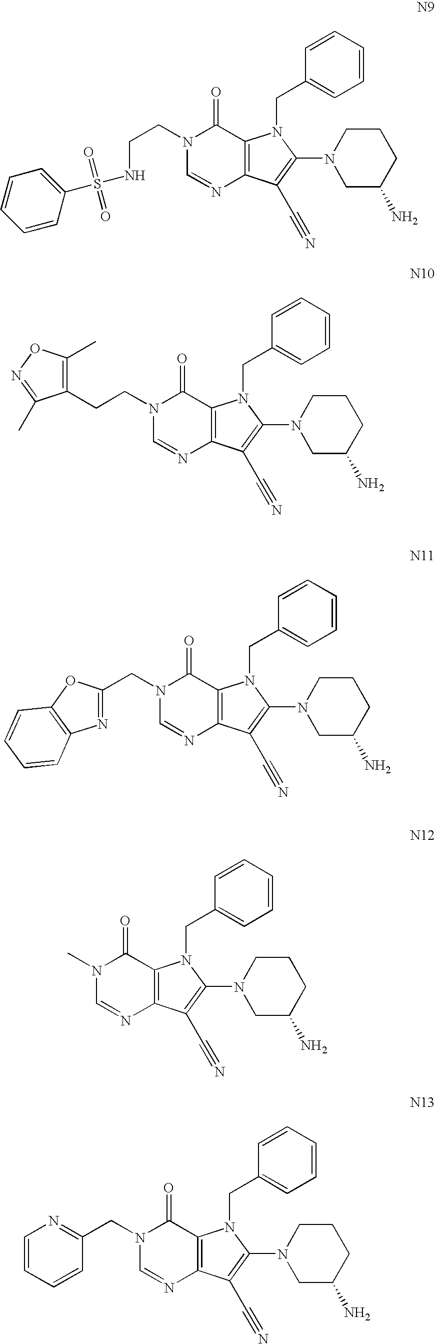

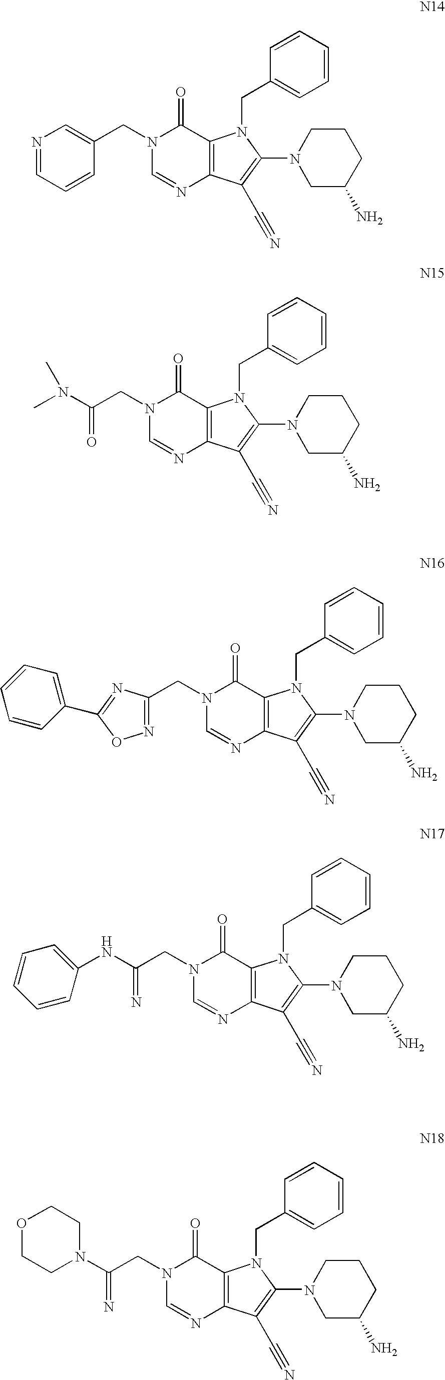

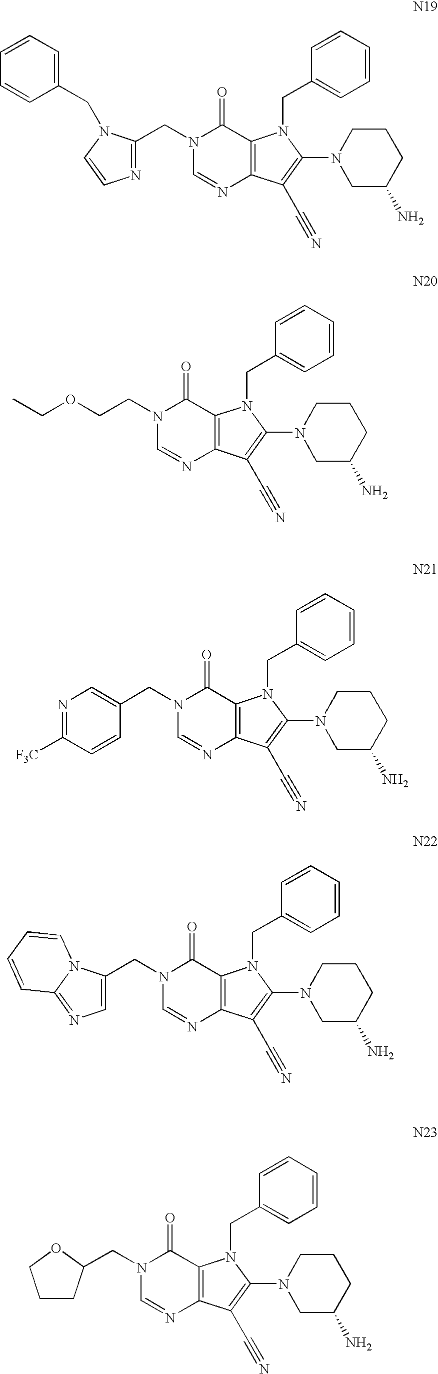

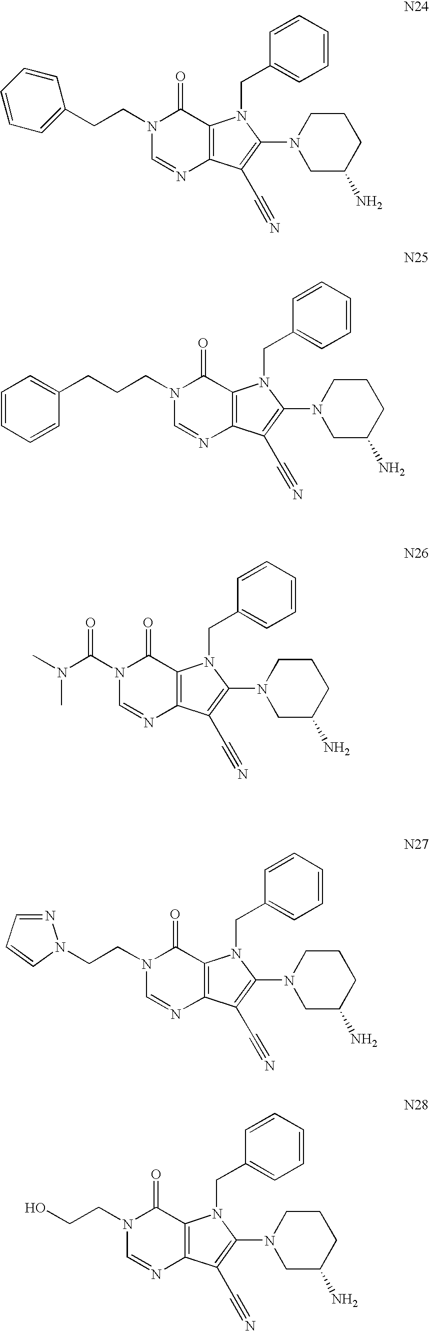

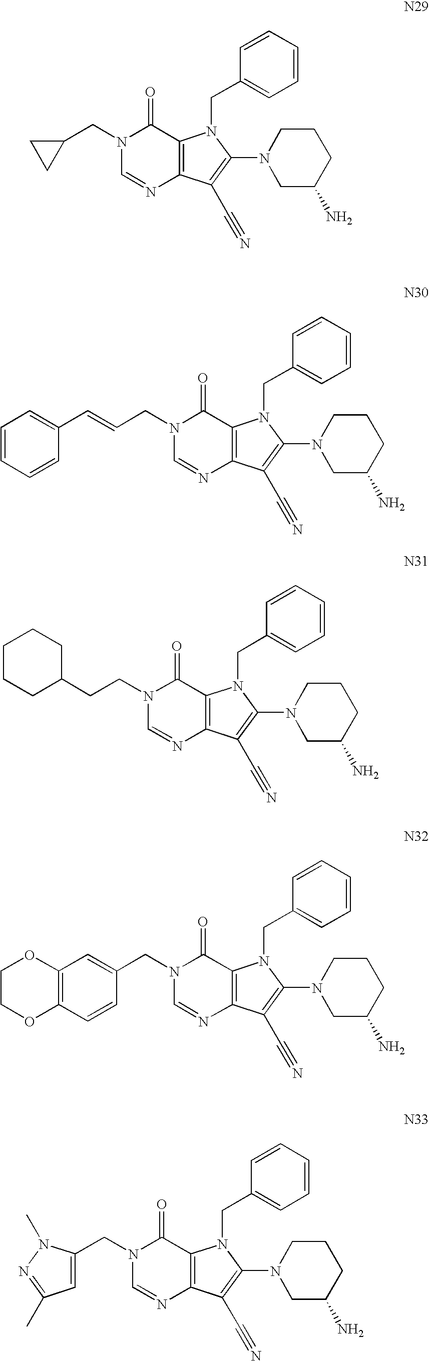

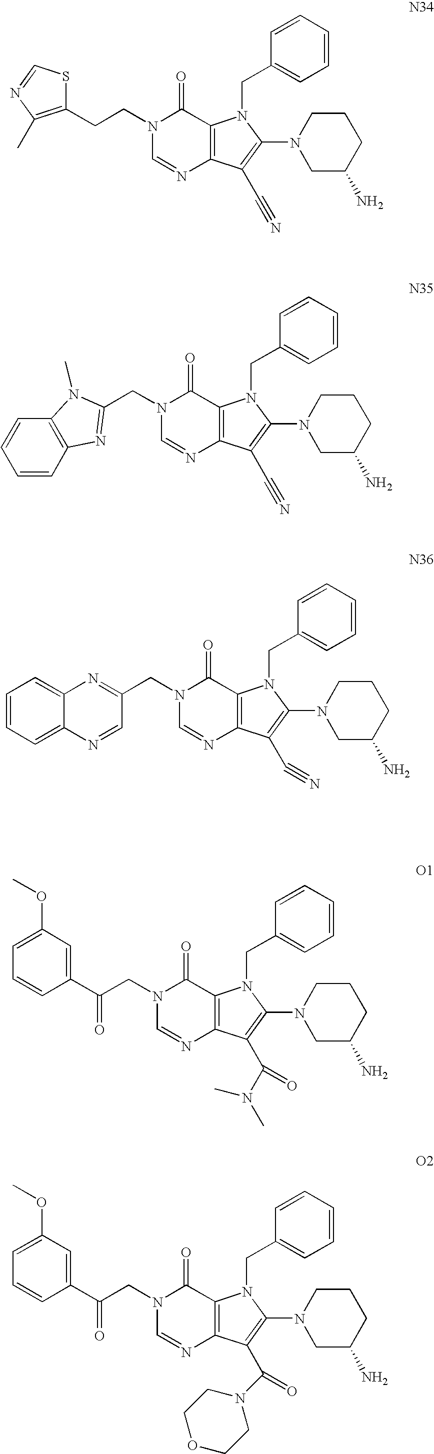

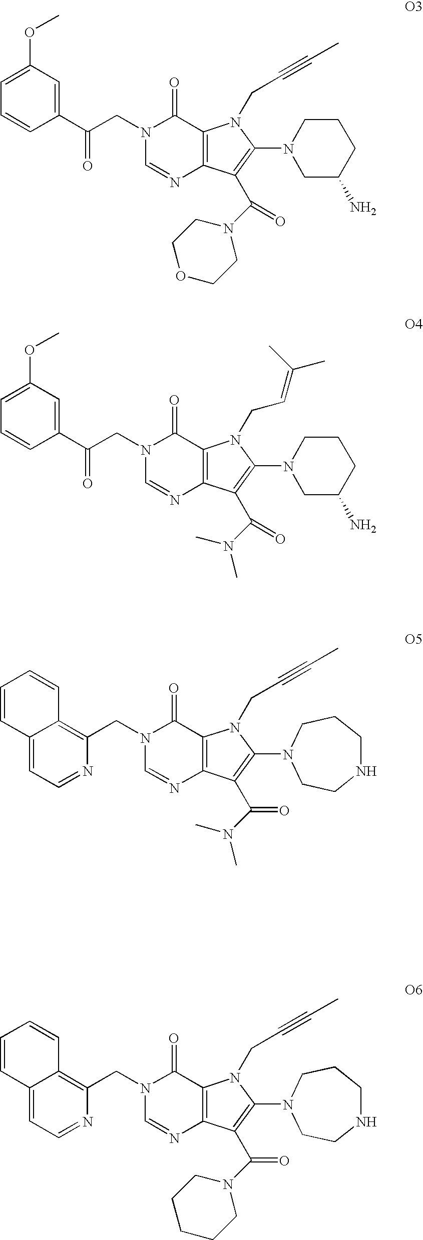

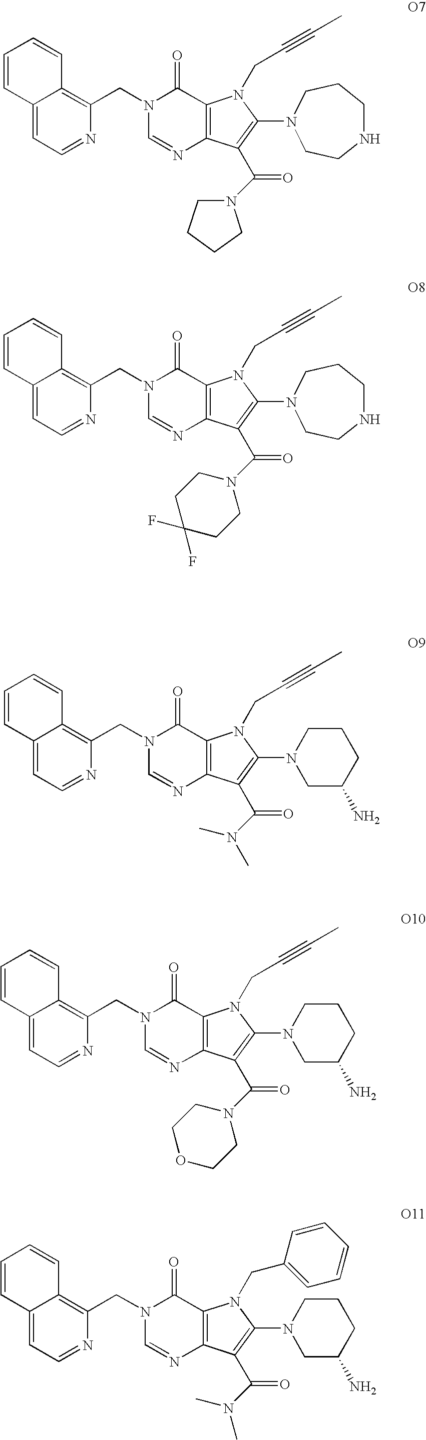

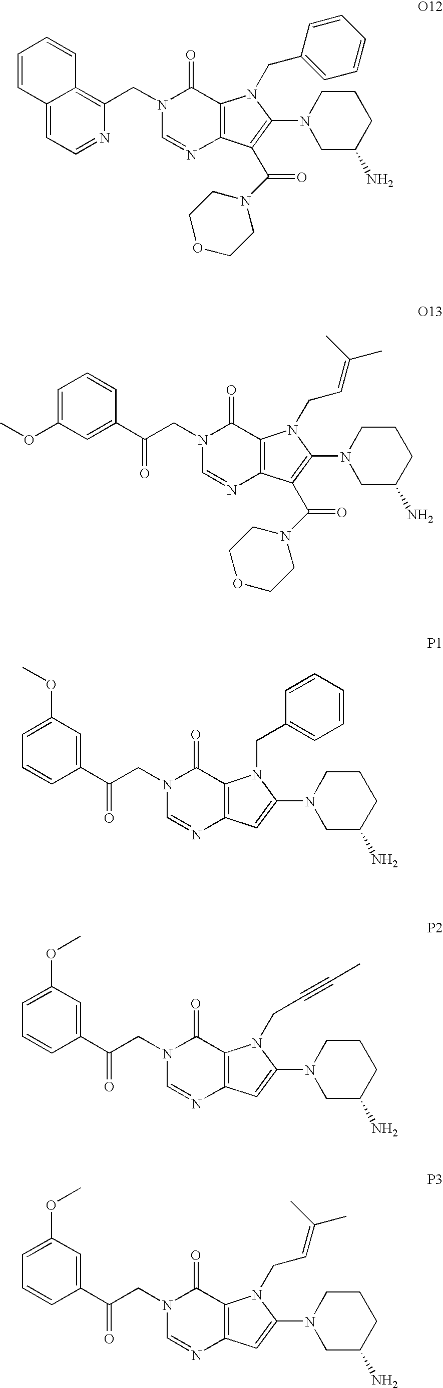

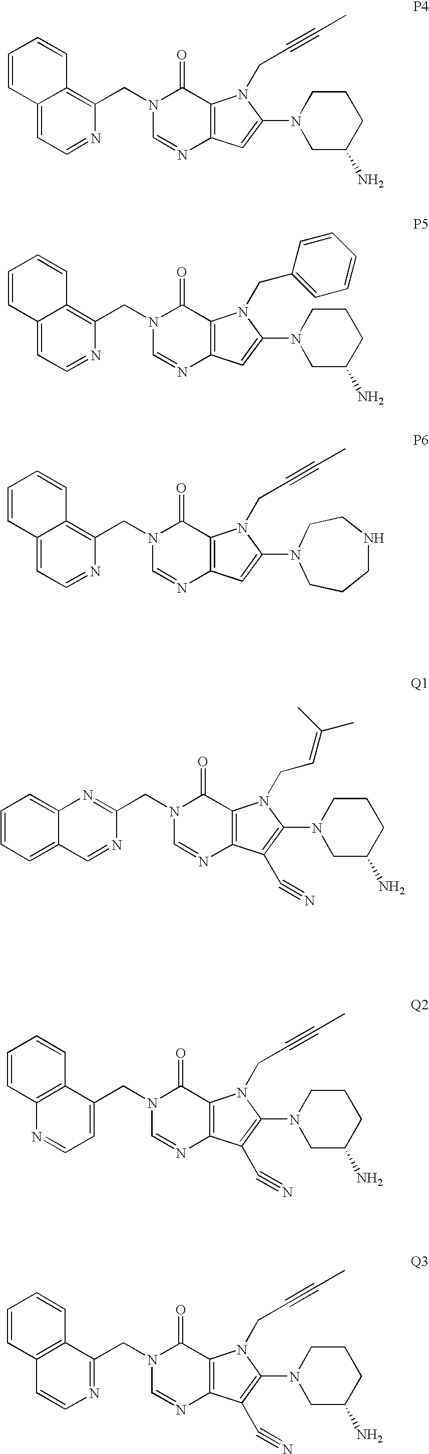

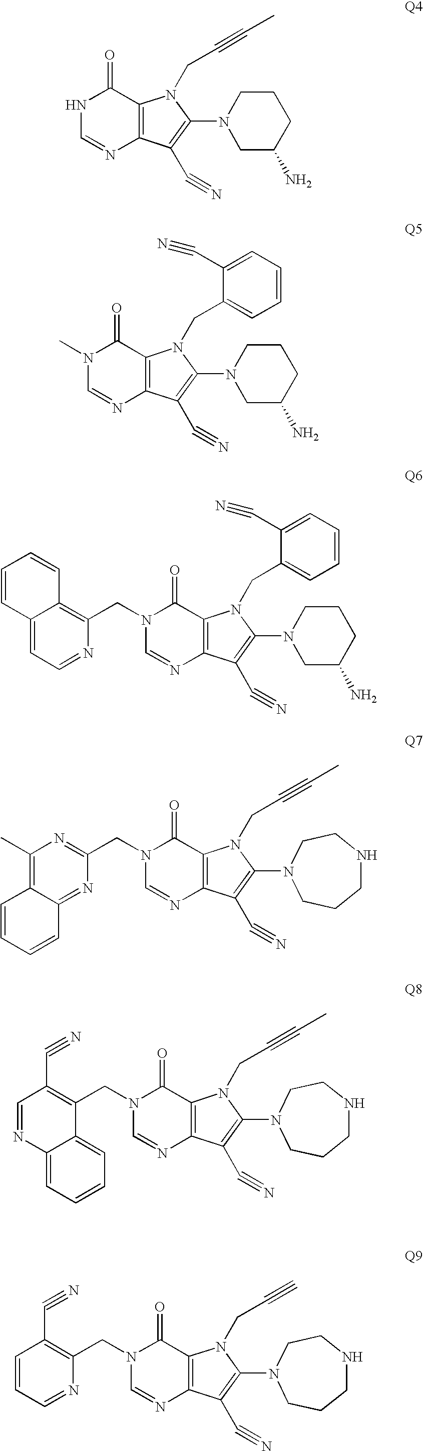

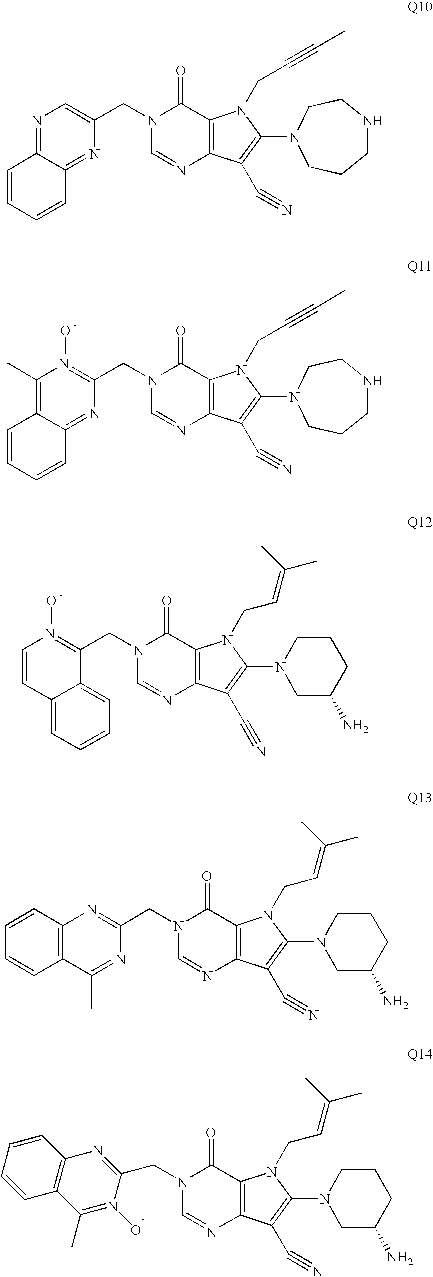

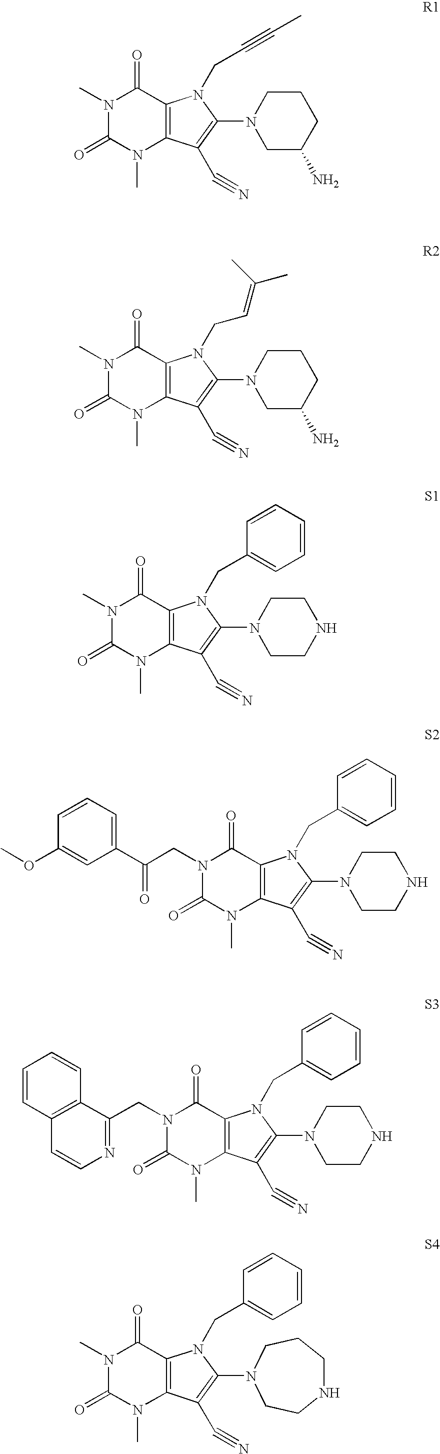

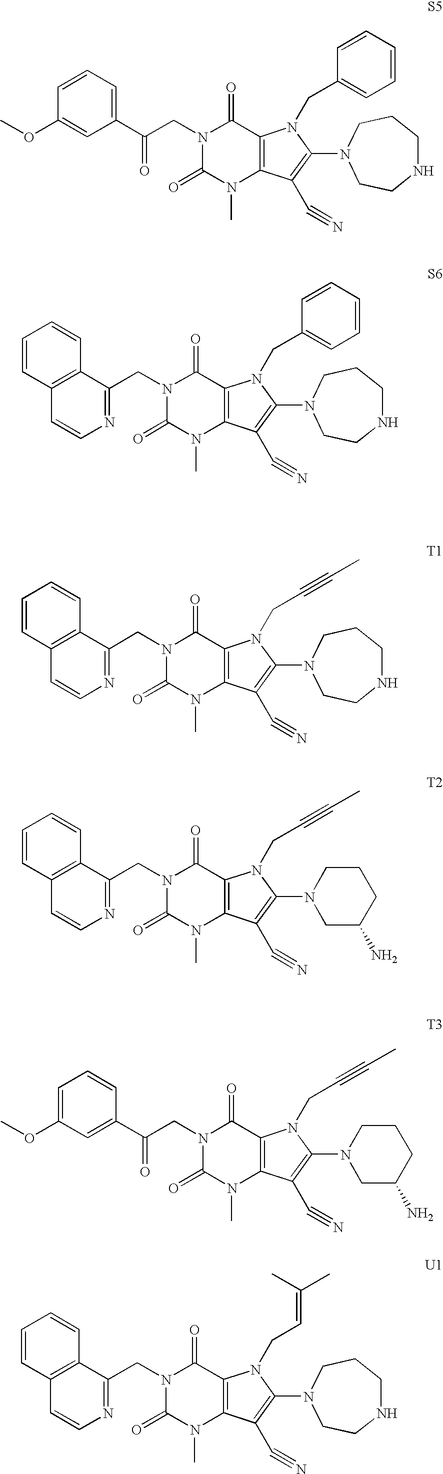

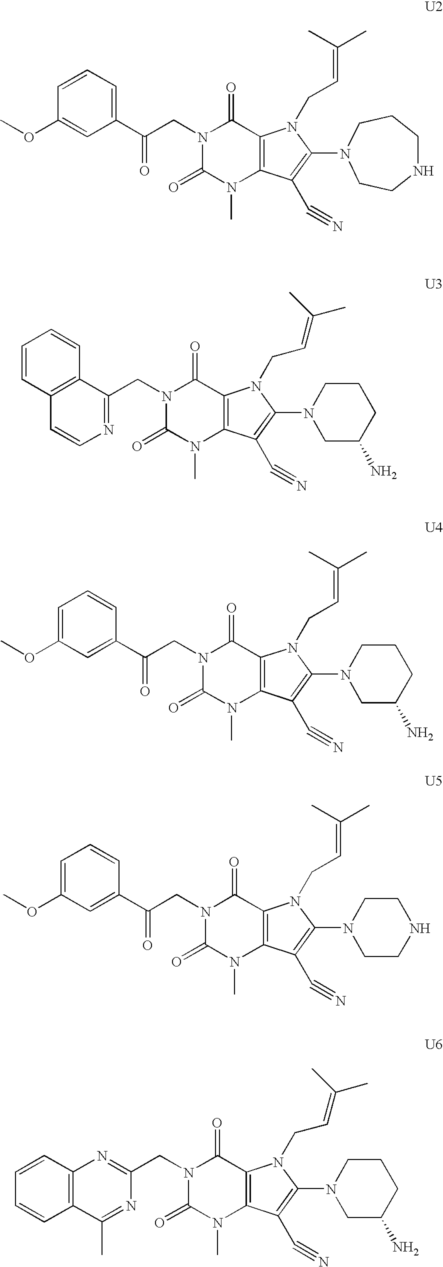

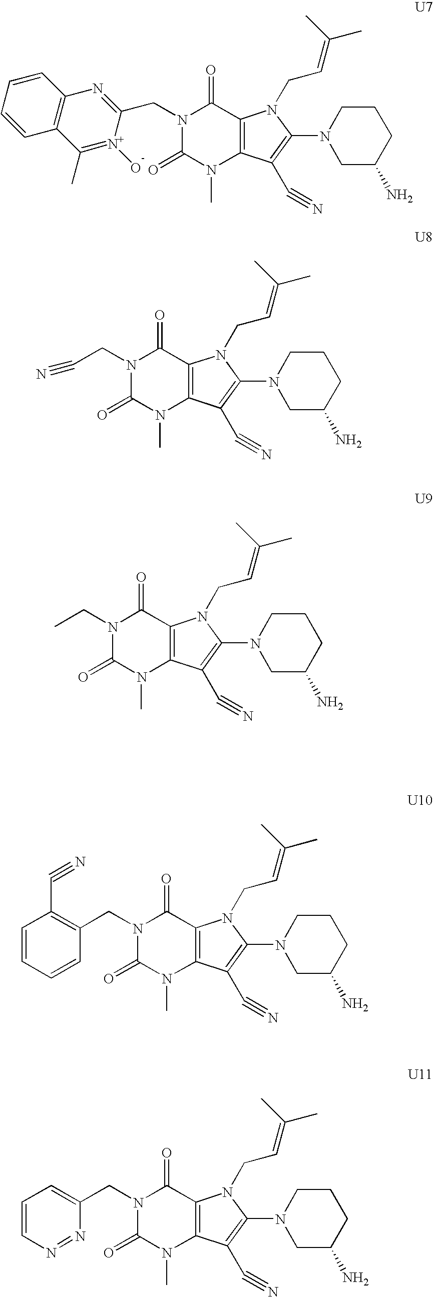

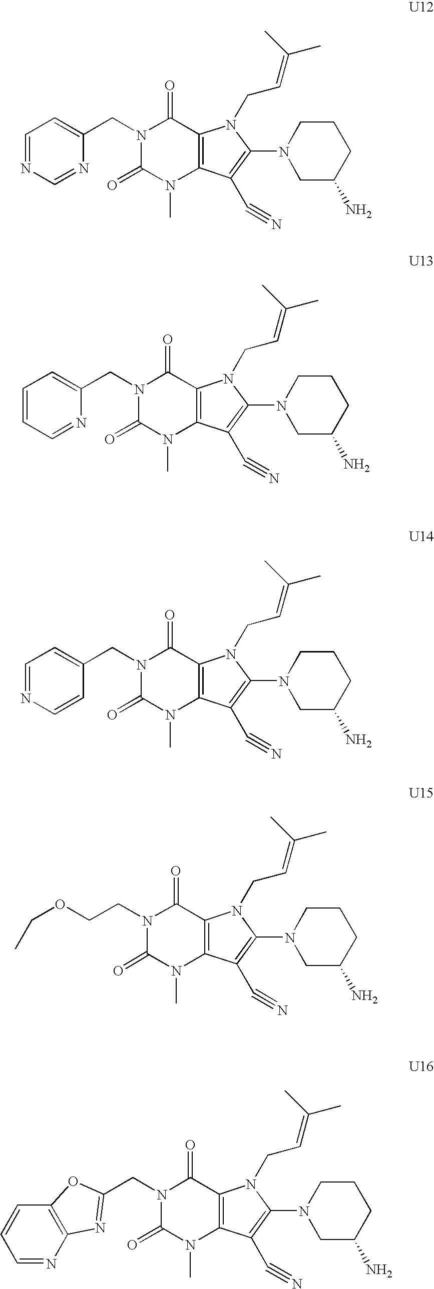

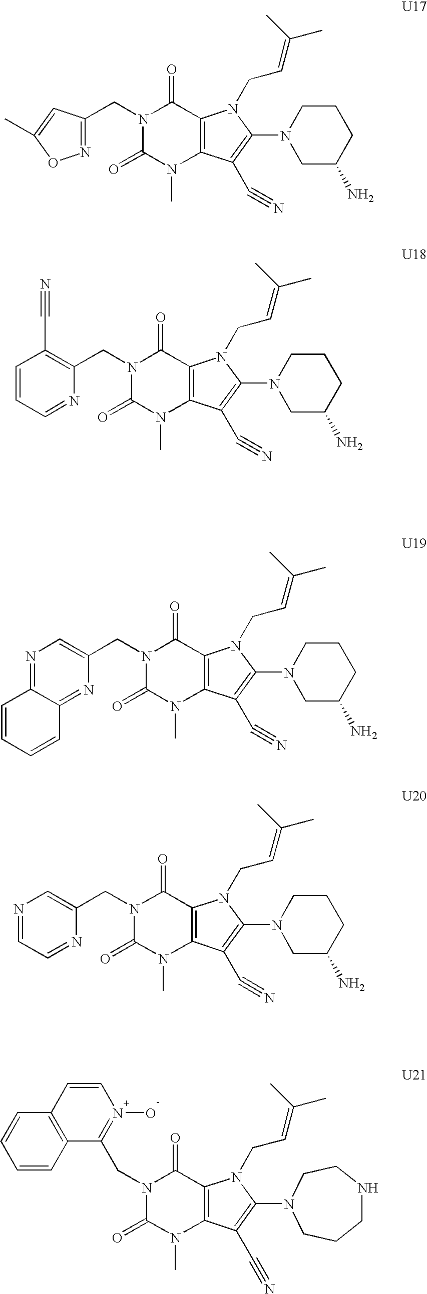

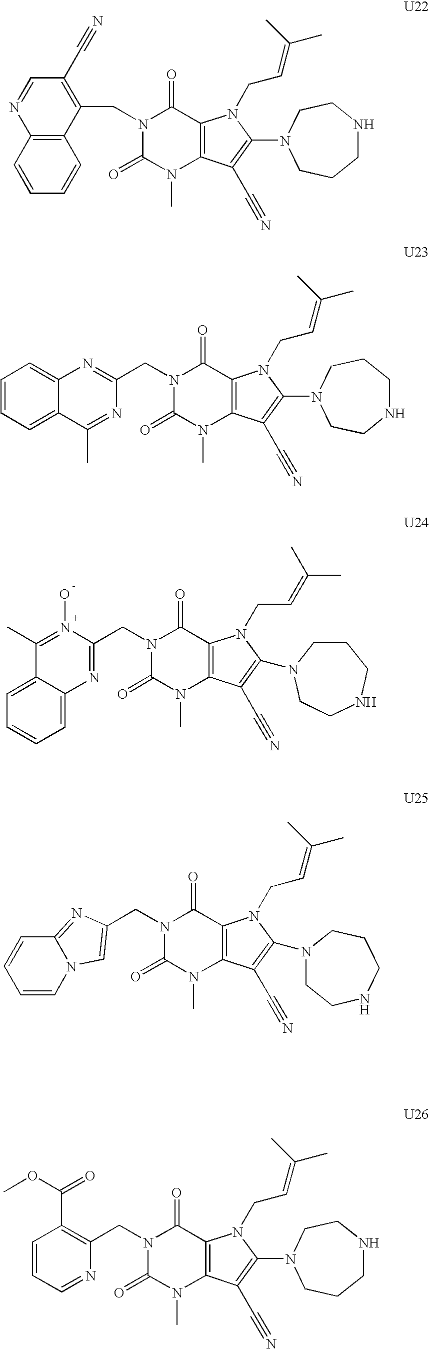

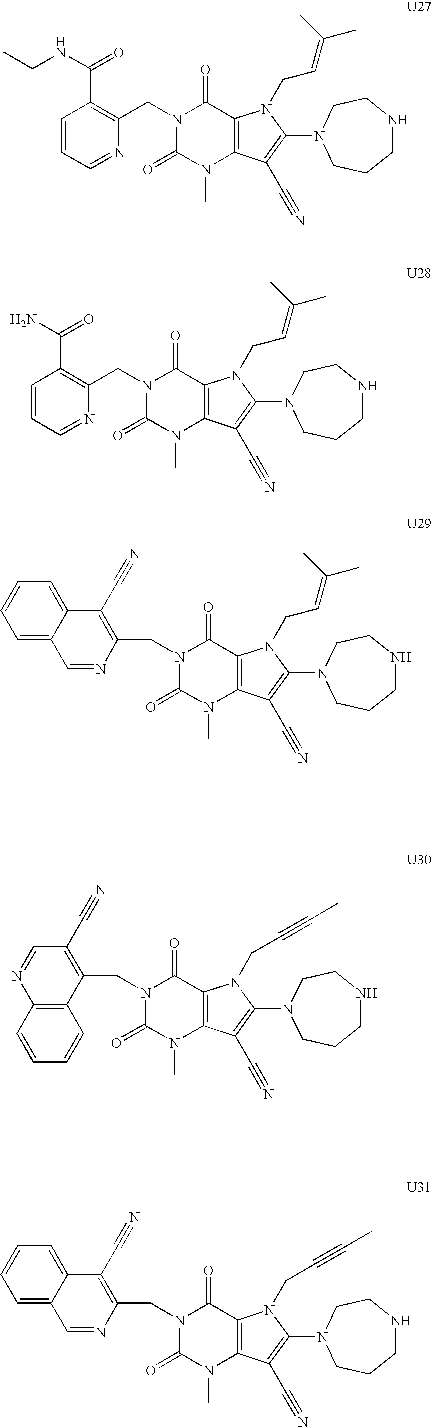

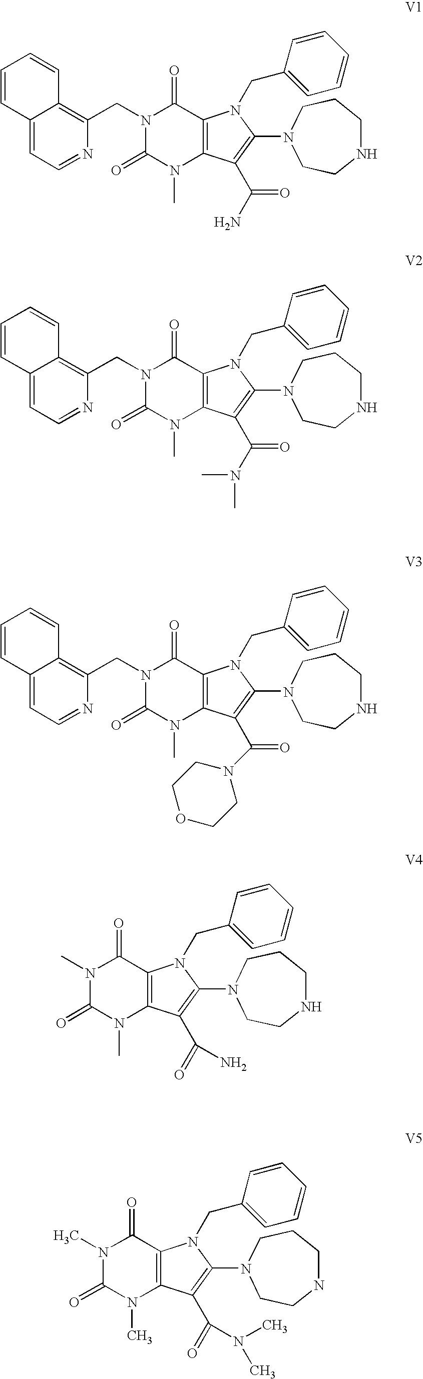

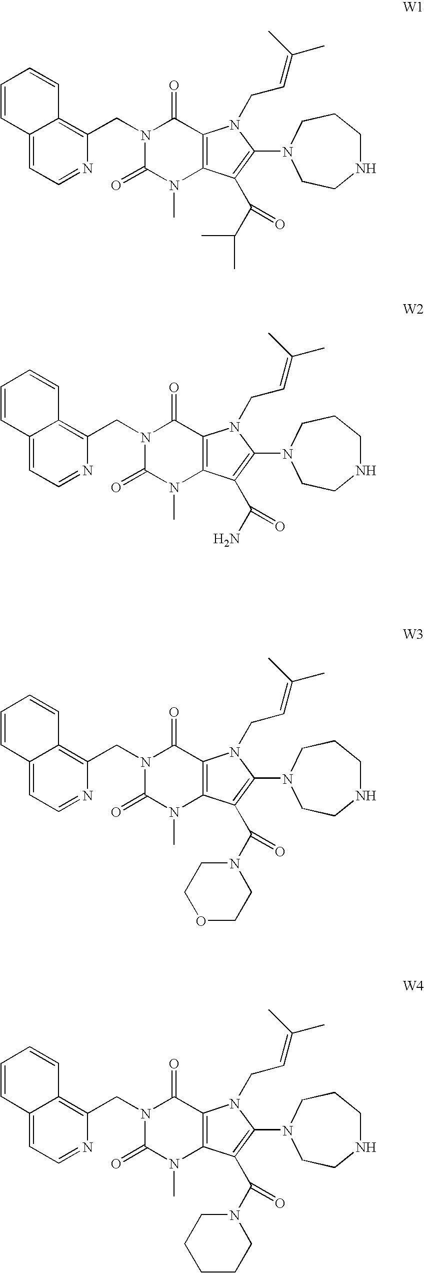

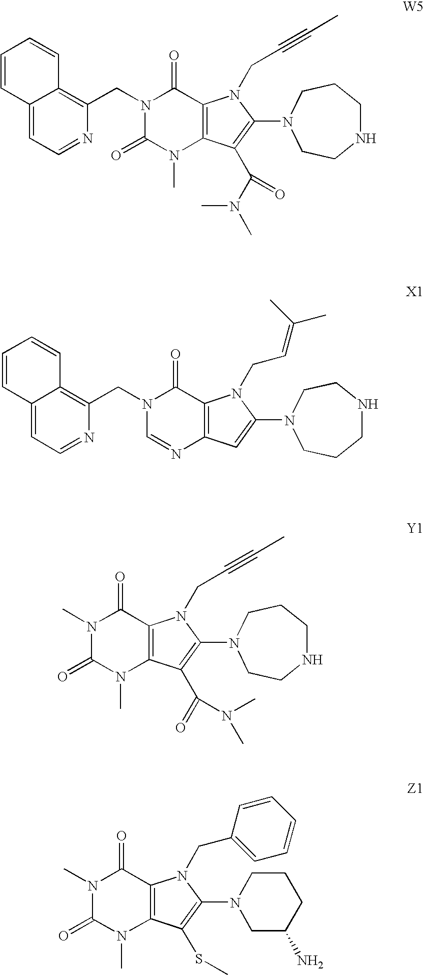

Further examples of compounds of the invention include the following compounds. Again, it will of course be appreciated that, where appropriate, each compound may be in the form of the free compound, an acid or base addition salt, or a prodrug.
5-But-2-yn-1-yl-6-(1,4-diazepan-1-yl)-3-(isoquinolin-1-ylmethyl)-1-methyl-7-(morpholin-4-ylcarbonyl)-1H-pyrrolo[3,2-d]pyrimidine-2,4(3H,5H)-dione;
5-But-2-yn-1-yl-6-(1,4-diazepan-1-yl)-3-(isoquinolin-1-ylmethyl)-1-methyl-7-(piperidin-1-ylcarbonyl)-1H-pyrrolo[3,2-d]pyrimidine-2,4(3H,5H)-dione;
5-But-2-yn-1-yl-6-(1,4-diazepan-1-yl)-1-methyl-3-[(2-oxidoisoquinolin-1-yl)methyl]-2,4-dioxo-2,3,4,5-tetrahydro-1H-pyrrolo[3,2-d]pyrimidine-7-carbonitrile;
4-{[5-But-2-yn-1-yl-7-cyano-6-(1,4-diazepan-1-yl)-1-methyl-2,4-dioxo-1,2,4,5-tetrahydro-3H-pyrrolo[3,2-d]pyrimidin-3-yl]methyl}isoquinoline-3-carbonitrile;
4-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}isoquinoline-3-carbonitrile;
5-But-2-yn-1-yl-6-(1,4-diazepan-1-yl)-1-methyl-3-[(4-methylquinazolin-2-yl)methyl]-2,4-dioxo-2,3,4,5-tetrahydro-1H-pyrrolo[3,2-d]pyrimidine-7-carbonitrile;
5-But-2-yn-1-yl-6-(1,4-diazepan-1-yl)-1-methyl-3-[(4-methyl-3-oxidoquinazolin-2-yl)methyl]-2,4-dioxo-2,3,4,5-tetrahydro-1H-pyrrolo[3,2-d]pyrimidine-7-carbonitrile; and
5-But-2-yn-1-yl-6-(1,4-diazepan-1-yl)-3-(imidazo[1,2-a]pyridin-2-ylmethyl)-1-methyl-2,4-dioxo-2,3,4,5-tetrahydro-1H-pyrrolo[3,2-d]pyrimidine-7-carbonitrile;
   the corresponding structures of which are shown below, respectively (ordered left to right):

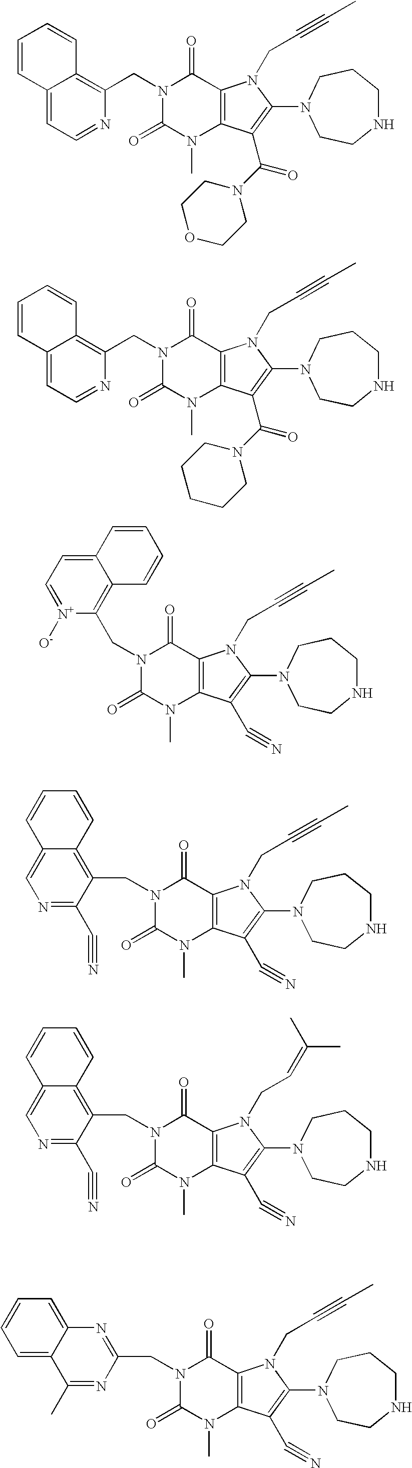

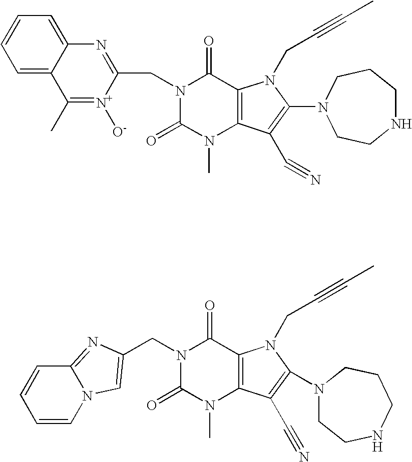

The compounds of the invention may be in the form of pharmaceutically acceptable salts. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The disclosure thus includes pharmaceutically-acceptable salts of the disclosed compounds wherein the parent compound is modified by making acid or base salts thereof. for example the conventional non-toxic salts or the quaternary ammonium salts which are formed, e.g. from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The invention includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:

Oxidative Activation
N- and O-dealkylation
Oxidative deamination
N-oxidation
Epoxidation

Reductive Activation
Azo reduction
Sulfoxide reduction
Disulfide reduction
Bioreductive alkylation
Nitro reduction.

Also to be mentioned as metabolic activations of prodrugs are nucleotide activation, phosphorylation activation and decarboxylation activation. For additional information, see “The Organic Chemistry of Drug Design and Drug Action”, R B Silverman (particularly Chapter 8, pages 497 to 546), incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, although protected derivatives of compounds of the disclosure may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the disclosure.

Many of the groups referred to or featured herein (especially those containing heteroatoms and conjugated bonds) can exist in tautomeric forms and all these tautomers are included in the scope of the disclosure. More generally, many species may exist in equilibrium, as for example in the case of organic acids and their counterpart anions; a reference herein to a species accordingly includes reference to all equilibrium forms thereof.

The compounds of the disclosure may also contain one or more asymmetric carbon atoms and may therefore exhibit optical and/or diastereoisomerism. All diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the disclosure. For example, compounds in which the carbocycle or heterocycle of R⁵ comprises an amino substituent may be present in (R) or (S) form. Where a single enantiomer or diastereomer is disclosed, the disclosure also covers the other enantiomers or diastereomers, and also racemates; in this regard, particular reference is made to the specific compounds listed herein.

Geometric isomers may also exist in the compounds of the present disclosure. The present disclosure contemplates the various geometric isomers and mixtures thereof resulting from the arrangement of substituents around a carbon-carbon double bond and designates such isomers as of the Z or E configuration, wherein the term “Z” represents substituents on the same side of the carbon-carbon double bond and the term “E” represents substituents on opposite sides of the carbon-carbon double bond.

The disclosure therefore includes all variant forms of the defined compounds, for example any tautomer or any pharmaceutically acceptable salt, ester, acid or other variant of the defined compounds and their tautomers as well as substances which, upon administration, are capable of providing directly or indirectly a compound as defined above or providing a species which is capable of existing in equilibrium with such a compound.

### Synthesis

By way of illustration, a compound of the invention may be prepared by one of the following reaction schemes:

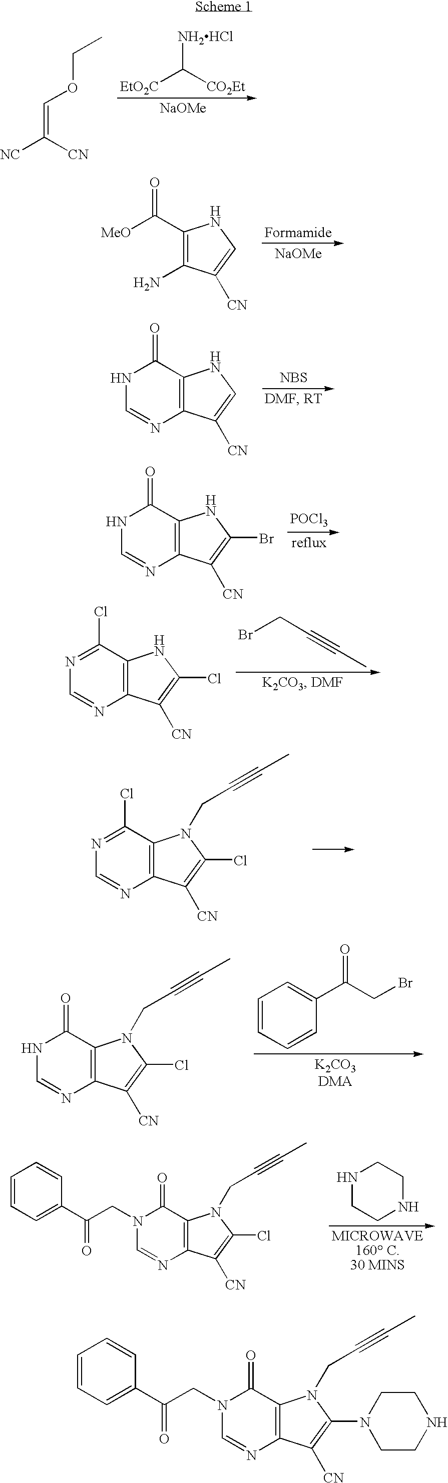

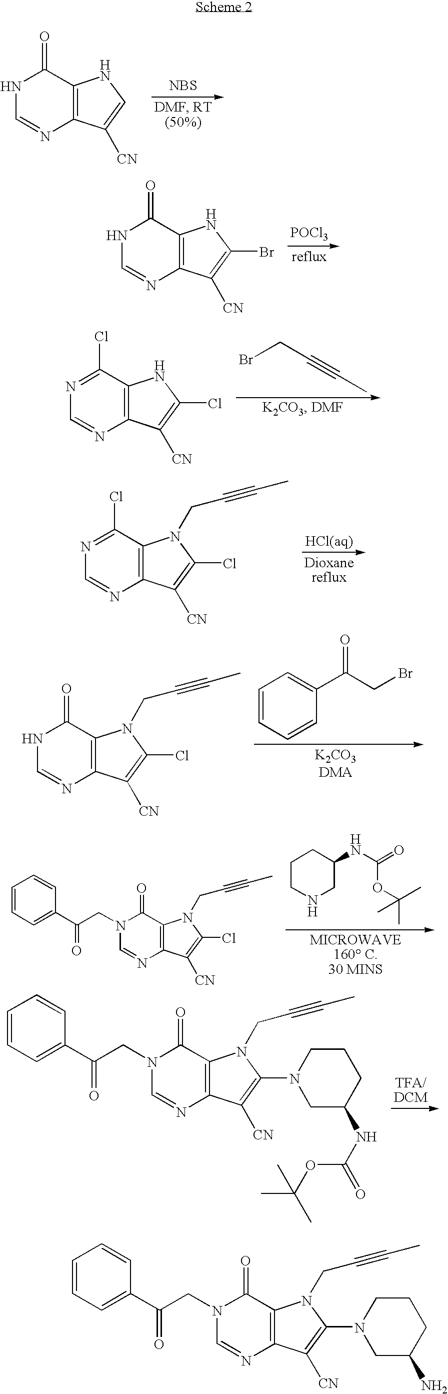

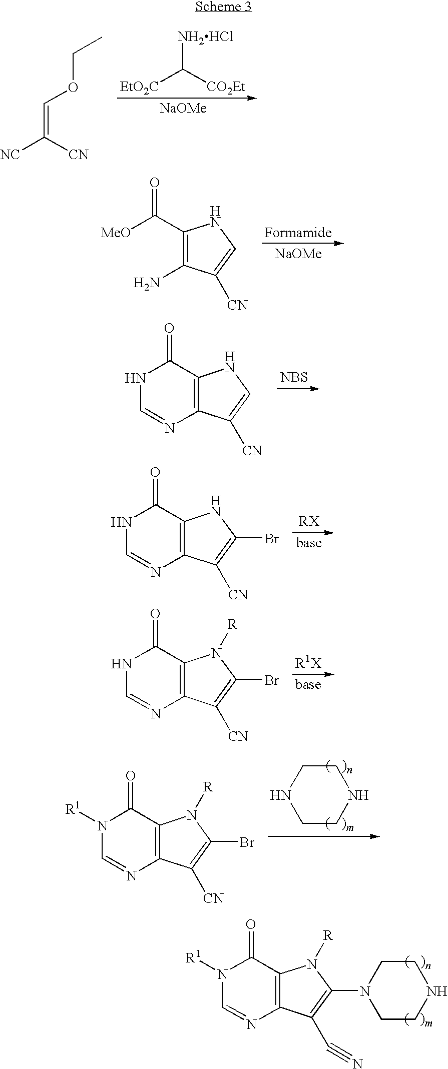

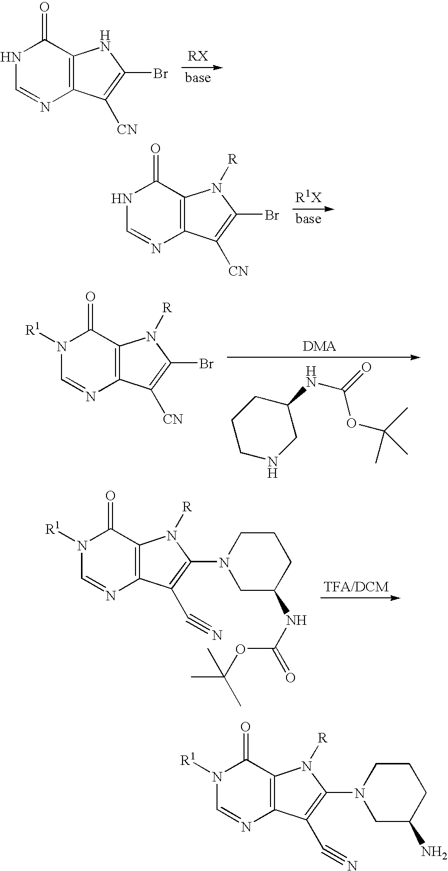

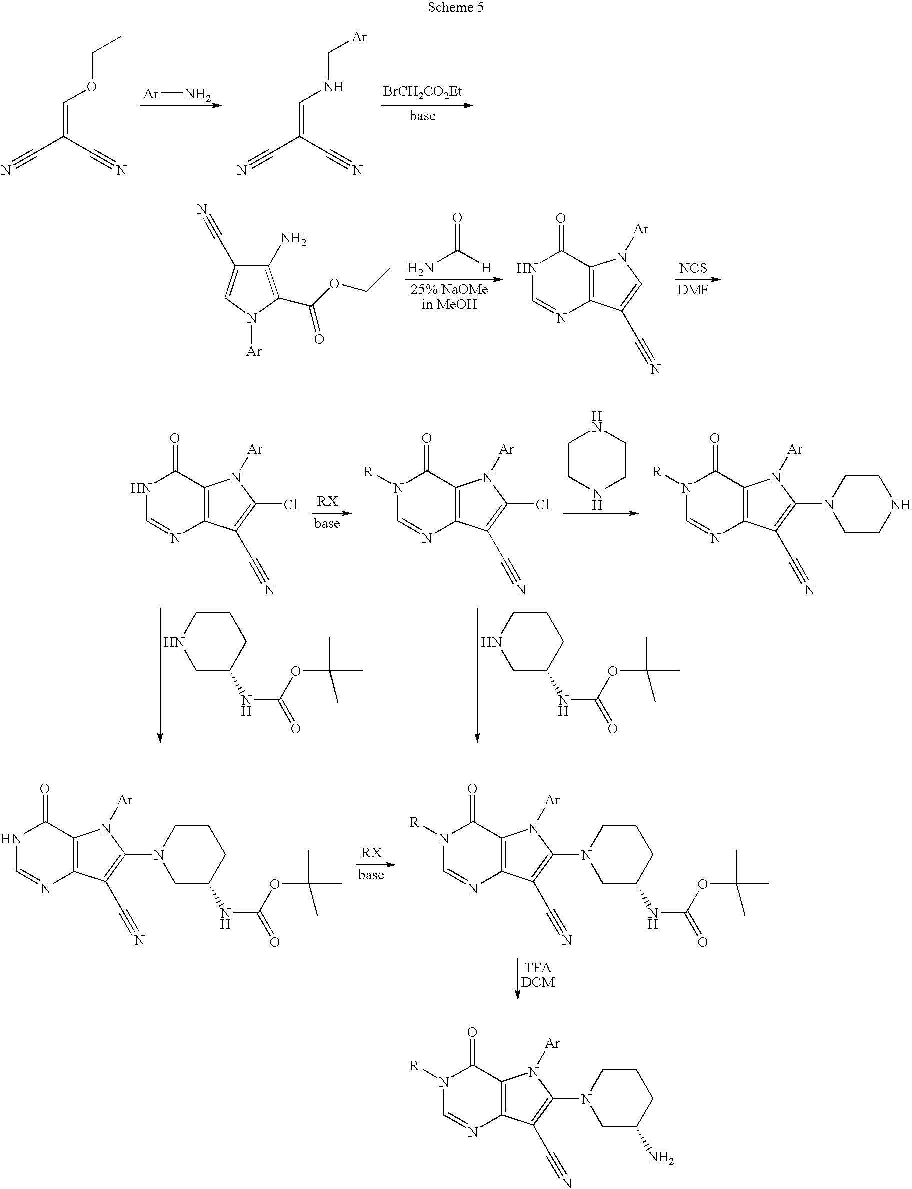

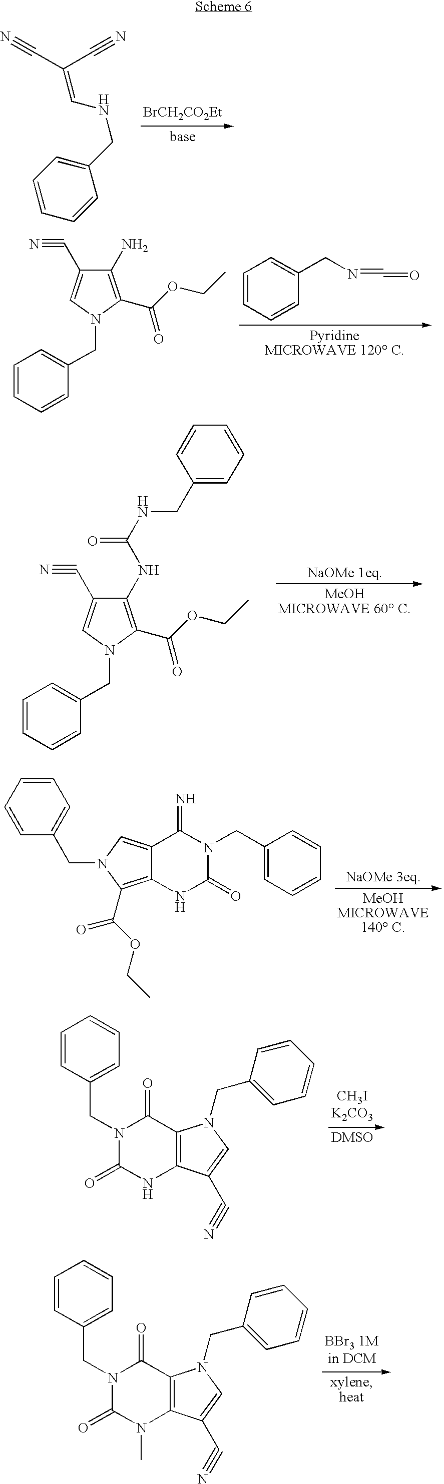

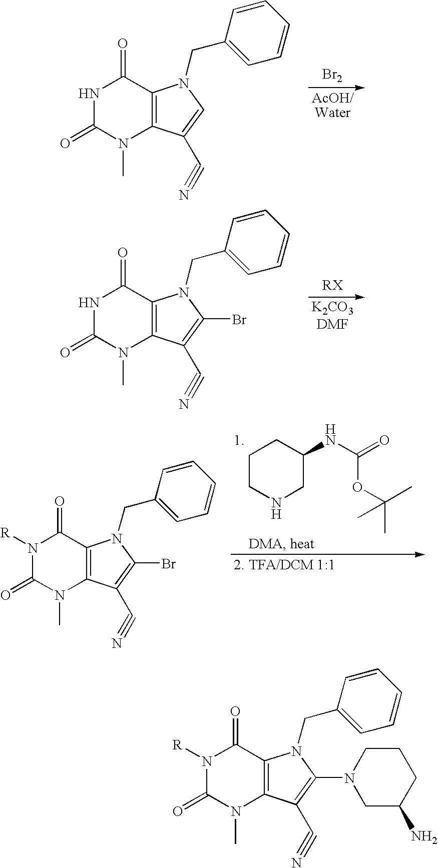

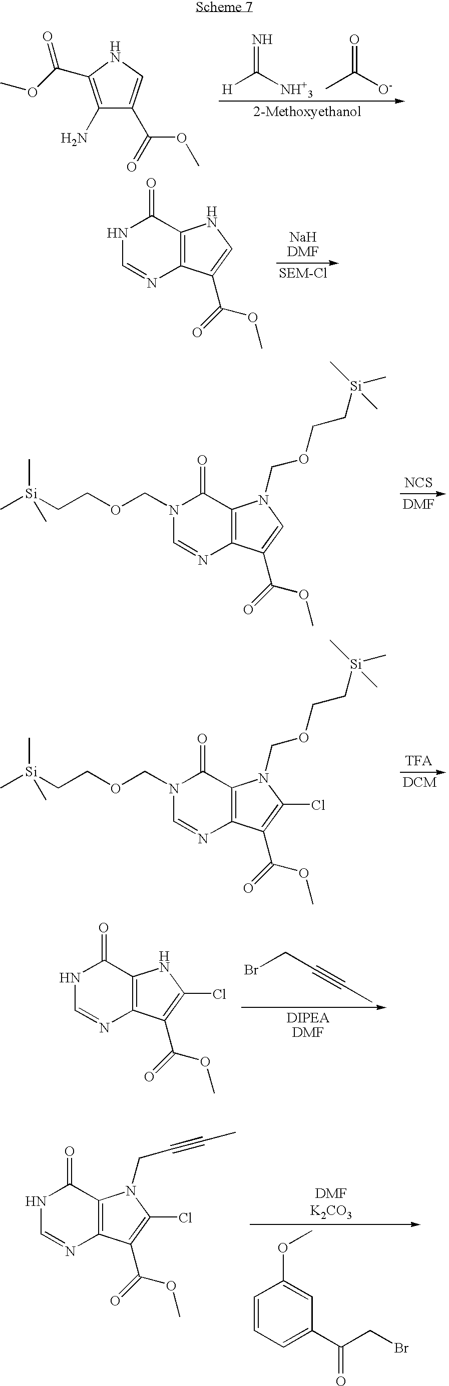

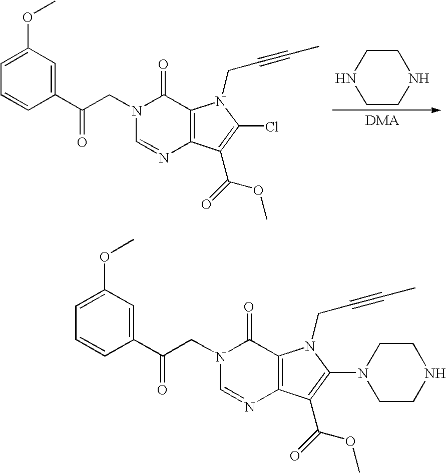

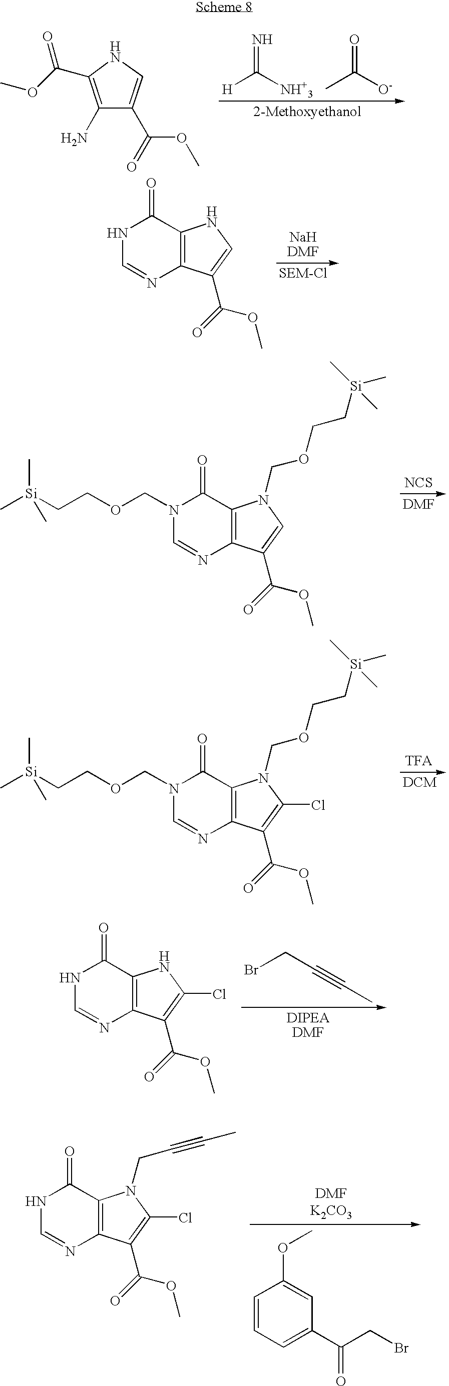

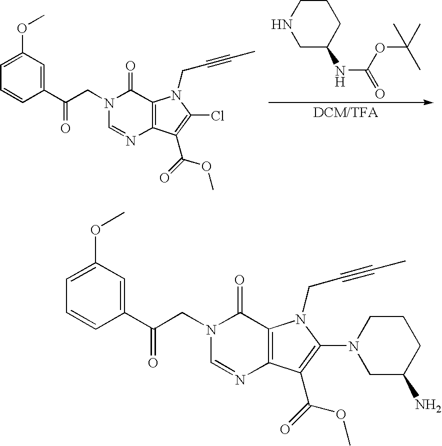

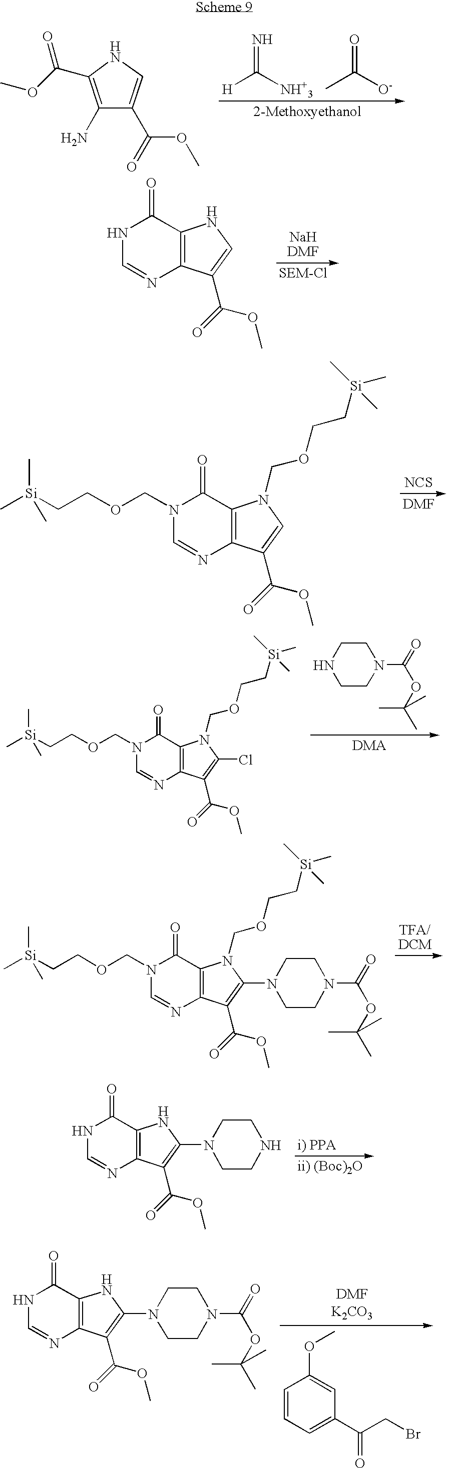

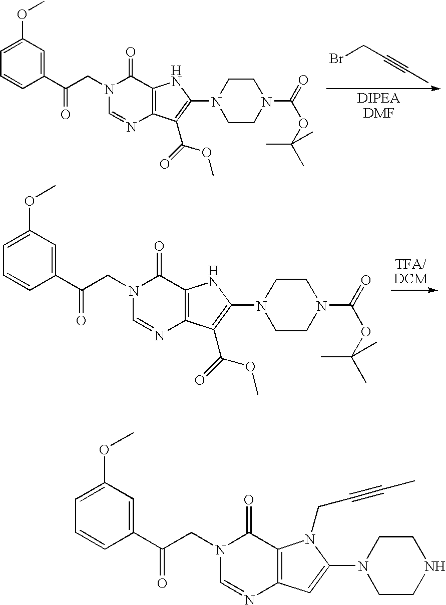

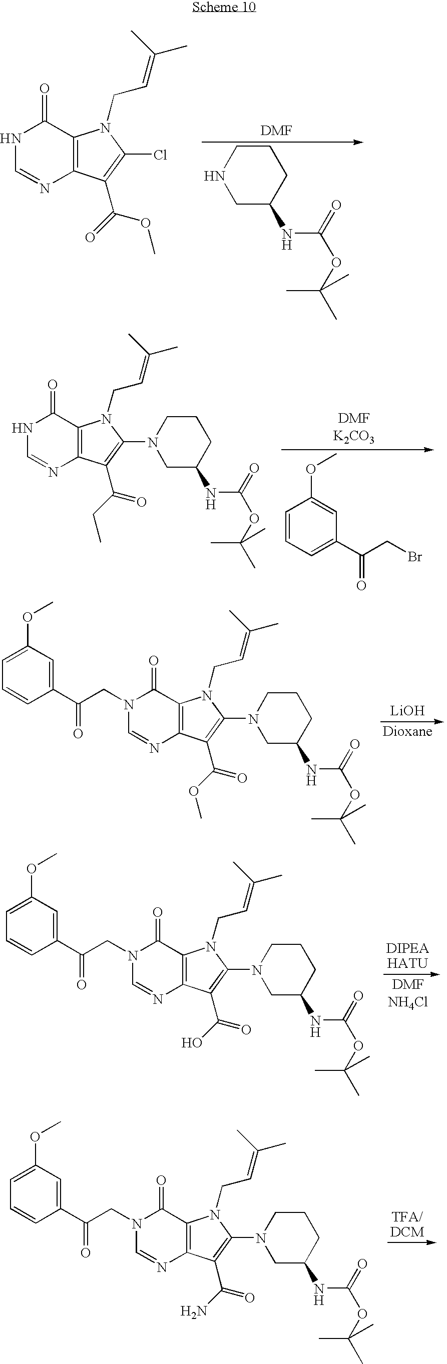

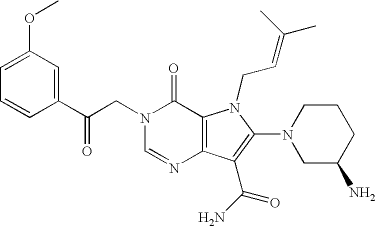

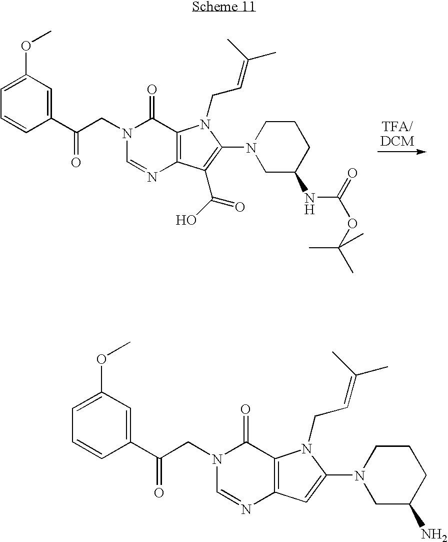

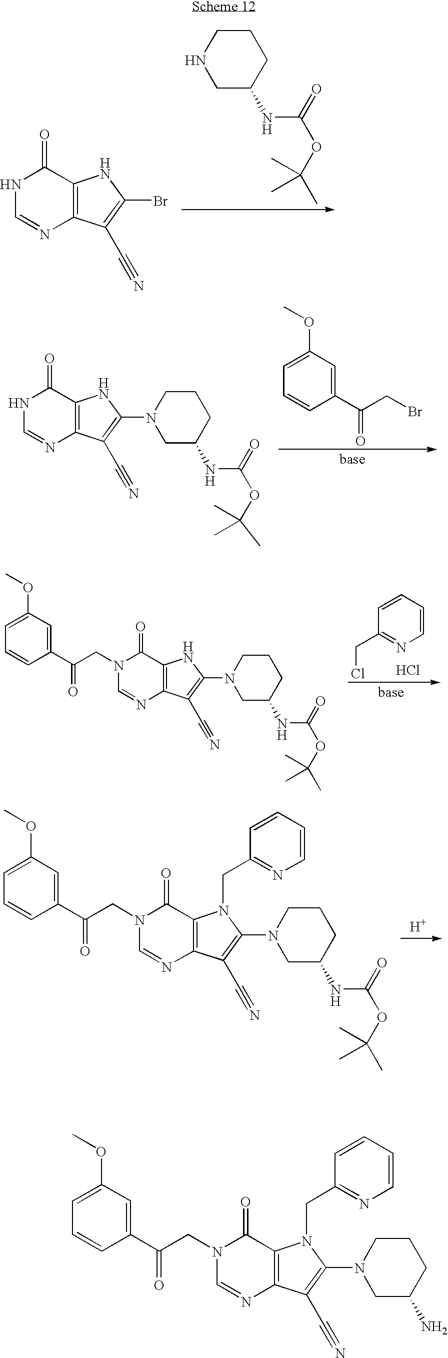

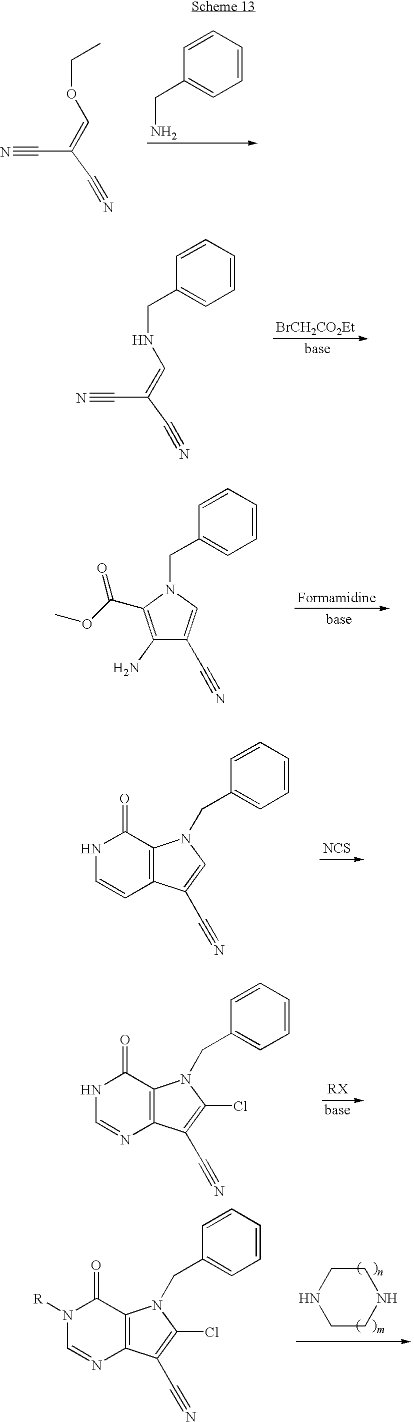

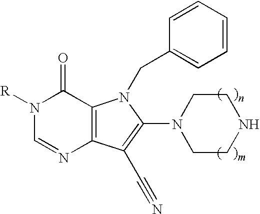

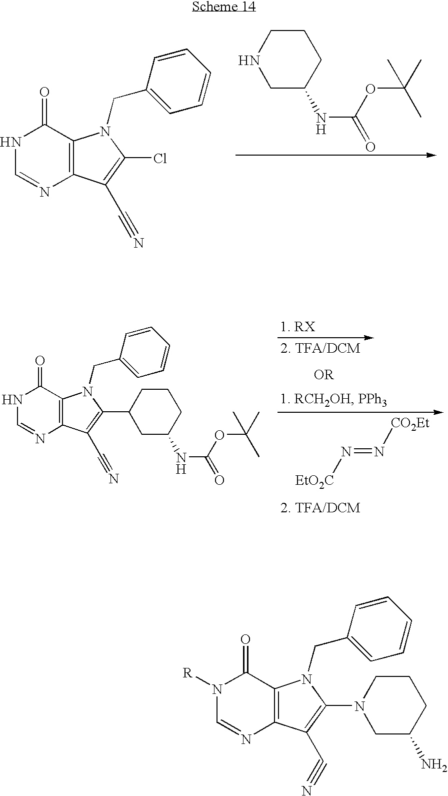

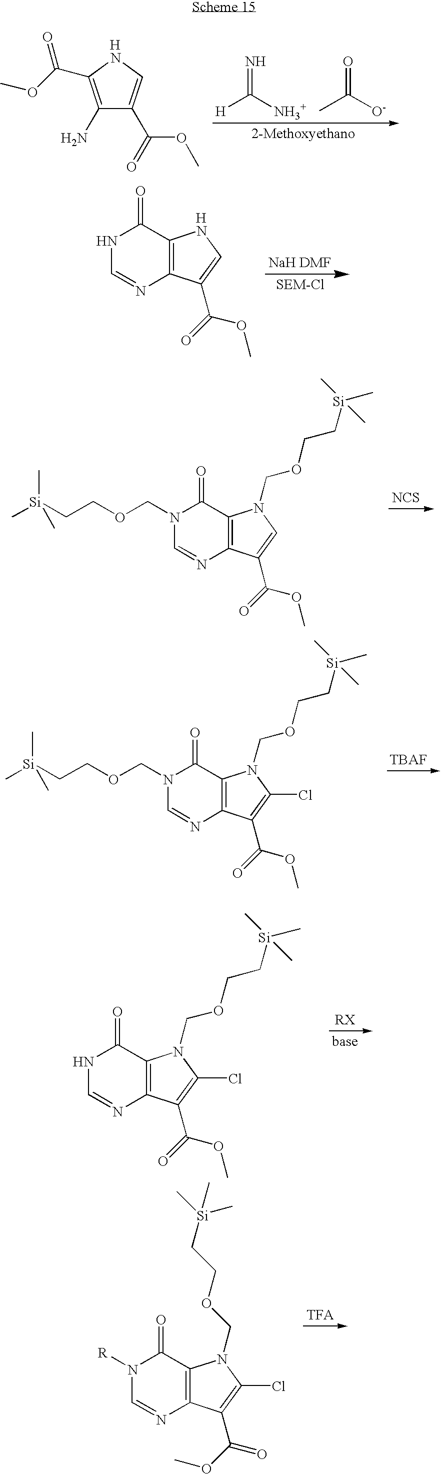

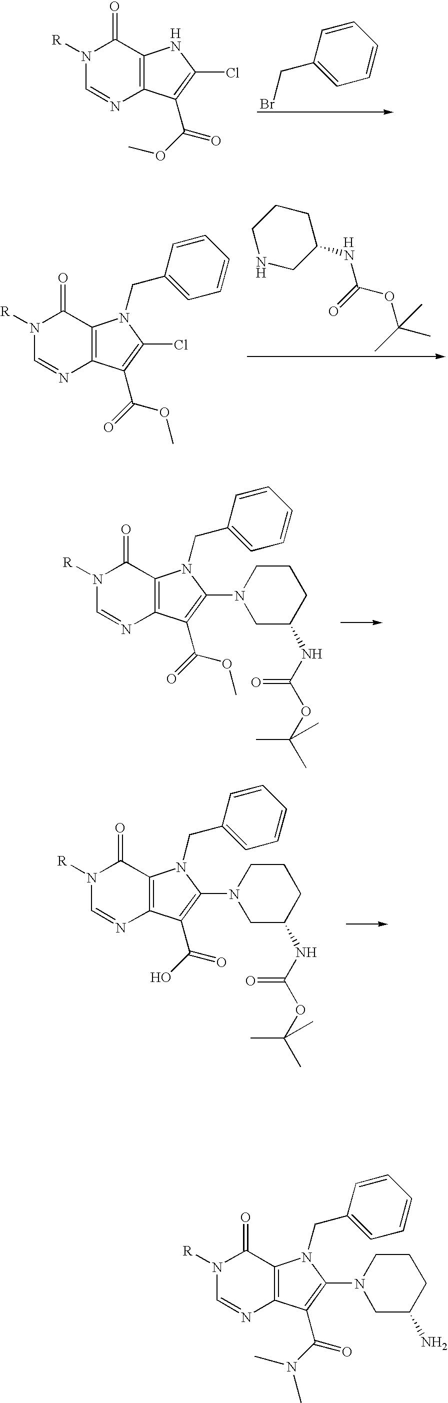

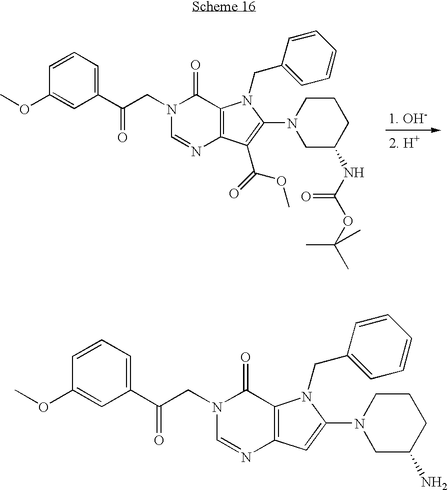

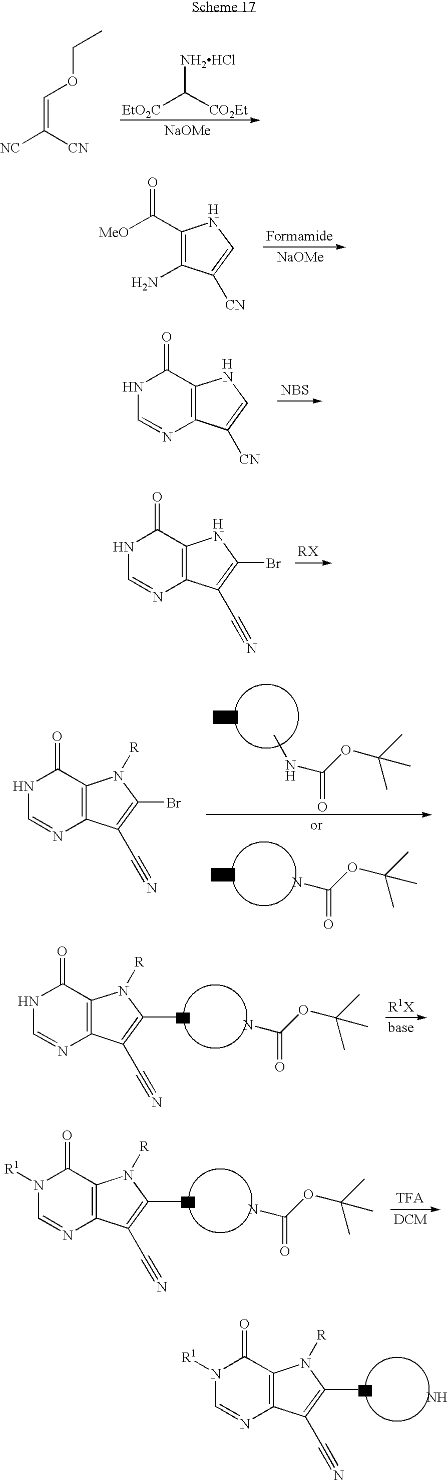

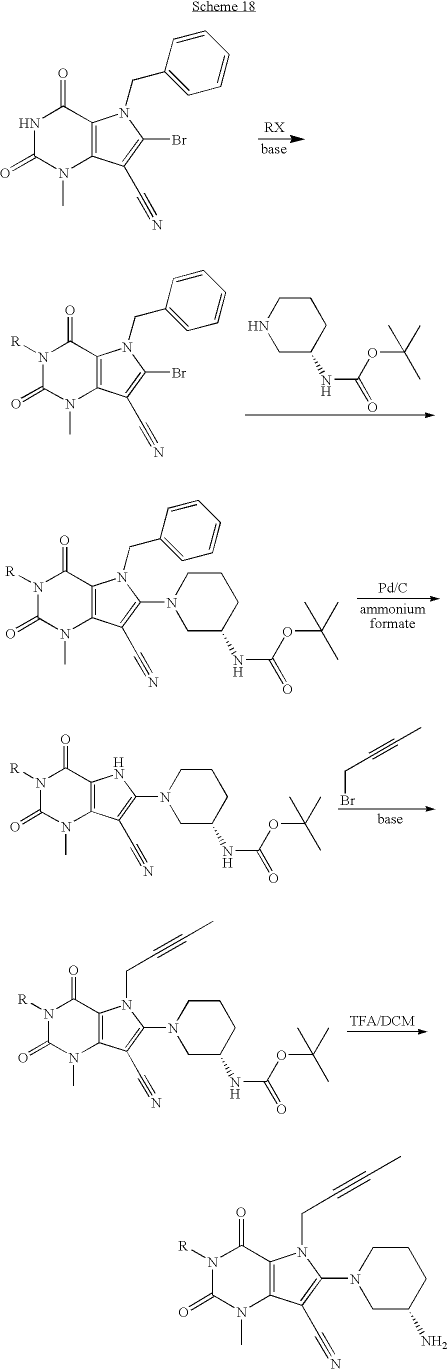

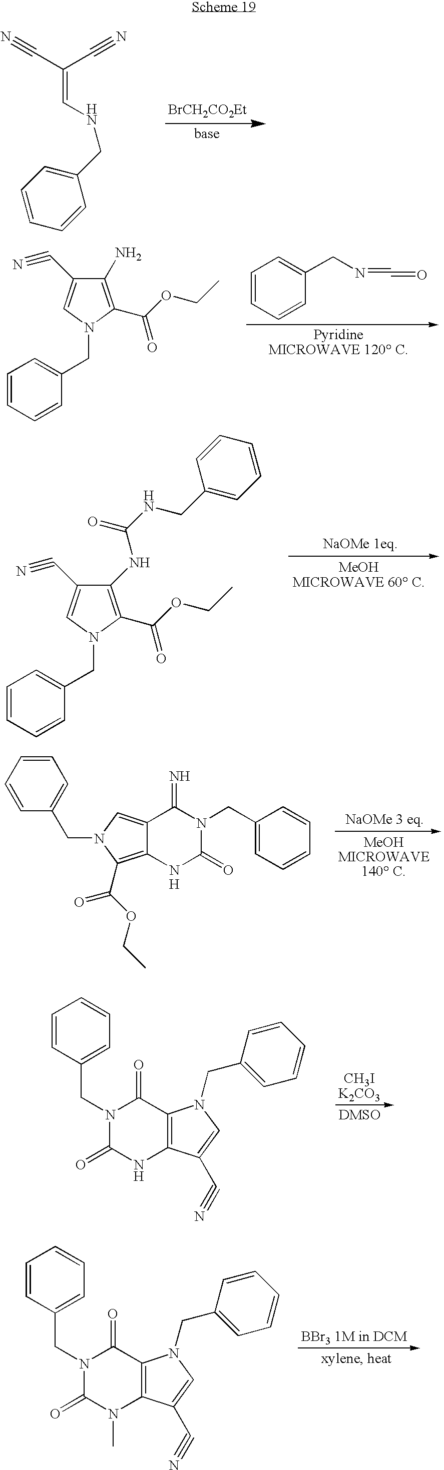

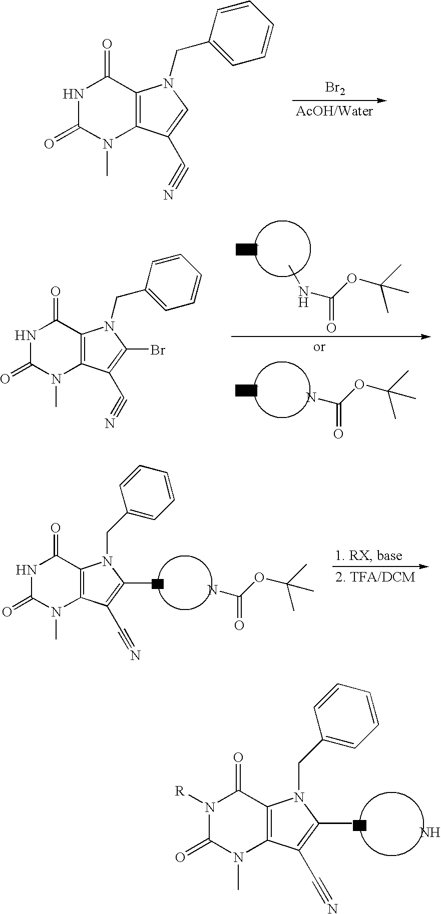

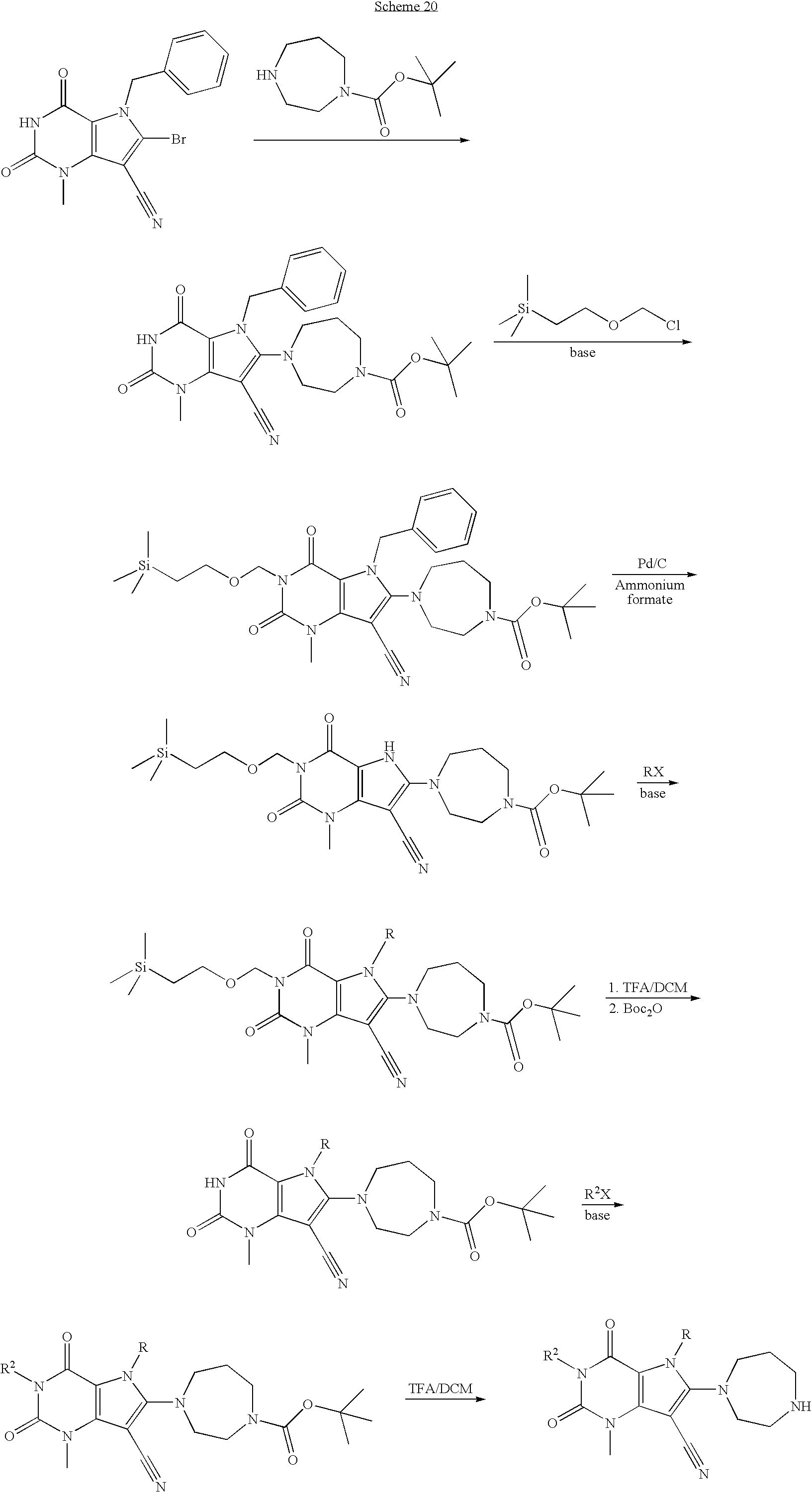

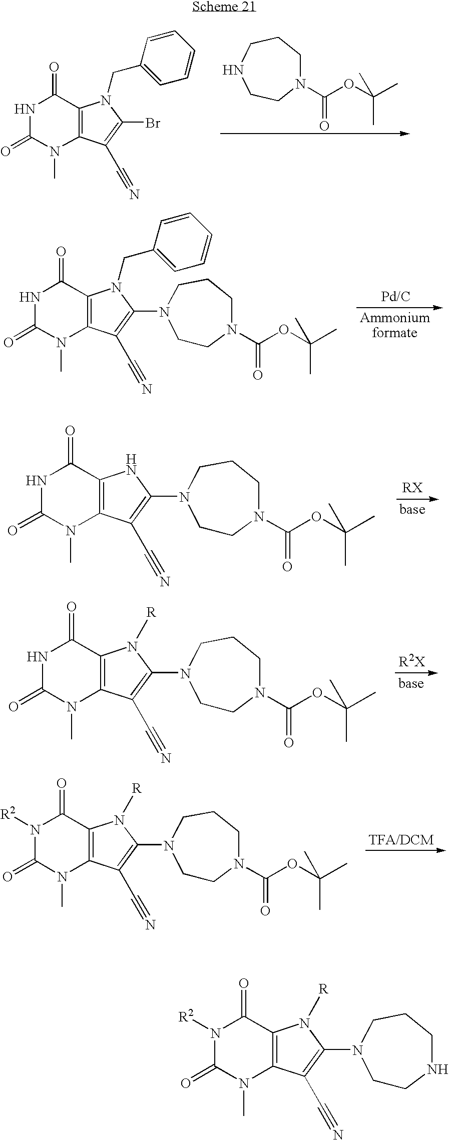

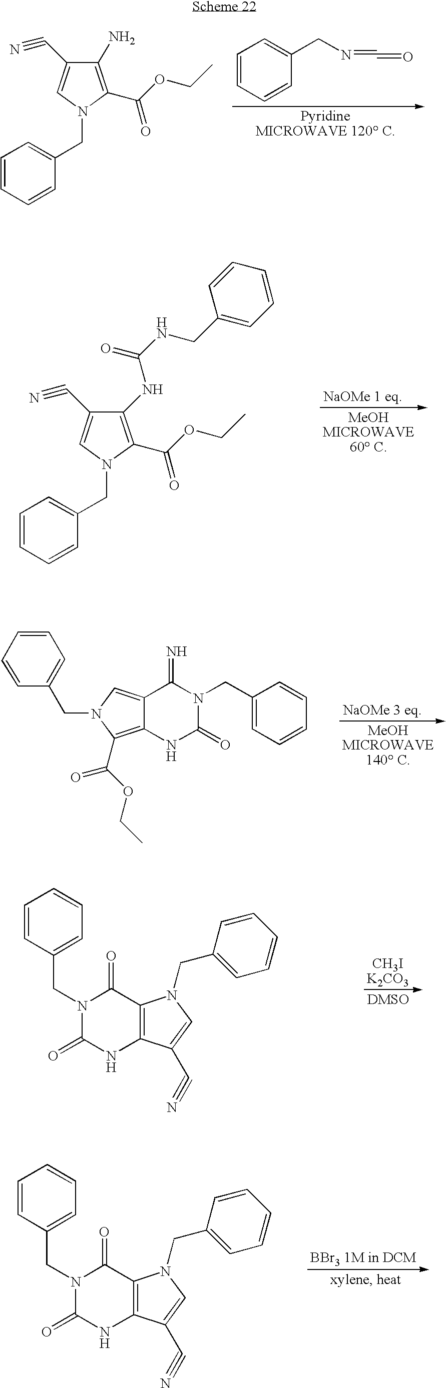

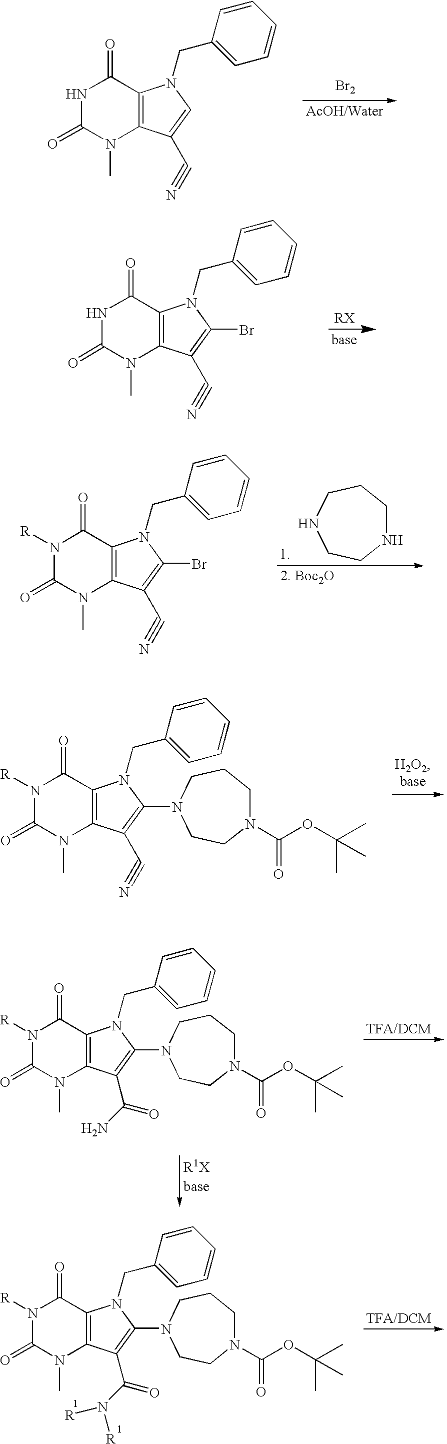

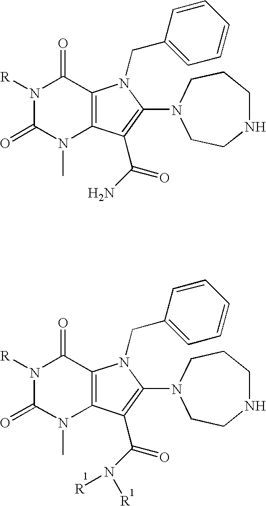

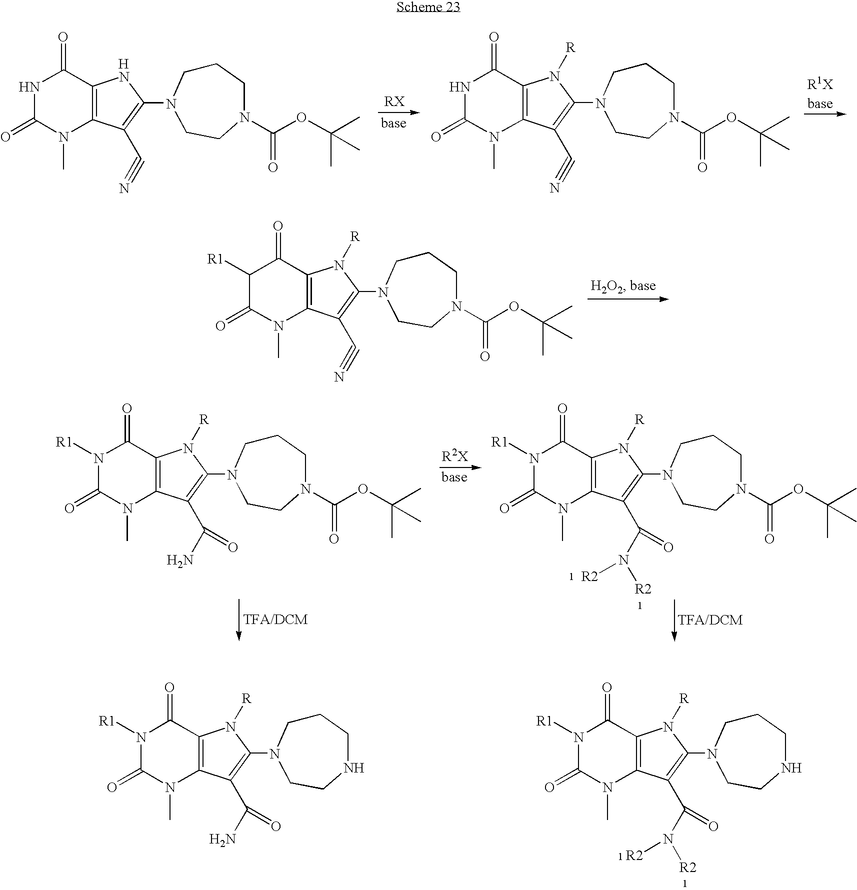

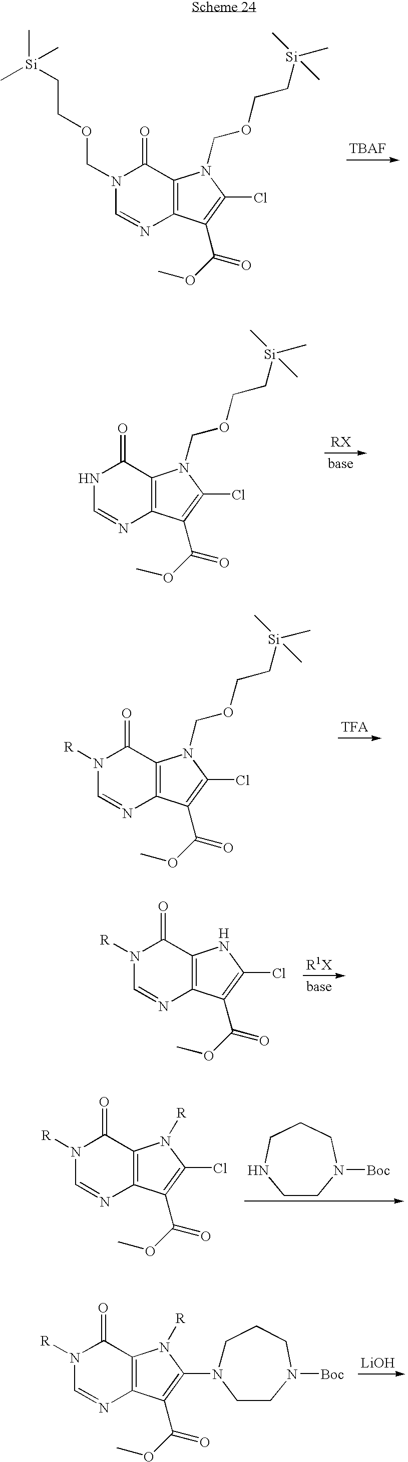

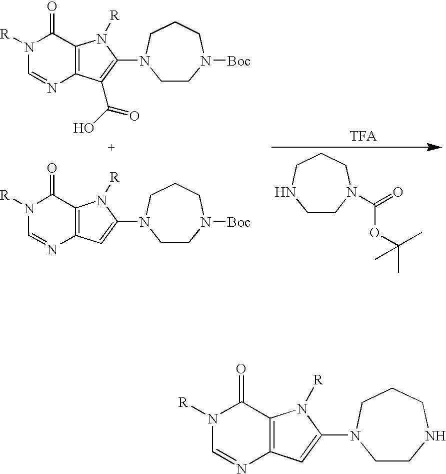

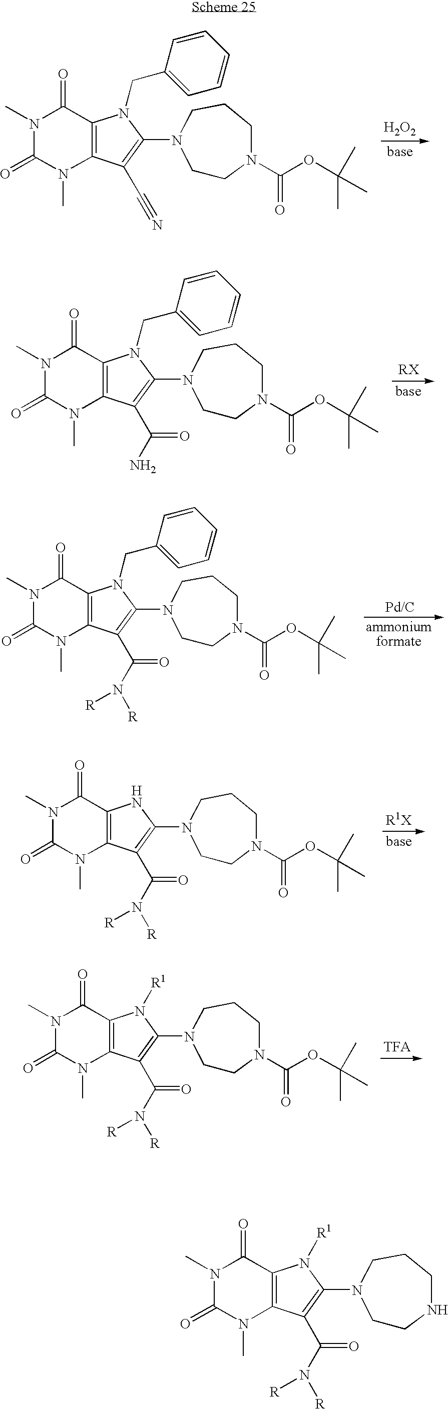

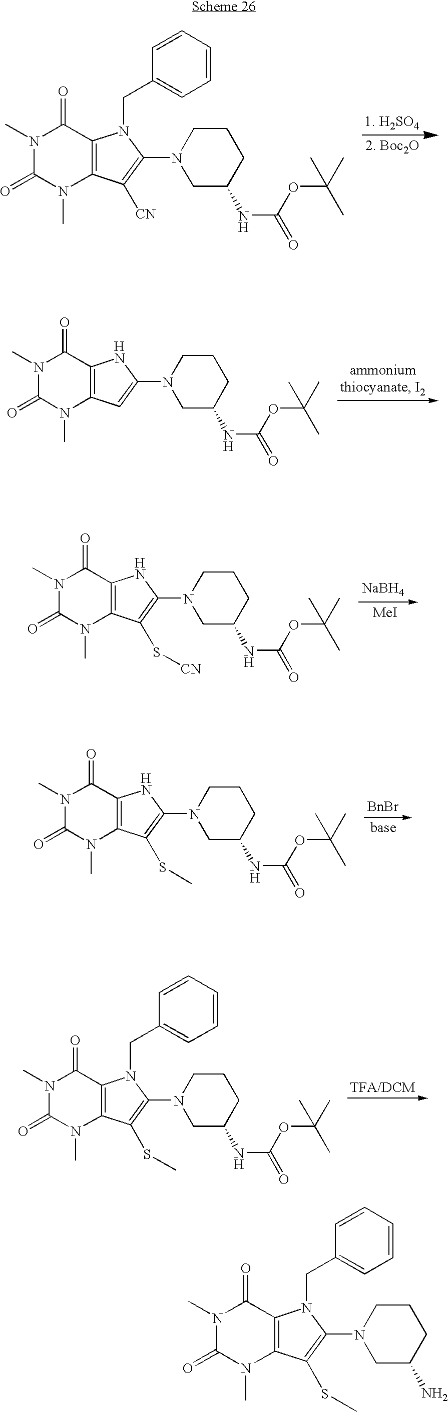

It will be understood that the processes detailed above are solely for the purpose of illustrating the invention and should not be construed as limiting. A process utilising similar or analogous reagents and/or conditions known to one skilled in the art may also be used to obtain a compound of the invention.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

### Administration and Pharmaceutical Formulations

The compounds of the invention will normally be administered orally, intravenously, subcutaneously, buccally, rectally, dermally, nasally, tracheally, bronchially, by any other parenteral route, as an oral or nasal spray or via inhalation, The compounds may be administered in the form of pharmaceutical preparations comprising prodrug or active compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion.) to a host to obtain an protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

In the treatment, prevention, control, amelioration, or reduction of risk of conditions which require inhibition of DPP-IV enzyme activity, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The compounds may be administered on a regimen of 1 to 4 times per day, preferably once or twice per day. The dosage regimen may be adjusted to provide the optimal therapeutic response.

According to a further aspect of the invention there is thus provided a pharmaceutical composition including a compound of the invention, in admixture with a pharmaceutically acceptable adjuvant, diluent or carrier.

Pharmaceutical compositions of this invention for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminum monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are suitably made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Suitably, oral formulations contain a dissolution aid. The dissolution aid is not limited as to its identity so long as it is pharmaceutically acceptable. Examples include nonionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in micro-encapsulated form, if appropriate, with one or more of the above-mentioned excipients.

The active compounds may be in finely divided form, for example it may be micronised.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Advantageously, the compounds of the invention may be orally active, have rapid onset of activity and low toxicity.

The compounds of the invention may have the advantage that they are more efficacious, less toxic, longer acting, have a broader range of activity, more potent, produce fewer side effects, more easily absorbed than, or have other useful pharmacological properties over, compounds known in the prior art.

### Combination Therapies

Compounds of the invention may be administered in combination with one or more therapeutic agents. Accordingly, the invention provides a pharmaceutical composition comprising an additional agent. The invention also provides a product comprising a compound of the invention and an agent; as a combined preparation for simultaneous, separate or sequential use in therapy.

In particular, a composition or product of the invention may further comprise a therapeutic agent selected from anti-diabetic agents, hypolipidemic agents, anti-obesity or appetite-regulating agents, anti-hypertensive agents, HDL-increasing agents, cholesterol absorption modulators, Apo-A1 analogues and mimetics, thrombin inhibitors, aldosterone inhibitors, inhibitors of platelet aggregation, estrogen, testosterone, selective estrogen receptor modulators, selective androgen receptor modulators, chemotherapeutic agents, and 5-HT₃ or 5-HT₄ receptor modulators; or pharmaceutically acceptable salts or prodrugs thereof.

Examples of anti-diabetic agents include insulin, insulin derivatives and mimetics; insulin secretagogues, for example sulfonylureas (e.g. glipizide, glyburide or amaryl); insulinotropic sulfonylurea receptor ligands, for example meglitinides (e.g. nateglinide or repaglinide); insulin sensitisers, for example protein tyrosine phosphatase-1B (PTP-1B) inhibitors (e.g. PTP-112); GSK3 (glycogen synthase kinase-3) inhibitors, for example SB-517955, SB-4195052, SB-216763, NN-57-05441 or NN-57-05445; RXR ligands, for example GW-0791 or AGN-194204; sodium-dependent glucose cotransporter inhibitors, for example T-1095; glycogen phosphorylase A inhibitors, for example BAY R3401; biguanides, for example metformin; alpha-glucosidase inhibitors, for example acarbose; GLP-1 (glucagon like peptide-1), GLP-1 analogues and mimetics, for example exendin-4; DPPIV (dipeptidyl peptidase IV) inhibitors, for example DPP728, LAF237 (vildagliptin), MK-0431, saxagliptin or GSK23A; AGE breakers; and thiazolidone derivatives, for example glitazone, pioglitazone, rosiglitazone or (R)-1-{4-[5-methyl-2-(4-trifluoromethyl-phenyl)-oxazol-4-ylmethoxy]-benzenesulfonyl}-2,3-dihydro-1H-indole-2-carboxylic acid (compound 4 of Example 19 of WO 03/043985) or a non-glitazone type PPAR-agonist (e.g. G1-262570); or pharmaceutically acceptable salts or prodrugs thereof.

Examples of hypolipidemic agents include 3-hydroxy-3-methyl-glutaryl coenzyme A (HMG-CoA) reductase inhibitors, for example lovastatin, pitavastatin, simvastatin, pravastatin, cerivastatin, mevastatin, velostatin, fluvastatin, dalvastatin, atorvastatin, rosuvastatin or rivastatin; squalene synthase inhibitors; FXR (farnesoid X receptor) ligands; LXR (liver X receptor) ligands; cholestyramine; fibrates; nicotinic acid; and aspirin; or pharmaceutically acceptable salts or prodrugs thereof.

Examples of anti-obesity/appetite-regulating agents include phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and cannabinoid receptor antagonists (rimanoban); or pharmaceutically acceptable salts or prodrugs thereof.

Examples of anti-hypertensive agents include loop diuretics, for example ethacrynic acid, furosemide or torsemide; diuretics, for example thiazide derivatives, chlorithiazide, hydrochlorothiazide or amiloride; angiotensin converting enzyme (ACE) inhibitors, for example benazepril, captopril, enalapril, fosinopril, lisinopril, moexipril, perinodopril, quinapril, ramipril or trandolapril; Na—K-ATPase membrane pump inhibitors, for example digoxin; neutralendopeptidase (NEP) inhibitors, for example thiorphan, terteo-thiorphan or SQ29072; ECE inhibitors, for example SLV306; dual ACE/NEP inhibitors, for example omapatrilat, sampatrilat or fasidotril; angiotensin II antagonists, for example candesartan, eprosartan, irbesartan, losartan, telmisartan or valsartan; renin inhibitors, for example aliskiren, terlakiren, ditekiren, RO-66-1132 or RO-66-1168; b-adrenergic receptor blockers, for example acebutolol, atenolol, betaxolol, bisoprolol, metoprolol, nadolol, propranolol, sotalol or timolol; inotropic agents, for example digoxin, dobutamine or milrinone; calcium channel blockers, for example amlodipine, bepridil, diltiazem, felodipine, nicardipine, nimodipine, nifedipine, nisoldipine or verapamil; aldosterone receptor antagonists; and aldosterone synthase inhibitors; or pharmaceutically acceptable salts or prodrugs thereof.

Examples of cholesterol absorption modulators include Zetia® and KT6-971, or pharmaceutically acceptable salts or prodrugs thereof.

Examples of aldosterone inhibitors include anastrazole, fadrazole and eplerenone, or pharmaceutically acceptable salts or prodrugs thereof.

Examples of inhibitors of platelet aggregation include aspirin or clopidogrel bisulfate, or pharmaceutically acceptable salts or prodrugs thereof.

Examples of chemotherapeutic agents include compounds decreasing the protein kinase activity, for example PDGF receptor tyrosine kinase inhibitors (e.g. imatinib or 4-methyl-N-[3-(4-methyl-imidazol-1-yl)-5-trifluoromethyl-phenyl]-3-(4-pyridin-3-yl-pyrimidin-2-ylamino)-benzamide), or pharmaceutically acceptable salts or prodrugs thereof.

Examples of 5-HT₃ or 5-HT₄ receptor modulators include tegaserod, tegaserod hydrogen maleate, cisapride or cilansetron, or pharmaceutically acceptable salts or prodrugs thereof.

The weight ratio of the compound of the present invention to the further active ingredient(s) may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200.

Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

### Use

Compounds of the invention may be useful in the therapy of a variety of diseases and conditions.

In particular, compounds of the invention may be useful in the treatment or prevention of a disease or condition selected from non-insulin-dependent diabetes mellitus, arthritis, obesity, allograft transplantation, osteoporosis, heart failure, impaired glucose metabolism or impaired glucose tolerance, neurodegenerative diseases (for example Alzheimer's disease or Parkinson disease), cardiovascular or renal diseases (for example diabetic cardiomyopathy, left or right ventricular hypertrophy, hypertrophic medial thickening in arteries and/or in large vessels, mesenteric vasculature hypertrophy or mesanglial hypertrophy), neurodegenerative or cognitive disorders, hyperglycemia, insulin resistance, lipid disorders, dyslipidemia, hyperlipidemia, hypertriglyceridemia, hypercholesterolemia, low HDL levels, high LDL levels, atherosclerosis, vascular restenosis, irritable bowel syndrome, inflammatory bowel disease (e.g. Crohn's disease or ulcerative colitis), pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis.

The compounds may also be useful in producing a sedative or anxiolytic effect, attenuating post-surgical catabolic changes or hormonal responses to stress, reducing mortality and morbidity after myocardial infarction, modulating hyperlipidemia or associated conditions; and lowering VLDL, LDL or Lp(a) levels.

### EXAMPLES

The following Examples illustrate the invention.

The abbreviations used in the Examples are as follows:

CH₃CN=acetonitrile

### DCM=DCM

### DMA=DMA

### DMF=DMF

DMSO=dimethyl sulphoxide
HATU=O-(7-Azabenzotriazol-1-yl)-N,N,N′,N′-tetramethyluronium hexafluorophosphate
MeOH=methanol
SCX-2=strong cation exchange resin
TFA=trifluoroacetic acid
THF=tetrahydrofuran

### Example A1

### 5-But-2-ynyl-4-oxo-3-(2-oxo-2-phenyl -ethyl)-6-piperazin-1-yl-4,5-dihydro-3H-pyrrolo[3,2-d]pyrimidine-7-carbonitrile

This compound was prepared according to scheme 1.

### A 3-Amino-4-cyano-1H-pyrrole-2-carboxylic Acid Methyl Ester

A solution of sodium methoxide (50.3 mL of a 25 wt % solution in MeOH) was added in one portion to a solution of diethyl aminomalonate hydrochloride (15.5 g, 73.2 mmol) in MeOH (140 mL). 2-Ethoxymethylene-malononitrile (8.94 g, 73.2 mmol) was added during 15 minutes keeping the temperature below 45° C. The mixture was heated at reflux for 4 hours. After cooling to ambient temperature, the mixture was neutralized with glacial acetic acid (9 mL), and concentrated in vacuo to a thick paste. Water was added with stirring, and the resulting slurry was extracted with ethyl acetate (2×250 mL). The combined organic extracts were washed with aqueous saturated sodium bicarbonate (300 mL) and brine (300 mL), dried (MgSO₄), filtered and concentrated in vacuo to give a orange solid. The solid was triturated with diethyl ether (50 mL) and collected by filtration to give the title compound as a tan solid.

MS: 166 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.74 min.

### B 4-Oxo-4,5-dihydro-3H-pyrrolo[3,2-d]pyrimidine-7-carbonitrile

A solution of 3-amino-4-cyano-1H-pyrrole-2-carboxylic acid methyl ester (6.0 g, 45.4 mmol) in formamide (48 mL) was treated with a solution of sodium methoxide (31.1 mL of a 25 wt % solution in MeOH). The resulting solution was heated to 100° C. for 20 hours, cooled to 0° C. and treated with 2M aqueous hydrochloric acid (80 mL). The solid was collected by filtration and oven dried in vacuo (1 mbar, 100° C.) for 2 hours to give the title compound as a beige solid.

MS: 161 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.22 min.

### C 6-Bromo-4-oxo-4,5-dihydro-3H-pyrrolo[3,2-d]pyrimidine-7-carbonitrile

4-Oxo-4,5-dihydro-3H-pyrrolo[3,2-d]pyrimidine-7-carbonitrile (4.1 g, 25.6 mmol.) was suspended in DMF and N-bromosuccinimide (11.7 g, 64.0 mmol.) was added. The mixture was stirred at room temperature for 20 hours. Another equivalent of N-bromosuccinimide was added and stirring was continued for a further 18 hours. Water (150 ml) was added and a solid was formed. The solid was collected, washed with water and dried under vacuum at 60° C. for 2 hr to give the title compound as an orange solid.

MS: 239 and 241 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.87 min.

### D 4,6-Dichloro-5H-pyrrolo[3,2-d]pyrimidine-7-carbonitrile

A suspension of 6-bromo-4-oxo-4,5-dihydro-3H-pyrrolo[3,2-d]pyrimidine-7-carbonitrile (1.0 g, 4.18 mmol) in phosphorus oxychloride (10 mL) was stirred at 110° C. for 8 hours. The reaction mixture was cooled, added to crushed ice and extracted into ethyl acetate (3×25 mL). The combined organic extracts were washed with brine (20 mL), dried (Na₂SO₄), filtered and concentrated in vacuo to afford the title product as an orange solid.

MS: 213[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.37 min.

### E 5-But-2-ynyl-4,6-dichloro-5H-pyrrolo[3,2-d]pyrimidine-7-carbonitrile

A mixture of 4,6-dichloro-5H-pyrrolo[3,2-d]pyrimidine-7-carbonitrile (480 mg, 1.86 mmol.), 1-bromo-but-2-yne (0.203 mL, 2.23 mmol.) and potassium carbonate (385 mg, 2.85 mmol.) in DMF (10 mL) was stirred at 60° C. for 18 hours. The reaction mixture was concentrated in vacuo and the residue was purified by flash chromatography (Silica, eluent: ethyl acetate/petrol (40-60° C.) 1/1) to give the title compound as an orange solid.

MS: 265[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.89 min.

### F 5-But-2-ynyl-6-chloro-4-oxo-4,5-dihydro-3H-pyrrolo[3,2-d]pyrimidine-7-carbonitrile

A solution of 5-but-2-ynyl-4,6-dichloro-5H-pyrrolo[3,2-d]pyrimidine-7-carbonitrile (150 mg, 0.49 mmol.) in dioxane (2.5 mL) was treated with 1M aq. hydrochloric acid (2.5 mL) and the mixture was heated at reflux for 2.5 hours The solution was neutralised by addition of saturated aq. sodium bicarbonate (2.5 mL) and concentrated in vacuo. The residue was partitioned between water (15 mL) and ethyl acetate (3×10 mL) and the combined organic extracts are washed with brine (20 mL), dried (MgSO₄), filtered and concentrated in vacuo. The residue was purified by flash chromatography (Silica, eluent: ethyl acetate) to afford the title compound as a beige solid.

MS: 247[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.64 min.

### G 5-But-2-ynyl-6-chloro-4-oxo-3-(2-oxo-2-phenyl-ethyl)-4,5-dihydro-3H-pyrrolo[3,2d]pyrimidine-7-carbonitrile

2-Bromo-1-phenyl-ethanone (53 mg, 0.27 mmol.) was added to a mixture of 5-but-2-ynyl-6-chloro-4-oxo-4,5-dihydro-3H-pyrrolo[3,2-d]pyrimidine-7-carbonitrile (60 mg, 0.24 mmol.) and potassium carbonate (40 mg, 0.29 mmol.) in DMF (1 mL) and the mixture was stirred at room temperature for 1 hour The DMF was evaporated in vacuo and the residue was triturated with water (5 mL). The solid was collected, washed with water and dried to afford the title compound as a beige solid.

MS: 365[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.55 min.

### H 5-But-2-ynyl-4-oxo-3-(2-oxo-2-phenyl-ethyl)-6-piperazin-1-yl-4,5-dihydro-3H-pyrrolo[3,2-d]pyrimidine-7-carbonitrile

A solution of 5-but-2-ynyl-6-chloro-4-oxo-3-(2-oxo-2-phenyl-ethyl)-4,5-dihydro-3H-pyrrolo[3,2d]pyrimidine-7-carbonitrile (70 mg, 0.19 mmol.) and piperazine (165 mg , 0.19 mmol.) in DMA (2.5 mL) was heated under microwave irradiation (Smith Microwave Synthesizer) for 5 mins at 160° C. The crude reaction mixture was partitioned between chloroform (3×20 mL) and water (20 ml), the combined extracts washed with water (40 mL) and brine (40 mL), filtered and evaporated in vacuo. The residue was purified by flash chromatography (Silica, eluent: DCM/MeOH/acetic acid/water 360/20/3/2) to afford the title compound as a beige solid.

MS: 415 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.99 min.

### Example A2

### 5-But-2-ynyl-4-oxo-6-piperazin-1-yl-3-quinolin-4-ylmethyl-4,5-dihydro-3H-pyrrolo[3,2-d]pyrimidine-7-carbonitrile

This compound was prepared according to scheme 1.

The title compound was prepared analogously as described in Example A1 using 4-chloromethylquinoline hydrochloride instead of 2-bromo-1-phenyl-ethanone.

### A 5-But-2-ynyl-6-chloro-4-oxo-3-quinolin-4-ylmethyl-4,5-dihydro-3H-pyrrolo[3,2-d]pyrimidine-7-carbonitrile

A solution of 5-but-2-ynyl-6-chloro-4-oxo-4,5-dihydro-3H-pyrrolo[3,2-d]pyrimidine-7-carbonitrile (55 mg, 0.223 mmol.), prepared by the methods described in Example A1, in N,N-DMA (5 mL) was treated sequentially with 4-chloromethyl-quinoline hydrochloride (57 mg, 0.268 mmol.) and potassium carbonate (46 mg, 0.333 mmol.). After stirring at room temperature for 2 hr, the mixture was concentrated in vacuo and the residue partitioned between water and ethyl acetate. The organic phase was washed with water and evaporated. The residue was purified by flash chromatography (silica gel, eluent: DCM/MeOH 19/1) to afford the title compound as a white solid.

MS: 388[M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.81 min.

### B 5-But-2-ynyl-4-oxo-6-piperazin-1-yl-3-quinolin-4-ylmethyl-4,5-dihydro-3H-pyrrolo[3,2-d]pyrimidine-7-carbonitrile

A solution of 5-but-2-ynyl-6-chloro-4-oxo-3-quinolin-4-ylmethyl-4,5-dihydro-3H-pyrrolo[3,2-d]pyrimidine-7-carbonitrile (50 mg, 0.129 mmol.) and piperazine (55 mg, 0.0639 mmol.) in N,N-DMA (3 mL) was heated by microwave irradiation (Smith Microwave Synthesizer) for 10 mins at 160° C. The volatiles were evaporated and the residue was partitioned between water and ethyl acetate. The organic phase was washed with water and the solvent evaporated. The residue was purified by flash chromatography (silica gel, eluent: DCM/MeOH 9/1) to afford the title compound as an off-white foam.

MS: 438 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 4.54 min.

### Example B1

### 6-((R)-3-Amino-piperidin-1-yl)-5-but-2-ynyl-4-oxo-3-(2-oxo-2-phenyl -ethyl)-4,5-dihydro-3H-pyrrolo[3,2-d]pyrimidine-7-carbonitrile

This compound was prepared according to scheme 2.

### A {(R)-1-[5-But-2-ynyl-7-cyano-4-oxo-3-(2-oxo-2-phenyl-ethyl)-4,5-dihydro-3H-pyrrolo[3,2-d]pyrimidin-6-yl]-piperidin-3-yl}-carbamic Acid tert-butyl Ester

A solution of 5-but-2-ynyl-6-chloro-4-oxo-3-(2-oxo-2-phenyl-ethyl)-4,5-dihydro-3H-pyrrolo[3,2d]pyrimidine-7-carbonitrile (120 mg, 0.33 mmol.), prepared by the methods described in Example A1, and (R)-piperidin-3-yl-carbamic acid tert-butyl ester (66 mg, 0.33 mmol.) in N,N-DMA (3.0 mL) was heated under microwave irradiation (Smith Microwave Synthesizer) for 70 mins at 160° C.

The mixture was concentrated in vacuo and the residue was partitioned between water and ethyl acetate. The organic layer was evaporated and the residue was purified by flash chromatography (silica gel, eluent: DCM/MeOH 9/1) to afford the title compound as a pale yellow gum.

MS: 529 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.59 min.

### B 6-((R)-3-Amino-piperidin-1-yl)-5-but-2-ynyl-4-oxo-3-(2-oxo-2-phenyl-ethyl)-4,5-dihydro-3H-pyrrolo[3,2-d]pyrimidine-7-carbonitrile

{(R)-1-[5-But-2-ynyl-7-cyano-4-oxo-3-(2-oxo-2-phenyl-ethyl)-4,5-dihydro-3H-pyrrolo[3,2-d]pyrimidin-6-yl]-piperidin-3-yl}-carbamic acid tert-butyl ester (118 mg, 0.22 mmol.) was dissolved in TFA (2 mL) and DCM (2 mL). After 1 hr the mixture was concentrated, the residue was resuspended in DCM and concentrated. The residue was purified by flash chromatography (silica gel, eluent: DCM/MeOH 9/1) to afford the title compound as a buff solid.

MS: 429 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.31 min.

### Example B2

### 6-((R)-3-Amino-piperidin-1-yl)-5-but-2-ynyl-4-oxo-3-quinolin-4-ylmethyl-4,5-dihydro-3H-pyrrolo[3,2-d]pyrimidine-7-carbonitrile

This compound was prepared according to scheme 2.

### A [(R)-1-(5-But-2-ynyl-7-cyano-4-oxo-3-quinolin-4-ylmethyl-4,5-dihydro-3H-pyrrolo[3,2-d]pyrimidin-6-yl)-piperidin-3-yl]-carbamic Acid tert-butyl Ester

A solution of 5-but-2-ynyl-6-chloro-4-oxo-3-quinolin-4-ylmethyl-4,5-dihydro-3H-pyrrolo[3,2-d]pyrimidine-7-carbonitrile (55 mg, 0.142 mmol.), prepared by the methods described in Example B1 using 4-chloromethyl quinoline hydrochloride instead of 2-bromo-1-phenyl-ethanone, and (R)-piperidin-3-yl-carbamic acid tert-butyl ester (85 mg, 0.425 mmol.) in N,N-DMA (3.0 mL) was heated under microwave irradiation (Smith Microwave Synthesizer) for 30 mins at 160° C. The mixture was concentrated, and the residue partitioned between water and ethyl acetate. The organic layer was separated, concentrated in vacuo, and the residue was purified by flash chromatography (silica gel, eluent: DCM/MeOH 19/1) to give the title compound as a white solid.

MS: 552 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.20 min.

### B 6-((R)-3-Amino-piperidin-1-yl)-5-but-2-ynyl-4-oxo-3-quinolin-4-ylmethyl-4,5-dihydro-3H-pyrrolo[3,2-d]pyrimidine-7-carbonitrile

A solution of [(R)-1-(5-but-2-ynyl-7-cyano-4-oxo-3-quinolin-4-ylmethyl-4,5-dihydro-3H-pyrrolo[3,2-d]pyrimidin-6-yl)-piperidin-3-yl]-carbamic acid tert-butyl ester (47 mg, 0.085 mmol.) in TFA (1 mL) and DCM (1 mL) was stirred at room temperature for 1 hour The volatiles were removed in vacuo and the residue was partitioned between ethyl acetate and saturated aqueous sodium bicarbonate solution. The organic phase was concentrated and the residue was purified by flash chromatography (silica gel, eluent: DCM/MeOH 9/1) to give the title compound as a buff solid.

MS: 452 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 5.03 min.

### Example C1

### 5-But-2-ynyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3

### A 3-Amino-4-cyano-1H-pyrrole-2-carboxylic Acid Methyl Ester

A solution of sodium methoxide (50.3 mL of a 25 wt % solution in MeOH) was added in one portion to a solution of diethyl aminomalonate hydrochloride (15.5 g, 73.2 mmol) in MeOH (140 mL). 2-Ethoxymethylene-malononitrile (8.94 g, 73.2 mmol) was added during 15 minutes keeping the temperature below 45° C. The mixture was heated at reflux for 4 hours. After cooling to ambient temperature, the mixture was neutralized with glacial acetic acid (9 mL), and concentrated in vacuo to a thick paste. Water was added with stirring, and the resulting slurry was extracted with ethyl acetate (2×250 mL). The combined organic extracts were washed with aqueous saturated sodium bicarbonate (300 mL) and brine (300 mL), dried (MgSO₄), filtered and concentrated in vacuo to give a orange solid. The solid was triturated with diethyl ether (50 mL) and collected by filtration to give the title compound as a tan solid.

MS: 166 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.74 min.

### B 4-Oxo-4,5-dihydro-3H-pyrrolo[3,2-d]pyrimidine-7-carbonitrile

A solution of 3-amino-4-cyano-1H-pyrrole-2-carboxylic acid methyl ester (6.0 g, 45.4 mmol) in formamide (48 mL) was treated with a solution of sodium methoxide (31.1 mL of a 25 wt % solution in MeOH). The resulting solution was heated to 100° C. for 20 hours, cooled to 0° C. and treated with 2M aqueous hydrochloric acid (80 mL). The solid was collected by filtration and oven dried in vacuo (1 mbar, 100° C.) for 2 hours to give the title compound as a beige solid.

MS: 161 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.22 min.

### C 6-Bromo-4-oxo-4,5-dihydro-3H-pyrrolo[3,2-d]pyrimidine-7-carbonitrile

4-Oxo-4,5-dihydro-3H-pyrrolo[3,2-d]pyrimidine-7-carbonitrile (4.1 g, 25.6 mmol.) was suspended in DMF and N-bromosuccinimide (11.7 g, 64.0 mmol.) was added. The mixture was stirred at room temperature for 20 hours. Another equivalent of N-bromosuccinimide was added and stirring was continued for a further 18 hours. Water (150 ml) was added and a solid was formed. The solid was collected, washed with water and dried under vacuum at 60° C. for 2 hr to give the title compound as an orange solid.

MS: 239 and 241 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.87 min.

### D 6-Bromo-5-but-2-ynyl-4-oxo-4,5-dihydro-3H-pyrrolo[3,2-d]pyrimidine-7-carbonitrile

To a solution of 6-bromo-4-oxo-4,5-dihydro-3H-pyrrolo[3,2-d]pyrimidine-7-carbonitrile (478 mg, 2 mmol) in DMF were added DIPEA (516 mg, 4 mmol) and dropwise 1-bromo-but-2-yne (293 mg, 2.2 mmol). After stirring at rt during 2 h, the solution was evaporated. The residue was dissolved with water and ethyl acetate before washing several times with water and evaporation of the organic phase to give a crude compound which was purified by flash chromatography (silica, DCM/MeOH 95/5 as eluent) to yield the title compound as a white foam.

MS: 291 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.39 min.

### E 6-Bromo-5-but-2-ynyl-3-[2-(3-methoxy-phenyl)-2-oxo-ethyl]-4-oxo-4,5-dihydro-3H-pyrrolo[3,2-d]pyrimidine-7-carbonitrile

3-Methoxy-2-bromoacetophenone (220 mg, 1.15 mmol.) was added to a mixture of 6-bromo-5-but-2-ynyl-4-oxo-4,5-dihydro-3H-pyrrolo[3,2-d]pyrimidine-7-carbonitrile (280 mg, 0.96 mmol.) and potassium carbonate (270 mg, 1.15 mmol.) in DMF (10 mL) and the mixture was stirred at room temperature for 16 hours After evaporation of the solvent, the residue was dissolved with water and ethyl acetate before washing several times with water and evaporation of the organic phase to give a crude compound which was purified by flash chromatography (silica, DCM/MeOH 98/2 as eluent) to yield the title compound as a white solid.

MS: 439.4 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.63 min.

### F 5-But-2-ynyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

A solution of 6-bromo-5-but-2-ynyl-3-[2-(3-methoxy-phenyl)-2-oxo-ethyl]-4-oxo-4,5-dihydro-3H-pyrrolo[3,2-d]pyrimidine-7-carbonitrile (93 mg, 0.21 mmol.) and piperazine (182 mg, 2.1 mmol.) in N,N-DMA (2 mL) was heated by microwave irradiation (Smith Microwave Synthesizer) for 15 mins at 160° C. The volatiles were evaporated and the residue was partitioned between water and ethyl acetate. The organic phase was washed with water and the solvent evaporated. The residue was purified by flash chromatography (silica gel, eluent: DCM/MeOH 9/1) to afford the title compound as a pale yellow solid.

MS: 445 [M+H]⁺.

T_{R} [HPLC, Higgins Clipeus 5 micron C18; 5-95% CH₃CN+0.1% Formic acid/H₂O+0.1% Formic acid for 20 min, flow 2.0 ml/min]: 6.47 min.

### Example C2

### 3-[2-(3-Methoxy-phenyl)-2-oxo-ethyl]-5-(3-methyl-but-2-enyl)-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 4-bromo-2-methylbutene instead of 1-bromo-but-2-yne.

MS: 461 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.9 min.

### Example C3

### 5-(2-Chloro-benzyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 2-chlorobenzylbromide instead of 1-bromo-but-2-yne.

MS: 517/518 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.09 min.

### Example C4

### 5-(2-Chloro-5-fluoro-benzyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 2-chloro-5-fluorobenzylbromide instead of 1-bromo-but-2-yne.

MS: 535 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.23 min.

### Example C5

### 5-(2-Methoxy-ethyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 1-bromo-2-methoxyethane instead of 1-bromo-but-2-yne.

MS: 451 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.14 min.

### Example C6

### 5-Benzyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using benzyl bromide instead of 1-bromo-but-2-yne.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.99 min.

### Example C7

### 3-[2-(3-Methoxy-phenyl)-2-oxo-ethyl]-5-methyl-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using iodomethane instead of 1-bromo-but-2-yne.

MS: 407.10 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.12 min.

### Example C8

### 3-[2-(3-Methoxy-phenyl)-2-oxo-ethyl]-5-(3-methyl-butyl)-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 1-bromo-3-methylbutane instead of 1-bromo-but-2-yne.

MS: 463.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.24 min.

### Example C9

### 5-Cyclopropyl methyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using cyclopropylmethyl bromide instead of 1-bromo-but-2-yne.

MS: 447.12 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.66 min.

### Example C10

### 5-Cyclobutyl methyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using bromomethyl(cyclobutane) instead of 1-bromo-but-2-yne.

MS: 461.14 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.98 min.

### Example C11

### 3-[2-(3-Methoxy-phenyl)-2-oxo-ethyl]-4-oxo-6-piperazin-1-yl-5-(tetrahydro-furan-2-ylmethyl)-4,5-dihydro-3H-pyrrolo[3,2-d]pyrimidine-7-carbonitrile

This compound was prepared according to scheme 3.

The title compound was prepared analogously as described in Example C1 using tetrahydrofurfuryl bromide instead of 1-bromo-but-2-yne.

MS: 477 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.28 min.

### Example C12

### 6-[1,4]-Diazepan-1-yl-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carbonitrile

This compound was prepared according to scheme 3.

### A 3-Amino-4-cyano-1H-pyrrole-2-carboxylic Acid Methyl Ester

A solution of sodium methoxide (50.3 mL of a 25 wt % solution in MeOH) was added in one portion to a solution of diethyl aminomalonate hydrochloride (15.5 g, 73.2 mmol) in MeOH (140 mL). 2-Ethoxymethylene-malononitrile (8.94 g, 73.2 mmol) was added during 15 minutes keeping the temperature below 45° C. The mixture was heated at reflux for 4 hours. After cooling to ambient temperature, the mixture was neutralized with glacial acetic acid (9 mL), and concentrated in vacuo to a thick paste. Water was added with stirring, and the resulting slurry was extracted with ethyl acetate (2×250 mL). The combined organic extracts were washed with aqueous saturated sodium bicarbonate (300 mL) and brine (300 mL), dried (MgSO₄), filtered and concentrated in vacuo to give a orange solid. The solid was triturated with diethyl ether (50 mL) and collected by filtration to give the title compound as a tan solid.

MS: 166 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.74 min.

### B 4-Oxo-4,5-dihydro-3H-pyrrolo[3,2-d]pyrimidine-7-carbonitrile

A solution of 3-amino-4-cyano-1H-pyrrole-2-carboxylic acid methyl ester (6.0 g, 45.4 mmol) in formamide (48 mL) was treated with a solution of sodium methoxide (31.1 mL of a 25 wt % solution in MeOH). The resulting solution was heated to 100° C. for 20 hours, cooled to 0° C. and treated with 2M aqueous hydrochloric acid (80 mL). The solid was collected by filtration and oven dried in vacuo (1 mbar, 100° C.) for 2 hours to give the title compound as a beige solid.

MS: 161 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.22 min.

### C 6-Bromo-4-oxo-4,5-dihydro-3H-pyrrolo[3,2-d]pyrimidine-7-carbonitrile

4-Oxo-4,5-dihydro-3H-pyrrolo[3,2-d]pyrimidine-7-carbonitrile (4.1 g, 25.6 mmol.) was suspended in DMF and N-bromosuccinimide (11.7 g, 64.0 mmol.) was added. The mixture was stirred at room temperature for 20 hours. Another equivalent of N-bromosuccinimide was added and stirring was continued for a further 18 hours. Water (150 ml) was added and a solid was formed. The solid was collected, washed with water and dried under vacuum at 60° C. for 2 hr to give the title compound as an orange solid.

MS: 239 and 241 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.87 min.

### D 6-Bromo-5-(3-methyl-but-2-enyl)-4-oxo-4,5-dihydro-3H-pyrrolo[3,2-d]pyrimidine-7-carbonitrile

A solution of 6-bromo-4-oxo-4,5-dihydro-3H-pyrrolo[3,2-d]pyrimidine-7-carbonitrile (3.0 g, 12.55 mmol) in DMF (100 mL) was treated with diisopropylethylamine (3.23 g, 25.1 mmol) and was then cooled in an ice bath . The 1-bromo-3-methyl-but-2-ene (1.87 g, 12.55 mmol) was dissolved in DMF (10 mL) and was then added dropwise over 30 mins, keeping the temperature below 5° C. The mixture was then stirred in an ice bath for 2 hours. The mixture was evaporated and the residue partitioned between water and ethyl acetate. The organic layer was washed several times with water then evaporated. The residue was then passed down a flash silica column eluting with 2% MeOH/DCM. Appropriate fractions were combined and evaporated to give the title compound as a pale yellow solid.

MS: 307/309[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.00 min.

### E 6-Bromo-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile

Potassium carbonate (572 mgs, 4.14 mmol) and 2-bromo-1-(3-methoxyphenyl)-ethanone (813 mg, 3.55 mmol) were added to a solution of 6-bromo-5-(3-methyl-but-2-enyl)-4-oxo-4,5-dihydro-3H-pyrrolo[3,2-d]pyrimidine-7-carbonitrile (910 mg, 2.96 mmol) in DMF (30 mL) and the mixture was stirred at room temperature for 3 hours. The reaction mixture was evaporated and the residue partitioned between water and ethyl acetate. The organic layer was washed with water and evaporated. The residue was purified by flash chromatography (Silica, eluting with 2% MeOH in DCM). After combining appropriate fractions, evaporation of the solvents afforded the title compound as a pale yellow solid.

MS: 455/457[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.98 min.

### F 6-[1,4]Diazepan-1-yl-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile

A solution of 6-bromo-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile (80 mg, 0.176 mmol) and [1,4]diazepane (88 mg, 0.879 mmol) in DMF (3 mL) was heated by microwave irradiation at 150° C. for 35 minutes. The mixture was evaporated and the residue partitioned between water and ethyl acetate. The organic layer was then washed several times with water and evaporated. The residue was purified by flash chromatography (Silica, eluting with 5% MeOH in DCM). Fractions containing the main UV visible spot were combined and evaporated to give the title compound as a pale yellow solid.

MS: 475 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.01 min.

### Example C13

### Mixture of 3-[2-(3-Methoxy-phenyl)-2-oxo-ethyl]-5-((1S,2S-2-methyl-cyclopropyl methyl)-4-oxo-6-piperazin-1-yl-4,5-dihydro-3H-pyrrolo[3,2-d]pyrimidine-7-carbonitrile and 3-[2-(3-methoxy-phenyl)-2-oxo-ethyl]-5-((1R,2R-2-methyl-cyclopropylmethyl)-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 1-bromomethyl-2-methyl cyclopropane instead of 1-bromo-but-2-yne.

MS: 461 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.01 min.

### Example C14

### 3-[2-(3-Methoxy-phenyl)-2-oxo-ethyl]-4-oxo-6-piperazin-1-yl-5-thiazol-4-ylmethyl-4,5-dihydro-3H-pyrrolo[3,2-d]pyrimidine-7-carbonitrile

This compound was prepared according to scheme 3.

The title compound was prepared analogously as described in Example C1 using 4-(chloromethyl)-1,3-thiazole hydrochloride instead of 1-bromo-but-2-yne.

MS: 490.07 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.20 min.

### Example C15

### 5-(2-Cyclopropyl-ethyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using (2-bromoethyl)-cyclopropane instead of 1-bromo-but-2-yne.

MS: 461.20 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.90 min.

### Example C16

### 5-Furan-3-yl methyl-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 3-bromomethyl-furan instead of 1-bromo-but-2-yne.

MS: 473.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.92 min.

### Example C17

### 6-(2-Amino-ethylamino)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile

This compound was prepared according to scheme 3.

The title compound was prepared analogously as described in Example C1 using ethylenediamine instead of piperazine.

MS: 435 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.80 min.

### Example C18

### 5-But-2-yn-1-yl-3-(isoquinolin-1-yl methyl)-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 1-(bromomethyl)-isoquinoline hydrobromide instead of 3-methoxy-2-bromoacetophenone.

MS: 438 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.15 min.

### Example C19

### 5-But-2-yn-1-yl-6-(1,4-diazepan-1-yl)-3-(isoquinolin-1-ylmethyl)-4-oxo-4,5-dihydro-3H-pyrrolo[3,2-d]pyrimidine-7-carbonitrile Dihydrochloride

This compound was prepared according to scheme 3.

The title compound was prepared analogously as described in Example C12 using 1-bromo-but-2-yne instead of 1-bromo-3-methyl-but-2-ene and using 1-(bromomethyl)-isoquinoline hydrobromide instead of 2-bromo-1-(3-methoxyphenyl)-ethanone.

MS: 452 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.27 min.

### Example C20

### 5-But-2-yn-1-yl-6-(1,4-diazepan-1-yl)-3-(2-(3-(methyloxy)phenyl)-2-oxoethyl)-4-oxo-4,5-dihydro-3H-pyrrolo[3,2-d]pyrimidine-7-carbonitrile Hydrochloride

This compound was prepared according to scheme 3.

The title compound was prepared analogously as described in Example C12 using 1-bromo-but-2-yne instead of 1-bromo-3-methyl-but-2-ene.

MS: 459 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.62 min.

### Example C21

### 5-But-2-yn-1-yl-6-(1,4-diazepan-1-yl)-3-methyl-4-oxo-4,5-dihydro-3H-pyrrolo[3,2-d]pyrimidine-7-carbonitrile Hydrochloride

This compound was prepared according to scheme 3.

The title compound was prepared analogously as described in Example C12 using 1-bromo-but-2-yne instead of 1-bromo-3-methyl-but-2-ene and using iodomethane instead of 2-bromo-1-(3-methoxyphenyl)-ethanone. The product was converted to the hydrochloride salt by dissolving in excess MeOHic hydrogen chloride (1.25M, 5-10 equivalents) and removal of volatiles.

MS: 325 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.53 min.

### Example C22

### 5-But-2-yn-1-yl-3-methyl-4-oxo-6-piperazin-1-yl-4,5-dihydro-3H-pyrrolo[3,2-d]pyrimidine-7-carbonitrile Hydrochloride

This compound was prepared according to scheme 3.

The title compound was prepared analogously as described in Example C1 using iodomethane instead of 2-bromo-1-(3-methoxyphenyl)-ethanone. The product was converted to the hydrochloride salt by dissolving in excess MeOHic hydrogen chloride (1.25M, 5-10 equivalents) and removal of volatiles.

MS: 311 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.25 min.

### Example C23

### 6-(1,4-Diazepan-1-yl)-3-(isoquinolin-1-ylmethyl)-5-(3-methylbut-2-en-1-yl)-4-oxo-4,5-dihydro-3H-pyrrolo[3,2-d]pyrimidine-7-carbonitrile

This compound was prepared according to scheme 3.

The title compound was prepared analogously as described in Example C12 using 1-(bromomethyl)-isoquinoline hydrobromide instead of 2-bromo-1-(3-methoxyphenyl)-ethanone.

MS: 468 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.88 min.

### Example C24

### 3-(Isoquinolin-1-ylmethyl)-5-(3-methylbut-2-en-1-yl)-4-oxo-6-piperazin-1-yl-4,5-dihydro-3H-pyrrolo[3,2-d]pyrimidine-7-carbonitrile

This compound was prepared according to scheme 3.

The title compound was prepared analogously as described in Example C1 using 1-bromo-3-methyl-but-2-ene instead of 1-bromo-but-2-yne and using 1-(bromomethyl)-isoquinoline hydrobromide instead of 2-bromo-1-(3-methoxyphenyl)-ethanone.

MS: 454 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.78 min.

### Example C25

### 6-(1,4-Diazepan-1-yl)-5-(3,3-dichloroprop-2-en-1-yl)-3-(isoquinolin-1-ylmethyl)-4-oxo-4,5-dihydro-3H-pyrrolo[3,2-d]pyrimidine-7-carbonitrile

This compound was prepared according to scheme 3.

The title compound was prepared analogously as described in Example C12 using 3-bromo-1,1-dichloro-propene instead of 1-bromo-3-methyl-but-2-ene and using 1-(bromomethyl)-isoquinoline hydrobromide instead of 2-bromo-1-(3-methoxyphenyl)-ethanone.

MS: 508 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.90 min.

### Example C26

### 3-((3-Cyanopyridin-2-yl)methyl)-6-(1,4-diazepan-1-yl)-5-(3,3-dichloroprop-2-en-1-yl)-4-oxo-4,5-dihydro-3H-pyrrolo[3,2-d]pyrimidine-7-carbonitrile

This compound was prepared according to scheme 3.

The title compound was prepared analogously as described in Example C12 using 3-bromo-1,1-dichloro-propene instead of 1-bromo-3-methyl-but-2-ene and using 2-chloromethyl-nicotinonitrile instead of 2-bromo-1-(3-methoxyphenyl)-ethanone.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.30 min.

### Example D1

### 6-((R)-3-Amino-piperidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carbonitrile

This compound was prepared according to scheme 4.

### A {(R)-1-[7-Cyano-3-[2-(3-methoxy-phenyl)-2-oxo-ethyl]-5-(3-methyl-but-2-enyl)-4-oxo-4,5-dihydro-3H-pyrrolo[3,2-d]pyrimidin-6-yl]-piperidin-3-yl}-carbamic Acid tert-butyl Ester

The title compound was prepared analogously using the methods described in Examples C1 and B1, using 4-bromo-2-methylbut-2-ene instead of 1-bromo-but-2-yne.

MS: 575 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.94 min.

### B 6-((R)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile

To a solution of {(R)-1-[7-cyano-3-[2-(3-methoxy-phenyl)-2-oxo-ethyl]-5-(3-methyl-but-2-enyl)-4-oxo-4,5-dihydro-3H-pyrrolo[3,2-d]pyrimidin-6-yl]-piperidin-3-yl}-carbamic acid tert-butyl ester (140 mg, 0.24 mmol) in DCM (3 mL) was added TFA (3 mL) before stirring during 1 h at rt. After evaporation of the solvents, the residue was dissolved with DCM and washed with water and brine. The organic phase was dried and evaporated to give a crude compound which was purified by flash chromatography on silica gel (DCM/MeOH 9/1) to yield the title compound as a white solid.

MS: 475 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.24 min.

### Example D2

### 6-((R)-3-Amino-piperidin-1-yl)-5-but-2-ynyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1.

MS: 459 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.60 min.

### Example D3

### 6-((R)-3-Amino-piperidin-1-yl)-3-[2-(3-methoxy-phenyl)-2-oxo-ethyl]-5-methyl-4-oxo-4,5-dihydro-3H-pyrrolo[3,2-d]pyrimidine-7-carbonitrile

This compound was prepared according to scheme 4.

The title compound was prepared analogously as described in Examples C1 and D1, using iodomethane instead of 1-bromo-but-2-yne.

MS: 421.09 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.00 min.

### Example D4

### 6-((R)-3-Amino-piperidin-1-yl)-3-[2-(3-methoxy-phenyl)-2-oxo-ethyl]-5-(3-methyl -butyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1, using 1-bromo-3-methylbutane instead of 1-bromo-but-2-yne.

MS: 477.15 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.41 min.

### Example D5

### 6-((R)-3-Amino-piperidin-1-yl)-5-cyclopropylmethyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1, using cyclopropylmethyl bromide instead of 1-bromo-but-2-yne.

MS: 461.2 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.90 min.

### Example D6

### 6-((R)-3-Amino-piperidin-1-yl)-5-cyclobutyl methyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1, using bromomethyl(cyclobutane) instead of 1-bromo-but-2-yne.

MS: 475.2 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.19 min.

### Example D7

### 6-(3-Amino-pyrrolidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carbonitrile

This compound was prepared according to scheme 4.

The title compound was prepared analogously as described in Example D1, using 4-bromo-2-methylbut-2-ene instead of 1-bromo-but-2-yne, and 3-(t-butoxycarbonylamino)pyrrolidine instead of (R)-piperidin-3-yl-carbamic acid tert-butyl ester.

MS: 461 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.92 min.

### Example D8

### 6-((R)-3-Amino-piperidin-1-yl)-3-[2-(3-methoxy-phenyl)-2-oxo-ethyl]-4-oxo-5-thiazol-4-ylmethyl-4,5-dihydro-3H-pyrrolo[3,2-d]pyrimidine-7-carbonitrile

This compound was prepared according to scheme 4.

The title compound was prepared analogously as described in Examples C1 and D1, using 4-(chloromethyl)-1,3-thiazole hydrochloride instead of 1-bromo-but-2-yne.

MS: 504.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.60 min.

### Example D9

### 6-((S)-3-Amino-piperidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carbonitrile

This compound was prepared according to scheme 4.

The title compound was prepared analogously as described in Example D1, using (S)-piperidin-3-yl-carbamic acid tert-butyl ester instead of (R)-piperidin-3-yl-carbamic acid tert-butyl ester and using 4-bromo-2-methylbut-2-ene instead of 1-bromo-but-2-yne.

MS: 475 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.25 min.

### Example D10

### A mixture of 6-((R)-3-Amino-piperidin-1-yl)-3-[2-(3-methoxy-phenyl)-2-oxo-ethyl]-5-((1S,2S)-2-methyl-cyclopropylmethyl)-4-oxo-4,5-dihydro-3H-pyrrolo[3,2-d]pyrimidine-7-carbonitrile and 6-((R)-3-Amino-piperidin-1-yl)-3-[2-(3-methoxy-phenyl)-2-oxo-ethyl]-5-((1R,2R)-2-methyl-cyclopropylmethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1, using 1-bromomethyl-2-methyl cyclopropane instead of 1-bromo-but-2-yne.

MS: 475 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.42 min.

### Example D11

### 6-((R)-3-Amino-piperidin-1-yl)-3,5-bis-(3-methyl-but-2-enyl)-4-oxo-4,5-dihydro-3H-pyrrolo[3,2-d]pyrimidine-7-carbonitrile

This compound was prepared according to scheme 4.

The title compound was prepared analogously as described in Example D1. It was isolated as a by-product of the alkylation step using 4-bromo-2-methylbutene instead of using 1-bromo-but-2-yne. The bis-alkylated product was treated with (R)-piperidin-3-yl-carbamic acid tert-butyl ester using the methods as described in Example D1.

MS: 395 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.22 min.

### Example D12

### 6-((R)-3-Amino-piperidin-1-yl)-3-[2-(3-methoxy-phenyl)-2-oxo-ethyl]-5-(2-methyl -thiazol-4-ylmethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 2-methyl-4-chloromethylthiazole hydrochloride instead of 1-bromo-but-2-yne.

MS: 518.10 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.04 min.

### Example D13

### 6-((R)-3-Amino-piperidin-1-yl)-5-(2-cyclopropyl-ethyl)-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (2-bromo-ethyl)-cyclopropane instead of 1-bromo-but-2-yne.

MS: 475.18 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.36 min.

### Example D14

### 6-((R)-3-Amino-piperidin-1-yl)-5-isoxazol -5-ylmethyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 5-chloromethyl-isoxazole instead of 1-bromo-but-2-yne.

MS: 488.14 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.64 min.

### Example D15

### 6-((S)-3-Amino-piperidin-1-yl)-5-benzyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using benzyl bromide instead of 1-bromo-but-2-yne and (S)-piperidin-3-yl-carbamic acid tert-butyl ester instead of (R)-piperidin-3-yl-carbamic acid tert-butyl ester.

MS: 497 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.30 min.

### Example D16

### 6-((R)-3-Amino-piperidin-1-yl)-5-furan-3-ylmethyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 3-bromomethyl-furan instead of 1-bromo-but-2-yne.

MS: 487.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.95 min.

### Example D17

### 6-((R)-3-Amino-piperidin-1-yl)-5-benzyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using benzyl bromide instead of 1-bromo-but-2-yne.

MS: 497 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.30 min.

### Example D18

### 6-((R)-3-Amino-piperidin-1-yl)-3-[2-(3-methoxy-phenyl)-2-oxo-ethyl]-4-oxo-5-thiophen-2-ylmethyl-4,5-dihydro-3H-pyrrolo[3,2-d]pyrimidine-7-carbonitrile

This compound was prepared according to scheme 4.

The title compound was prepared analogously as described in Example C1 and D1 using 2-bromomethyl thiophene instead of 1-bromo-but-2-yne.

MS: 503.17 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.17 min.

### Example D19

### 6-((S)-3-Amino-piperidin-1-yl)-3-[2-(3-methoxy-phenyl)-2-oxo-ethyl]-4-oxo-5-thiophen-2-ylmethyl-4,5-dihydro-3H-pyrrolo[3,2-d]pyrimidine-7-carbonitrile

This compound was prepared according to scheme 4.

The title compound was prepared analogously as described in Examples C1 and D1 using 2-bromomethyl thiophene instead of 1-bromo-but-2-yne and (S)-piperidin-3-yl-carbamic acid tert-butyl ester instead of (R)-piperidin-3-yl-carbamic acid tert-butyl ester.

MS: 503.16 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.20 min.

### Example D20

### 6-((R)-3-Amino-piperidin-1-yl)-3-[2-(3-methoxy-phenyl)-2-oxo-ethyl]-4-oxo-5-thiophen-3-ylmethyl-4,5-dihydro-3H-pyrrolo[3,2-d]pyrimidine-7-carbonitrile

This compound was prepared according to scheme 4.

The title compound was prepared analogously as described in Examples C1 and D1 using 3-bromomethyl thiophene instead of 1-bromo-but-2-yne.

MS: 503.15 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.18 min.

### Example D21

### 6-((S)-3-Amino-piperidin-1-yl)-3-[2-(3-methoxy-phenyl)-2-oxo-ethyl]-4-oxo-5-thiophen-3-ylmethyl-4,5-dihydro-3H-pyrrolo[3,2-d]pyrimidine-7-carbonitrile

This compound was prepared according to scheme 4.

The title compound was prepared analogously as described in Examples C1 and D1 using 3-bromomethyl thiophene instead of 1-bromo-but-2-yne and (S)-piperidin-3-yl-carbamic acid tert-butyl ester instead of (R)-piperidin-3-yl-carbamic acid tert-butyl ester.

MS: 503.15 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.17 min.

### Example D22

### 6-((S)-3-Amino-piperidin-1-yl)-5-(3-methyl-but-2-enyl)-4-oxo-3-quinolin-4-ylmethyl-4,5-dihydro-3H-pyrrolo[3,2-d]pyrimidine-7-carbonitrile

This compound was prepared according to scheme 4.

The title compound was prepared analogously as described in Example D1 using (S)-piperidin-3-yl-carbamic acid tert-butyl ester instead of (R)-piperidin-3-yl-carbamic acid tert-butyl ester and 4-chloromethyl quinoline instead of 2-bromo-1-(3-methoxyphenyl)-ethanone and 4-bromo-2-methylbutene instead of using 1-bromo-but-2-yne.

MS: 468 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.90 min.

### Example D23

### 6-((R)-3-Amino-piperidin-1-yl)-5-(3-methyl-but-2-enyl)-4-oxo-3-quinolin-4-ylmethyl-4,5-dihydro-3H-pyrrolo[3,2-d]pyrimidine-7-carbonitrile

This compound was prepared according to scheme 4.

The title compound was prepared analogously as described in Example D1 using 4-chloromethylquinoline instead of 2-bromo-1-(3-methoxyphenyl)-ethanone and 4-bromo-2-methylbutene instead of using 1-bromo-but-2-yne.

MS: 468 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.88 min.

### Example D24

### 6-(3-Amino-azetidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile

This compound was prepared according to scheme 4.

The title compound was prepared analogously as described in Example D1 using azetidin-3-yl-carbamic acid tert-butyl ester instead of (R)-piperidin-3-yl-carbamic acid tert-butyl ester and 2-methoxyethanol instead of DMA and 4-bromo-2-methylbutene instead of using 1-bromo-but-2-yne.

MS: 447 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.74 min.

### Example D25

### 6-((S)-3-Amino-piperidin-1-yl)-5-(3-methyl-but-2-enyl)-4-oxo-3-quinolin-6-ylmethyl-4,5-dihydro-3H-pyrrolo[3,2-d]pyrimidine-7-carbonitrile

This compound was prepared according to scheme 4.

The title compound was prepared analogously as described in Example D1 using (S)-piperidin-3-yl-carbamic acid tert-butyl ester instead of (R)-piperidin-3-yl-carbamic acid tert-butyl ester and 6-chloromethylquinoline hydrochloride instead of 2-bromo-1-(3-methoxyphenyl)-ethanone and 4-bromo-2-methylbutene instead of using 1-bromo-but-2-yne.

MS: 468 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.59 min.

### Example D26

### 6-((S)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carbonitrile

This compound was prepared according to scheme 4.

The title compound was prepared analogously as described in Example D1 using (S)-piperidin-3-yl-carbamic acid tert-butyl ester instead of (R)-piperidin-3-yl-carbamic acid tert-butyl ester and 4-bromo-2-methylbutene instead of using 1-bromo-but-2-yne.

MS: 475.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.26 min.

### Example D27

### 6-((S)-3-Amino-piperidin-1-yl)-5-butyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and bromobutane instead of 1-bromo-but-2-yne.

MS: 463.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.08 min.

### Example D28

### 6-((R)-3-Amino-piperidin-1-yl)-5-butyl-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using bromobutane instead of 1-bromo-but-2-yne.

MS: 463.13 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.10 min.

### Example D29

### 6-((R)-3-Amino-piperidin-1-yl)-3-[2-(3-methoxy-phenyl)-2-oxo-ethyl]-4-oxo-5-(4,4,4-trifluoro-butyl)-4,5-dihydro-3H -pyrrolo[3,2-d]pyrimidine-7-carbonitrile

This compound was prepared according to scheme 4.

The title compound was prepared analogously as described in Examples C1 and D1 using 1-bromo-4,4,4-trifluorobutane instead of 1-bromo-but-2-yne.

MS: 517.10 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.28 min.

### Example D30

### 6-((S)-3-Amino-piperidin-1-yl)-5-(3,4-difluoro-benzyl)-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and 3,4-difluorobenzyl bromide instead of 1-bromo-but-2-yne.

MS: 533 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.42 min.

### Example D31

### 6-((S)-3-Amino-piperidin-1-yl)-3-[2-(3-methoxy-phenyl)-2-oxo-ethyl]-4-oxo-5-(2,4,5-trifluoro-benzyl)-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and 1-bromomethyl-2,4,5-trifluoro-benzene instead of 1-bromo-but-2-yne.

MS: 551 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.57 min.

### Example D32

### 6-((R)-3-Amino-piperidin-1-yl)-5-(3,4-difluoro-benzyl)-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 4-bromomethyl-1,2-difluoro-benzene instead of 1-bromo-but-2-yne.

MS: 533 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.45 min.

### Example D33

### 6-((S)-3-Amino-piperidin-1-yl)-3-isoquinolin-1-ylmethyl-5-(3-methyl-but-2-enyl)-4-oxo-4,5-dihydro-3H-pyrrolo[3,2-d]pyrimidine-7-carbonitrile

This compound was prepared according to scheme 4.

The title compound was prepared analogously as described in Example D1 using (S)-piperidin-3-yl-carbamic acid tert-butyl ester instead of (R)-piperidin-3-yl-carbamic acid tert-butyl ester and 4-bromo-2-methylbutene instead of 1-bromo-but-2-yne and 1-bromomethyl-isoquinoline hydrobromide instead of 2-bromo-1-(3-methoxyphenyl)-ethanone.

MS: 468 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.03 min.

### Example D34

### 6-((R)-3-Amino-piperidin-1-yl)-3-[2-(3-methoxy-phenyl)-2-oxo-ethyl]-4-oxo-5-(4,4,4-trifluoro-butyl)-4,5-dihydro-3H -pyrrolo[3,2-d]pyrimidine-7-carbonitrile

This compound was prepared according to scheme 4.

The title compound was prepared analogously as described in Examples C1 and D1 using 1-bromo-4,4,4-trifluorobutane instead of 1-bromo-but-2-yne.

MS: 517.10 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.28 min.

### Example D35

### 6-((S)-3-Amino-piperidin-1-yl)-5-(3,4-difluoro-benzyl)-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and 3,4-difluorobenzyl bromide instead of 1-bromo-but-2-yne.

MS: 533 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.42 min.

### Example D36

### 6-((S)-3-Amino-piperidin-1-yl)-5-(4-fluoro-benzyl)-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and 4-fluorobenzyl bromide instead of 1-bromo-but-2-yne.

MS: 515 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.30 min.

### Example D37

### 6-((R)-3-Amino-piperidin-1-yl)-5-(4-fluoro-benzyl)-3-[2-(3-methoxy-phenyl)-2-oxo-ethyl]-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 4-fluorobenzyl bromide instead of 1-bromo-but-2-yne.

MS: 515 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.34 min.

### Example D38

### 6-((S)-3-Amino-piperidin-1-yl)-5-benzyl-4-oxo-3-quinolin-4-ylmethyl-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and benzyl bromide instead of 1-bromo-but-2-yne and 4-chloromethyl-quinoline hydrochloride instead of 2-bromo-1-(3-methoxyphenyl)-ethanone.

MS: 490.10 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.77 min.

### Example D39

### 6-((S)-3-Amino-piperidin-1-yl)-5-benzyl-4-oxo-3-quinolin-4-ylmethyl-4,5-dihydro-3H-pyrrolo[3,2-d]pyrimidine-7-carbonitrile Hydrochloride

This compound was prepared according to scheme 4.

The title compound was prepared analogously as described in Example D38. The free-base was converted to the hydrochloride salt by treatment with 1M aqueous hydrochloric acid in acetonitrile and was freeze-dried for 18 hours to afford the title compound as the hydrochloride salt.

MS: 490.06 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.79 min.

### Example D40

### 6-((S)-3-Amino-piperidin-1-yl)-5-benzyl-3-[2-(3-methoxy-phenyl)-2-oxo-ethyl]-4-oxo-4,5-dihydro-3H-pyrrolo[3,2-d]pyrimidine-7-carbonitrile Hydrochloride

This compound was prepared according to scheme 4.

The title compound was prepared analogously as described in Example D15. The free-base was converted to the hydrochloride salt by treatment with 1M aqueous hydrochloric acid in acetonitrile and was freeze-dried for 18 hours to afford the title compound as the hydrochloride salt.

MS: 497.08 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.15 min.

### Example D41

### 6-((S)-3-Aminopiperidin-1-yl)-5-((3-fluorophenyl)methyl)-3-(2-(3-(methyloxy)phenyl)-2-oxoethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and 3-fluorobenzyl bromide instead of 1-bromo-but-2-yne.

MS: 515 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.27 min.

### Example D42

### 6-((S)-3-Aminopiperidin-1-yl)-3-(2-(3-(methyloxy)phenyl)-2-oxoethyl)-4-oxo-5-(pyridin-3-ylmethyl)-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and 3-bromomethylpyridine instead of 1-bromo-but-2-yne.

MS: 498 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.37 min.

### Example D43

### 6-((S)-3-Aminopiperidin-1-yl)-5-but-2-yn-1-yl-3-methyl-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester and using iodomethane instead of 2-bromo-1-(3-methoxyphenyl)-ethanone.

MS: 325 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.82 min.

### Example D44

### 6-((R)-3-Aminopiperidin-1-yl)-5-but-2-yn-1-yl-3-(isoquinolin-1-ylmethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 1-(bromomethyl)-isoquinoline hydrobromide instead of 2-bromo-1-(3-methoxyphenyl)-ethanone.

MS: 452 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.61 min.

### Example D45

### 6-((S)-3-Aminopiperidin-1-yl)-3-methyl-5-(3-methylbut-2-en-1-yl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 1-bromo-3-methyl-but-2-ene instead of 1-bromo-but-2-yne and using iodomethane instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 341 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.72 min.

### Example D46

### 6-((S)-3-Aminopiperidin-1-yl)-5-(3,3-dichloroprop-2-en-1-yl)-3-methyl-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 3-bromo-1,1-dichloro-propene instead of 1-bromo-but-2-yne and using iodomethane instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 381 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.10 min.

### Example D47

### 6-((S)-3-Aminopiperidin-1-yl)-3-((3-cyanopyridin-2-yl)methyl)-5-(3,3-dichloroprop-2-en-1-yl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 3-bromo-1,1-dichloro-propene instead of 1-bromo-but-2-yne and using 2-(chloromethyl)-nicotinonitrile instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.24 min.

### Example D48

### 6-((S)-3-Aminopiperidin-1-yl)-5-(3,3-dichloroprop-2-en-1-yl)-3-(2-(3-(methyloxy)phenyl)-2-oxoethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 3-bromo-1,1-dichloro-propene instead of 1-bromo-but-2-yne and using (S)-piperidin-3-yl-carbamic acid tert-butyl ester instead of (R)-piperidin-3-yl-carbamic acid tert-butyl ester.

MS: 515 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.64 min.

### Example D49

### 6-((S)-3-Aminopiperidin-1-yl)-5-(3,3-dichloroprop-2-en-1-yl)-3-(isoquinolin-1-ylmethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 3-bromo-1,1-dichloro-propene instead of 1-bromo-but-2-yne and using 1-(bromomethyl)-isoquinoline hydrobromide instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 508[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.48 min.

### Example D50

### 6-((S)-3-Aminopiperidin-1-yl)-5-buta-2,3-dien-1-yl-3-(2-(3-(methyloxy)phenyl)-2-oxoethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 4-bromo-buta-1,2-diene instead of 1-bromo-but-2-yne and using (S)-piperidin-3-yl-carbamic acid tert-butyl ester instead of (R)-piperidin-3-yl-carbamic acid tert-butyl ester.

MS: 459 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.74 min.

### Example D51

### 6-((S)-3-aminopiperidin-1-yl)-5-buta-2,3-dien-1-yl-3-(isoquinolin-1-ylmethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using 4-bromo-buta-1,2-diene instead of 1-bromo-but-2-yne and using 1-(bromomethyl)-isoquinoline hydrobromide instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 452[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.73 min.

### Example D52

### A mixture of 6-((S)-3-aminopiperidin-1-yl)-5-((E)-3-chloroprop-2-en-1-yl)-3-(2-(3-(methyloxy)phenyl)-2-oxoethyl)-4-oxo-4,5-dihydro-3H-pyrrolo[3,2-d]pyrimidine-7-carbonitrile and 6-((S)-3-aminopiperidin-1-yl)-5-((Z)-3-chloroprop-2-en-1-yl)-3-(2-(3-(methyloxy)phenyl)-2-oxoethyl)-4-oxo-4,5-dihydro-3H-pyrrolo[3,2-d]pyrimidine-7-carbonitrile

This compound was prepared according to scheme 4.

The title compounds were prepared analogously as described in Examples C1 and D1 using a mixture of (E and Z)-1,3-dichloro-propene instead of 1-bromo-but-2-yne and using (S)-piperidin-3-yl-carbamic acid tert-butyl ester instead of (R)-piperidin-3-yl-carbamic acid tert-butyl ester.

MS: 481/483[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.45 and 6.63 min.

### Example D53

### A mixture of 6-((S)-3-aminopiperidin-1-yl)-5-((E)-3-chlorobut-2-en-1-yl)-3-(2-(3-(methyloxy)phenyl)-2-oxoethyl)-4-oxo-4,5-dihydro-3H-pyrrolo[3,2-d]pyrimidine-7-carbonitrile and 6-((S)-3-aminopiperidin-1-yl)-5-((Z)-3-chlorobut-2-en-1-yl)-3-(2-(3-(methyloxy)phenyl)-2-oxoethyl)-4-oxo-4,5-dihydro-3H-pyrrolo[3,2-d]pyrimidine-7-carbonitrile

This compound was prepared according to scheme 4.

The title compounds were prepared analogously as described in Examples C1 and D1 using a mixture of (E and Z)-1,3-dichloro-but-2-ene instead of 1-bromo-but-2-yne and using (S)-piperidin-3-yl-carbamic acid tert-butyl ester instead of (R)-piperidin-3-yl-carbamic acid tert-butyl ester.

MS: 495/497[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.76 and 6.93 min.

### Example D54

### A mixture of 6-((S)-3-aminopiperidin-1-yl)-5-((E)-3-chloroprop-2-en-1-yl)-3-(isoquinolin-1-ylmethyl)-4-oxo-4,5-dihydro-3H-pyrrolo[3,2-d]pyrimidine-7-carbonitrile and 6-((S)-3-aminopiperidin-1-yl)-5-((Z)-3-chloroprop-2-en-1-yl)-3-(isoquinolin-1-ylmethyl)-4-oxo-4,5-dihydro-3H-pyrrolo[3,2-d]pyrimidine-7-carbonitrile

This compound was prepared according to scheme 4.

The title compounds were prepared analogously as described in Examples C1 and D1 using a mixture of (E and Z)-1,3-dichloro-propene instead of 1-bromo-but-2-yne and using 1-(bromomethyl)-isoquinoline hydrobromide instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 474[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.21 and 6.42 min.

### Example D55

### A mixture of 6-((S)-3-aminopiperidin-1-yl)-5-((E)-3-chlorobut-2-en-1-yl)-3-(isoquinolin-1-ylmethyl)-4-oxo-4,5-dihydro-3H-pyrrolo[3,2-d]pyrimidine-7-carbonitrile and 6-((S)-3-aminopiperidin-1-yl)-5-((Z)-3-chlorobut-2-en-1-yl)-3-(isoquinolin-1-ylmethyl)-4-oxo-4,5-dihydro-3H-pyrrolo[3,2-d]pyrimidine-7-carbonitrile

This compound was prepared according to scheme 4.

The title compounds were prepared analogously as described in Examples C1 and D1 using a mixture of (E and Z)-1,3-dichloro-but-2-ene instead of 1-bromo-but-2-yne and using 1-(bromomethyl)-isoquinoline hydrobromide instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 488[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.21 and 6.42 min.

### Example D56

### 6-((S)-3-Aminopiperidin-1-yl)-5-but-2-yn-1-yl-3-(2-(3-(methyloxy)phenyl)-2-oxoethyl)-4-oxo-4,5-dihydro-3H-pyrrolo[3,2-d]pyrimidine-7-carbonitrile

This compound was prepared according to scheme 4.

The title compound was prepared analogously as described in Examples C1 and D1 using (S)-piperidin-3-yl-carbamic acid tert-butyl ester instead of (R)-piperidin-3-yl-carbamic acid tert-butyl ester.

MS: 459[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.46 min.

### Example D57

### 6-[(3S)-3-Aminopiperidin-1-yl]-5-(3,3-difluoroprop-2-en-1-yl)-3-[2-(3-methoxyphenyl)-2-oxoethyl]-4-oxo-4,5-dihydro-3H-pyrrolo[3,2-d]pyrimidine-7-carbonitrile

This compound was prepared according to scheme 4.

The title compound was prepared analogously as described in Example D1 using 3-iodo-1,1-difluoro-propene instead of 1-bromo-3-methyl-but-2-ene and using (S)-piperidin-3-yl-carbamic acid tert-butyl ester instead of (R)-piperidin-3-yl-carbamic acid tert-butyl ester.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.87 min.

### Example D58

### 6-[(3S)-3-Aminopiperidin-1-yl]-5-(2-cyclopropylidene-ethyl)-3-(isoquinolin-1-ylmethyl)-4-oxo-4,5-dihydro-3H-pyrrolo[3,2-d]pyrimidine-7-carbonitrile

This compound was prepared according to scheme 4.

### A 6-Bromo-5-(2-cyclopropylidene-ethyl)-4-oxo-4,5-dihydro-3H-pyrrolo[3,2-d]pyrimidine-7-carbonitrile

Sodium hydride (20 mg, 0.50 mmol of a 60% dispersion in oil) was added to a solution of 6-bromo-4-oxo-4,5-dihydro-3H-pyrrolo[3,2-d]pyrimidine-7-carbonitrile (120 mg, 0.50 mmol) in dimethylformamide (3 mL) and the mixture was stirred under a nitrogen atmosphere. Tetrakis-(triphenylphosphine)palladium(0) complex (30 mg, 0.025 mmol) was then added followed by toluene-4-sulfonic acid 1-vinyl-cyclopropyl ester (120 mg, 0.50 mmol). The reaction mixture was then stirred at room temperature overnight. The mixture was evaporated and the residue partioned between water and ethyl acetate. The organic layer was then washed with water and evaporated. The residue was purified by flash chromatography (silica, eluting with 2% methanol in dichloromethane). Appropriate fractions were combined, evaporated and dried to afford the title compound as an off-white foam.

MS: 305/307 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.81 min.

### B 6-[(3S)-3-Aminopiperidin-1-yl]-5-(2-cyclopropylidene-ethyl)-3-(isoquinolin-1-ylmethyl)-4-oxo-4,5-dihydro-3H-pyrrolo[3,2-d]pyrimidine-7-carbonitrile

The title compound was prepared analogously as described in Example D1 from 6-bromo-5-(2-cyclopropylidene-ethyl)-4-oxo-4,5-dihydro-3H-pyrrolo[3,2-d]pyrimidine-7-carbonitrile using 1-(bromomethyl)-isoquinoline hydrobromide instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 466 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.34 min.

### Example E1

### 5-(2-Chloro-phenyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

This compound was prepared according to scheme 5.

### A 2-[(2-Chloro-phenylamino)-methylene]-malononitrile

A solution of ethoxymethylenemalononitrile (10 g, 82.0 mmol) and 2-chloroaniline (12.9 mL, 12.3 mmol) in absolute ethanol (50 mL) was heated at reflux for 2 hours then cooled. The precipitate was collected by filtration, washed with cold ethanol to give, and dried at 80° C. in vacuo to give the title compound as a pale yellow solid.

MS: 204 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.12 min.

### B 3-Amino-1-(2-chloro-phenyl)-4-cyano-1H-pyrrole-2-Carboxylic Acid Ethyl Ester

A mixture of 2-[(2-chloro-phenylamino)-methylene-malononitrile (9.79 g, 48.1 mmol), ethyl bromoacetate (7.99 mL, 72.1 mmol) and potassium carbonate (13.25 g, 96.2 mmol) in DMF (160 mL) was heated at 90° C. for 50 minutes. After cooling, a freshly prepared solution of sodium ethoxide in ethanol (62.37 mL of a 1M solution) was added dropwise. When the addition was complete the mixture was heated at 90° C. for 25 minutes. Glacial acetic acid (5 ml) was added, and the solvent removed. The residue was partitioned between ethyl acetate (2×250 mL) and water (250 mL) and the combined organic phases were washed with water (250 mL) and brine (250 mL) and dried (Na₂SO₄). Evaporation of the solvent gave a dark coloured solid. The crude product was purified by flash chromatography on silica (450 mL), loading in DCM and eluting with 20% ethyl acetate/petrol (40-60° C.) to neat ethyl acetate. Recrystallisation from hot ethyl acetate and gave the title compound as a cream coloured solid.

MS: 290 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.49 min.

### C 5-(2-Chlorophenyl)-4-oxo-4,5-dihydro-3H-pyrrolo[3,2-d]pyrimidine-7-carbonitrile

A 25% solution of sodium methoxide in MeOH (9.5 mL) was added to a stirred suspension of 3-amino-1-(2-chloro-phenyl)-4-cyano-1H-pyrrole-2-carboxylic acid ethyl ester (1.5 g, 5.18 mmol) in formamide (9 mL). After stirring at room temperature for 15 mins, the mixture was heated to 100° C. for 2 hours. The mixture was cooled and poured onto iced water (50 mL) containing concentrated aqueous hydrochloric acid (5 mL). A pale yellow precipitate was formed and after stirring for 10 mins, the solid was collected by filtration, washed with water (2×15 mL) and dried in vacuo at 40° C. to give the title compound.

MS: 271/273 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.61 min.

### D 6-Chloro-5-(2-chloro-phenyl)-4-oxo-4,5-dihydro-3H-pyrrolo[3,2-d]pyrimidine-7-carbonitrile

A mixture of 5-(2-chloro-phenyl)-4-oxo-4,5-dihydro-3H-pyrrolo[3,2-d]pyrimidine-7-carbonitrile (0.9 g, 3.33 mmol) and N-chlorosuccinimide (1.32 g, 9.95 mmol) in DMF (13.5 mL) was stirred at room temperature for 96 hours. The reaction mixture was poured onto iced water (150 mL). The solid was filtered off, dissolved in chloroform (300 mL), dried (MgSO₄) and concentrated in vacuo to afford the title compound as a pale yellow solid.

MS: 305/307/309 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.99 min.

### E 6-Chloro-5-(2-chloro-phenyl)-3-[2-(3-methoxy-phenyl)-2-oxo-ethyl]-4-oxo-4,5-dihydro-3H-pyrrolo[3,2-d]pyrimidine-7-carbonitrile

6-Chloro-5-(2-chloro-phenyl)-4-oxo-4,5-dihydro-3H-pyrrolo[3,2-d]pyrimidine-7-carbonitrile (0.6 g, 1.97 mmol) was dissolved in DMF (20 mL). To this was added potassium carbonate (0.326 g, 2.36 mmol) followed by 2-bromo-1-(3-methoxyphenyl)-ethanone (0.495 g, 2.16 mmol). The reaction mixture was stirred at room temperature for 1 hours The solvent was concentrated in vacuo and the residue was triturated with water (ca. 25 mL). The solid was collected by filtration and purified by flash chromatography (Silica, eluent: DCM to 20% ethyl acetate in DCM ) to give the title compound as an orange foam.

MS: 453.12 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.85 min.

### F 5-(2-Chloro-phenyl)-3-[2-(3-methoxy-phenyl)-2-oxo-ethyl]-4-oxo-6-piperazin-1-yl-4,5-dihydro-3H-pyrrolo[3,2-d]pyrimidine-7-carbonitrile

A mixture of 6-Chloro-5-(2-chloro-phenyl)-3-[2-(3-methoxy-phenyl)-2-oxo-ethyl]-4-oxo-4,5-dihydro-3H-pyrrolo[3,2-d]pyrimidine-7-carbonitrile (0.1 g, 0.22 mmol) and piperazine (0.19 g, 2.21 mmol) in DMA (2 mL) was treated with microwave irradiation (Smith Microwave Synthesizer) at 160° C. for 15 mins. The solvent was removed in vacuo and the residue was suspended in DCM (50 mL). The suspension was washed with water (50 mL), saturated sodium hydrogen carbonate solution (50 mL) and brine (50 mL), dried (MgSO4) and concentrated in vacuo. The residue was purified by flash chromatography (Silica, eluent: DCM to 5% MeOH in DCM ) to give the title compound as a pale yellow foam.

MS: 503.09 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.64 min.

### Example E2

### 6-((S)-3-Aminopiperidin-1-yl)-3-(2-(3-(methyloxy)phenyl)-2-oxoethyl)-4-oxo-5-phenyl-4,5-dihydro-3H-pyrrolo[3,2-d]pyrimidine-7-carbonitrile

This compound was prepared according to scheme 5.

### A 6-Chloro-3-[2-(3-methoxy-phenyl)-2-oxo-ethyl]-4-oxo-5-phenyl-4,5-dihydro-3H-pyrrolo[3,2-d]pyrimidine-7-carbonitrile

The title compound was prepared analogously as described in Example E1, steps A to E, using aniline instead of 2-chloroaniline.

MS: 419/421 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.71 min.

### B 6-((S)-3-Aminopiperidin-1-yl)-3-(2-(3-(methyloxy)phenyl)-2-oxoethyl)-4-oxo-5-phenyl-4,5-dihydro-3H-pyrrolo[3,2-d]pyrimidine-7-carbonitrile

A mixture of 6-chloro-3-[2-(3-methoxy-phenyl)-2-oxo-ethyl]-4-oxo-5-phenyl-4,5-dihydro-3H-pyrrolo[3,2-d]pyrimidine-7-carbonitrile (100 mg, 0.24 mmol) and (S)-piperidin-3-yl-carbamic acid tert-butyl ester (240 mg, 1.19 mmol) in DMA (4 ml) was heated at 160° C. for 30 minutes. The reaction was concentrated in vacuo, and the residue was purified by reversed phase preparative HPLC, using a CH₃CN/H₂O/0.1% TFA mobile phase. Recovered fractions were concentrated in vacuo, and the residue was dissolved in a solution of TFA (2 ml) in DCM (2 ml). After 1 hour at room temperature, the reaction mixture was concentrated to dryness, redissolved in DCM and the solution was evaporated to dryness. The residue was redissolved again in DCM and the solution was washed (×2) with saturated aqueous sodium bicarbonate, dried (Na₂SO₄), and concentrated and dried at 60° C. in vacuo to give the title compound as a pale yellow solid.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.68 min.

### Example E3

### 6-((R)-3-Aminopiperidin-1-yl)-3-(2-(3-(methyloxy)phenyl)-2-oxoethyl)-4-oxo-5-phenyl-4,5-dihydro-3H-pyrrolo[3,2-d]pyrimidine-7-carbonitrile

This compound was prepared according to scheme 5.

The title compound was prepared analogously as described in Example E2 using (R)-piperidin-3-yl-carbamic acid tert-butyl ester instead of (S)-piperidin-3-yl-carbamic acid tert-butyl ester.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.78 min.

### Example E4

### 6-((S)-3-Aminopiperidin-1-yl)-3-(isoquinolin-1-ylmethyl)-4-oxo-5-phenyl-4,5-dihydro-3H-pyrrolo[3,2-d]pyrimidine-7-carbonitrile Hydrochloride

This compound was prepared according to scheme 5.

### A 6-Chloro-5-phenyl-4-oxo-4,5-dihydro-3H-pyrrolo[3,2-d]pyrimidine-7-carbonitrile

The title compound was prepared analogously as described in Example E1, steps A to D, using aniline instead of 2-chloroaniline.

MS: 271/273 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.77 min.

### B [(S)-1-(7-Cyano-4-oxo-5-phenyl-4,5-dihydro-3H-pyrrolo[3,2-d]pyrimidin-6-yl)-piperidin-3-yl]-carbamic Acid ter-butyl Ester

A mixture of 6-chloro-5-phenyl-4-oxo-4,5-dihydro-3H-pyrrolo[3,2-d]pyrimidine-7-carbonitrile (360 mg, 1.33 mmol) and (S)-piperidin-3-yl-carbamic acid tert-butyl ester (800 mg, 3.99 mmol) in DMA (5 mL) was heated by microwave irradiation at 150° C. for 45 minutes. The mixture was evaporated and the residue was partitioned between water and ethyl acetate. The organic layer was evaporated and purified by flash chromatography (Silica, eluting with 5% MeOH/DCM). Fractions containing the main component were combined and evaporated to give the title compound a colourless oil.

MS: 435 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.14 min.

### C [(S)-1-(7-Cyano-3-isoquinolin-1-ylmethyl-4-oxo-5-phenyl-4,5-dihydro-3H-pyrrolo[3,2-d]pyrimidin-6-yl)-piperidin-3-yl]-carbamic Acid tert-butyl Ester

Potassium carbonate (135 mg, 0.97 mmol) and 1-(bromomethyl)-isoquinoline hydrobromide (97 mg, 0.32 mmol) were added to a solution of [(S)-1-(7-cyano-4-oxo-5-phenyl-4,5-dihydro-3H-pyrrolo[3,2-d]pyrimidin-6-yl)-piperidin-3-yl]-carbamic acid tert-butyl ester (140 mg, 0.32 mmol) in DMF and the mixture was stirred at room temperature for 2 hours. The mixture was evaporated and the residue partitioned between water and ethyl acetate. The organic layer was evaporated and the residue purified by flash chromatography (Silica, eluting with 2% MeOH/DCM). Fractions containing the main component were combined and evaporated to give the title compound as a white foam.

MS: 576 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 3.87 min.

### D 6-((S)-3-Aminopiperidin-1-yl)-3-(isoquinoline-1-ylmethyl)-4-oxo-5-phenyl-4,5-dihydro-3H-pyrrolo[3,2-d]pyrimidine-7-carbonitrile Hydrochloride

[(S)-1-(7-Cyano-3-isoquinolin-1-ylmethyl-4-oxo-5-phenyl-4,5-dihydro-3H-pyrrolo[3,2d]pyrimidin-6-yl)-piperidin-3-yl]-carbamic acid tert-butyl ester (78 mg, 0.136 mmol) was dissolved in a mixture of TFA (1 mL) and DCM (1 mL) and was stirred at room temperature for 2 hours. The mixture was evaporated and the residue evaporated several times from toluene. The residue was then passed down a SCX column eluting firstly with MeOH and then 2M ammonia in MeOH. Appropriate fractions were combined and evaporated to give the free base of the title compound as an oil. The oil was dissolved in MeOH and excess 1.25M hydrogen chloride in MeOH was added. The mixture was evaporated by blow down to give the title compound as a buff solid.

MS: 476 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.40 min.

### Example E5

### 6-((S)-3-Aminopiperidin-1-yl)-3-methyl-4-oxo-5-phenyl-4,5-dihydro-3H-pyrrolo[3,2-d]pyrimidine-7-carbonitrile Hydrochloride

This compound was prepared according to scheme 5.

The title compound was prepared analogously as described in Example E4 using iodomethane instead of 1-(bromomethyl)-isoquinoline hydrobromide.

MS: 349 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.74 min.

### Example E6

### 6-((S)-3-Aminopiperidin-1-yl)-5-(4-fluorophenyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 4-fluoroaniline instead of 2-chloroaniline.

MS: 494 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.70 min.

### Example E7

### 6-((S)-3-Aminopiperidin-1-yl)-5-(4-fluorophenyl)-3-methyl-4-oxo-4,5-dihydro-3H-pyrrolo[3,2-d]pyrimidine-7-carbonitrile hydrochloride

This compound was prepared according to scheme 5.

The title compound was prepared analogously as described in Example E4 using 4-fluoroaniline instead of 2-chloroaniline and using iodomethane instead of 1-(bromomethyl)-isoquinoline hydrobromide.

MS: 367 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.97 min.

### Example E8

### 6-((S)-3-Aminopiperidin-1-yl)-5-(3,4-difluorophenyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 3,4-difluoroaniline instead of 2-chloroaniline.

MS: 512 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.92 min.

### Example E9

### 6-((S)-3-Aminopiperidin-1-yl)-5-(3,4-difluorophenyl)-3-methyl-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 3,4-difluoroaniline instead of 2-chloroaniline and using iodomethane instead of 1-(bromomethyl)-isoquinoline hydrobromide.

MS: 385 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.23 min.

### Example E10

### 6-((S)-3-Aminopiperidin-1-yl)-5-(3-fluorophenyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 3-fluoroaniline instead of 2-chloroaniline.

MS: 494 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.70 min.

### Example E11

### 6-((S)-3-Aminopiperidin-1-yl)-5-(3-fluorophenyl)-3-methyl-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 3-fluoroaniline instead of 2-chloroaniline and using iodomethane instead of 1-(bromomethyl)-isoquinoline hydrobromide.

MS: 367[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.04 min.

### Example E12

### 6-((S)-3-Aminopiperidin-1-yl)-5-(4-chlorophenyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 4-chloroaniline instead of 2-chloroaniline.

MS: 510 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.14 min.

### Example E13

### 6-((S)-3-Aminopiperidin-1-yl)-5-(4-chlorophenyl)-3-methyl-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 4-chloroaniline instead of 2-chloroaniline and using iodomethane instead of 1-(bromomethyl)-isoquinoline hydrobromide.

MS: 383[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.54 min.

### Example E14

### 6-((S)-3-Aminopiperidin-1-yl)-5-(2,4-difluorophenyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 2,4-difluoroaniline instead of 2-chloroaniline.

MS: 512 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.80/6.92 min.

### Example E15

### 6-((S)-3-Aminopiperidin-1-yl)-5-(2,4-difluorophenyl)-3-methyl-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 2,4-difluoroaniline instead of 2-chloroaniline and using iodomethane instead of 1-(bromomethyl)-isoquinoline hydrobromide.

MS: 385 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.13/5.35 min.

### Example E16

### 6-((S)-3-Aminopiperidin-1-yl)-5-(2-chloro-4-fluorophenyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 5.

The title compound was prepared analogously as described in Example E4 using 2-chloro-4-fluoroaniline instead of 2-chloroaniline.

MS: 528 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.22/6.39 min.

### Example E17

### 6-((S)-3-Aminopiperidin-1-yl)-3-(2-(3-(methyloxy)phenyl)-2-oxoethyl)-4-oxo-5-pyridin-2-yl-4,5-dihydro-3H-pyrrolo[3,2-d]pyrimidine-7-carbonitrile Hydrochloride

The title compound was prepared by adaptation of the route depicted in scheme 5.

### A 2-Amino-4-cyano-1-pyridin-2-yl-1H-pyrrole-3-carboxylic Acid Methyl Ester

A mixture of 2-amino-4-cyano-1H-pyrrole-3-carboxylic acid methyl ester (2.5 g, 15.1 mmol), (1S,2S)—N,N′-dimethyl-cyclohexane-1,2-diamine (432 mg, 3.03 mmol), cesium carbonate (10.36 g, 31.8 mmol), 2-bromo-pyridine (2.63 g, 16.6 mmol) and copper (1) iodode (144 mg, 0.76 mmol) in 1,2-dimethoxyethane (10 mL) was heated by microwave irradiation at 160° C. for 15 min. The reaction mixture was concentrated in vacuo and the residue was partitioned between DCM (2×200 mL) and saturated aqueous sodium bicarbonate (100 mL). The DCM phase was washed with water and brine, and dried (Na₂SO₄). After evaporation of the solvent the residual oil was purified by flash chromatography (Silica, eluting with ethyl acetate/petrol (40-60° C.) [1:4]) to give the title compound as a cream coloured solid.

MS: 243 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.39 min.

### B 6-Chloro-4-oxo-5-pyridin-2-yl-4,5-dihydro-3H-pyrrolo[3,2-d]pyrimidine-7-carbonitrile

The title compound was prepared by the methods described in Example E1, steps C and D, using 2-amino-4-cyano-1-pyridin-2-yl-1H-pyrrole-3-carboxylic acid methyl ester instead of 3-amino-1-(2-chloro-phenyl)-4-cyano-1H-pyrrole-2-carboxylic acid ethyl ester.

MS: 272 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.23 min.

### C 6-((S)-3-Aminopiperidin-1-yl)-3-(2-(3-(methyloxy)phenyl)-2-oxoethyl)-4-oxo-5-Pyridin-2-yl-4,5-dihydro-3H-pyrrolo[3,2-d]pyrimidine-7-carbonitrile Hydrochloride

The title compound was prepared by the methods described in Example E4, steps B to D, using 6-chloro-4-oxo-5-pyridin-2-yl-4,5-dihydro-3H-pyrrolo[3,2-d]pyrimidine-7-carbonitrile instead of 6-chloro-5-phenyl-4-oxo-4,5-dihydro-3H-pyrrolo[3,2-d]pyrimidine-7-carbonitrile and using 2-bromo-1-(3-methoxy-phenyl)-ethanone instead of 1-(bromomethyl)-isoquinoline hydrobromide.

MS: 484 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.11 min.

### Example F1

### 6-((R)-3-Aminopiperidin-1-yl)-5-benzyl-3-[2-(3-methoxyphenyl)-2-oxoethyl]-1-methyl-2,4-dioxo-2,3,4,5-tetrahydro-1H-pyrrolo[3,2-d]pyrimidine-7-carbonitrile

This compound was prepared according to scheme 6.

### A 3-Amino-1-benzyl-4-cyano-1H-pyrrole-2-carboxylic Acid Ethyl Ester

A mixture of 2-(benzylamino-methylene)-malononitrile (10.94 g, 59.8 mmol), ethyl bromoacetate (9.94 mL, 89.7 mmol) and potassium carbonate (16.5 g, 119.6 mmol) were in DMF (200 mL) was heated at 90° C. for 50 mins. After cooling to 40° C., sodium ethoxide (77.7 mL of a 1M solution in ethanol) was added dropwise during 10 mins. The reaction mixture was heated to 90° C. for 25 mins. Glacial acetic acid (6.2 mL) was added and the reaction mixture was left to cool. The DMF was removed in vacuo and the residue was partitioned between ethyl acetate (200 mL) and water (200 mL). The layers were separated and the organic layer was washed with water (200 mL) and brine (200 mL), and dried (Na₂SO₄) Concentration gave a dark orange solid which was purified by flash chromatography (Silica, gradient elution with 10% ethyl acetate in cyclohexane to ethyl acetate). Fractions containing pure material were combined and concentrated to afford the title compound as a yellow solid.

MS: 270 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.30 min.

### B 1-Benzyl-3-(3-benzyl-ureido)-4-cyano-1H-pyrrole-2-carboxylic Acid Ethyl Ester

A mixture of 3-amino-1-benzyl-4-cyano-1H-pyrrole-2-carboxylic acid ethyl ester (3.67 g, 13.6 mmol.) and benzyl isocyanate (2.53 mL, 20.5 mmol.) in pyridine (73 mL) was treated with microwave irradiation (Emrys Optimizer) at 120° C. for 30 mins. The reaction mixture was partitioned between ethyl acetate (100 mL) and 1M aq. hydrochloric acid (4×100 mL). The organic extract was dried (Na₂SO₄), filtered, concentrated in vacuo and the residue was purified by trituration with diethyl ether (50 mL), filtration and drying in a vacuum at 40° for 24 hours to afford the title compound as an off-white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### C 3,6-Dibenzyl-4-imino-2-oxo-2,3,4,6-tetrahydro-1H-pyrrolo[3,4-d]pyrimidine-7-carboxylic Acid Ethyl Ester

A mixture of 1-benzyl-3-(3-benzyl-ureido)-4-cyano-1H-pyrrole-2-carboxylic acid ethyl ester (2 g, 5 mmol.) and sodium methoxide (0.27 g, 5 mmol.) in MeOH (60 mL) was treated with microwave irradiation (Emrys Optimizer) at 60° C. for 5 mins. The solid that was formed was collected by filtration, washed with MeOH (20 mL) and air-dried to afford the title compound as a white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.13 min.

### D 3,5-Dibenzyl-2,4-dioxo-2,3,4,5-tetrahydro-1H-pyrrolo[3,2-d]pyrimidine-7-carbonitrile

A suspension of 3,6-dibenzyl-4-imino-2-oxo-2,3,4,6-tetrahydro-1H-pyrrolo[3,4-d]pyrimidine-7-carboxylic acid ethyl ester (1.13 g, 2.8 mmol.) and sodium methoxide (0.46 g, 8.4 mmol.) in MeOH (30 mL) was treated with microwave irradiation (Emrys Optimizer) at 140° C. for 20 mins. The reaction mixture was concentrated in vacuo and the solid obtained was triturated with water (10 mL), filtered and dried under vacuum at 40° C. for 24 hours to afford the title compound as a white solid.

MS: 357 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.36 min.

### E 3,5-Dibenzyl-1-methyl-2,4-dioxo-2,3,4,5-tetrahydro-1H-pyrrolo[3,2-d]pyrimidine-7-carbonitrile

3,5-Dibenzyl-2,4-dioxo-2,3,4,5-tetrahydro-1H-pyrrolo[3,2-d]pyrimidine-7-carbonitrile (0.95 g, 2.7 mmol.) was dissolved in DMSO (10 mL). To this was added potassium carbonate (0.74 g, 5.3 mmol.) followed by methyl iodide (0.25 mL, 4.0 mmol.). The reaction mixture was stirred at room temperature for 3 hours. A dense white precipitate was formed and the reaction mixture was diluted with water (20 mL). The solid was collected by filtration, washed with water (10 mL) and dried under vacuum at 40° C. for 72 hours to afford the title compound as a white solid.

MS: no mass ion.

T_{R} [HPLC, Phenomenex Luna 3 micron C18; 5-95% CH₃CN+0.1% Formic acid/H₂O+0.1% Formic acid for 5 min, flow 2.0 ml/min]: 3.86 min.

### F 5-Benzyl-1-methyl-2,4-dioxo-2,3,4,5-tetrahydro-1H-pyrrolo[3,2-d]pyrimidine-7-carbonitrile

A mixture of 3,5-dibenzyl-1-methyl-2,4-dioxo-2,3,4,5-tetrahydro-1H-pyrrolo[3,2-d]pyrimidine-7-carbonitrile (0.90 g, 2.4 mmol.) and boron tribromide (12.16 mL, 12.2 mmol.) in xylene (50 mL) was stirred at 140° C. for 5 hours. The reaction mixture was cooled, MeOH (15 mL) was added and the mixture was stirred at room temperature for 30 mins. The solvents were evaporated in vacuo and the residue was partitioned between ethyl acetate (100 mL) and saturated aq. sodium hydrogen carbonate (200 mL). The ethyl acetate suspension was concentrated in vacuo and the residue was triturated with diethyl ether (100 mL), filtered and air-dried to afford the title compound as a beige solid.

MS: 281 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.74 min.

### G 5-Benzyl-6-bromo-1-methyl-2,4-dioxo-2,3,4,5-tetrahydro-1H-pyrrolo[3,2-d]pyrimidine-7-carbonitrile

5-Benzyl-1-methyl-2,4-dioxo-2,3,4,5-tetrahydro-1H-pyrrolo[3,2-d]pyrimidine-7-carbonitrile (0.37 g, 1.3 mmol.) was suspended in acetic acid (8 mL) and warmed to 45° C. To this was added bromine (0.10 mL, 2.0 mmol.) dropwise, in acetic acid (2 mL). Once the addition was complete, water (3 mL) was added and the reaction mixture was stirred at 45° C. for 18 hours. Another 1.5 equivalents of bromine, in 3 mL acetic acid was added and the reaction mixture was stirred at 45° C. for 4 hours. Another 2 equivalents of bromine and 10 mL acetic acid were added and the reaction mixture was stirred at 70° C. for 72 hours. The solvents were removed in vacuo and the residue was triturated with saturated aq. sodium thiosulphite solution (20 mL), followed by water (10 mL). The solid was collected by filtration and was dried under vacuum at 40° C. for 18 hours to obtain the title compound as a fawn coloured solid.

MS: 359 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.07 min.

### H 5-Benzyl-6-bromo-3-[2-(3-methoxyphenyl)-2-oxo-ethyl]-1-methyl-2,4-dioxo-2,3,4,5-tetrahydro-1H-pyrrolo[3,2-d]pyrimidine-7-carbonitrile

5-Benzyl-6-bromo-1-methyl-2,4-dioxo-2,3,4,5-tetrahydro-1H-pyrrolo[3,2-d]pyrimidine-7-carbonitrile (0.2 g, 0.56 mmol.) was dissolved in DMF (10 mL). To this was added potassium carbonate (0.092 g, 0.67 mmol.), followed by 2-bromo-1-(3-methoxyphenyl)-ethanone (0.14 g, 0.61 mmol.). The reaction mixture was stirred at room temperature for 2.5 hours. Another 0.2 equivalents of 2-Bromo-3′methoxyacetophenone was added and the reaction mixture was stirred at room temperature for 1 hours DCM (15 mL) was added and the potassium carbonate was filtered off. The solvents were concentrated in vacuo and the residue was triturated with water (30 mL), filtered and dried under vacuum at 40° C. for 18 hours, to afford the title compound as a mustard coloured solid.

MS: no mass ion.

T_{R} [HPLC, Phenomenex Luna 3 micron C18; 5-95% CH₃CN+0.1% Formic acid/H₂O+0.1% Formic acid for 5 min, flow 2.0 ml/min]: 3.99 min.

### I 6-((R)-3-Aminopiperidin-1-yl)-5-benzyl-3-[2-(3-methoxyphenyl)-2-oxoethyl]-1-methyl-2,4-dioxo-2,3,4,5-tetrahydro-1H-pyrrolo[3,2-d]pyrimidine-7-carbonitrile

A mixture of 5-benzyl-6-bromo-3-[2-(3-methoxyphenyl)-2-oxo-ethyl]-1-methyl-2,4-dioxo-2,3,4,5-tetrahydro-1H-pyrrolo[3,2-d]pyrimidine-7-carbonitrile (0.1 g, 0.20 mmol.) and (R)-piperidin-3-yl-carbamic acid tert-butyl ester (0.12 g, 0.59 mmol.) in DMA (6 mL) was stirred at 130° C. for 18 hours. The DMA was concentrated in vacuo to give an orange gum. This was treated with TFA:DCM (1.5 mL:1.5 mL) at room temperature for 30 mins. The solvents were removed in vacuo and the residue was purified by reverse phase HPLC, eluting on a gradient of 10%-60% CH₃CN+0.1% Formic acid/H₂O+0.1% Formic acid, over 50 mins, with a flowrate of 5 mL/min. The obtained solid was purified by flash chromatography (SCX-2 column, eluting with 1:1 MeOH:DCM, MeOH and 2M NH3 solution in MeOH) and concentrated in vacuo to afford the title compound as a yellow solid.

MS: 527 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 7.72 min.

### Example F2

### 6-((S)-3-Aminopiperidin-1-yl)-5-benzyl-3-[2-(3-methoxyphenyl)-2-oxoethyl]-1-methyl-2,4-dioxo-2,3,4,5-tetrahydro-1H-pyrrolo[3,2-d]pyrimidine-7-carbonitrile

This compound was prepared according to scheme 6.

The title compound was prepared analogously as described in Example F1 using (S)-piperidin-3-yl-carbamic acid tert-butyl ester instead of (R)-piperidin-3-yl-carbamic acid tert-butyl ester.

MS: 527 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 7.63 min.

### Example F3

### 6-((S)-3-Aminopiperidin-1-yl)-1,3-dimethyl-2,4-dioxo-5-(phenylmethyl)-2,3,4,5-tetrahydro-1H-pyrrolo[3,2-d]pyrimidine-7-carbonitrile

This compound was prepared according to scheme 6.

The title compound was prepared analogously as described in Example F1 using iodomethane instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 393 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 6.09 min.

### Example F4

### 6-((S)-3-Aminopiperidin-1-yl)-3-(isoquinolin-1-ylmethyl)-1-methyl-2,4-dioxo-5-(phenylmethyl)-2,3,4,5-tetrahydro-1H-pyrrolo[3,2-d]pyrimidine-7-carbonitrile

This compound was prepared according to scheme 6.

The title compound was prepared analogously as described in Example F1 using 1-(bromomethyl)-isoquinoline hydrobromide instead of 2-bromo-1-(3-methoxyphenyl)-ethanone and using (S)-piperidin-3-yl-carbamic acid tert-butyl ester instead of (R)-piperidin-3-yl-carbamic acid tert-butyl ester.

MS: 520 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 6.81 min.

### Example F5

### 6-((S)-3-Aminopiperidin-1-yl)-1-methyl-2,4-dioxo-5-(phenylmethyl)-2,3,4,5-tetrahydro-1H-pyrrolo[3,2-d]pyrimidine-7-carbonitrile Hydrochloride

This compound was prepared according to scheme 6.

The title compound was prepared analogously as described in Example F1, steps A, B, C, D, E, F and H, using (S)-piperidin-3-yl-carbamic acid tert-butyl ester instead of (R)-piperidin-3-yl-carbamic acid tert-butyl ester. The salt was formed by dissolving the free base in excess MeOHic hydrogen chloride and removing the volatiles.

MS: 379 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 5.46 min.

### Example F6

### 1-methyl-2,4-dioxo-5-(phenylmethyl)-6-piperazin-1-yl-2,3,4,5-tetrahydro-1H-pyrrolo[3,2-d]pyrimidine-7-carbonitrile Hydrochloride

This compound was prepared by adapting the method of scheme 6.

A mixture of 5-benzyl-1-methyl-2,4-dioxo-2,3,4,5-tetrahydro-1H-pyrrolo[3,2-d]pyrimidine-7-carbonitrile (100 mg, 0.278 mmol) and piperazine (239 mg, 2.78 mmol) in DMA (2 mL) was heated at 160° C. in microwave for 20 min. The solvent was removed in vacuo and the residue was triturated with water to give a solid. The solid was purified by flash chromatography (Silica, gradient elution with DCM to 5% MeOH in DCM) to give a pale yellow solid. The residue was dissolved in MeOH (2 mL), treated with hydrogen chloride (1.25 M in MeOH; 0.835 mmol, 0.67 mL) and the mixture was concentrated in vacuo. The residue was dried in vacuo at 45° C. for 18 hours to afford the title compound as a cream coloured solid.

MS: 365 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 5.15 min.

### Example G1

### 5-But-2-ynyl-3-[2-(3-methoxy-phenyl)-2-oxo-ethyl]-4-oxo-6-piperazin-1-yl-4,5-dihydro-3H-pyrrolo[3,2-d]pyrimidine-7-carboxylic Acid Methyl Ester

This compound was prepared according to scheme 7.

### A 4-oxo-4,5-dihydro-3H-pyrrolo[3,2-d]pyrimidine-7-carboxylic Acid Methyl Ester

A mixture of 3-amino-1H-pyrrole-2,4-dicarboxylic acid dimethyl ester (69.82 g, 352 mmol) and formamidine acetate (73.3 g, 705 mmol) in 2-methoxyethanol (250 mL) were heated at 125° C., under an atmosphere of argon, for 6 hours. During warming, the reagents dissolve and within a few minutes, a precipitate forms, which corresponds to the title compound. The reaction mixture was cooled to room temperature and MeOH (150 mL) was added. After stirring for a further few minutes, the precipitate was recovered by vacuum filtration and was washed with MeOH (ca. 50 mL). This was dried in vacuo, at 120° C. for 48 hours to afford the title compound as a light grey solid.

MS: 194.15 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 1.54 min.

### B 4-oxo-3,5-bis-(2-trimethylsilanyl-ethoxymethyl)-4,5-dihydro-3H-pyrrolo[3,2-d]pyrimidine-7-carboxylic Acid Methyl Ester

Anhydrous DMF (750 mL) was added to NaH, 60% dispersion in mineral oil, (23.8 g, 596 mmol), followed by 4-oxo-4,5-dihydro-3H-pyrrolo[3,2-d]pyrimidine-7-carboxylic acid methyl ester (46.07 g, 239 mmol). The suspension was stirred at 50° C. for 1 hr, until hydrogen evolution has ceased. The reaction mixture was cooled in an ice-bath and SEM-Cl (105.7 mL, 596 mmol) was added dropwise over 15 mins. The resulting solution was allowed to stir at room temperature for 2 hours. The DMF was removed in vacuo and the residue was partitioned between ethyl acetate (ca. 500 mL) and water (500 mL). The organic phase was washed with water (ca. 3×500 mL) and concentrated in vacuo to give a pale yellow-orange solid. This was triturated with petrol (40-60° C.) and the fine white solid was filtered and washed with petrol (40-60° C.) to afford the title compound.

MS: 454.2 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.47 min.

### C 6-Chloro-4-oxo-3,5-bis-(2-trimethylsilanyl-ethoxymethyl)-4,5-dihydro-3H-pyrrolo[3,2-d]pyrimidine-7-carboxylic Acid Methyl Ester

A solution of 4-oxo-3,5-bis-(2-trimethylsilanyl-ethoxymethyl)-4,5-dihydro-3H-pyrrolo[3,2-d]pyrimidine-7-carboxylic acid methyl ester (41.4 g, 91.3 mmol) in DMF (500 mL) was treated with N-chlorosuccinimide (18.3 g, 137 mmol) and stirred at room temperature for 18 hours, under an atmosphere of argon. The solid was collected by filtration, washed with DMF (ca. 50 mL) and then dissolved in DCM (300 mL), washed with water (3×200 mL), brine (400 mL) and dried (Na₂SO₄) to afford the title compound as a white powder.

MS: 488.17 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.74 min.

### D 6-Chloro-4-oxo-4,5-dihydro-3H-pyrrolo[3,2-d]pyrimidine-7-carboxylic Acid Methyl Ester

A solution of 6-chloro-4-oxo-3,5-bis-(2-trimethylsilanyl-ethoxymethyl)-4,5-dihydro-3H-pyrrolo[3,2-d]pyrimidine-7-carboxylic acid methyl ester (15 g, 31 mmol) in DCM (150 mL) was treated with TFA (150 mL) and stirred at room temperature for 1 hours The solvents were concentrated in vacuo and the oily residue was azeotroped with toluene (3×50 mL). The residue was triturated with diethyl ether to afford the title compound as a cream solid.

MS: 228.08 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.00 min.

### E 5-But-2-ynyl-6-chloro-4-oxo-4,5-dihydro-3H-pyrrolo[3,2-d]pyrimidine-7-carboxylic Acid Methyl Ester

A solution of 6-chloro-4-oxo-4,5-dihydro-3H-pyrrolo[3,2-d]pyrimidine-7-carboxylic acid methyl ester (2 g, 8.79 mmol) in DMF (20 mL) was treated with methyl-propargyl bromide (0.8 mL, 8.79 mmol) and DIPEA (4.60 mL, 26.4 mmol) and the reaction mixture was stirred at room temperature for 18 hours. The mixture was concentrated in vacuo and the residue was purified by flash chromatography (Silica, eluent: 60% ethyl acetate in petrol (40-60° C.) to 100% ethyl acetate) to give the title compound as a beige solid.

MS: 280.06 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.63 min.

### F 5-But-2-ynyl-6-chloro-3-[2-(3-methoxy-phenyl)-2-oxo-ethyl]-4-oxo-4,5-dihydro-3H-pyrrolo[3,2-d]pyrimidine-7-carboxylic Acid Methyl Ester

A solution of 5-but-2-ynyl-6-chloro-4-oxo-4,5-dihydro-3H-pyrrolo[3,2-d]pyrimidine-7-carboxylic acid methyl ester (0.29 g, 1.04 mmol) and 2-bromo-1-(3-methoxyphenyl)-ethanone (0.238 g, 1.04 mmol) in DMF (7.5 mL) was treated with potassium carbonate (0.216 g, 1.56 mmol) and stirred at room temperature for 1 hours The DMF was removed in vacuo and the residue was triturated with water (ca. 5 mL). The solid formed was collected by filtration, dried in vacuo at 50° C. for 2 hours and purified by flash chromatography (Silica, eluent: 60% ethyl acetate in petrol (40-60° C.) to 100% ethyl acetate) to give the title compound as a white solid.

MS: 428.16 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### G 5-But-2-ynyl-6-chloro-3-[2-(3-methoxy-phenyl)-2-oxo-ethyl]-4-oxo-6-piperazin-1-yl-4,5-dihydro-3H-pyrrolo[3,2-d]pyrimidine-7-carboxylic Acid Methyl Ester

A solution of 5-but-2-ynyl-6-chloro-3-[2-(3-methoxy-phenyl)-2-oxo-ethyl]-4-oxo-4,5-dihydro-3H-pyrrolo[3,2-d]pyrimidine-7-carboxylic acid methyl ester (0.09 g, 0.21 mmol) and piperazine (0.09 g, 1.05 mmol) in DMA (4 mL) was treated with microwave irradiation (Smith Synthesiser), at 160° C. for 10 mins. Further piperazine (0.09 g, 1.05 mmol) was added and the reaction mixture was treated as before for 45 mins. The DMA was concentrated in vacuo and the residue was purified by flash chromatography (Silica, eluent: 5% MeOH in DCM) to give the title compound as an amber oil.

MS: 478.14 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.30 min.

### Example G2

### 3-[2-(3-methoxy-phenyl)-2-oxo-ethyl]-5-(3-methyl -but-2-enyl)-4-oxo-6-piperazin-1-yl-4,5-dihydro-3H-pyrrolo[3,2-d]pyrimidine-7-carboxylic Acid Methyl Ester

This compound was prepared according to scheme 7.

The title compound was prepared analogously as described in Example G1 using 1-bromo-3-methyl-but-2-ene instead of methyl-propargyl bromide.

MS: 494.18 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.84 min.

### Example H1

### 6-((R)-3-amino-piperidin-1-yl)-5-but-2-ynyl-3-[2-(3-methoxy-phenyl)-2-oxo-ethyl]-4-oxo-4,5-dihydro-3H-pyrrolo[3,2-d]pyrimidine-7-carboxylic Acid Methyl Ester

This compound was prepared according to scheme 8.

A solution of 5-but-2-ynyl-6-chloro-3-[2-(3-methoxy-phenyl)-2-oxo-ethyl]-4-oxo-4,5-dihydro-3H-pyrrolo[3,2-d]pyrimidine-7-carboxylic acid methyl ester (0.19 g, 0.456 mmol), prepared by the methods described in Example G1, and (R)-piperidin-3-yl-carbamic acid tert-butyl ester (0.273 g, 1.37 mmol) in DMA (6.5 mL) was heated at 130° C. for 18 hours. Further (R)-piperidin-3-yl-carbamic acid tert-butyl ester (0.273 g, 1.37 mmol) was added and the reaction mixture continued to stir at 130° C. for 4 hours. The reaction mixture was concentrated in vacuo and the residue was triturated with water (ca. 10 mL). The solid obtained was collected by filtration, dried and purified by flash chromatography (Silica, eluent: 50% ethyl acetate in petrol (40-60° C.)) to afford 6-((R)-3-tert-butoxycarbonylamino-piperidin-1-yl)-5-but-2-ynyl-3-[2-(3-methoxy-phenyl)-2-oxo-ethyl]-4-oxo-4,5-dihydro-3H-pyrrolo[3,2-d]pyrimidine-7-carboxylic acid methyl ester as a tan foam. This was dissolved in DCM (5 mL) and treated with TFA (5 mL), with stirring, at room temperature for 1 hours The solvents were concentrated in vacuo and azeotroped with toluene. The residue was passed down a 5 g SCX-2 cartridge, eluting with MeOH followed by 2M ammonia in MeOH solution. The residue obtained was purified by flash chromatography (Silica, eluent: 3% MeOH in DCM), followed by reverse-phase HPLC, wasocratically at 30% acetonitrile (0.1% TFA)/water (0.1% TFA). The salt of the title compound obtained was passed down a 5 g SCX-2 cartridge, eluting with MeOH followed by 2M ammonia in MeOH solution to afford the title compound as a cream foam.

MS: 492.18 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.84 min.

### Example H2

### 6-((R)-3-amino-piperidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carboxylic Acid Methyl Ester

This compound was prepared according to scheme 8.

The title compound was prepared analogously as described in Example H1 using 1-bromo-3-methyl-but-2-ene instead of methyl-propargyl bromide.

MS: 508.15 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.20 min.

### Example 11

### 5-But-2-ynyl-3-[2-(3-methoxy-phenyl)-2-oxo-ethyl]-6-piperazin-1-yl -3,5-dihydro-pyrrolo[3,2-d]pyrimidin-4-one

This compound was prepared according to scheme 9.

### A 6-(4-tert-butoxycarbonyl-piperazin-1-yl)-4-oxo-3,5-bis-(2-trimethylsilanyl-ethoxymethyl)-4,5-dihydro-3H-pyrrolo[3,2-d]pyrimidine-7-carboxylic Acid Methyl Ester

A solution of 6-chloro-4-oxo-3,5-bis-(2-trimethylsilanyl-ethoxymethyl)-4,5-dihydro-3H-pyrrolo[3,2-d]pyrimidine-7-carboxylic acid methyl ester (10 g, 20.4 mmol), prepared by the methods described in Example G1, and tert-butyl 1-piperazinecarboxylate (19.04 g, 102 mmol) in DMA (50 mL) was heated at 130° C. for 18 hours, under an atmosphere of argon. The reaction mixture was concentrated in vacuo and the residue was triturated with diethyl ether to remove excess tert-butyl 1-piperazinecarboxylate. The mother liquor was diluted with DCM (300 mL) and washed with 20% v/v aqueous acetic acid (200 mL) and brine (400 mL), dried (Na₂SO₄), filtered and concentrated in vacuo. The residue was purified by flash silica-gel chromatography (eluent: 60% ethyl acetate in petrol (40-60° C.) to 100% ethyl acetate) to afford the title compound as an amber oil.

MS: 638.26 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 5.10 min.

### B 4-oxo-6-piperazin-1-yl-4,5-dihydro-3H-pyrrolo[3,2-d]pyrimidine-7-carboxylic Acid Methyl Ester

A solution of 6-(4-tert-butoxycarbonyl-piperazin-1-yl)-4-oxo-3,5-bis-(2-trimethylsilanyl-ethoxymethyl)-4,5-dihydro-3H-pyrrolo[3,2-d]pyrimidine-7-carboxylic acid methyl ester (6.35 g, 9.95 mmol) in DCM (50 mL) was treated with TFA (50 mL) and stirred at room temperature for 2 hours. The reaction mixture was concentrated in vacuo and azeotroped with toluene to afford the title compound as a beige solid.

MS: 278.19 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 0.31/0.49 min.

### C 4-(4-oxo-4,5-dihydro-3H-pyrrolo[3,2-d]pyrimidin-6-yl)-piperazine-1-carboxylic Acid tert-butyl Ester

Hot (mobile) polyphosphoric acid (50 g) was added to 4-oxo-6-piperazin-1-yl-4,5-dihydro-3H-pyrrolo[3,2-d]pyrimidine-7-carboxylic acid methyl ester (2.76 g, 9.95 mmol) and the reaction mixture was heated at 180° C. for 2 hours. The reaction mixture was dissolved in water (50 mL), cooled in an ice bath and the pH was adjusted to ca. 8, via the addition of 18M aqueous KOH (ca. 45 mL). Dioxane (100 mL) was added and the reaction mixture was treated with di-tert-butyl dicarbonate (4.34 g, 20 mmol) and stirred at room temperature for 45 hours. The reaction mixture was concentrated in vacuo and the aqueous residue was extracted with ethyl acetate (4×150 mL), DCM (2×100 mL) and 10% MeOH in ethyl acetate (200 mL). The solid formed at the solvent interface was filtered off and washed with 15% MeOH in DCM. The organic extract was concentrated in vacuo and the residue was triturated with diethyl ether/DCM (50 mL/10 mL) to afford the title compound as a tan solid.

MS: 320.29 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 2.13 min.

### D 4-{3-[2-(3-methoxy-phenyl)-2-oxo-ethyl]-4-oxo-4,5-dihydro-3H-pyrrolo[3,2-d]pyrimidin-6-yl}-piperazine-1-carboxylic Acid tert-butyl Ester

A mixture of 4-(4-oxo-4,5-dihydro-3H-pyrrolo[3,2-d]pyrimidin-6-yl)-piperazine-1-carboxylic acid tert-butyl ester (0.5 g, 1.57 mmol) and DMF (10 mL) was treated with potassium carbonate (0.325 g, 2.34 mmol) followed by 2-bromo-1-(3-methoxyphenyl)-ethanone (0.394 g, 1.72 mmol) and the reaction mixture was heated at 50° C. for 1 hours The reaction mixture was diluted with DMF (15 mL) and the reaction mixture was stirred at room temperature for 72 hours. Further 2-bromo-1-(3-methoxyphenyl)-ethanone (0.075 g, 0.3 mmol) was added and the reaction mixture was stirred at room temperature for 3 hours. The DMF was concentrated in vacuo and the residue was triturated with diethyl ether to afford a gummy solid. The crude material was purified by flash chromatography (Silica, eluent: 1% MeOH in DCM), to afford the title compound as a tan solid.

MS: 468.19 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.16 min.

### E 4-{5-but-2-ynyl-3-[2-(3-methoxy-phenyl)-2-oxo-ethyl]-4-oxo-4,5-dihydro-3H-pyrrolo[3,2-d]pyrimidin-6-yl}-piperazine-1-carboxylic Acid tert-butyl Ester

A solution of 4-{3-[2-(3-methoxy-phenyl)-2-oxo-ethyl]-4-oxo-4,5-dihydro-3H-pyrrolo[3,2-d]pyrimidin-6-yl}-piperazine-1-carboxylic acid tert-butyl ester (0.32 g, 0.685 mmol) and methyl propargyl bromide (0.068 mL, 0.753 mmol) in DMF (40 mL) was treated with potassium carbonate (0.118 g, 0.856 mmol) and stirred at room temperature for 74 hours. Further methyl propargyl bromide (0.019 mL, 0.21 mmol) was added and the reaction mixture was heated at 35° C. for 18 hours. Further potassium carbonate (0.045 g, 0.326 mmol) was added and the reaction mixture was heated at 60° C. for 3 hours. The reaction mixture was filtered and concentrated in vacuo and the residue was purified by flash chromatography (Silica, eluent: 100% DCM to 2% MeOH in DCM) to afford the title compound as a tan foam.

MS: 520.35 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.80 min.

### F 5-But-2-ynyl-3-[2-(3-methoxy-phenyl)-2-oxo-ethyl]-6-piperazin-1-yl-3,5-dihydro-pyrrolo[3,2-d]pyrimidin-4-one

A solution of 4-{5-but-2-ynyl-3-[2-(3-methoxy-phenyl)-2-oxo-ethyl]-4-oxo-4,5-dihydro-3H-pyrrolo[3,2-d]pyrimidin-6-yl}-piperazine-1-carboxylic acid tert-butyl ester (0.135 g, 0.259 mmol) in DCM (5 mL) was treated with TFA (5 mL) and stirred at room temperature for 30 mins. The reaction mixture was concentrated in vacuo and the residue was azeotroped with toluene. The crude product was purified by flash chromatography (SCX-2 cartridge, eluting with MeOH and 2M ammonia in MeOH solution prior to being purified by flash chromatography (Silica, eluent: 4% MeOH in DCM to 6% MeOH in DCM) to afford the title compound as a tan foam.

MS: 420.29 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 2.05 min.

### Example J1

### 6-((R)-3-Amino-piperidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carboxylic Acid Amide

This compound was prepared according to scheme 10.

### A 6-((R)-3-tert-Butoxycarbonylamino-piperidin-1-yl)-5-(3-methyl-but-2-enyl)-4-oxo-4,5-dihydro-3H-pyrrolo[3,2-d]pyrimidine-7-carboxylic Acid Methyl Ester

A solution of 6-chloro-5-(3-methyl-but-2-enyl)-4-oxo-4,5-dihydro-3H-pyrrolo[3,2-d]pyrimidine-7-carboxylic acid methyl ester (0.95 g, 3.21 mmol), prepared by the methods described in Example G1, using 1-bromo-3-methyl-but-2-ene instead of 1-bromo-but-2-yne, and (R)-piperidin-3-yl-carbamic acid tert-butyl ester (1.79 g, 9.63 mmol) in DMA (20 mL) was heated at 130° C. for 114 hours. The DMA was concentrated in vacuo and the residue was treated with DCM (300 mL) and washed with 20% v/v aqueous acetic acid (200 mL) and brine (400 mL), dried (Na₂SO₄), filtered and concentrated in vacuo. The residue was purified by flash chromatography (Silica, eluent: DCM to 2% MeOH in DCM) to afford the title compound as a tan solid.

MS: 460.14 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.16 min.

### B 6-((R)-3-tert-Butoxycarbonylamino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic Acid Methyl Ester

A solution of 6-((R)-3-tert-butoxycarbonylamino-piperidin-1-yl)-5-(3-methyl-but-2-enyl)-4-oxo-4,5-dihydro-3H-pyrrolo[3,2-d]pyrimidine-7-carboxylic acid methyl ester (0.62 g, 1.35 mmol) and 2-bromo-1-(3-methoxyphenyl)-ethanone (0.34 g, 1.48 mmol) in DMF (20 mL) was treated with potassium carbonate (0.28 g, 2.02 mmol) and stirred at room temperature for 18 hours under argon. The DMF was concentrated in vacuo and the residue was purified by flash chromatography (Silica, eluent: 1% MeOH in DCM to 5% MeOH in DCM) to afford the title compound as a tan foam.

MS: 608.34 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.11 min.

### C 6-((R)-3-tert-Butoxycarbonylamino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic Acid

A solution of 6-((R)-3-tert-butoxycarbonylamino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid methyl ester (0.525 g, 0.863 mmol) in 1,4-dioxane (19.5 mL) was treated with 0.5M aqueous lithium hydroxide (6.83 mL) and was stirred at 60° C. for 2 hours 45 mins. The reaction mixture was concentrated in vacuo to 20% of the original volume and this was treated with saturated aqueous ammonium chloride (5 mL) to give a solid precipitate, which was collected by filtration, washed with water (20 mL) and dried in vacuo at 60° C. to afford the title compound as a straw coloured solid.

MS: 594.36 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.14 min.

### D {(R)-1-[7-Carbamoyl-3-[2-(3-methoxy-phenyl)-2-oxo-ethyl]-5-(3-methyl-but-2-enyl)-4-oxo-4,5-dihydro-3H-pyrrolo[3,2-d]-pyrimidin-6-yl]-piperidin-3-yl}-carbamic Acid tert-butyl Ester

A solution of 6-((R)-3-tert-butoxycarbonylamino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid (0.09 g, 0.152 mmol) and ammonium chloride (0.0165 g, 0.304 mmol) in DMF (5 mL) was treated with HATU (0.064 g, 0.167 mmol) and stirred at room temperature for 1 hours The DMF was concentrated in vacuo and the residue was partitioned between DCM (15 mL) and saturated aqueous sodium bicarbonate (10 mL). The organic extract was washed with brine (10 mL), dried (Na₂SO₄), filtered and concentrated in vacuo. The residue was purified by flash chromatography (Silica, eluent: 0.5% MeOH in DCM to 1% MeOH in DCM) to afford the title compound as a beige foam.

MS: 593.38 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.05 min.

### E 6-((R)-3-Amino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic Acid Amide

A solution of {(R)-1-[7-carbamoyl-3-[2-(3-methoxy-phenyl)-2-oxo-ethyl]-5-(3-methyl-but-2-enyl)-4-oxo-4,5-dihydro-3H-pyrrolo[3,2-d]-pyrimidin-6-yl]-piperidin-3-yl}-carbamic acid tert-butyl ester (0.03 g, 0.05 mmol) in DCM (2 mL) was treated with TFA (2 mL) and stirred at room temperature for 1 hours The reaction mixture was concentrated in vacuo and the residue was azeotroped with toluene to remove excess TFA. The crude reaction mixture was loaded onto a 2 g SCX-2 cartridge, washed with MeOH and then compound was eluted with a 2M solution of ammonia in MeOH. This was purified by flash chromatography (Silica, eluent: 5% MeOH in DCM to 10% MeOH in DCM) to afford the title compound as a cream foam.

MS: 493.25 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.32 min.

### Example J2

### 6-((R)-3-Amino-piperidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carboxylic Acid Methylamide

This compound was prepared according to scheme 10.

The title compound was prepared analogously as described in Example J1 using methylamine hydrochloride instead of ammonium chloride.

MS: 507.6 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.49 min.

### Example J3

### 6-((R)-3-Amino-piperidin-1-yl)-3-[2-(3-methoxy-phenyl)-2-oxo-ethyl]-5-(3-methyl -but-2-enyl)-4-oxo-4,5-dihydro-3H-pyrrolo[3,2-d]pyrimidine-7-carboxylic Acid Dimethylamide

This compound was prepared according to scheme 10.

The title compound was prepared analogously as described in Example J1 using dimethylamine hydrochloride instead of ammonium chloride.

MS: 521 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.82 min.

### Example J4

### 6-((S)-3-Aminopiperidin-1-yl)-5-but-2-yn-1-yl-N,N-dimethyl-3-(2-(3-(methyloxy)phenyl)-2-oxoethyl)-4-oxo-4,5-dihydro-3H-pyrrolo[3,2-d]pyrimidine-7-carboxamide

This compound was prepared according to scheme 10.

The title compound was prepared analogously as described in Example J1 using 1-bromo-but-2-yne, (S)-piperidin-3-yl-carbamic acid tert-butyl ester instead of (R)-piperidin-3-yl-carbamic acid tert-butyl ester, and dimethylamine hydrochloride instead of ammonium chloride.

MS: 505 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.30 min.

### Example K1

### 6-((R)-3-Amino-piperidin-1-yl)-3-[2-(3-methoxy-phenyl)-2-oxo-ethyl]-5-(3-methyl -but-2-enyl)-3,5-dihydro-pyrrolo[3,2-d]pyrimidin-4-one

This compound was prepared according to scheme 11.

A solution of 6-((R)-3-tert-butoxycarbonylamino-piperidin-1-yl)-3-[2-(3-methoxy-phenyl)-2-oxo-ethyl]-5-(3-methyl-but-2-enyl)-4-oxo-4,5-dihydro-3H-pyrrolo[3,2-d]pyrimidine-7-carboxylic acid (0.065 g, 0.110 mmol), prepared by the methods described in Example J1, in DCM (2 mL) was treated with TFA (2 mL) and the reaction mixture was stirred at room temperature for 30 mins. The reaction mixture was concentrated in vacuo and azeotroped with toluene to remove excess TFA. This was left to stand at room temperature for 10 days to allow decarboxylation to occur. The crude material was purified by reverse-phase HPLC, eluting on a gradient of 30% CH₃CN+0.1% TFA/H₂O+0.1% TFA to 35% CH₃CN+0.1% TFA/H₂O+0.1% TFA over 20 mins to afford the title compound as a cream solid.

MS: 450.28 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.96 min.

### Example L1

### 6-((S)-3-Aminopiperidin-1-yl)-3-(2-(3-(methyloxy)phenyl)-2-oxoethyl)-4-oxo-5-(pyridin-2-ylmethyl)-4,5-dihydro-3H-pyrrolo[3,2-d]pyrimidine-7-carbonitrile Dihydrochloride

This compound was prepared according to scheme 12.

### A [(S)-1-(7-Cyano-4-oxo-4,5-dihydro-3H-pyrrolo[3,2-d]pyrimidin-6-yl)-piperidin-3-yl]-carbamic Acid tert-butyl Ester

6-Bromo-4-oxo-4,5-dihydro-3H-pyrrolo[3,2-d]pyrimidine-7-carbonitrile (0.25 g) and (S)-piperidin-3-yl-carbamic acid tert-butyl ester (1.0 g) were dissolved in DMA (2.5 ml) with warming and the solution was heated in a microwave vial at 160° C. for 1 hour. The solution was concentrated and the residue partitioned between ethyl acetate and water the aqueous phase was adjusted to pH˜6 by adding dilute (1N) aqueous hydrochloric acid. The ethyl acetate layer was separated and concentrated to give a dark red residue which was purified by flash chromatography on silica using MeOH/DCM (5:95) as eluent to afford the title compound as a pale yellow solid.

MS: 359 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.53 min.

### B ((S)-1-{7-Cyano-3-[2-(3-methoxy-phenyl)-2-oxo-ethyl]-4-oxo-4,5-dihydro-3H-pyrrolo[3,2-d]pyrimidin-6-yl}-piperidin-3-yl)-carbamic Acid tert-butyl Ester

2-bromo-1-(3-methoxyphenyl)-ethanone (56 mg, 2.5 mmol) in dimethylformamide (2 mL) was added to a stirred mixture of [(S)-1-(7-cyano-4-oxo-4,5-dihydro-3H-pyrrolo[3,2-d]pyrimidin-6-yl)-piperidin-3-yl]-carbamic acid tert-butyl ester (80 mg, 2.2 mmol) and potassium carbonate (92 mg, 6.7 mmol) in DMF (2 mL). The reaction was concentrated, and the residue purified by preparative HPLC, using a CH3CN/H2O/TFA mobile phase of on a Genesis C18 column. Recovered fractions were adjusted to pH˜7 by the addition of sodium bicarbonate, partially concentrated to remove most of the acetonitrile, and the product was extracted into ethyl acetate. The ethyl acetate solution was washed with a little water, concentrated to dryness, and further dried at 60° C. in vacuo to afford the title compound as a pale yellow solid.

MS: 507 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.31 min.

### C ((S)-1-{7-Cyano-3-[2-(3-methoxy-phenyl)-2-oxo-ethyl]-4-oxo-5-pyridin-2-ylmethyl-4,5-dihydro-3H-pyrrolo[3,2-d]pyrimidin-6-yl}-piperidin-3-yl)-carbamic Acid tert-butyl Ester

A mixture of ((S)-1-{7-cyano-3-[2-(3-methoxy-phenyl)-2-oxo-ethyl]-4-oxo-4,5-dihydro-3H-pyrrolo[3,2-d]pyrimidin-6-yl}-piperidin-3-yl)-carbamic acid tert-butyl ester (50 mg, 0.1 mmol), potassium iodide (49 mg, 0.2 mmol), 2-(chloromethyl)pyridine hydrochloride (32 mg, 0.2 mmol) and diisopropylethylamine (140 μL, 0.8 mmol) in DMF (2 ml) was stirred at 80° C. for 7 hour. The reaction mixture was concentrated, partitioned between ethyl acetate and water and sodium bicarbonate was added to adjust the aqueous phase to pH˜7. The ethyl acetate layer was separated, concentrated and the crude product was purified by flash chromatography on silica using MeOH/DCM (2:98) then further purified by flash chromatography on silica using ethyl acetate/n-heptane (3:1) as eluent to afford the title compound as a pale yellow solid.

MS: 598 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.68 min.

### D 6-((S)-3-Aminopiperidin-1-yl)-3-(2-(3-(methyloxy)phenyl)-2-oxoethyl)-4-oxo-5-(pyridin-2-ylmethyl)-4,5-dihydro-3H-pyrrolo[3,2-d]pyrimidine-7-carbonitrile Dihydrochloride

((S)-1-{7-Cyano-3-[2-(3-methoxy-phenyl)-2-oxo-ethyl]-4-oxo-5-pyridin-2-ylmethyl-4,5-dihydro-3H-pyrrolo[3,2-d]pyrimidin-6-yl}-piperidin-3-yl)-carbamic acid tert-butyl ester (24 mg) was dissolved in a mixture of TFA (1 ml) in DCM (1 ml) and left to stand for 1 hour at room temperature. The reaction was concentrated, redissolved in DCM, and concentrated again. The residue was dissolved in of 1N aqueous hydrochloric acid (0.5 mL) and freeze-dried to give the title compound as a pale yellow solid.

MS: 498 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.37 min.

### Example M1

### 3-(Isoquinolin-1-ylmethyl)-4-oxo-5-(phenylmethyl)-6-piperazin-1-yl-4,5-dihydro-3H-pyrrolo[3,2-d]pyrimidine-7-carbonitrile Dihydrochloride

This compound was prepared according to scheme 13.

### A 2-(Benzylamino-methylene)-malononitrile

Benzylamine (39.4 ml, 0.361 mol) was added to a suspension of ethoxymethylenemalononitrile (40 g, 0.3274 mol) in ethanol (200 ml) giving an exotherm to 50° C. The mixture was then heated at reflux for 2 hours. After cooling in an ice bath for 1 hour, the solids were collected, washed with chilled ethanol (50 mL), diethyl ether (2×80 ml) and sucked dry. The solid was finally dried in vacuo at 44° C. to afford the title compound.

MS: 182 [M−H]⁻

T_{R} [HPLC, Phenomenex Luna 3 micron C18; 5-95% CH₃CN+0.1% Formic acid/H₂O+0.1% Formic acid for 5 min, flow 2.0 ml/min]: 2.87 min.

### B 3-Amino-1-benzyl-4-cyano-1H-pyrrole-2-carboxylic Acid Ethyl Ester

Ethyl bromoacetate (26 ml, 0.235 mol) was added to a suspension of 2-(benzylamino-methylene)-malononitrile (34.1 g, 0.186 mol) and potassium carbonate (51.4 g, 0.372 mol) in DMF (620 ml) and the mixture was stirred and heated at 90° C. for 1.5 hours. Meanwhile, a 1 molar solution of sodium ethoxide in ethanol was prepared from the reaction of freshly cut sodium metal (5.75 g, 0.25 mol) and ethanol (250 ml). The stirred reaction mixture was allowed to cool to 40° C. and the solution of sodium methoxide in ethanol was added dropwise during 20 min with stirring under a nitrogen atmosphere. The reaction mixture was reheated at 90° C. for 1 hour, then allowed to cool and stand at room temperature overnight. The stirred mixture was acidified to pH=6 by the dropwise addition of glacial acetic acid (30 ml) and after stirring for 1 hour the mixture was evaporated at 45° C. in vacuo to remove the solvents to leave a final volume of ˜100 ml. The residue was diluted with iced water (1000 ml) and stirred for 1 hour at room temperature. The precipitate was collected, washed with water (2×100 ml) and sucked dry leaving a pink solid. Final drying in vacuo at 45° C. gave the title compound.

MS: 270 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### C 5-Benzyl-4-oxo-4,5-dihydro-3H-pyrrolo[3,2-d]pyrimidine-7-carbonitrile

Sodium methoxide (67 mL of a 25 wt % solution in MeOH) was added slowly at room temperature to a stirred suspension of 3-amino-1-benzyl-4-cyano-1H-pyrrole-2-carboxylic acid ethyl ester (9.8 g, 38.39 mmol) in formamide (60 mL). When the addition was complete, the mixture was heated at reflux for 4 hours. The mixture was cooled and poured onto iced water containing concentrated hydrochloric acid (35 ml). The precipitate was collected, washed thoroughly with water and dried to afford the title compound as a brown solid.

MS: 251 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.60 min.

### D 5-Benzyl-6-chloro-4-oxo-4,5-dihydro-3H-pyrrolo[3,2-d]pyrimidine-7-carbonitrile

A mixture of 5-benzyl-4-oxo-4,5-dihydro-3H-pyrrolo[3,2-d]pyrimidine-7-carbonitrile (200 mg, 0.8 mmol) and N-chlorosuccinimide (320 mg, 2.4 mmol) in DMF (10 mL) was stirred at room temperature for 24 hours The mixture was poured into water and the solids collected and dried to afford the title compound as a yellow solid.

### E 5-Benzyl-6-chloro-3-isoquinolin-1-ylmethyl-4-oxo-4,5-dihydro-3H-pyrrolo[3,2-d]pyrimidine-7-carbonitrile

A mixture of 5-benzyl-6-chloro-4-oxo-4,5-dihydro-3H-pyrrolo[3,2-d]pyrimidine-7-carbonitrile (300 mg, 1.05 mmol) and potassium carbonate (580 mg, 4.2 mmol) in DMF (5 mL) was treated with a suspension of 1-(bromomethyl)-isoquinoline hydrobromide (350 mg, 1.16 mmol) in DMF (10 mL) and the reaction was stirred at room temperature for 2 hours. The mixture was concentrated and the residue partitioned between water and ethyl acetate; the organic layer was washed twice with water and then concentrated to dryness. The residue was purified by flash chromatography on silica, using MeOH/DCM (2:98) as eluent to give the title compound as a yellow solid.

MS: 426 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.81 min.

### F 3-(isoquinolin-1-ylmethyl)-4-oxo-5-(phenylmethyl)-6-piperazin-1-yl-4,5-dihydro-3H-pyrrolo[3,2-d]pyrimidine-7-carbonitrile Dihydrochloride

5-Benzyl-6-chloro-3-isoquinolin-1-ylmethyl-4-oxo-4,5-dihydro-3H-pyrrolo[3,2-d]pyrimidine-7-carbonitrile (0.14 g, 0.33 mmol) and piperazine (0.14 g, 1.64 mmol) were dissolved in DMA (3 ml) and heated (microwave) to 160° C. for 30 minutes. The reaction mixture was concentrated and partitioned between ethyl acetate and water The organic phase was washed twice with water and then concentrated to dryness. The crude product was purified by flash chromatography on silica; using a gradient elution MeOH/DCM (2:98) to MeOH/DCM (10:90). The recovered product was dissolved in a little MeOH and the solution acidified by the addition of hydrogen chloride in MeOH (1.25M). Concentration afforded to the title compound as a pale yellow solid.

MS: 476 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.78 min.

### Example M2

### 6-(1,4-Diazepan-1-yl)-3-(isoquinolin-1-ylmethyl)-4-oxo-5-(phenylmethyl)-4,5-dihydro-3H-pyrrolo[3,2-d]pyrimidine-7-carbonitrile Dihydrochloride

This compound was prepared according to scheme 13.

### A 2-(Benzylamino-methylene)-malononitrile

Benzylamine (39.4 ml, 0.361 mol) was added to a suspension of ethoxymethylenemalononitrile (40 g, 0.3274 mol) in ethanol (200 ml) giving an exotherm to 50° C. The mixture was then heated at reflux for 2 hours. After cooling in an ice bath for 1 hour, the solids were collected, washed with chilled ethanol (50 mL), diethyl ether (2×80 ml) and sucked dry. The solid was finally dried in vacuo at 44° C. to afford the title compound.

MS: 182 [M−H]⁻

T_{R} [HPLC, Phenomenex Luna 3 micron C18; 5-95% CH₃CN+0.1% Formic acid/H₂O+0.1% Formic acid for 5 min, flow 2.0 ml/min]: 2.87 min.

### B 3-Amino-1-benzyl-4-cyano-1H-pyrrole-2-carboxylic Acid Ethyl Ester

Ethyl bromoacetate (26 ml, 0.235 mol) was added to a suspension of 2-(benzylamino-methylene)-malononitrile (34.1 g, 0.186 mol) and potassium carbonate (51.4 g, 0.372 mol) in DMF (620 ml) and the mixture was stirred and heated at 90° C. for 1.5 hours. Meanwhile, a 1 molar solution of sodium ethoxide in ethanol was prepared from the reaction of freshly cut sodium metal (5.75 g, 0.25 mol) and ethanol (250 ml). The stirred reaction mixture was allowed to cool to 40° C. and the solution of sodium methoxide in ethanol was added dropwise during 20 min with stirring under a nitrogen atmosphere. The reaction mixture was reheated at 90° C. for 1 hour, then allowed to cool and stand at room temperature overnight. The stirred mixture was acidified to pH=6 by the dropwise addition of glacial acetic acid (30 ml) and after stirring for 1 hour the mixture was evaporated at 45° C. in vacuo to remove the solvents to leave a final volume of approximately 100 ml. The residue was diluted with iced water (1000 ml) and stirred for 1 hour at room temperature. The precipitate was collected, washed with water (2×100 ml) and sucked dry leaving a pink solid. Final drying in vacuo at 45° C. gave the title compound.

MS: 270 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### C 5-Benzyl-4-oxo-4,5-dihydro-3H-pyrrolo[3,2-d]pyrimidine-7-carbonitrile

Sodium methoxide (67 mL of a 25 wt % solution in MeOH) was added slowly at room temperature to a stirred suspension of 3-amino-1-benzyl-4-cyano-1H-pyrrole-2-carboxylic acid ethyl ester (9.8 g, 38.39 mmol) in formamide (60 mL). When the addition was complete, the mixture was heated at reflux for 4 hours. The mixture was cooled and poured onto iced water containing concentrated hydrochloric acid (35 ml). The precipitate was collected, washed thoroughly with water and dried to afford the title compound as a brown solid.

MS: 251 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.60 min.

### D 5-Benzyl-6-chloro-4-oxo-4,5-dihydro-3H-pyrrolo[3,2-d]pyrimidine-7-carbonitrile

A mixture of 5-benzyl-4-oxo-4,5-dihydro-3H-pyrrolo[3,2-d]pyrimidine-7-carbonitrile (200 mg, 0.8 mmol) and N-chlorosuccinimide (320 mg, 2.4 mmol) in DMF (10 mL) was stirred at room temperature for 24 hours The mixture was poured into water and the solids collected and dried to afford the title compound as a yellow solid.

### E 5-Benzyl-6-chloro-3-isoquinolin-1-ylmethyl-4-oxo-4,5-dihydro-3H-pyrrolo[3,2-d]pyrimidine-7-carbonitrile

A mixture of 5-benzyl-6-chloro-4-oxo-4,5-dihydro-3H-pyrrolo[3,2-d]pyrimidine-7-carbonitrile (300 mg, 1.05 mmol) and potassium carbonate (580 mg, 4.2 mmol) in DMF (5 mL) was treated with a suspension of 1-(bromomethyl)-isoquinoline hydrobromide (350 mg, 1.16 mmol) in DMF (10 mL) and the reaction was stirred at room temperature for 2 hours. The mixture was concentrated and the residue partitioned between water and ethyl acetate; the organic layer was washed twice with water and then concentrated to dryness. The residue was purified by flash chromatography on silica, using MeOH/DCM (2:98) as eluent to give the title compound as a yellow solid.

MS: 426 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.81 min.

### F 6-(1,4-Diazepan-1-yl)-3-(isoquinolin-1-ylmethyl)-4-oxo-5-(phenylmethyl)-4,5-dihydro-3H-pyrrolo[3,2-d]pyrimidine-7-carbonitrile Hydrochloride

5-Benzyl-6-chloro-3-isoquinolin-1-ylmethyl-4-oxo-4,5-dihydro-3H-pyrrolo[3,2-d]pyrimidine-7-carbonitrile (0.14 g, 0.33 mmol) and [1,4]diazepane (0.16 g, 1.64 mmol) were dissolved in DMA (3 ml) and heated by microwave irradiation to 160° C. for 90 minutes. The reaction mixture was concentrated and partitioned between ethyl acetate and water. The organic phase was washed twice with water and then concentrated to dryness. The crude product was purified by flash chromatography (silica; using a gradient elution MeOH/DCM (2:98) to (10:90)). The recovered product was dissolved in a little MeOH and the solution acidified by the addition of excess 1.25M hydrogen chloride in MeOH. Evaporation to dryness afforded the title compound as a pale yellow solid.

MS: 490 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.87 min.

### Example M3

### 6-((S)-Aminopiperidin-1-yl)-4-oxo-5-(phenylmethyl)-3-(quinolin-6-ylmethyl)-4,5-dihydro-3H-pyrrolo[3,2-d]pyrimidine-7-carbonitrile

This compound was prepared according to scheme 13.

The title compound was prepared analogously as described in Example M1 using 6-(chloromethyl)-quinoline hydrochloride instead of 1-(bromomethyl)-isoquinoline hydrobromide and using (S)-piperidin-3-yl-carbamic acid tert-butyl ester instead of piperazine. The removal of the Boc group was as described in Example J1.

MS: 490 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.47 min.

### Example M4

### 6-((S)-3-Aminopiperidin-1-yl)-3-(isoquinolin-1-ylmethyl)-4-oxo-5-(phenylmethyl)-4,5-dihydro-3H-pyrrolo[3,2-d]pyrimidine-7-carbonitrile

This compound was prepared according to scheme 13.

The title compound was prepared analogously as described in Example M1 using (S)-piperidin-3-yl-carbamic acid tert-butyl ester instead of piperazine. The removal of the Boc group was as described in Example J1.

MS: 490 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.07 min.

### Example M5

### 6-((S)-3-Aminopiperidin-1-yl)-4-oxo-5-(phenylmethyl)-4,5-dihydro-3H-pyrrolo[3,2-d]pyrimidine-7-carbonitrile Hydrochloride

This compound was prepared according to scheme 13.

The title compound was prepared analogously as described in Example M1, steps A and C, using (S)-piperidin-3-yl-carbamic acid tert-butyl ester instead of piperazine. The removal of the tert-butyloxycarbonyl protecting group was as described in Example L1.

MS: 349 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.15 min.

### Example M6

### 6-(1,4-Diazepan-1-yl)-3-methyl-4-oxo-5-(phenylmethyl)-4,5-dihydro-3H-pyrrolo[3,2-d]pyrimidine-7-carbonitrile Hydrochloride

This compound was prepared according to scheme 13.

The title compound was prepared analogously as described in Example M2 using iodomethane instead of 1-(bromomethyl)-isoquinoline hydrobromide.

MS: 363 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.24 min.

### Example M7

### 3-Methyl-4-oxo-5-(phenylmethyl)-6-piperazin-1-yl-4,5-dihydro-3H-pyrrolo[3,2-d]pyrimidine-7-carbonitrile Hydrochloride

This compound was prepared according to scheme 13.

The title compound was prepared analogously as described in Example M1 using iodomethane instead of 1-(bromomethyl)-isoquinoline hydrobromide.

MS: 349 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.00 min.

### Example M8

### 6-((S)-3-Aminopiperidin-1-yl)-4-oxo-5-(phenylmethyl)-3-(2,2,2-trifluoroethyl)-4,5-dihydro-3H-pyrrolo[3,2-d]pyrimidine-7-carbonitrile Hydrochloride

This compound was prepared according to scheme 13.

The title compound was prepared analogously as described in Example M1 using 1,1,1-trifluor-2-iodo-ethane instead of 1-(bromomethyl)-isoquinoline hydrobromide and using (S)-piperidin-3-yl-carbamic acid tert-butyl ester instead of piperazine. The Boc protecting group was removed and hydrochloride salt formed by the method described in Example N26.

MS: 431 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.70 min.

### Example N1

### 6-((S)-3-Aminopiperidin-1-yl)-4-oxo-5-(phenylmethyl)-3-(quinolin-8-ylmethyl)-4,5-dihydro-3H-pyrrolo[3,2-d]pyrimidine-7-carbonitrile hydrochloride

This compound was prepared according to scheme 14.

### A [(S)-1-(5-Benzyl-7-cyano-4-oxo-4,5-dihydro-3H-pyrrolo[3,2-d]pyrimidin-6-yl)-piperidin-3-yl]-carbamic Acid tert-butyl Ester

5-Benzyl-6-chloro-4-oxo-4,5-dihydro-3H-pyrrolo[3,2-d]pyrimidine-7-carbonitrile (1.046 g, 3.67 mmol) and 3S-(boc-amino)piperidine (2.2 g, 11.0 mmol) in DMA (10 mL) was heated at 160° C. for 1.5 hours. The crude reaction mixture was partitioned between DCM (2×50 mL) and water (100 mL) and the combined organic extracts were washed with brine (100 mL) and dried (Na2SO4), After concentrating in vacuo, the residue was purified by flash chromatography (Silica, eluting with DCM/MeOH (49/1) to (4/1)). The product was triturated with water and dried in vacuo at 50° C. overnight to afford the title compound.

MS: 449[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.28 min.

### B 6-((S)-3-Aminopiperidin-1-yl)-4-oxo-5-(phenylmethyl)-3-(quinolin-8-ylmethyl)-4,5-dihydro-3H-pyrrolo[3,2-d]pyrimidine-7-carbonitrile Hydrochloride

A mixture of [(S)-1-(5-benzyl-7-cyano-4-oxo-4,5-dihydro-3H-pyrrolo[3,2-d]pyrimidin-6-yl)-piperidin-3-yl]-carbamic acid tert-butyl ester (75 mg, 0.167 mmol), potassium carbonate (60 mg, 0.434 mmol) and 8-(bromomethyl)-isoquinoline (39 mg, 0.176 mmol) in DMF was stirred at ambient temperature overnight. The solvent was evaporated and the residue was treated with a mixture of DCM (1 mL) and TFA (0.5 mL) for 1.5 hours The reaction mixture was loaded on to an SCX-2 cartridge and washed with DCM and MeOH before eluting with a 2 Molar solution of ammonia in MeOH. The crude product was finally purified by flash chromatography (Silica, eluting with DCM/MeOH (50/1) to (20/1)). The product was converted to the hydrochloride salt by treatment with excess 1M aqueous hydrochloric acid (10 equivalents) in MeOH (1.5 mL) and freeze drying to afford the title compound as a white solid.

MS: 490 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.22 min.

### Example N2

### 6-((S)-3-Aminopiperidin-1-yl)-4-oxo-3-((3-phenyl isoxazol-5-yl)methyl)-5-(phenylmethyl)-4,5-dihydro-3H-pyrrolo[3,2-d]pyrimidine-7-carbonitrile Hydrochloride

This compound was prepared according to scheme 14.

The title compound was prepared by adapting the procedure described in Example N1 using 5-chloromethyl-3-phenyl-isoxazole instead of 8-(bromomethyl)-isoquinoline.

MS: 506 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.45 min.

### Example N3

### 6-((S)-3-Aminopiperidin-1-yl)-4-oxo-5-(phenylmethyl)-3-(2-(phenyloxy)ethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using (2-chloroethoxy)-benzene instead of 8-(bromomethyl)-isoquinoline.

MS: 469 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.30 min.

### Example N4

### 6-((S)-3-Aminopiperidin-1-yl)-3-((2-methyl-1,3-thiazol-4-yl)methyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 4-chloromethyl-2-methyl-thiazole instead of 8-(bromomethyl)-isoquinoline and heating at 50° C. for 3 hours in the presence of 1 equivalent of potassium iodide.

MS: 460 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.28 min.

### Example N5

### 6-((S)-3-Aminopiperidin-1-yl)-3-(2-morpholin-4-ylethyl)-4-oxo-5-(phenylmethyl)-4,5-dihydro-3H-pyrrolo[3,2-d]pyrimidine-7-carbonitrile Dihydrochloride

This compound was prepared according to scheme 14.

The title compound was prepared by the procedure described in Example N1 using 4-(2-chloro-ethyl)-morpholine instead of 8-(bromomethyl)-isoquinoline and heating at 50° C. for 21 hours in the presence of 1 equivalent of potassium iodide.

MS: 462 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.25 min.

### Example N6

### 6-((S)-3-Aminopiperidin-1-yl)-3-(2,3-dihydro-1,4-benzodioxin-2-ylmethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-bromomethyl-2,3-dihydro-benzo[1,4]dioxine instead of 8-(bromomethyl)-isoquinoline and heating at 50° C. for 3 hours in the presence of 1 equivalent of potassium iodide.

MS: 497 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.50 min.

### Example N7

### 6-((S)-3-Aminopiperidin-1-yl)-3-((2-cyanophenyl)methyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-(bromomethyl)-benzonitrile instead of 8-(bromomethyl)-isoquinoline.

MS: 464[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.80 min.

### Example N8

### 6-((S)-3-Aminopiperidin-1-yl)-3-((5-methyl-1,3,4-oxadiazol-2-yl)methyl)-4-oxo-5-(phenylmethyl)-4,5-dihydro-3H-pyrrolo[3,2-d]pyrimidine-7-carbonitrile

This compound was prepared according to scheme 14.

The title compound was prepared by the procedure described in Example N1 using 2-chloromethyl-5-methyl-[1,3,4]oxadazole instead of 8-(bromomethyl)-isoquinoline, and was isolated as the free base.

MS: 445[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.41 min.

### Example N9

### N-(2-(6-((S)-3-Aminopiperidin-1-yl)-7-cyano-4-oxo-5-(phenylmethyl)-4,5-dihydro-3H-pyrrolo[3,2-d]pyrimidin-3-yl)ethyl)benzenesulfonamide

This compound was prepared according to scheme 14.

The title compound was prepared by the procedure described in Example N1 using N-(2-bromo-ethyl)-benzenesulfonamide instead of 8-(bromomethyl)-isoquinoline, and was isolated as the free base.

MS: 532[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.37 min.

### Example N10

### 6-((S)-3-Aminopiperidin-1-yl)-3-(2-(3,5-dimethylisoxazol-4-yl)ethyl)-4-oxo-5-(phenylmethyl)-4,5-dihydro-3H-pyrrolo[3,2-d]pyrimidine-7-carbonitrile

This compound was prepared according to scheme 14.

The title compound was prepared by the procedure described in Example N1 using 4-(2-chloro-ethyl)-3,5-dimethyl-isoxazole instead of 8-(bromomethyl)-isoquinoline and heating at 50° C. for 3 hours in the presence of 1 equivalent of potassium iodide. The product was isolated as the free base.

MS: 472[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.45 min.

### Example N11

### 6-((S)-3-Aminopiperidin-1-yl)-3-(1,3-benzoxazol-2-ylmethyl)-4-oxo-5-(phenylmethyl)-4,5-dihydro-3H-pyrrolo[3,2-d]pyrimidine-7-carbonitrile

This compound was prepared according to scheme 14.

The title compound was prepared by the procedure described in Example N1 using 2-chloromethyl-benzooxazole instead of 8-(bromomethyl)-isoquinoline and the product was isolated as the free base.

MS: 480[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.87 min.

### Example N12

### 6-((S)-3-Aminopiperidin-1-yl)-3-methyl-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using iodomethane instead of 8-(bromomethyl)-isoquinoline.

MS: 363[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.55 min.

### Example N13

### 6-((S)-3-Aminopiperidin-1-yl)-4-oxo-5-(phenylmethyl)-3-(pyridin-2-ylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-(chloromethyl)-pyridine hydrochloride instead of 8-(bromomethyl)-isoquinoline.

MS: 440[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.78 min.

### Example N14

### 6-((S)-3-Aminopiperidin-1-yl)-4-oxo-5-(phenylmethyl)-3-(pyridin-3-ylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 3-(chloromethyl)-pyridine hydrochloride instead of 8-(bromomethyl)-isoquinoline.

MS: 440[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.85 min.

### Example N15

### 2-(6-((S)-3-Aminopiperidin-1-yl)-7-cyano-4-oxo-5-(phenylmethyl)-4,5-dihydro-3H-pyrrolo[3,2-d]pyrimidin-3-yl)-N,N-DMA Hydrochloride

This compound was prepared according to scheme 14.

The title compound was prepared by the procedure described in Example N1 using 2-chloro-N,N-dimethyl-acetamide instead of 8-(bromomethyl)-isoquinoline.

MS: 434[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.50 min.

### Example N16

### 6-((S)-3-Aminopiperidin-1-yl)-4-oxo-5-(phenylmethyl)-3-((5-phenyl-1,2,4-oxadiazol-3-yl)methyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 3-chloromethyl-5-phenyl-[1,2,4]oxadiazole instead of 8-(bromomethyl)-isoquinoline.

MS: 507[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.34 min.

### Example N17

### 2-(6-((S)-3-Aminopiperidin-1-yl)-7-cyano-4-oxo-5-(phenylmethyl)-4,5-dihydro-3H-pyrrolo[3,2-d]pyrimidin-3-yl)-N-phenylacetamide Hydrochloride

This compound was prepared according to scheme 14.

The title compound was prepared by the procedure described in Example N1 using 2-chloro-N-phenyl-acetamide instead of 8-(bromomethyl)-isoquinoline.

MS: 483[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.62 min.

### Example N18

### 6-((S)-3-Aminopiperidin-1-yl)-3-(2-morpholin-4-yl-2-oxoethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-chloro-1-morpholin-4-yl-ethanone instead of 8-(bromomethyl)-isoquinoline.

MS: 476[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.49 min.

### Example N19

### 6-((S)-3-Aminopiperidin-1-yl)-4-oxo-5-(phenylmethyl)-3-((1-(phenylmethyl)-1H-imidazol-2-yl)methyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 1-benzyl-2-chloromethyl-1H-imidazole hydrochloride instead of 8-(bromomethyl)-isoquinoline.

MS: 519[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.12 min.

### Example N20

### 6-((S)-3-Aminopiperidin-1-yl)-3-(2-(ethyloxy)ethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 1-bromo-2-ethoxy-ethane instead of 8-(bromomethyl)-isoquinoline.

MS: 421[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.22 min.

### Example N21

### 6-((S)-3-Aminopiperidin-1-yl)-4-oxo-5-(phenylmethyl)-3-((6-(trifluoromethyl)pyridin-3-yl)methyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 5-chloromethyl-2-trifluoromethyl-pyridine instead of 8-(bromomethyl)-isoquinoline.

MS: 508[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.94 min.

### Example N22

### 6-((S)-3-Aminopiperidin-1-yl)-3-(imidazo[1,2-a]pyridin-3-ylmethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 3-chloromethyl-imidazo[1,2-a]pyridine hydrochloride instead of 8-(bromomethyl)-isoquinoline.

MS: 479[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.65 min.

### Example N23

### 6-((S)-3-Aminopiperidin-1-yl)-4-oxo-5-(phenylmethyl)-3-(tetrahydrofuran-2-ylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-bromomethyl-tetrahydro-furan instead of 8-(bromomethyl)-isoquinoline and heating at 75° C. for 18 hours.

MS: 433[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.24 min.

### Example N24

### 6-((S)-3-Aminopiperidin-1-yl)-4-oxo-3-(2-phenylethyl)-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using (2-bromo-ethyl)-benzene instead of 8-(bromomethyl)-isoquinoline.

MS: 453[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.34 min.

### Example N25

### 6-((S)-3-Aminopiperidin-1-yl)-4-oxo-3-(3-phenylpropyl)-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using (3-bromo-propyl)-benzene instead of 8-(bromomethyl)-isoquinoline.

MS: 467[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.72 min.

### Example N26

### 6-((S)-3-Aminopiperidin-1-yl)-7-cyano-N,N-dimethyl-4-oxo-5-(phenylmethyl)-4,5-dihydro-3H-pyrrolo[3,2-d]pyrimidine-3-carboxamide Hydrochloride

This compound was prepared by adapting scheme 14.

Triethylamine (58 μL, 0.416 mmol) and N,N-dimethyl-carbamoyl chloride (17 μL, 0.185 mmol) were added to a solution of [(S)-1-(5-benzyl-7-cyano-4-oxo-4,5-dihydro-3H-pyrrolo[3,2-d]pyrimidin-6-yl)-piperidin-3-yl]-carbamic acid tert-butyl ester (75 mg, 0.167 mmol) in DMF (1.5 mL) and the mixture was stirred at room temperature for 20 hours. The mixture was concentrated and the residue was triturated with water. The resulting solid was filtered off, dried at 50° C. and treated with DCM (1 mL) and TFA (0.3 mL). After stirring at ambient temperature for 1 hour the reaction mixture was loaded on to an SCX-2 cartridge and washed with DCM and MeOH before eluting with 2M ammonia in MeOH. The crude product was purified by flash chromatography (Silica, 5% MeOH/DCM as eluent) and the appropriate fractions evaporated. The residue was treated with excess 1M hydrogen chloride in MeOH and evaporated to give the title compound as a white solid.

MS: 420[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.57 min.

### Example N27

### 6-((S)-3-Aminopiperidin-1-yl)-4-oxo-5-(phenylmethyl)-3-(2-(1H-pyrazol-1-yl)ethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 1-(2-chloro-ethyl)-1H-pyrazole instead of 8-(bromomethyl)-isoquinoline.

MS: 443[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.84 min.

### Example N28

### 6-((S)-3-Aminopiperidin-1-yl)-3-(2-hydroxyethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-bromoethanol instead of 8-(bromomethyl)-isoquinoline.

MS: 393[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.23 min.

### Example N29

### 6-((S)-3-Aminopiperidin-1-yl)-3-(cyclopropyl methyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using bromomethyl-cyclopropane instead of 8-(bromomethyl)-isoquinoline.

MS: 403[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.57 min.

### Example N30

### 6-((S)-3-Aminopiperidin-1-yl)-4-oxo-5-(phenylmethyl)-3-((2E)-3-phenylprop-2-en-1-yl)-4,5-dihydro-3H-pyrrolo[3,2-d]pyrimidine-7-carbonitrile Hydrochloride

This compound was prepared according to scheme 14.

The title compound was prepared by the procedure described in Example N1 using ((E)-3-bromo-propenyl)-benzene instead of 8-(bromomethyl)-isoquinoline.

MS: 465[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.68 min.

### Example N31

### 6-((S)-3-Aminopiperidin-1-yl)-3-(2-cyclohexylethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using (2-bromo-ethyl)-cyclohexane instead of 8-(bromomethyl)-isoquinoline.

MS: 459[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 8.43 min.

### Example N32

### 6-((S)-3-Aminopiperidin-1-yl)-3-(2,3-dihydro-1,4-benzodioxin-6-ylmethyl)-4-oxo-5-(phenylmethyl)-4,5-dihydro-3H-pyrrolo[3,2-d]pyrimidine-7-carbonitrile Hydrochloride

This compound was prepared according to scheme 14.

A {(S)-1-[5-Benzyl-7-cyano-3-(2,3-dihydro-benzo[1,4]dioxin-6-ylmethyl)-4-oxo-4,5-dihydro-3H-pyrrolo[3,2-d]pyrimidin-6-yl]-piperidin-3-yl}-carbamic Acid tert-butyl Ester

Triphenyl phosphine (52.4 mg, 0.20 mmol) followed by diethyl azodicarboxylate (31 μL, 0.20 mmol) were added to a solution of [(S)-1-(5-benzyl-7-cyano-4-oxo-4,5-dihydro-3H-pyrrolo[3,2-d]pyrimidin-6-yl)-piperidin-3-yl]-carbamic acid tert-butyl ester (75 mg, 0.167 mmol) and (2,3-dihydro-benzo[1,4]dioxin-6-yl)-MeOH (27.7 mg, 0.167 mmol) in THF (2 mL) and the mixture was stirred at room temperature for 18 hours then heated to 45° C. for 6 hours. The solvent was removed under a stream of nitrogen at 30° C. and the residue was partially purified by flash chromatography (Silica, gradient elution using DCM to 30% ethyl acetate in DCM) and finally purified by reversed phase HPLC (5% to 95% MeCN in water+0.1% HCO2H at 5 mL/min) to give the title compound which was used directly in the next step.

### B 6-((S)-3-aminopiperidin-1-yl)-3-(2,3-dihydro-1,4-benzodioxin-6-ylmethyl)-4-oxo-5-(phenylmethyl)-4,5-dihydro-3H-pyrrolo[3,2-d]pyrimidine-7-carbonitrile Hydrochloride

{(S)-1-[5-Benzyl-7-cyano-3-(2,3-dihydro-benzo[1,4]dioxin-6-ylmethyl)-4-oxo-4,5-dihydro-3H-pyrrolo[3,2-d]pyrimidin-6-yl]-piperidin-3-yl}-carbamic acid tert-butyl ester from step A was treated with TFA/DCM (2 mL, 1:1) for 1 h at room temperature. The reaction mixture was then purified by flash chromatography (SCX-2, washing with DCM, ethyl acetate and MeOH and eluting with 1M ammonia in MeOH). The resulting residue was converted to hydrochloride salt by treatment with excess 1.25M hydrogen chloride in MeOH followed by evaporation to give the title compound as a solid.

MS: 497[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.16 min.

### Example N33

### 6-((S)-3-Aminopiperidin-1-yl)-3-((1,3-dimethyl-1H-pyrazol-5-yl)methyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N32 using (2,5-dimethyl-2H-pyrazol-3-yl)-MeOH instead of (2,3-dihydro-benzo[1,4]dioxin-6-yl)-MeOH.

MS: 457[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.12 min.

### Example N34

### 6-((S)-3-Aminopiperidin-1-yl)-3-(2-(4-methyl-1,3-thiazol-5-yl)ethyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N32 using 2-(4-methyl-thiazol-5-yl)-ethanol instead of (2,3-dihydro-benzo[1,4]dioxin-6-yl)-MeOH.

MS: 474[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.20 min.

### Example N35

### 6-((S)-3-Aminopiperidin-1-yl)-3-((1-methyl-1H-benzimidazol-2-yl)methyl)-4-oxo-5-(phenylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-bromomethyl-1-methyl-1H-benzoimidazole instead of 8-(bromomethyl)-isoquinoline.

MS: 493[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.82 min.

### Example N36

### 6-((S)-3-Aminopiperidin-1-yl)-4-oxo-5-(phenylmethyl)-3-(quinazolin-2-ylmethyl)-4,5-dihydro-3H-pyrrolo[3,2-d]pyrimidine-7-carbonitrile Hydrochloride

This compound was prepared according to scheme 14.

The title compound was prepared by the procedure described in Example N1 using 2-(chloromethyl)-quinazoline instead of 8-(bromomethyl)-isoquinoline.

MS: 491[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.53 min.

### Example O1

### 6-((S)-3-Aminopiperidin-1-yl)-N,N-dimethyl-3-(2-(3-(methyloxy)phenyl)-2-oxoethyl)-4-oxo-5-(phenylmethyl)-4,5-dihydro-3H-pyrrolo[3,2-d]pyrimidine-7-carboxamide Hydrochloride

This compound was prepared according to scheme 15.

### A 4-oxo-4,5-dihydro-3H-pyrrolo[3,2-d]pyrimidine-7-carboxylic Acid Methyl Ester

A mixture of 3-amino-1H-pyrrole-2,4-dicarboxylic acid dimethyl ester (69.82 g, 352 mmol) and formamidine acetate (73.3 g, 705 mmol) in 2-methoxyethanol (250 mL) were heated at 125° C., under an atmosphere of argon, for 6 hours. During warming, the reagents dissolve and within a few minutes, a precipitate forms, which corresponds to the title compound. The reaction mixture was cooled to room temperature and MeOH (150 mL) was added. After stirring for a further few minutes, the precipitate was recovered by vacuum filtration and was washed with MeOH (ca. 50 mL). This was dried in vacuo, at 120° C. for 48 hours to afford the title compound as a light grey solid.

MS: 194.15 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 1.54 min.

### B 4-oxo-3,5-bis-(2-trimethylsilanyl-ethoxymethyl)-4,5-dihydro-3H-pyrrolo[3,2-d]pyrimidine-7-carboxylic Acid Methyl Ester

Anhydrous DMF (750 mL) was added to NaH, 60% dispersion in mineral oil, (23.8 g, 596 mmol), followed by 4-oxo-4,5-dihydro-3H-pyrrolo[3,2-d]pyrimidine-7-carboxylic acid methyl ester (46.07 g, 239 mmol). The suspension was stirred at 50° C. for 1 hr, until hydrogen evolution has ceased. The reaction mixture was cooled in an ice-bath and SEM-Cl (105.7 mL, 596 mmol) was added dropwise over 15 mins. The resulting solution was allowed to stir at room temperature for 2 hours. The DMF was removed in vacuo and the residue was partitioned between ethyl acetate (ca. 500 mL) and water (500 mL). The organic phase was washed with water (ca. 3×500 mL) and concentrated in vacuo to give a pale yellow-orange solid. This was triturated with petrol (40-60° C.) and the fine white solid was filtered and washed with petrol (40-60° C.) to afford the title compound.

MS: 454.2 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.47 min.

### C 6-Chloro-4-oxo-3,5-bis-(2-trimethylsilanyl-ethoxymethyl)-4,5-dihydro-3H-pyrrolo[3,2-d]pyrimidine-7-carboxylic Acid Methyl Ester

A solution of 4-oxo-3,5-bis-(2-trimethylsilanyl-ethoxymethyl)-4,5-dihydro-3H-pyrrolo[3,2-d]pyrimidine-7-carboxylic acid methyl ester (41.4 g, 91.3 mmol) in DMF (500 mL) was treated with N-chlorosuccinimide (18.3 g, 137 mmol) and stirred at room temperature for 18 hours, under an atmosphere of argon. The solid was collected by filtration, washed with DMF (ca. 50 mL) and then dissolved in DCM (300 mL), washed with water (3×200 mL), brine (400 mL) and dried (Na₂SO₄) to afford the title compound as a white powder.

MS: 488.17 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.74 min.

### D 6-Chloro-4-oxo-5-(2-trimethylsilanyl-ethoxymethyl)-4,5-dihydro-3H-pyrrolo[3,2-d]pyrimidine-7-carboxylic Acid Methyl Ester

A solution of 6-chloro-4-oxo-3,5-bis-(2-trimethylsilanyl-ethoxymethyl)-4,5-dihydro-3H-pyrrolo[3,2-d]pyrimidine-7-carboxylic acid methyl ester (5.0 g, 10.24 mmol) in THF (250 mL) was treated with TBAF (103 mL of a 2M solution in THF) and heated at 60° C. for 2 hours The THF was evaporated and the residue was triturated with water and recrystallised from MeOH to give the title compound.

MS: 358[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.21 min.

### E 6-Chloro-3-[2-(3-methoxy-phenyl)-2-oxo-ethyl]-4-oxo-5-(2-trimethylsilanyl-ethoxymethyl)-4,5-dihydro-3H-pyrrolo[3,2-d]pyrimidine-7-carboxylic Acid Methyl Ester

A mixture of 6-chloro-4-oxo-5-(2-trimethylsilanyl-ethoxymethyl)-4,5-dihydro-3H-pyrrolo[3,2-d]pyrimidine-7-carboxylic acid methyl ester (3.5 g, 9.8 mmol), 2-bromo-1-(3-methoxyphenyl)-ethanone (2.47, 10.8 mmol) and potassium carbonate (2.02 g, 14.6 mmol) in DMF (20 mL) was stirred at room temperature for 1 hour The mixture was concentrated and the residue was triturated with water. The solid was collected and dried to afford the title compound as a beige solid.

MS: 506[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.94 min.

### F 6-Chloro-3-[2-(3-methoxy-phenyl)-2-oxo-ethyl]-4-oxo-4,5-dihydro-3H-pyrrolo[3,2-d]pyrimidine-7-carboxylic Acid Methyl Ester

A solution of 6-Chloro-3-[2-(3-methoxy-phenyl)-2-oxo-ethyl]-4-oxo-5-(2-trimethylsilanyl-ethoxymethyl)-4,5-dihydro-3H-pyrrolo[3,2-d]pyrimidine-7-carboxylic acid methyl ester (4.9 g, 9.7 mmol) in DCM (25 mL) was treated with TFA (25 mL) and the mixture was stirred at room temperature for 1 hour The volatiles were removed in vacuo, and the residue was chased with toluene to remove last traces of TFA. The residue was triturated with saturated sodium bicarbonate (25 mL), filtered and drying in vacuo (18 h, 75° C.) to afford the title compound as a cream solid.

MS: 376[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.78 min.

### G 5-Benzyl-6-chloro-3-[2-(3-methoxy-phenyl)-2-oxo-ethyl]-4-oxo-4,5-dihydro-3H-pyrrolo[3,2-d]pyrimidine-7-carboxylic Acid Methyl Ester

Diisopropylethylamine (0.82 mL, 4.70 mmol) and benzyl bromide (0.31 mL, 2.61 mmol) were added to a suspension of 6-chloro-3-[2-(3-methoxy-phenyl)-2-oxo-ethyl]-4-oxo-4,5-dihydro-3H-pyrrolo[3,2-d]pyrimidine-7-carboxylic acid methyl ester (0.8 g, 2.13 mmol) in DMF (3 mL) and the resulting solution was stirred at ambient temperature for 1 hour The resulting solid was collected, washed with water and dried to give the title compound as a yellow solid.

MS: 466[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.60 min.

### H 5-Benzyl-6-((S)-3-tert-butoxycarbonylamino-piperidin-1-yl)-3-[2-(3-methoxy-phenyl)-2-oxo-ethyl]-4-oxo-4,5-dihydro-3H-pyrrolo[3,2-d]pyrimidine-7-carboxylic Acid Methyl Ester

A mixture of 5-benzyl-6-chloro-3-[2-(3-methoxy-phenyl)-2-oxo-ethyl]-4-oxo-4,5-dihydro-3H-pyrrolo[3,2-d]pyrimidine-7-carboxylic acid methyl ester (0.9 g, 1.93 mmol) and (S)-3-N-boc-aminopiperidine (1.35 g, 6.74 mmol) in DMA (18 mL) was stirred at 130° C. for 89 hours The DMA was evaporated and the residue was partitioned between DCM and 20% v/v acetic acid. The organic phase was washed with water, sodium bicarbonate (aq., sat.) and brine, and dried (MgSO₄) and evaporated to dryness. The residue was purified by flash chromatography (Silica, 2% MeOH/DCM as eluent) to give the title compound as a gum.

MS: 630[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.96 min.

### I 5-Benzyl-6-((S)-3-tert-butoxycarbonylamino-piperidin-1-yl)-3-[2-(3-methoxy-phenyl)-2-oxo-ethyl]-4-oxo-4,5-dihydro-3H-pyrrolo[3,2-d]pyrimidine-7-carboxylic Acid

A solution of 5-benzyl-6-((S)-3-tert-butoxycarbonylamino-piperidin-1-yl)-3-[2-(3-methoxy-phenyl)-2-oxo-ethyl]-4-oxo-4,5-dihydro-3H-pyrrolo[3,2-d]pyrimidine-7-carboxylic acid methyl ester (1.3 g, 2.06 mmol) in dioxane (45 mL) was treated with aqueous lithium hydroxide (13 mL of 0.5M solution) and the resulting mixture stirred at 55° C. for 3 hours The reaction mixture was concentrated by evaporation and treated with saturated aqueous ammonium chloride and extracted with Ethyl acetate. The organic phase was dried (MgSO₄) and evaporated to dryness to give the title compound.

MS: 614[M−H]⁻

T_{R} [HPLC, Phenomenex Luna 3 micron C18; 5-95% CH₃CN+0.1% Formic acid/H₂O+0.1% Formic acid for 5 min, flow 2.0 ml/min]: 3.98 min.

### J 6-((S)-3-aminopiperidin-1-yl)-N,N-dimethyl-3-(2-(3-(methyloxy)phenyl)-2-oxoethyl)-4-oxo-5-(phenylmethyl)-4,5-dihydro-3H-pyrrolo[3,2-d]pyrimidine-7-carboxamide Hydrochloride

A solution of 5-benzyl-6-((S)-3-tert-butoxycarbonylamino-piperidin-1-yl)-3-[2-(3-methoxy-phenyl)-2-oxo-ethyl]-4-oxo-4,5-dihydro-3H-pyrrolo[3,2-d]pyrimidine-7-carboxylic acid (277 mg, 0.45 mmol), HATU (184 mg, 0.48 mmol) and diisopropylethylamine in DMF (17 mL) was stirred at ambient temperature for 5 min. A solution of dimethylamine (260 μL of a 2 molar solution in THF) was added and stirring was continued for 1 hour. The DMF was evaporated and the residue was purified by flash chromatography (Silica, 2% MeOH/DCM as eluent). The appropriate fractions were combined and were dissolved in DCM (4 mL) and treated with TFA (1 mL). After stirring at ambient temperature for 1 h, the reaction mixture was loaded onto SCX-2 cartridge and was washed with DCM and MeOH before eluting with a 2M solution ammonia in MeOH. Final purification by flash chromatography (Silica, 5% MeOH/DCM as eluent) followed by treatment with hydrogen chloride in MeOH and evaporation gave the title compound as a white solid.

MS: 543 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.11 min.

### Example O2

### 6-((S)-3-Aminopiperidin-1-yl)-3-(2-(3-(methyloxy)phenyl)-2-oxoethyl)-7-(morpholin-4-ylcarbonyl)-5-(phenylmethyl)-3,5-dihydro-4H-pyrrolo[3,2-d]pyrimidin-4-one Hydrochloride

This compound was prepared according to scheme 15.

This compound was prepared analogously as described in Example O1 using morpholine instead of dimethylamine.

MS: 585 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.12 min.

### Example O3

### 6-((S)-3-Aminopiperidin-1-yl)-5-but-2-yn-1-yl-3-(2-(3-(methyloxy)phenyl)-2-oxoethyl)-7-(morpholin-4-ylcarbonyl)-3,5-dihydro-4H-pyrrolo[3,2-d]pyrimidin-4-one Hydrochloride

This compound was prepared according to scheme 15.

This compound was prepared analogously as described in Example O1 using 1-bromo-but-2-yne instead of benzyl bromide and using morpholine instead of dimethylamine.

MS: 547 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.45 min.

### Example O4

### 6-((S)-3-Aminopiperidin-1-yl)-N,N-dimethyl-5-(3-methylbut-2-en-1-yl)-3-(2-(3-(methyloxy)phenyl)-2-oxoethyl)-4-oxo-4,5-dihydro-3H-pyrrolo[3,2-d]pyrimidine-7-carboxamide

This compound was prepared according to scheme 15.

This compound was prepared analogously as described in Example O1 using 1-bromo-3-methyl-but-2-ene instead of benzyl bromide.

MS: 521 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.04 min.

### Example O5

### 5-But-2-yn-1-yl-6-(1,4-diazepan-1-yl)-3-(isoquinolin-1-ylmethyl)-N,N-dimethyl-4-oxo-4,5-dihydro-3H-pyrrolo[3,2-d]pyrimidine-7-carboxamide Hydrochloride

This compound was prepared according to scheme 15.

### A 4-Oxo-4,5-dihydro-3H-pyrrolo[3,2-d]pyrimidine-7-carboxylic Acid Methyl Ester

A mixture of 3-amino-1H-pyrrole-2,4-dicarboxylic acid dimethyl ester (69.82 g, 352 mmol) and formamidine acetate (73.3 g, 705 mmol) in 2-methoxyethanol (250 mL) were heated at 125° C., under an atmosphere of argon, for 6 hours. During warming, the reagents dissolve and within a few minutes, a precipitate forms, which corresponds to the title compound. The reaction mixture was cooled to room temperature and MeOH (150 mL) was added. After stirring for a further few minutes, the precipitate was recovered by vacuum filtration and was washed with MeOH (ca. 50 mL). This was dried in vacuo, at 120° C. for 48 hours to afford the title compound as a light grey solid.

MS: 194.15 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 1.54 min.

### B 4-Oxo-3,5-bis-(2-trimethylsilanyl-ethoxymethyl)-4,5-dihydro-3H-pyrrolo[3,2-d]pyrimidine-7-carboxylic Acid Methyl Ester

Anhydrous DMF (750 mL) was added to sodium hydride (23.8 g, 596 mmol of a 60% dispersion in mineral oil), followed by 4-oxo-4,5-dihydro-3H-pyrrolo[3,2-d]pyrimidine-7-carboxylic acid methyl ester (46.07 g, 239 mmol). The suspension was stirred at 50° C. for 1 hr, until hydrogen evolution has ceased. The reaction mixture was cooled in an ice-bath and (2-chloromethoxy-ethyl)-trimethyl-silane (105.7 mL, 596 mmol) was added dropwise over 15 mins. The resulting solution was allowed to stir at room temperature for 2 hours. The DMF was removed in vacuo and the residue was partitioned between ethyl acetate (ca. 500 mL) and water (500 mL). The organic phase was washed with water (ca. 3×500 mL) and concentrated in vacuo to give a pale yellow-orange solid. This was triturated with petrol (40-60° C.) and the fine white solid was filtered and washed with petrol (40-60° C.) to afford the title compound.

MS: 454.2 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.47 min.

### C 6-Chloro-4-oxo-3,5-bis-(2-trimethylsilanyl-ethoxymethyl)-4,5-dihydro-3H-pyrrolo[3,2-d]pyrimidine-7-carboxylic Acid Methyl Ester

A solution of 4-oxo-3,5-bis-(2-trimethylsilanyl-ethoxymethyl)-4,5-dihydro-3H-pyrrolo[3,2-d]pyrimidine-7-carboxylic acid methyl ester (41.4 g, 91.3 mmol) in DMF (500 mL) was treated with N-chlorosuccinimide (18.3 g, 137 mmol) and stirred at room temperature for 18 hours, under an atmosphere of argon. The solid was collected by filtration, washed with DMF (ca. 50 mL) and then dissolved in DCM (300 mL), washed with water (3×200 mL), brine (400 mL) and dried (Na₂SO₄) to afford the title compound as a white powder.

MS: 488.17 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.74 min.

### D 6-Chloro-4-oxo-5-(2-trimethylsilanyl-ethoxymethyl)-4,5-dihydro-3H-pyrrolo[3,2-d]pyrimidine-7-carboxylic Acid Methyl Ester

A solution of 6-chloro-4-oxo-3,5-bis-(2-trimethylsilanyl-ethoxymethyl)-4,5-dihydro-3H-pyrrolo[3,2-d]pyrimidine-7-carboxylic acid methyl ester (5.0 g, 10.24 mmol) in THF (250 mL) was treated with tetrabutylammonium fluoride (103 mL of a 2M solution in THF) and heated at 60° C. for 2 hours The THF was evaporated and the residue was triturated with water and recrystallised from MeOH to give the title compound.

MS: 358[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.21 min.

### E 6-Chloro-3-isoquinolin-1-ylmethyl-4-oxo-5-(2-trimethylsilanyl-ethoxymethyl)-4,5-dihydro-3H-pyrrolo[3,2-d]pyrimidine-7-carboxylic Acid Methyl Ester

A mixture of 6-chloro-4-oxo-5-(2-trimethylsilanyl-ethoxymethyl)-4,5-dihydro-3H-pyrrolo[3,2-d]pyrimidine-7-carboxylic acid methyl ester (2.61 g, 7.29 mmol), 1-(bromomethyl)-isoquinoline hydrobromide (2.21 g, 7.29 mmol) and potassium carbonate (2.02 g, 14.65 mmol) in DMF (30 mL) was stirred at room temperature for 1 hour. The mixture was concentrated and the residue was dissolve in DCM and the solution washed with water and brine and then dried (MgSO₄). Evaporation of the solvent afforded the title compound as a gum.

MS: 499/501 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.10 min.

### F 6-Chloro-3-isoquinolin-1-ylmethyl-4-oxo-4,5-dihydro-3H-pyrrolo[3,2-d]pyrimidine-7-carboxylic Acid Methyl Ester

A solution of 6-chloro-3-isoquinolin-1-ylmethyl-4-oxo-5-(2-trimethylsilanyl-ethoxymethyl)-4,5-dihydro-3H-pyrrolo[3,2-d]pyrimidine-7-carboxylic acid methyl ester (from step E) was dissolved in a mixture of DCM (100 mL) and TFA (50 mL) and the mixture was stirred at room temperature for 1 hour. The volatiles were removed in vacuo, and the residue was purified by flash chromatography (Silica, gradient elution with 2% MeOH in DCM to 5% MeOH in DCM) to afford the title compound as a tan solid.

MS: 368/370[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.14 min.

### G 5-But-2-ynyl-6-chloro-3-isoquinolin-1-ylmethyl-4-oxo-4,5-dihydro-3H-pyrrolo[3,2-d]pyrimidine-7-carboxylic Acid Methyl Ester

Diisopropylethylamine (625 μL, 3.58 mmol) and 1-bromo-but-2-yne (190 μL, 2.17 mmol) were added to a solution of 6-chloro-3-isoquinolin-1-ylmethyl-4-oxo-4,5-dihydro-3H-pyrrolo[3,2-d]pyrimidine-7-carboxylic acid methyl ester (660 mg, 1.79 mmol) in DMF (8 mL) and the resulting solution was stirred at ambient temperature for 18 hours. The DMF was evaporated and the residue was triturated with water, then dried in vacuo at 40° C. to give the title compound as a tan solid.

MS: 421[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.26 min.

### H 6-(4-tert-Butoxycarbonyl-[1,4]diazepan-1-yl)-5-but-2-ynyl)-3-isoquinolin-1-ylmethyl-4-oxo-4,5-dihydro-3H-pyrrolo[3,2-d]pyrimidine-7-carboxylic Acid Methyl Ester

A mixture of 5-but-2-ynyl-6-chloro-3-isoquinolin-1-ylmethyl-4-oxo-4,5-dihydro-3H-pyrrolo[3,2-d]pyrimidine-7-carboxylic acid methyl ester (510 mg, 1.21 mmol) and [1,4]diazepine-1-carboxylic acid tert-butyl ester (1.0 mL, 5.07 mmol) in DMA (3 mL) was heated by microwave irradiation at 160° C. for 75 min. Another aliquot [1,4]diazepine-1-carboxylic acid tert-butyl ester (200 μL) was added and heating was continued for 30 min. The DMA was evaporated and the residue was dissolved in DCM and the solution was washed with water, 20% v/v aqueous acetic acid, saturated aqueous sodium bicarbonate and brine. After drying (MgSO₄) the mixture was evaporated to dryness. The residue was purified by flash chromatography (Silica, gradient elution with DCM to 2% MeOH in DCM as eluent) to give the title compound.

MS: 585[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.80 min.

### I 6-(4-tert-Butoxycarbonyl-[1,4]diazepan-1-yl)-5-but-2-ynyl)-3-isoquinolin-1-ylmethyl-4-oxo-4,5-dihydro-3H-pyrrolo[3,2-d]pyrimidine-7-carboxylic Acid

A solution of 6-(4-tert-butoxycarbonyl-[1,4]diazepan-1-yl)-5-but-2-ynyl)-3-isoquinolin-1-ylmethyl-4-oxo-4,5-dihydro-3H-pyrrolo[3,2-d]pyrimidine-7-carboxylic acid methyl ester (650 mg, 1.11 mmol) in dioxane (35 mL) was treated with aqueous lithium hydroxide (10 mL of 0.5M solution) and the resulting mixture stirred at 60° C. for 3 hours. The reaction mixture was concentrated by evaporation and treated with saturated aqueous ammonium chloride NH₄Cl then extracted with ethyl acetate. The organic phase was dried (MgSO₄) and evaporated to dryness to give the title compound as an oil.

MS: 571[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.83 min.

### J 4-(5-But-2-ynyl-7-dimethylcarbamoyl-3-isoquinolin-1-ylmethyl-4-oxo-4,5-dihydro-3H-pyrrolo[3,2-d]pyrimidin-6-yl)-[1,4]diazepane-1-carboxylic Acid tert-butyl Ester

A solution of 6-(4-tert-butoxycarbonyl-[1,4]diazepan-1-yl)-5-but-2-ynyl)-3-isoquinolin-1-ylmethyl-4-oxo-4,5-dihydro-3H-pyrrolo[3,2-d]pyrimidine-7-carboxylic acid (85 mg, 0.149 mmol), HATU (68 mg, 0.178 mmol) and diisopropylethylamine (65 μL, 0.346 mmol) in DMF (3 mL) was treated with a solution of dimethylamine (82 μL of a 2 molar solution in THF) and was stirred for 2 hour. The DMF was evaporated and the residue was partitioned between DCM and aqueous sodium bicarbonate. The organic phase was washed with brine, dried (MgSO₄) and evaporated to dryness. The residue was purified by flash chromatography (Silica, sequential elution with 80% ethyl acetate in pentane, DCM and 5% MeOH in DCM) to give the title compound as a pale yellow foam.

MS: 598[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.50 min.

### K 5-But-2-yn-1-yl-6-(1,4-diazepan-1-yl)-3-(isoquinolin-1-ylmethyl)-N N-dimethyl-4-oxo-4,5-dihydro-3H-pyrrolo[3,2-d]pyrimidine-7-carboxamide Hydrochloride

A solution of 4-(5-but-2-ynyl-7-dimethylcarbamoyl-3-isoquinolin-1-ylmethyl-4-oxo-4,5-dihydro-3H-pyrrolo[3,2-d]pyrimidin-6-yl)-[1,4]diazepane-1-carboxylic acid tert-butyl ester (55 mg, 0.092 mmol) in DCM (2 mL) and treated with TFA (1 mL) and the mixture was stirred at ambient temperature for 2 hours. The reaction mixture was loaded on to an SCX-2 cartridge and the cartridge was washed with DCM and a small amount of MeOH before eluting with 2M ammonia in MeOH. The appropriate fractions were collected and evaporated to dryness. The residue was purified by flash chromatography (Silica, gradient elution with DCM to 10% MeOH in DCM) to give the free base of the title compound. The free base was dissolved in MeOH and treated with conc. hydrochloric acid (200 μL) and blown down to dryness to afford the title compound.

MS: 498[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.17 min.

### Example O6

### 5-But-2-yn-1-yl-6-(1,4-diazepan-1-yl)-3-(isoquinolin-1-ylmethyl)-7-(piperidin-1-ylcarbonyl)-3,5-dihydro-4H-pyrrolo[3,2-d]pyrimidin-4-one Hydrochloride

This compound was prepared according to scheme 15.

This compound was prepared analogously as described in Example O5 using piperidine instead of dimethylamine.

MS: 538[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.94 min.

### Example O7

### 5-But-2-yn-1-yl-6-(1,4-diazepan-1-yl)-3-(isoquinolin-1-ylmethyl)-7-(pyrrolidin-1-ylcarbonyl)-3,5-dihydro-4H-pyrrolo[3,2-d]pyrimidin-4-one Hydrochloride

This compound was prepared according to scheme 15.

This compound was prepared analogously as described in Example O5 using pyrrolidine instead of dimethylamine.

MS: 524[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.62 min.

### Example O8

### 5-But-2-yn-1-yl-6-(1,4-diazepan-1-yl)-7-((4,4-difluoropiperidin-1-yl)carbonyl)-3-(isoquinolin-1-ylmethyl)-3,5-dihydro-4H-pyrrolo[3,2-d]pyrimidin-4-one Hydrochloride

This compound was prepared according to scheme 15.

This compound was prepared analogously as described in Example O5 using 4,4-difluoro-piperidine instead of dimethylamine.

MS: 574[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.33 min.

### Example O9

### 6-((S)-3-Aminopiperidin-1-yl)-5-but-2-yn-1-yl-3-(isoquinolin-1-ylmethyl)-N,N-dimethyl-4-oxo-4,5-dihydro-3H-pyrrolo[3,2-d]pyrimidine-7-carboxamide Hydrochloride

This compound was prepared according to scheme 15.

This compound was prepared analogously as described in Example O5 using (S)-piperidin-3-yl-carbamic acid tert-butyl ester instead of [1,4]diazepine-1-carboxylic acid tert-butyl ester.

MS: 498[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.69 min.

### Example O10

### 6-((S)-3-Aminopiperidin-1-yl)-5-but-2-yn-1-yl-3-(isoquinolin-1-ylmethyl)-7-(morpholin-4-ylcarbonyl)-3,5-dihydro-4H-pyrrolo[3,2-d]pyrimidin-4-one Hydrochloride

This compound was prepared according to scheme 15.

This compound was prepared analogously as described in Example O5 using (S)-piperidin-3-yl-carbamic acid tert-butyl ester instead of [1,4]diazepine-1-carboxylic acid tert-butyl ester and using morpholine instead of dimethylamine.

MS: 540[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.85 min.

### Example O11

### 6-((S)-3-Aminopiperidin-1-yl)-3-(isoquinolin-1-ylmethyl)-N,N-dimethyl-4-oxo-5-(phenylmethyl)-4,5-dihydro-3H-pyrrolo[3,2-d]pyrimidine-7-carboxamide Hydrochloride

This compound was prepared according to scheme 15.

This compound was prepared analogously as described in Example O5 using benzyl bromide instead of 1-bromo-but-2-yne and using (S)-piperidin-3-yl-carbamic acid tert-butyl ester instead of [1,4]diazepine-1-carboxylic acid tert-butyl ester.

MS: 536[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.88 min.

### Example O12

### 6-((S)-3-Aminopiperidin-1-yl)-5-(3-methylbut-2-en-1-yl)-3-(2-(3-(methyloxy)phenyl)-2-oxoethyl)-7-(morpholin-4-ylcarbonyl)-3,5-dihydro-4H-pyrrolo[3,2-d]pyrimidin-4-one Hydrochloride

This compound was prepared according to scheme 15.

This compound was prepared analogously as described in Example O5 using benzyl bromide instead of 1-bromo-but-2-yne and using (S)-piperidin-3-yl-carbamic acid tert-butyl ester instead of [1,4]diazepine-1-carboxylic acid tert-butyl ester.

MS: 578[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.93 min.

### Example O13

### 6-((S)-3-Aminopiperidin-1-yl)-3-(isoquinolin-1-ylmethyl)-7-(morpholin-4-ylcarbonyl)-5-(phenylmethyl)-3,5-dihydro-4H-pyrrolo[3,2-d]pyrimidin-4-one Hydrochloride

This compound was prepared according to scheme 15.

This compound was prepared analogously as described in Example O5 using 2-bromo-1-(3-methoxy-phenyl)-ethanone instead of 1-(bromomethyl)-isoquinoline hydrobromide and using 1-bromo-3-methyl-but-2-ene instead of 1-bromo-but-2-yne and using (S)-piperidin-3-yl-carbamic acid tert-butyl ester instead of [1,4]diazepine-1-carboxylic acid tert-butyl ester and using morpholine instead of dimethylamine.

MS: 563[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.26 min.

### Example P1

### 6-((S)-3-Aminopiperidin-1-yl)-3-(2-(3-(methyloxy)phenyl)-2-oxoethyl)-5-(Phenylmethyl)-3,5-dihydro-4H-pyrrolo[3,2-d]pyrimidin-4-one

This compound was prepared according to scheme 16.

A solution of 5-benzyl-6-((S)-3-tert-butoxycarbonylamino-piperidin-1-yl)-3-[2-(3-methoxy-phenyl)-2-oxo-ethyl]-4-oxo-4,5-dihydro-3H-pyrrolo[3,2-d]pyrimidine-7-carboxylic acid methyl ester (Example O1) (140 mg, 0.22 mmol) in dioxane (5 mL) was treated with aqueous lithium hydroxide (1.4 mL of a 0.5M solution) was heated at 60° C. for 2 hours The reaction mixture was concentrated, treated with NH₄Cl (sat., aq.) and extracted into ethyl acetate. The extracts were dried, evaporated and the residue was dissolved in a mixture of DCM (2 mL) and TFA (1 mL). and stirred at ambient temperature for 3 hours The reaction mixture was loaded on to an SCX-2 cartridge, washed with DCM and MeOH and the product was eluted with 2M ammonia in MeOH. Combination of the appropriate fractions afforded the title compound.

MS: 472 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.09 min.

### Example P2

### 6-((S)-3-Aminopiperidin-1-yl)-5-but-2-yn-1-yl-3-(2-(3-(methyloxy)phenyl)-2-oxoethyl)-3,5-dihydro-4H-pyrrolo[3,2-d]pyrimidin-4-one

This compound was prepared according to scheme 16.

The title compound was prepared analogously as described in Example P1 from 6-((S)-3-tert-butoxycarbonylamino-piperidin-1-yl)-5-but-2-ynyl-3-[2-(3-methoxy-phenyl)-2-oxo-ethyl]-4-oxo-4,5-dihydro-3H-pyrrolo[3,2-d]pyrimidine-7-carboxylic acid methyl ester.

MS: 434 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.49 min.

### Example P3

### 6-((S)-3-Aminopiperidin-1-yl)-5-(3-methylbut-2-en-1-yl)-3-(2-(3-(methyloxy)phenyl)-2-oxoethyl)-3,5-dihydro-4H-pyrrolo[3,2-d]pyrimidin-4-one

This compound was prepared according to scheme 16.

The title compound was prepared analogously as described in Example P1 from 6-((S)-3-tert-butoxycarbonylamino-piperidin-1-yl)-5-(3-methyl-but-2-en-1-yl)-3-[2-(3-methyloxy-phenyl)-2-oxo-ethyl]-4-oxo-4,5-dihydro-3H-pyrrolo[3,2-d]pyrimidine-7-carboxylic acid methyl ester.

MS: 450 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.15 min

### Example P4

### 6-((S)-3-Aminopiperidin-1-yl)-5-but-2-yn-1-yl-3-(isoquinolin-1-ylmethyl)-3,5-dihydro-4H-pyrrolo[3,2-d]pyrimidin-4-one

This compound was prepared according to scheme 16.

The title compound was prepared analogously as described in Example P1 from 6-((S)-3-tert-butoxycarbonylamino-piperidin-1-yl)-5-but-2-ynyl-3-isoquinolin-1-ylmethyl-4-oxo-4,5-dihydro-3H-pyrrolo[3,2-d]pyrimidine-7-carboxylic acid methyl ester.

MS: 427 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.77 min

### Example P5

### 6-((S)-3-Aminopiperidin-1-yl)-3-(isoquinolin-1-ylmethyl)-5-(phenylmethyl)-3,5-dihydro-4H-pyrrolo[3,2-d]pyrimidin-4-one

This compound was prepared according to scheme 16.

The title compound was prepared analogously as described in Example P1 from 5-benzyl-6-((S)-3-tert-butoxycarbonylamino-piperidin-1-yl)-3-isoquinolin-1-ylmethyl-4-oxo-4,5-dihydro-3H-pyrrolo[3,2-d]pyrimidine-7-carboxylic acid methyl ester.

MS: 465 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.97 min.

### Example P6

### 5-But-2-yn-1-yl-6-(1,4-diazepan-1-yl)-3-(isoquinolin-1-ylmethyl)-3,5-dihydro-4H-pyrrolo[3,2-d]pyrimidin-4-one

This compound was prepared according to scheme 16.

The title compound was prepared analogously as described in Example P1 from 6-(4-tert-butoxycarbonyl-[1,4]diazepan-1-yl)-5-but-2-ynyl)-3-isoquinolin-1-ylmethyl-4-oxo-4,5-dihydro-3H-pyrrolo[3,2-d]pyrimidine-7-carboxylic acid [Example O5].

MS: 427 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.76 min.

### Example Q1

### 6-((S)-3-Aminopiperidin-1-yl)-5-(3-methylbut-2-en-1-yl)-4-oxo-3-(quinazolin-2-ylmethyl)-4,5-dihydro-3H-pyrrolo[3,2-d]pyrimidine-7-carbonitrile

This compound was prepared according to scheme 17.

### A 3-Amino-4-cyano-1H-pyrrole-2-carboxylic Acid Methyl Ester

A solution of sodium methoxide (50.3 mL of a 25 wt % solution in MeOH) was added in one portion to a solution of diethyl aminomalonate hydrochloride (15.5 g, 73.2 mmol) in MeOH (140 mL). 2-Ethoxymethylene-malononitrile (8.94 g, 73.2 mmol) was added during 15 minutes keeping the temperature below 45° C. The mixture was heated at reflux for 4 hours. After cooling to ambient temperature, the mixture was neutralized with glacial acetic acid (9 mL), and concentrated in vacuo to a thick paste. Water was added with stirring, and the resulting slurry was extracted with ethyl acetate (2×250 mL). The combined organic extracts were washed with aqueous saturated sodium bicarbonate (300 mL) and brine (300 mL), dried (MgSO₄), filtered and concentrated in vacuo to give a orange solid. The solid was triturated with diethyl ether (50 mL) and collected by filtration to give the title compound as a tan solid.

MS: 166 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.74 min.

### B 4-Oxo-4,5-dihydro-3H-pyrrolo[3,2-d]pyrimidine-7-carbonitrile

A solution of 3-amino-4-cyano-1H-pyrrole-2-carboxylic acid methyl ester (6.0 g, 45.4 mmol) in formamide (48 mL) was treated with a solution of sodium methoxide (31.1 mL of a 25 wt % solution in MeOH). The resulting solution was heated to 100° C. for 20 hours, cooled to 0° C. and treated with 2M aqueous hydrochloric acid (80 mL). The solid was collected by filtration and oven dried in vacuo (1 mbar, 100° C.) for 2 hours to give the title compound as a beige solid.

MS: 161 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.22 min.

### C 6-Bromo-4-oxo-4,5-dihydro-3H-pyrrolo[3,2-d]pyrimidine-7-carbonitrile

4-Oxo-4,5-dihydro-3H-pyrrolo[3,2-d]pyrimidine-7-carbonitrile (4.1 g, 25.6 mmol.) was suspended in DMF and N-bromosuccinimide (11.7 g, 64.0 mmol.) was added. The mixture was stirred at room temperature for 20 hours. Another equivalent of N-bromosuccinimide was added and stirring was continued for a further 18 hours. Water (150 ml) was added and a solid was formed. The solid was collected, washed with water and dried under vacuum at 60° C. for 2 hr to give the title compound as an orange solid.

MS: 239 and 241 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.87 min.

### D 6-Bromo-5-(3-methyl-but-2-enyl)-4-oxo-4,5-dihydro-3H-pyrrolo[3,2-d]pyrimidine-7-carbonitrile

A solution of 6-bromo-4-oxo-4,5-dihydro-3H-pyrrolo[3,2-d]pyrimidine-7-carbonitrile (3.0 g, 12.55 mmol) in DMF (100 mL) was treated with diisopropylethylamine (3.23 g, 25.1 mmol) and was then cooled in an ice bath . The 1-bromo-3-methyl-bute-2-ene (1.87 g, 12.55 mmol) was dissolved in DMF (10 mL) and was then added dropwise over 30 mins, keeping the temperature below 5° C. The mixture was then stirred in an ice bath for 2 hours. The mixture was evaporated and the residue partitioned between water and ethyl acetate. The organic layer was washed several times with water then evaporated. The residue was then passed down a flash silica column eluting with 2% MeOH/DCM. Appropriate fractions were combined and evaporated to give the title compound as a pale yellow solid.

MS: 307/309[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.00 min.

### E {(S)-1-[7-Cyano-5-(3-methyl-but-2-enyl)-4-oxo-4,5-dihydro-3H-pyrrolo[3,2-d]pyrimidin-6-yl]-piperidin-3-yl}-carbamic Acid tert-butyl Ester

(S)-piperidin-3-yl-carbamic acid tert-butyl ester (587 mg, 2.93 mmol) was added to a suspension of 6-bromo-5-(3-methyl-but-2-enyl)-4-oxo-4,5-dihydro-3H-pyrrolo[3,2-d]pyrimidine-7-carbonitrile (300 mg, 0.98 mmol) in methoxyethanol (3 mL) and the mixture was heated at 140° C. for 75 mins in the microwave. The mixture was then heated thermally at 130° C. overnight. The mixture was evaporated and the residue was passed down a flash column eluting with 5% MeOH/DCM. Appropriate fractions were combined and evaporated to give the title compound as a foam.

MS: 427[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.36 min.

### F {(S)-1-[7-Cyano-5-(3-methyl-but-2-enyl)-4-oxo-3-quinazolin-2-ylmethyl-4,5-dihydro-3H-pyrrolo[3,2-d]pyrimidin-6-yl]-piperidin-3-yl}-carbamic Acid tert-butyl Ester

A mixture of {(S)-1-[7-cyano-5-(3-methyl-but-2-enyl)-4-oxo-4,5-dihydro-3H-pyrrolo[3,2-d]pyrimidin-6-yl]-piperidin-3-yl}-carbamic acid tert-butyl ester (157 mg, 0.37 mmol), 2-(chloromethyl)-quinazoline (79 mg, 0.44 mmol) and potassium carbonate (102 mg, 0.74 mmol) in DMF (5 mL) was stirred at room temperature overnight. The residue was partitioned between water and ethyl acetate. The organic layer was washed with water and evaporated to give the title compound.

MS: 569[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.92 min.

### G 6-((S)-3-Aminopiperidin-1-yl)-5-(3-methylbut-2-en-1-yl)-4-oxo-3-(quinazolin-2-ylmethyl)-4,5-dihydro-3H-pyrrolo[3,2-d]pyrimidine-7-carbonitrile

{(S)-1-[7-Cyano-5-(3-methyl-but-2-enyl)-4-oxo-3-quinazolin-2-ylmethyl-4,5-dihydro-3H-pyrrolo[3,2-d]pyrimidin-6-yl]-piperidin-3-yl}-carbamic acid tert-butyl ester (88 mg, 0.155 mmol) was dissolved in DCM (2 mL) and TFA (2 mL) was added. The mixture was then stirred at room temperature for 2 hours. The mixture was evaporated and the residue was partitioned between saturated sodium bicarbonate solution and ethyl acetate. The organic layer was washed with water and evaporated. The residue was passed down a flash silica column eluting first with 5% and then 10% MeOH/DCM. Appropriate fractions were combined and evaporated to give the title compound as an oil.

MS: 468 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.48 min

### Example Q2

### 6-((S)-3-Aminopiperidin-1-yl)-5-but-2-yn-1-yl-4-oxo-3-(quinolin-4-ylmethyl)-4,5-dihydro-3H-pyrrolo[3,2-d]pyrimidine-7-carbonitrile

This compound was prepared according to scheme 17.

The title compound was prepared analogously as described in Example Q1 using 1-bromo-but-2-yne instead of 1-bromo-3-methyl-but-2-ene and using 4-(bromomethyl)-quinoline instead of 2-(chloromethyl)-quinazoline.

MS: 452 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.12 min

### Example Q3

### 6-((S)-3-Aminopiperidin-1-yl)-5-but-2-yn-1-yl-3-(isoquinolin-1-ylmethyl)-4-oxo-4,5-dihydro-3H-pyrrolo[3,2-d]pyrimidine-7-carbonitrile

This compound was prepared according to scheme 17.

The title compound was prepared analogously as described in Example Q1 using 1-bromo-but-2-yne instead of 1-bromo-3-methyl-but-2-ene and using 1-(bromomethyl)-isoquinoline instead of 2-(chloromethyl)-quinazoline.

MS: 452 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.41 min

### Example Q4

### 6-((S)-3-Aminopiperidin-1-yl)-5-but-2-yn-1-yl-4-oxo-4,5-dihydro-3H-pyrrolo[3,2-d]pyrimidine-7-carbonitrile

This compound was prepared according to scheme 17.

The title compound was prepared analogously as described in Example Q1, steps A, B and D, using 1-bromo-but-2-yne instead of 1-bromo-3-methyl-but-2-ene.

MS: 311 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.32 min

### Example Q5

### 6-((S)-3-Aminopiperidin-1-yl)-5-((2-cyanophenyl)methyl)-3-methyl-4-oxo-4,5-dihydro-3H-pyrrolo[3,2-d]pyrimidine-7-carbonitrile Hydrochloride

This compound was prepared according to scheme 17.

The title compound was prepared analogously as described in Example Q1, using 2-bromo-methyl-benzonitrile instead of 1-bromo-3-methyl-but-2-ene and using iodomethane instead of 2-(chloromethyl)-quinazoline. The hydrochloride was prepared by treatment of the free base with hydrogen chloride in MeOH and removal of volatiles.

MS: 388 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.34 min

### Example Q6

### 6-((S)-3-Aminopiperidin-1-yl)-5-((2-cyanophenyl)methyl)-3-(isoquinolin-1-ylmethyl)-4-oxo-4,5-dihydro-3H-pyrrolo[3,2-d]pyrimidine-7-carbonitrile Hydrochloride

This compound was prepared according to scheme 17.

The title compound was prepared analogously as described in Example Q1, using 2-bromo-methyl-benzonitrile instead of 1-bromo-3-methyl-but-2-ene and using 1-(bromomethyl)-isoquinoline hydrobromide instead of 2-(chloromethyl)-quinazoline. The hydrochloride was prepared by treatment of the free base with hydrogen chloride in MeOH and removal of volatiles.

MS: 515 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.96 min

### Example Q7

### 5-But-2-yn-1-yl-6-(1,4-diazepan-1-yl)-3-((4-methylquinazolin-2-yl)methyl)-4-oxo-4,5-dihydro-3H-pyrrolo[3,2-d]pyrimidine-7-carbonitrile

This compound was prepared according to scheme 17.

### A 3-Amino-4-cyano-1H-pyrrole-2-carboxylic Acid Methyl Ester

A solution of sodium methoxide (50.3 mL of a 25 wt % solution in MeOH) was added in one portion to a solution of diethyl aminomalonate hydrochloride (15.5 g, 73.2 mmol) in MeOH (140 mL). 2-Ethoxymethylene-malononitrile (8.94 g, 73.2 mmol) was added during 15 minutes keeping the temperature below 45° C. The mixture was heated at reflux for 4 hours. After cooling to ambient temperature, the mixture was neutralized with glacial acetic acid (9 mL), and concentrated in vacuo to a thick paste. Water was added with stirring, and the resulting slurry was extracted with ethyl acetate (2×250 mL). The combined organic extracts were washed with aqueous saturated sodium bicarbonate (300 mL) and brine (300 mL), dried (MgSO₄), filtered and concentrated in vacuo to give a orange solid. The solid was triturated with diethyl ether (50 mL) and collected by filtration to give the title compound as a tan solid.

MS: 166 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.74 min.

### B 4-Oxo-4,5-dihydro-3H-pyrrolo[3,2-d]pyrimidine-7-carbonitrile

A solution of 3-amino-4-cyano-1H-pyrrole-2-carboxylic acid methyl ester (6.0 g, 45.4 mmol) in formamide (48 mL) was treated with a solution of sodium methoxide (31.1 mL of a 25 wt % solution in MeOH). The resulting solution was heated to 100° C. for 20 hours, cooled to 0° C. and treated with 2M aqueous hydrochloric acid (80 mL). The solid was collected by filtration and oven dried in vacuo (1 mbar, 100° C.) for 2 hours to give the title compound as a beige solid.

MS: 161 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.22 min.

### C 6-Bromo-4-oxo-4,5-dihydro-3H-pyrrolo[3,2-d]pyrimidine-7-carbonitrile

4-Oxo-4,5-dihydro-3H-pyrrolo[3,2-d]pyrimidine-7-carbonitrile (4.1 g, 25.6 mmol.) was suspended in DMF and N-bromosuccinimide (11.7 g, 64.0 mmol.) was added. The mixture was stirred at room temperature for 20 hours. Another equivalent of N-bromosuccinimide was added and stirring was continued for a further 18 hours. Water (150 ml) was added and a solid was formed. The solid was collected, washed with water and dried under vacuum at 60° C. for 2 hours to give the title compound as an orange solid.

MS: 239 and 241 [M+H]⁺.

T_{R} [HPLC, Phenomenex Luna 3 micron C18; 5-95% CH₃CN+0.1% Formic acid/H₂O+0.1% Formic acid for 5 min, flow 2.0 ml/min]: 1.87 min.

### D 6-Bromo-5-(but-2-ynyl)-4-oxo-4,5-dihydro-3H-pyrrolo[3,2-d]pyrimidine-7-carbonitrile

A solution of 6-bromo-4-oxo-4,5-dihydro-3H-pyrrolo[3,2-d]pyrimidine-7-carbonitrile (5.0 g, 21.0 mmol) in DMF (100 mL) was treated with a solution of diisopropylethylamine (4.02 mL, 23.0 mmol) and 1-bromo-but-2-yne (1.89 g, 21.0 mmol) in DMF (10 mL) during 35 min. The mixture was then stirred at room temperature for 18 hours. The mixture was evaporated and the residue partitioned between water and ethyl acetate and a precipitate formed. The solid was collected washed with ethyl acetate and dried to give the title compound as a tan solid.

MS: 293[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.61 min.

### E 4-(5-But-2-ynyl-7-cyano-4-oxo-4,5-dihydro-3H-pyrrolo[3,2-d]pyrimidin-6-yl)-[1,4]diazepane-1-carboxylic Acid tert-butyl Ester

A mixture of [1,4]diazepane-1-carboxylic acid tert-butyl ester (2.0 g, 3.44 mmol) and 6-bromo-5-(but-2-ynyl)-4-oxo-4,5-dihydro-3H-pyrrolo[3,2-d]pyrimidine-7-carbonitrile (6.8 g, 17.23 mmol) in DMA (20 mL) was heated under microwave irradiation at 160° C. for 60 mins. The mixture was evaporated and the residue was dissolved in DCM and washed with aqueous sodium bicarbonate and brine. After drying (MgSO₄), the solvent was evaporated and the residue was purified by flash chromatography (Silica, sequential elution with DCM, 30% ethyl acetate in DCM and ethyl acetate). The appropriate fractions were combined and evaporated and the residue dissolved in DCM. The solution was washed with 20% aqueous acetic acid, saturated aqueous sodium bicarbonate and brine. After drying (MgSO₄), the solvent was evaporated to afford the title compound as a foam.

MS: 411[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.08 min.

### F 5-But-2-yn-1-yl-6-(1,4-diazepan-1-yl)-3-((4-methylquinazolin-2-yl)methyl)-4-oxo-4,5-dihydro-3H-pyrrolo[3,2-d]pyrimidine-7-carbonitrile

A mixture of 4-(5-but-2-ynyl-7-cyano-4-oxo-4,5-dihydro-3H-pyrrolo[3,2-d]pyrimidin-6-yl)-[1,4]diazepane-1-carboxylic acid tert-butyl ester (100 mg, 0.244 mmol), 2-chloromethyl-4-methyl-quinazoline (52 mg, 0.270 mmol) and potassium carbonate (67 mg, 0.485 mmol) in DMF (1.5 mL) was stirred at room temperature overnight. The solvent was evaporated and the residue was triturated with water to give a cream coloured solid. The solid was dissolved in DCM (2 mL) and TFA (1 mL) and stirred at room temperature for 2 hours. The reaction mixture was loaded on to an SCX-2 cartridge and washed with DCM and a small amount of MeOH before eluting with 2M ammonia in MeOH. Appropriate fractions were combined and evaporated to dryness. The residue was purified by flash chromatography (Silca, gradient elution with DCM to 10% MeOH in DCM) to give the title compound as a yellow solid.

MS: 467 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.46 min

### Example Q8

### 4-((5-But-2-yn-1-yl-7-cyano-6-(1,4-diazepan-1-yl)-4-oxo-4,5-dihydro-3H-pyrrolo[3,2-d]pyrimidin-3-yl)methyl)-quinoline-3-carbonitrile

This compound was prepared according to scheme 17.

The title compound was prepared analogously as described in Example Q7, using 4-(chloromethyl)-quinoline-3-carbonitrile instead of 2-chloromethyl-4-methyl-quinazoline.

MS: 477 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.25 min.

### Example Q9

### 5-But-2-yn-1-yl-3-((3-cyanopyridin-2-yl)methyl)-6-(1,4-diazepan-1-yl)-4-oxo-4,5-dihydro-3H-pyrrolo[3,2-d]pyrimidine-7-carbonitrile Hydrochloride

This compound was prepared according to scheme 17.

The title compound was prepared analogously as described in Example Q7, using 2-(chloromethyl)-nicotinonitrile instead of 2-chloromethyl-4-methyl-quinazoline. The hydrochloride was prepared by treatment of the free base with hydrogen chloride in MeOH and removal of volatiles.

MS: 427 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.49 min.

### Example Q10

### 5-But-2-yn-1-yl-6-(1,4-diazepan-1-yl)-4-oxo-3-(quinoxalin-2-ylmethyl)-4,5-dihydro-3H-pyrrolo[3,2-d]pyrimidine-7-carbonitrile Hydrochloride

This compound was prepared according to scheme 17.

The title compound was prepared analogously as described in Example Q7, using 2-(chloromethyl)-quinoxaline instead of 2-chloromethyl-4-methyl-quinazoline. The hydrochloride was prepared by treatment of the free base with hydrogen chloride in MeOH and removal of volatiles.

MS: 453 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.97 min.

### Example Q11

### 5-But-2-yn-1-yl-6-(1,4-diazepan-1-yl)-3-((4-methyl-3-oxidoquinazolin-2-yl)methyl)-4-oxo-4,5-dihydro-3H-pyrrolo[3,2-d]pyrimidine-7-carbonitrile Hydrochloride

This compound was prepared according to scheme 17.

The title compound was prepared analogously as described in Example Q7, using 2-chloromethyl-4-methyl-quinazoline-3-oxide instead of 2-chloromethyl-4-methyl-quinazoline. The hydrochloride was prepared by treatment of the free base with hydrogen chloride in MeOH and removal of volatiles.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.46 min.

### Example Q12

### 6-((S)-3-Aminopiperidin-1-yl)-5-(3-methylbut-2-en-1-yl)-3-((2-oxidoisoquinolin-1-yl)methyl)-4-oxo-4,5-dihydro-3H-pyrrolo[3,2-d]pyrimidine-7-carbonitrile

This compound was prepared according to scheme 17.

The title compound was prepared analogously as described in Example Q1 using 1-(bromomethyl)-isoquinoline-2-oxide instead of 2-(chloromethyl)-quinazoline.

MS: 484 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.87 min

### Example Q13

### 6-((S)-3-Aminopiperidin-1-yl)-5-(3-methylbut-2-en-1-yl)-3-((4-methylquinazolin-2-yl)methyl)-4-oxo-4,5-dihydro-3H-pyrrolo[3,2-d]pyrimidine-7-carbonitrile

This compound was prepared according to scheme 17.

The title compound was prepared analogously as described in Example Q1 using 2-(chloromethyl)-4-methyl-quinazoline instead of 2-(chloromethyl)-quinazoline.

MS: 483 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.43 min

### Example Q14

### 6-((S)-3-Aminopiperidin-1-yl)-5-(3-methylbut-2-en-1-yl)-3-((4-methyl-3-oxidoquinazolin-2-yl)methyl)-4-oxo-4,5-dihydro-3H-pyrrolo[3,2-d]pyrimidine-7-carbonitrile

This compound was prepared according to scheme 17.

The title compound was prepared analogously as described in Example Q1 using 2-(chloromethyl)-4-methyl-quinazoline-3-oxide instead of 2-(chloromethyl)-quinazoline.

MS: 499 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.74 min

### Example R1

### 6-((S)-3-Aminopiperidin-1-yl)-5-but-2-yn-1-yl-1,3-dimethyl-2,4-dioxo-2,3,4,5-tetrahydro-1H-pyrrolo[3,2-d]pyrimidine-7-carbonitrile

This compound was prepared according to scheme 18.

### A 5-Benzyl-6-bromo-1,3-dimethyl-2,4-dioxo-2,3,4,5-tetrahydro-1H-pyrrolo[3,2-d]pyrimidine-7-carbonitrile

A stirred solution of 5-benzyl-6-bromo-2,4-dioxo-1-methyl-2,3,4,5-tetrahydro-1H-pyrrolo[3,2-d]pyrimidine-7-carbonitrile (830 mg, 2.31 mmol) in DMF (30 mL) was treated with iodomethane (328 mg, 2.31 mmol) and potassium carbonate (638 mg, 4.62 mmol) and stirring was continued for 18 h at room temperature. The solvent was then removed in vacuo and the residue was triturated (water) to give an off-white solid. The solid was purified by flash chromatography (Silica, gradient elution with DCM to 5% ethyl acetate in DCM) to afford the title compound as a white solid.

### B [(S)-1-(5-Benzyl-7-cyano-1,3-dimethyl-2,4-dioxo-2,3,4,5-tetrahydro-1H-pyrrolo[3,2-d]pyrimidin-6-yl)-piperidin-3-yl]-carbamic Acid tert-butyl Ester

A mixture of 5-benzyl-6-bromo-1,3-dimethyl-2,4-dioxo-2,3,4,5-tetrahydro-1H-pyrrolo[3,2-d]pyrimidine-7-carbonitrile (410 mg, 1.10 mmol) and (S)-piperidin-3-yl-carbamic acid tert-butyl ester (660 mg, 3.30 mmol) in DMA (10 mL) was heated to 160° C. in the microwave for 40 min. The solvent was removed in vacuo and the residue was purified by flash chromatography (Silica, gradient elution with DCM to 7.5% ethyl acetate in DCM). Combination of the appropriate fractions and removal of volatiles gave the title compound as an off-white solid.

MS: 493[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.85 min.

### C [(S)-1-(7-Cyano-1,3-dimethyl-2,4-dioxo-2,3,4,5-tetrahydro-1H-pyrrolo[3,2-d]pyrimidin-6-yl)-piperidin-3-yl]-carbamic Acid tert-butyl Ester

A mixture of [(S)-1-(5-benzyl-7-cyano-1,3-dimethyl-2,4-dioxo-2,3,4,5-tetrahydro-1H-pyrrolo[3,2-d]pyrimidin-6-yl)-piperidin-3-yl]-carbamic acid tert-butyl ester (200 mg, 0.41 mmol) 10% palladium on charcoal (200 mg) and ammonium formate (130 mg, 2.03 mmol) in ethanol (20 mL) was stirred at 70° C. for 60 min. After cooling, the mixture was filtered through a pad of diatomaceous earth, the pad was washed with MeOH and DCM. The combined filtrate and washings were concentrated in vacuo to give the title compound as a pale yellow solid.

MS: 403[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.99 min.

### D [(S)-1-(5-But-2-ynyl-7-cyano-1,3-dimethyl-2,4-dioxo-2,3,4,5-tetrahydro-1H-pyrrolo[3,2-d]pyrimidin-6-yl)-piperidin-3-yl]-carbamic Acid tert-butyl Ester

Diisopropylethylamine (31 μL, 0.179 mmol) was added to a stirred solution of [(S)-1-(7-cyano-1,3-dimethyl-2,4-dioxo-2,3,4,5-tetrahydro-1H-pyrrolo[3,2-d]pyrimidin-6-yl)-piperidin-3-yl]-carbamic acid tert-butyl ester (36 mg, 0.0894 mmol) and 1-bromo-but-2-yne (7.8 μL, 0.0894 mmol) in DMF (1 mL) at room temperature. The mixture was stirred for 18 hours then heated to 50° C. for 24 hours and 70° C. for a further 24 hours. A further quantity of 1-bromo-but-2-yne (7.8 μL, 0.0894 mmol) and diisopropylethylamine (62 μL, 0.358 mmol) were added and heating was continued for a further 2 hours. The solvent was removed in vacuo and the resulting residue was purified by flash chromatography (Silica, gradient elution with DCM to 10% ethyl acetate in DCM) to give the title compound as a gum.

MS: 455[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.60 min.

### E 6-((S)-3-Aminopiperidin-1-yl)-5-but-2-yn-1-yl-1,3-dimethyl-2,4-dioxo-2,3,4,5-tetrahydro-1H-pyrrolo[3,2-d]pyrimidine-7-carbonitrile

A mixture of [(S)-1-(5-but-2-ynyl-7-cyano-1,3-dimethyl-2,4-dioxo-2,3,4,5-tetrahydro-1H-pyrrolo[3,2-d]pyrimidin-6-yl)-piperidin-3-yl]-carbamic acid tert-butyl ester (12.6, 0.028 mmol), TFA (1 mL) and DCM (1 mL) was stirred at room temperature for 1 hour. The mixture was purified by flash chromatography (SCX-2, Washing with MeOH and then eluting with 2M ammonia in MeOH/MeOH (1:15)) and after removal of the volatiles the residue was dried in vacuo at 40° C. to afford the title compound as an orange gum.

MS: 355[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.50 min.

### Example R2

### 6-((S)-3-Aminopiperidin-1-yl)-1,3-dimethyl-5-(3-methylbut-2-en-1-yl)-2,4-dioxo-2,3,4,5-tetrahydro-1H-pyrrolo[3,2-d]pyrimidine-7-carbonitrile

This compound was prepared according to scheme 18.

The title compound was prepared analogously as described in Example R1 using 1-bromo-3-methyl-but-2-ene instead of 1-bromo-but-2-yne.

MS: 371 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.28 min.

### Example S1

### 1,3-Dimethyl-2,4-dioxo-5-(phenylmethyl)-6-piperazin-1-yl-2,3,4,5-tetrahydro-1H-pyrrolo[3,2-d]pyrimidine-7-carbonitrile Hydrochloride

This compound was prepared according to scheme 19.

### A 3-Amino-1-benzyl-4-cyano-1H-pyrrole-2-carboxylic Acid Ethyl Ester

A mixture of 2-(benzylamino-methylene)-malononitrile (10.94 g, 59.8 mmol), ethyl bromoacetate (9.94 mL, 89.7 mmol) and potassium carbonate (16.5 g, 119.6 mmol) were in DMF (200 mL) was heated at 90° C. for 50 mins. After cooling to 40° C., sodium ethoxide (77.7 mL of a 1M solution in ethanol) was added dropwise during 10 mins. The reaction mixture was heated to 90° C. for 25 mins. Glacial acetic acid (6.2 mL) was added and the reaction mixture was left to cool. The DMF was removed in vacuo and the residue was partitioned between ethyl acetate (200 mL) and water (200 mL). The layers were separated and the organic layer was washed with water (200 mL) and brine (200 mL), and dried (Na₂SO₄) Concentration gave a dark orange solid which was purified by flash chromatography (Silica, gradient elution with 10% ethyl acetate in cyclohexane to ethyl acetate). Fractions containing pure material were combined and concentrated to afford the title compound as a yellow solid.

MS: 270 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.30 min.

### B1-Benzyl-3-(3-benzyl-ureido)-4-cyano-1H-pyrrole-2-carboxylic Acid Ethyl Ester

A mixture of 3-amino-1-benzyl-4-cyano-1H-pyrrole-2-carboxylic acid ethyl ester (3.67 g, 13.6 mmol.) and benzyl isocyanate (2.53 mL, 20.5 mmol.) in pyridine (73 mL) was treated with microwave irradiation (Emrys Optimizer) at 120° C. for 30 mins. The reaction mixture was partitioned between ethyl acetate (100 mL) and 1M aq. hydrochloric acid (4×100 mL). The organic extract was dried (Na₂SO₄), filtered, concentrated in vacuo and the residue was purified by trituration with diethyl ether (50 mL), filtration and drying in a vacuum at 400 for 24 hours to afford the title compound as an off-white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### C 3,6-Dibenzyl-4-imino-2-oxo-2,3,4,6-tetrahydro-1H-pyrrolo[3,4-d]pyrimidine-7-carboxylic Acid Ethyl Ester

A mixture of 1-benzyl-3-(3-benzyl-ureido)-4-cyano-1H-pyrrole-2-carboxylic acid ethyl ester (2 g, 5 mmol.) and sodium methoxide (0.27 g, 5 mmol.) in MeOH (60 mL) was treated with microwave irradiation (Emrys Optimizer) at 60° C. for 5 mins. The solid that was formed was collected by filtration, washed with MeOH (20 mL) and air-dried to afford the title compound as a white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.13 min.

### D 3,5-Dibenzyl-2,4-dioxo-2,3,4,5-tetrahydro-1H-pyrrolo[3,2-d]pyrimidine-7-carbonitrile

A suspension of 3,6-dibenzyl-4-imino-2-oxo-2,3,4,6-tetrahydro-1H-pyrrolo[3,4-d]pyrimidine-7-carboxylic acid ethyl ester (1.13 g, 2.8 mmol.) and sodium methoxide (0.46 g, 8.4 mmol.) in MeOH (30 mL) was treated with microwave irradiation (Emrys Optimizer) at 140° C. for 20 mins. The reaction mixture was concentrated in vacuo and the solid obtained was triturated with water (10 mL), filtered and dried under vacuum at 40° C. for 24 hours to afford the title compound as a white solid.

MS: 357 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.36 min.

### E 3,5-Dibenzyl-1-methyl-2,4-dioxo-2,3,4,5-tetrahydro-1H-pyrrolo[3,2-d]pyrimidine-7-carbonitrile

3,5-Dibenzyl-2,4-dioxo-2,3,4,5-tetrahydro-1H-pyrrolo[3,2-d]pyrimidine-7-carbonitrile (0.95 g, 2.7 mmol.) was dissolved in DMSO (10 mL). To this was added potassium carbonate (0.74 g, 5.3 mmol.) followed by methyl iodide (0.25 mL, 4.0 mmol.). The reaction mixture was stirred at room temperature for 3 hours. A dense white precipitate was formed and the reaction mixture was diluted with water (20 mL). The solid was collected by filtration, washed with water (10 mL) and dried under vacuum at 40° C. for 72 hours to afford the title compound as a white solid.

MS: no mass ion.

T_{R} [HPLC, Phenomenex Luna 3 micron C18; 5-95% CH₃CN+0.1% Formic acid/H₂O+0.1% Formic acid for 5 min, flow 2.0 ml/min]: 3.86 min.

### F 5-Benzyl-1-methyl-2,4-dioxo-2,3,4,5-tetrahydro-1H-pyrrolo[3,2-d]pyrimidine-7-carbonitrile

A mixture of 3,5-dibenzyl-1-methyl-2,4-dioxo-2,3,4,5-tetrahydro-1H-pyrrolo[3,2-d]pyrimidine-7-carbonitrile (0.90 g, 2.4 mmol.) and boron tribromide (12.16 mL, 12.2 mmol.) in xylene (50 mL) was stirred at 140° C. for 5 hours. The reaction mixture was cooled, MeOH (15 mL) was added and the mixture was stirred at room temperature for 30 mins. The solvents were evaporated in vacuo and the residue was partitioned between ethyl acetate (100 mL) and saturated aq. sodium hydrogen carbonate (200 mL). The ethyl acetate suspension was concentrated in vacuo and the residue was triturated with diethyl ether (100 mL), filtered and air-dried to afford the title compound as a beige solid.

MS: 281 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.74 min.

### G 5-Benzyl-6-bromo-1-methyl-2,4-dioxo-2,3,4,5-tetrahydro-1H-pyrrolo[3,2-d]pyrimidine-7-carbonitrile

5-Benzyl-1-methyl-2,4-dioxo-2,3,4,5-tetrahydro-1H-pyrrolo[3,2-d]pyrimidine-7-carbonitrile (0.37 g, 1.3 mmol.) was suspended in acetic acid (8 mL) and warmed to 45° C. To this was added bromine (0.10 mL, 2.0 mmol.) dropwise, in acetic acid (2 mL). Once the addition was complete, water (3 mL) was added and the reaction mixture was stirred at 45° C. for 18 hours. Another 1.5 equivalents of bromine, in 3 mL acetic acid was added and the reaction mixture was stirred at 45° C. for 4 hours. Another 2 equivalents of bromine and 10 mL acetic acid were added and the reaction mixture was stirred at 70° C. for 72 hours. The solvents were removed in vacuo and the residue was triturated with saturated aq. sodium thiosulphite solution (20 mL), followed by water (10 mL). The solid was collected by filtration and was dried under vacuum at 40° C. for 18 hours to obtain the title compound as a fawn coloured solid.

MS: 359 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.07 min.

### H 4-(5-Benzyl-7-cyano-1-methyl-2,4-dioxo-2,3,4,5-tetrahydro-1H-pyrrolo[3,2-d]pyrimidin-6-yl)-piperazine-1-carboxylic Acid tert-butyl Ester

A mixture of 5-benzyl-6-bromo-1-methyl-2,4-dioxo-2,3,4,5-tetrahydro-1H-pyrrolo[3,2-d]pyrimidine-7-carbonitrile (500 mg, 1.39 mmol) and piperazine-1-carboxylic acid tert-butyl ester (777 mg, 4.17 mmol) in DMA (10 mL) was heated at 160° C. using microwave irradiation for a total of 80 min. An additional amount of piperazine-1-carboxylic acid tert-butyl ester (518 mg, 2.78 mmol) was added and the mixture was heated under microwave irradiation for a further 60 min at 160° C. The solvent was removed in vacuo and the residue was purified by flash chromatography (Silica, with gradient elution using DCM to 20% ethyl acetate in DCM). The appropriate fractions were combined, concentrated and the residue was extracted with DCM. The extracts were concentrated to give the title compound as a light brown solid.

MS: 465[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.49 min.

### I 1,3-Dimethyl-2,4-dioxo-5-(phenylmethyl)-6-piperazin-1-yl-2,3,4,5-tetrahydro-1H-pyrrolo[3,2-d]pyrimidine-7-carbonitrile Hydrochloride

A mixture of 4-(5-benzyl-7-cyano-1-methyl-2,4-dioxo-2,3,4,5-tetrahydro-1H-pyrrolo[3,2-d]pyrimidin-6-yl)-piperazine-1-carboxylic acid tert-butyl ester (120 mg, 0.258 mmol), iodomethane (32.2 μL, 0.517 mmol) and potassium carbonate (107 mg, 0.775) in DMF (3 mL) was stirred at room temperature under nitrogen for 3 hours. The solvent was removed in vacuo and the resulting residue was triturated with water to give a pale yellow solid. The solid was treated with TFA/DCM (1:1, 6 mL) for 1 hour at room temperature. The mixture was purified by flash chromatography (SCX-2 column, washing with MeOH and eluting with a mixture of 2M ammonia in MeOH/MeOH (1:4)). The relevant fractions were combined and concentrated in vacuo and the residue was re-purified by flash chromatography (Silica, gradient elution using DCM to 4% MeOH in DCM) to give the free base of the title compound. The free base was dissolved in MeOH (2 mL) and treated with hydrogen chloride (1.25 M in MeOH; 3 eq) and the solution was concentrated in vacuo and the residue was dried in vacuo at 45° C. for 18 h to afford the title compound as an off-white solid.

MS: 379[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.75 min.

### Example S2

### 1,3-Dimethyl-2,4-dioxo-5-(phenylmethyl)-6-piperazin-1-yl-2,3,4,5-tetrahydro-1H-pyrrolo[3,2-d]pyrimidine-7-carbonitrile Hydrochloride

This compound was prepared according to scheme 19.

The title compound was prepared analogously as described in Example S1 using 2-bromo-1-(3-methoxy-phenyl)-ethanone instead of iodomethane.

MS: 513 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.56 min

### Example S3

### 3-(Isoquinolin-1-ylmethyl)-1-methyl-2,4-dioxo-5-(phenylmethyl)-6-piperazin-1-yl-2,3,4,5-tetrahydro-1H-pyrrolo[3,2-d]pyrimidine-7-carbonitrile Hydrochloride

This compound was prepared according to scheme 19.

The title compound was prepared analogously as described in Example S1 using 1-(bromomethyl)-isoquinoline hydrobromide instead of iodomethane.

MS: 506 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.61 min

### Example S4

### 6-(1,4-Diazepan-1-yl)-1,3-dimethyl-2,4-dioxo-5-(phenylmethyl)-2,3,4,5-tetrahydro-1H-pyrrolo[3,2-d]pyrimidine-7-carbonitrile Hydrochloride

This compound was prepared according to scheme 19.

### A 3-Amino-1-benzyl-4-cyano-1H-pyrrole-2-carboxylic Acid Ethyl Ester

A mixture of 2-(benzylamino-methylene)-malononitrile (10.94 g, 59.8 mmol), ethyl bromoacetate (9.94 mL, 89.7 mmol) and potassium carbonate (16.5 g, 119.6 mmol) were in DMF (200 mL) was heated at 90° C. for 50 mins. After cooling to 40° C., sodium ethoxide (77.7 mL of a 1M solution in ethanol) was added dropwise during 10 mins. The reaction mixture was heated to 90° C. for 25 mins. Glacial acetic acid (6.2 mL) was added and the reaction mixture was left to cool. The DMF was removed in vacuo and the residue was partitioned between ethyl acetate (200 mL) and water (200 mL). The layers were separated and the organic layer was washed with water (200 mL) and brine (200 mL), and dried (Na₂SO₄) Concentration gave a dark orange solid which was purified by flash chromatography (Silica, gradient elution with 10% ethyl acetate in cyclohexane to ethyl acetate). Fractions containing pure material were combined and concentrated to afford the title compound as a yellow solid.

MS: 270 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.30 min.

### B1-Benzyl-3-(3-benzyl-ureido)-4-cyano-1H-pyrrole-2-carboxylic Acid Ethyl Ester

A mixture of 3-amino-1-benzyl-4-cyano-1H-pyrrole-2-carboxylic acid ethyl ester (3.67 g, 13.6 mmol.) and benzyl isocyanate (2.53 mL, 20.5 mmol.) in pyridine (73 mL) was treated with microwave irradiation (Emrys Optimizer) at 120° C. for 30 mins. The reaction mixture was partitioned between ethyl acetate (100 mL) and 1M aq. hydrochloric acid (4×100 mL). The organic extract was dried (Na₂SO₄), filtered, concentrated in vacuo and the residue was purified by trituration with diethyl ether (50 mL), filtration and drying in a vacuum at 40° for 24 hours to afford the title compound as an off-white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### C 3,6-Dibenzyl-4-imino-2-oxo-2,3,4,6-tetrahydro-1H-pyrrolo[3,4-d]pyrimidine-7-carboxylic Acid Ethyl Ester

A mixture of 1-benzyl-3-(3-benzyl-ureido)-4-cyano-1H-pyrrole-2-carboxylic acid ethyl ester (2 g, 5 mmol.) and sodium methoxide (0.27 g, 5 mmol.) in MeOH (60 mL) was treated with microwave irradiation at 60° C. for 5 mins. The solid that was formed was collected by filtration, washed with MeOH (20 mL) and air-dried to afford the title compound as a white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.13 min.

### D 3,5-Dibenzyl-2,4-dioxo-2,3,4,5-tetrahydro-1H-pyrrolo[3,2-d]pyrimidine-7-carbonitrile

A suspension of 3,6-dibenzyl-4-imino-2-oxo-2,3,4,6-tetrahydro-1H-pyrrolo[3,4-d]pyrimidine-7-carboxylic acid ethyl ester (1.13 g, 2.8 mmol.) and sodium methoxide (0.46 g, 8.4 mmol.) in MeOH (30 mL) was treated with microwave irradiation at 140° C. for 20 mins. The reaction mixture was concentrated in vacuo and the solid obtained was triturated with water (10 mL), filtered and dried under vacuum at 40° C. for 24 hours to afford the title compound as a white solid.

MS: 357 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.36 min.

### E 3,5-Dibenzyl-1-methyl-2,4-dioxo-2,3,4,5-tetrahydro-1H-pyrrolo[3,2-d]pyrimidine-7-carbonitrile

3,5-Dibenzyl-2,4-dioxo-2,3,4,5-tetrahydro-1H-pyrrolo[3,2-d]pyrimidine-7-carbonitrile (0.95 g, 2.7 mmol.) was dissolved in DMSO (10 mL). To this was added potassium carbonate (0.74 g, 5.3 mmol.) followed by methyl iodide (0.25 mL, 4.0 mmol.). The reaction mixture was stirred at room temperature for 3 hours. A dense white precipitate was formed and the reaction mixture was diluted with water (20 mL). The solid was collected by filtration, washed with water (10 mL) and dried under vacuum at 40° C. for 72 hours to afford the title compound as a white solid.

MS: no mass ion.

T_{R} [HPLC, Phenomenex Luna 3 micron C18; 5-95% CH₃CN+0.1% Formic acid/H₂O+0.1% Formic acid for 5 min, flow 2.0 ml/min]: 3.86 min.

### F 5-Benzyl-1-methyl-2,4-dioxo-2,3,4,5-tetrahydro-1H-pyrrolo[3,2-d]pyrimidine-7-carbonitrile

A mixture of 3,5-dibenzyl-1-methyl-2,4-dioxo-2,3,4,5-tetrahydro-1H-pyrrolo[3,2-d]pyrimidine-7-carbonitrile (0.90 g, 2.4 mmol.) and boron tribromide (12.16 mL, 12.2 mmol.) in xylene (50 mL) was stirred at 140° C. for 5 hours. The reaction mixture was cooled, MeOH (15 mL) was added and the mixture was stirred at room temperature for 30 mins. The solvents were evaporated in vacuo and the residue was partitioned between ethyl acetate (100 mL) and saturated aq. sodium hydrogen carbonate (200 mL). The ethyl acetate suspension was concentrated in vacuo and the residue was triturated with diethyl ether (100 mL), filtered and air-dried to afford the title compound as a beige solid.

MS: 281 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.74 min.

### G 5-Benzyl-6-bromo-1-methyl-2,4-dioxo-2,3,4,5-tetrahydro-1H-pyrrolo[3,2-d]pyrimidine-7-carbonitrile

5-Benzyl-1-methyl-2,4-dioxo-2,3,4,5-tetrahydro-1H-pyrrolo[3,2-d]pyrimidine-7-carbonitrile (0.37 g, 1.3 mmol.) was suspended in acetic acid (8 mL) and warmed to 45° C. To this was added bromine (0.10 mL, 2.0 mmol.) dropwise, in acetic acid (2 mL). Once the addition was complete, water (3 mL) was added and the reaction mixture was stirred at 45° C. for 18 hours. Another 1.5 equivalents of bromine, in 3 mL acetic acid was added and the reaction mixture was stirred at 45° C. for 4 hours. Another 2 equivalents of bromine and acetic acid (10 mL) were added and the reaction mixture was stirred at 70° C. for 72 hours. The solvents were removed in vacuo and the residue was triturated with saturated aqueous sodium thiosulphite solution (20 mL), followed by water (10 mL). The solid was collected by filtration and was dried in vacuo at 40° C. for 18 hours to obtain the title compound as a fawn coloured solid.

MS: 359 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.07 min.

### H 4-(5-Benzyl-7-cyano-1-methyl-2,4-dioxo-2,3,4,5-tetrahydro-1H-pyrrolo[3,2-d]pyrimidin-6-yl)-[1,4]diazepane-1-carboxylic Acid tert-butyl Ester

A mixture of 5-benzyl-6-bromo-1-methyl-2,4-dioxo-2,3,4,5-tetrahydro-1H-pyrrolo[3,2-d]pyrimidine-7-carbonitrile (500 mg, 1.39 mmol) and [1,4]diazepane-1-carboxylic acid tert-butyl ester (835 mg, 4.17 mmol) in DMA (10 mL) was heated at 160° C. using microwave irradiation for a total of 90 min. An additional amount of [1,4]diazepane-1-carboxylic acid tert-butyl ester (557 mg, 2.78 mmol) was added and the mixture was heated under microwave irradiation for a further 210 min at 160° C. The solvent was removed in vacuo and the residue was purified by flash chromatography (Silica, eluting with 20% ethyl acetate in DCM). The appropriate fractions were combined, concentrated and the residue was triturated with diethyl ether and dried to afford the title compound as an off-white solid.

MS: 479[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.53 min.

### I 6-(1,4-Diazepan-1-yl)-1,3-dimethyl-2,4-dioxo-5-(phenylmethyl)-2,3,4,5-tetrahydro-1H-pyrrolo[3,2-d]pyrimidine-7-carbonitrile Hydrochloride

A mixture of 4-(5-benzyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester (100 mg, 0.209 mmol), iodomethane (26.0 μL, 0.418 mmol) and potassium carbonate (86.5 mg, 0.627) in DMF (3 mL) was stirred at room temperature under nitrogen for 3 hours. The solvent was removed in vacuo and the resulting residue was triturated with water to give a cream coloured solid. The solid was treated with TFA (3 mL) and DCM (3 mL) for 1 hour at room temperature. The mixture was passed through an SCX-2 column (10 g, washing with MeOH and eluting with a mixture of 2M ammonia in MeOH/MeOH (1:4)). The relevant fractions were combined and concentrated in vacuo and the residue was re-purified by flash chromatography (Silica, gradient elution using DCM to 4% MeOH in DCM) to give the free base of the title compound. The free base was dissolved in MeOH (2 mL) and treated with hydrogen chloride (1.25 M in MeOH; 3 eq) and the solution was concentrated in vacuo and the residue was dried in vacuo at 45° C. for 18 hours to afford the title compound as a white solid.

MS: 393[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.88 min.

### Example S5

### 6-(1,4-Diazepan-1-yl)-1-methyl-3-(2-(3-(methyloxy)phenyl)-2-oxoethyl)-2,4-dioxo-5-(phenylmethyl)-2,3,4,5-tetrahydro-1H-pyrrolo[3,2-d]pyrimidine-7-carbonitrile Hydrochloride

This compound was prepared according to scheme 19.

The title compound was prepared analogously as described in Example S4 using 2-bromo-1-(3-methoxy-phenyl)-ethanone instead of iodomethane.

MS: 527[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.55 min.

### Example S6

### 6-(1,4-Diazepan-1-yl)-3-(isoquinolin-1-ylmethyl)-1-methyl-2,4-dioxo-5-(phenylmethyl)-2,3,4,5-tetrahydro-1H-pyrrolo[3,2-d]pyrimidine-7-carbonitrile Hydrochloride

This compound was prepared according to scheme 19.

The title compound was prepared analogously as described in Example S4 using 1-(bromomethyl)-isoquinoline hydrobromide instead of iodomethane.

MS: 520[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.65 min.

### Example T1

### 5-But-2-yn-1-yl-6-(1,4-diazepan-1-yl)-3-(isoquinolin-1-ylmethyl)-1-methyl-2,4-dioxo-2,3,4,5-tetrahydro-1H-pyrrolo[3,2-d]pyrimidine-7-carbonitrile Hydrochloride

This compound was prepared according to scheme 20.

### A 3-Amino-1-benzyl-4-cyano-1H-pyrrole-2-carboxylic Acid Ethyl Ester

A mixture of 2-(benzylamino-methylene)-malononitrile (10.94 g, 59.8 mmol), ethyl bromoacetate (9.94 mL, 89.7 mmol) and potassium carbonate (16.5 g, 119.6 mmol) were in DMF (200 mL) was heated at 90° C. for 50 mins. After cooling to 40° C., sodium ethoxide (77.7 mL of a 1M solution in ethanol) was added dropwise during 10 mins. The reaction mixture was heated to 90° C. for 25 mins. Glacial acetic acid (6.2 mL) was added and the reaction mixture was left to cool. The DMF was removed in vacuo and the residue was partitioned between ethyl acetate (200 mL) and water (200 mL). The layers were separated and the organic layer was washed with water (200 mL) and brine (200 mL), and dried (Na₂SO₄) Concentration gave a dark orange solid which was purified by flash chromatography (Silica, gradient elution with 10% ethyl acetate in cyclohexane to ethyl acetate). Fractions containing pure material were combined and concentrated to afford the title compound as a yellow solid.

MS: 270 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.30 min.

### B1-Benzyl-3-(3-benzyl-ureido)-4-cyano-1H-pyrrole-2-carboxylic Acid Ethyl Ester

A mixture of 3-amino-1-benzyl-4-cyano-1H-pyrrole-2-carboxylic acid ethyl ester (3.67 g, 13.6 mmol.) and benzyl isocyanate (2.53 mL, 20.5 mmol.) in pyridine (73 mL) was treated with microwave irradiation (Emrys Optimizer) at 120° C. for 30 mins. The reaction mixture was partitioned between ethyl acetate (100 mL) and 1M aq. hydrochloric acid (4×100 mL). The organic extract was dried (Na₂SO₄), filtered, concentrated in vacuo and the residue was purified by trituration with diethyl ether (50 mL), filtration and drying in a vacuum at 40° for 24 hours to afford the title compound as an off-white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### C 3,6-Dibenzyl-4-imino-2-oxo-2,3,4,6-tetrahydro-1H-pyrrolo[3,4-d]pyrimidine-7-carboxylic Acid Ethyl Ester

A mixture of 1-benzyl-3-(3-benzyl-ureido)-4-cyano-1H-pyrrole-2-carboxylic acid ethyl ester (2 g, 5 mmol.) and sodium methoxide (0.27 g, 5 mmol.) in MeOH (60 mL) was treated with microwave irradiation at 60° C. for 5 mins. The solid that was formed was collected by filtration, washed with MeOH (20 mL) and air-dried to afford the title compound as a white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.13 min.

### D 3,5-Dibenzyl-2,4-dioxo-2,3,4,5-tetrahydro-1H-pyrrolo[3,2-d]pyrimidine-7-carbonitrile

A suspension of 3,6-dibenzyl-4-imino-2-oxo-2,3,4,6-tetrahydro-1H-pyrrolo[3,4-d]pyrimidine-7-carboxylic acid ethyl ester (1.13 g, 2.8 mmol.) and sodium methoxide (0.46 g, 8.4 mmol.) in MeOH (30 mL) was treated with microwave irradiation at 140° C. for 20 mins. The reaction mixture was concentrated in vacuo and the solid obtained was triturated with water (10 mL), filtered and dried under vacuum at 40° C. for 24 hours to afford the title compound as a white solid.

MS: 357 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.36 min.

### E 3,5-Dibenzyl-1-methyl-2,4-dioxo-2,3,4,5-tetrahydro-1H-pyrrolo[3,2-d]pyrimidine-7-carbonitrile

3,5-Dibenzyl-2,4-dioxo-2,3,4,5-tetrahydro-1H-pyrrolo[3,2-d]pyrimidine-7-carbonitrile (0.95 g, 2.7 mmol.) was dissolved in DMSO (10 mL). To this was added potassium carbonate (0.74 g, 5.3 mmol.) followed by methyl iodide (0.25 mL, 4.0 mmol.). The reaction mixture was stirred at room temperature for 3 hours. A dense white precipitate was formed and the reaction mixture was diluted with water (20 mL). The solid was collected by filtration, washed with water (10 mL) and dried under vacuum at 40° C. for 72 hours to afford the title compound as a white solid.

MS: no mass ion.

T_{R} [HPLC, Phenomenex Luna 3 micron C18; 5-95% CH₃CN+0.1% Formic acid/H₂O+0.1% Formic acid for 5 min, flow 2.0 ml/min]: 3.86 min.

### F 5-Benzyl-1-methyl-2,4-dioxo-2,3,4,5-tetrahydro-1H-pyrrolo[3,2-d]pyrimidine-7-carbonitrile

A mixture of 3,5-dibenzyl-1-methyl-2,4-dioxo-2,3,4,5-tetrahydro-1H-pyrrolo[3,2-d]pyrimidine-7-carbonitrile (0.90 g, 2.4 mmol.) and boron tribromide (12.16 mL, 12.2 mmol.) in xylene (50 mL) was stirred at 140° C. for 5 hours. The reaction mixture was cooled, MeOH (15 mL) was added and the mixture was stirred at room temperature for 30 mins. The solvents were evaporated in vacuo and the residue was partitioned between ethyl acetate (100 mL) and saturated aq. sodium hydrogen carbonate (200 mL). The ethyl acetate suspension was concentrated in vacuo and the residue was triturated with diethyl ether (100 mL), filtered and air-dried to afford the title compound as a beige solid.

MS: 281 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.74 min.

### G 5-Benzyl-6-bromo-1-methyl-2,4-dioxo-2,3,4,5-tetrahydro-1H-pyrrolo[3,2-d]pyrimidine-7-carbonitrile

5-Benzyl-1-methyl-2,4-dioxo-2,3,4,5-tetrahydro-1H-pyrrolo[3,2-d]pyrimidine-7-carbonitrile (0.37 g, 1.3 mmol.) was suspended in acetic acid (8 mL) and warmed to 45° C. To this was added bromine (0.10 mL, 2.0 mmol.) dropwise, in acetic acid (2 mL). Once the addition was complete, water (3 mL) was added and the reaction mixture was stirred at 45° C. for 18 hours. Another 1.5 equivalents of bromine, in 3 mL acetic acid was added and the reaction mixture was stirred at 45° C. for 4 hours. Another 2 equivalents of bromine and acetic acid (10 mL) were added and the reaction mixture was stirred at 70° C. for 72 hours. The solvents were removed in vacuo and the residue was triturated with saturated aqueous sodium thiosulphite solution (20 mL), followed by water (10 mL). The solid was collected by filtration and was dried in vacuo at 40° C. for 18 hours to obtain the title compound as a fawn coloured solid.

MS: 359 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.07 min.

### H 4-(5-Benzyl-7-cyano-1-methyl-2,4-dioxo-2,3,4,5-tetrahydro-1H-pyrrolo[3,2-d]pyrimidin-6-yl)-[1,4]diazepane-1-carboxylic Acid tert-butyl Ester

A mixture of 5-benzyl-6-bromo-1-methyl-2,4-dioxo-2,3,4,5-tetrahydro-1H-pyrrolo[3,2-d]pyrimidine-7-carbonitrile (500 mg, 1.39 mmol) and [1,4]diazepane-1-carboxylic acid tert-butyl ester (835 mg, 4.17 mmol) in DMA (10 mL) was heated at 160° C. using microwave irradiation for a total of 90 min. An additional amount of [1,4]diazepane-1-carboxylic acid tert-butyl ester (557 mg, 2.78 mmol) was added and the mixture was heated under microwave irradiation for a further 210 min at 160° C. The solvent was removed in vacuo and the residue was purified by flash chromatography (Silica, eluting with 20% ethyl acetate in DCM). The appropriate fractions were combined, concentrated and the residue was triturated with diethyl ether and dried to afford the title compound as an off-white solid.

MS: 479[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.53 min.

### I 4-[5-Benzyl-7-cyano-1-methyl-2,4-dioxo-3-(2-trimethylsilanyl-ethoxymethyl)-2,3,4,5-tetrahydro-1H-pyrrolo[3,2-d]pyrimidin-6-yl]-[1,4]diazepane-1-carboxylic Acid tert-butyl Ester

Sodium hydride (74.0 mg, 1.85 mmol of a 60% dispersion in mineral oil) was added to a solution of 4-(5-benzyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester (590 mg, 1.23 mmol) in dry DMA (25 mL) under nitrogen at room temperature. The mixture was stirred at 50° C. for 1 hour. After cooling to room temperature, 2-(trimethylsilyl)ethoxymethyl chloride (327.4 μL, 1.85 mmol) was added and the resulting mixture was stirred at room temperature for 18 hours. The reaction was quenched by the addition of water (1 mL) and the resulting mixture was concentrated in vacuo. The residue was purified by flash chromatography (Silica, gradient elution with DCM to 10% ethyl acetate in DCM) to afford the title compound as a colourless viscous oil.

MS: 609[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.78 min.

### J 4-[7-Cyano-1-methyl-2,4-dioxo-3-(2-trimethylsilanyl-ethoxymethyl)-2,3,4,5-tetrahydro-1H-pyrrolo[3,2-d]pyrimidin-6-yl]-[1,4]diazepane-1-carboxylic Acid tert-butyl Ester

A mixture of 4-[5-benzyl-7-cyano-1-methyl-2,4-dioxo-3-(2-trimethylsilanyl-ethoxymethyl)-2,3,4,5-tetrahydro-1H-pyrrolo[3,2-d]pyrimidin-6-yl]-[1,4]diazepane-1-carboxylic acid tert-butyl ester (390 mg, 0.640 mmol), 10% palladium on charcoal (390 mg) and ammonium formate (403 mg, 6.40 mmol) in DMF (25 mL) was heated to 70° C. for 1 hour. The mixture was filtered through a pad of diatomaceous earth and the filter cake was washed with DMF (2×30 mL)). The combined filtrate and washings were concentrated in vacuo and the residue was triturated with water and dried to give the title compound as an off-white solid.

MS: 519[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.94 min.

### K 4-[5-But-2-ynyl-7-cyano-1-methyl-2,4-dioxo-3-(2-trimethylsilanyl-ethoxymethyl)-2,3,4,5-tetrahydro-1H-pyrrolo[3,2-d]pyrimidin-6-yl]-[1,4]diazepane-1-carboxylic Acid tert-butyl Ester

A mixture of 4-[7-cyano-1-methyl-2,4-dioxo-3-(2-trimethylsilanyl-ethoxymethyl)-2,3,4,5-tetrahydro-1H-pyrrolo[3,2-d]pyrimidin-6-yl]-[1,4]diazepane-1-carboxylic acid tert-butyl ester (275 mg, 0.53 mmol), 1-bromo-but-2-yne (186 μL, 2.12 mmol) and diisopropylethylamine (454 μL, 2.65 mmol) in dimethylformamide (6 mL) was stirred at room temperature for 18 hours under nitrogen. The solvent was removed in vacuo and the residue was purified by flash chromatography (Silica, gradient elution with DCM to 10% ethyl acetate in DCM) to afford the title compound as a colourless glass.

MS: 571[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.61 min.

### L 4-[5-But-2-ynyl-7-cyano-1-methyl-2,4-dioxo-2,3,4,5-tetrahydro-1H-pyrrolo[3,2-d]pyrimidin-6-yl]-[1,4]diazepane-1-carboxylic Acid tert-butyl Ester

4-[5-But-2-ynyl-7-cyano-1-methyl-2,4-dioxo-3-(2-trimethylsilanyl-ethoxymethyl)-2,3,4,5-tetrahydro-1H-pyrrolo[3,2-d]pyrimidin-6-yl]-[1,4]diazepane-1-carboxylic acid tert-butyl ester (132 mg, 0.231 mmol) was treated with TFA (4 mL) and DCM (2 mL) at room temperature for 2 hours. The mixture was concentrated in vacuo and the residue was purified by flash chromatography (SCX-2, washing with MeOH and eluting with 2M ammonia in MeOH/MeOH (1:15)). The relevant fractions were combined and concentrated. The residue was dissolved in DCM (2.5 mL) and treated with diisopropylethylamine (296 μL, 1.73 mmol) and di-tert-butyl carbonate at room temperature for 16 hours. The solvent was removed in vacuo and the residue was purified by flash chromatography (Silica, gradient elution with DCM to 20% ethyl acetate in DCM) to give the title compound as a pale yellow solid.

MS: 441[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.30 min.

### M 4-(5-But-2-ynyl-7-cyano-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic Acid tert-butyl Ester

1-(Bromomethyl)-isoquinoline hydrobromide (37.0 mg, 0.122 mmol) and potassium carbonate (42.2 mg, 0.306 mmol) were added to a solution of 4-[5-but-2-ynyl-7-cyano-1-methyl-2,4-dioxo-2,3,4,5-tetrahydro-1H-pyrrolo[3,2-d]pyrimidin-6-yl]-[1,4]diazepane-1-carboxylic acid tert-butyl ester (45 mg, 0.102 mmol) in DMF (1 mL) and the mixture was stirred for 18 hours at room temperature. The solvent was removed in vacuo and the residue was dissolved in DCM (20 mL) and was washed with water (20 mL), and brine (20 mL). The organic phase was dried (MgSO₄) and concentrated. The crude product was purified by flash chromatography (Silica, gradient elution with DCM to 10% ethyl acetate in DCM) to give the title compound as a pale yellow solid.

MS: 582[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.88 min.

### N 5-But-2-yn-1-yl-6-(1,4-diazepan-1-yl)-3-(isoquinolin-1-ylmethyl)-1-methyl-2,4-dioxo-2,3,4,5-tetrahydro-1H-pyrrolo[3,2-d]pyrimidine-7-carbonitrile Hydrochloride

4-(5-But-2-ynyl-7-cyano-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic acid tert-butyl ester (51 mg 0.088 mmol) was treated with TFA/DCM (1:1, 4 mL) at room temperature for 2 hours. The solvent was removed in vacuo and the residue was purified by flash chromatography (SCX-2, Washing with MeOH and eluting with 2M ammonia in MeOH/MeOH (1:10)). The free base of the title compound was dried in vacuo and was then converted to the hydrochloride salt by treatment with excess hydrogen chloride (1.25 M in MeOH). The solution was evaporated and the residue was dried in vacuo at 40° C. for 6 hours. The title compound was isolated as a yellow solid.

MS: 482[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.40 min.

### Example T2

### 6-((S)-3-Aminopiperidin-1-yl)-5-but-2-yn-1-yl-3-(isoquinolin-1-ylmethyl)-1-methyl-2,4-dioxo-2,3,4,5-tetrahydro-1H-pyrrolo[3,2-d]pyrimidine-7-carbonitrile Hydrochloride

This compound was prepared according to scheme 20.

The title compound was prepared analogously as described in Example T1 using (S)-piperidin-3-yl-carbamic acid tert-butyl ester instead of [1,4]diazepane-1-carboxylic acid tert-butyl ester.

MS: 482[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.33 min.

### Example T3

### 6-((S)-3-Aminopiperidin-1-yl)-5-but-2-yn-1-yl-1-methyl-3-(2-(3-(methyloxy)phenyl)-2-oxoethyl)-2,4-dioxo-2,3,4,5-tetrahydro-1H-pyrrolo[3,2-d]pyrimidine-7-carbonitrile Hydrochloride

This compound was prepared according to scheme 20.

The title compound was prepared analogously as described in Example T1 using (S)-piperidin-3-yl-carbamic acid tert-butyl ester instead of [1,4]diazepane-1-carboxylic acid tert-butyl ester and using 2-bromo-1-(3-methoxy-phenyl)-ethanone instead of 1-(bromomethyl)-isoquinoline hydrobromide.

MS: 489[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.33 min.

### Example U1

### 6-(1,4-Diazepan-1-yl)-3-(isoquinolin-1-ylmethyl)-1-methyl-5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

### A 4-(7-Cyano-1-methyl-2,4-dioxo-2,3,4,5-tetrahydro-1H-pyrrolo[3,2-d]pyrimidin-6-yl)-[1,4]diazepane-1-carboxylic Acid tert-butyl Ester

A mixture of 4-(5-benzyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester (430 mg, 0.898 mmol) [from example T1], 10% palladium on charcoal (107.5 mg) and ammonium formate (566 mg, 8.98 mmol) in DMF (25 mL) was heated to 75° C. for 1 hour. After cooling, more ammonium formate (283 mg, 4.99 mmol) was added and the mixture was heated for at 75° C. for a further 1 hour. The cooled mixture was filtered through a pad of diatomaceous earth and the filter cake was washed with DMF (2×50 mL)). The combined filtrate and washings were concentrated in vacuo and the residue was triturated with water and dried to give the title compound as an off-white solid.

MS: 389[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.64 min.

### B 4-(7-Cyano-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic Acid tert-butyl Ester

A mixture of 4-(7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester (168 mg, 0.433 mmol), 1-bromo-3-methyl-but-2-ene (100 μL, 0.866 mmol) and diisopropylethylamine (297 μL, 1.732 mmol) was stirred at 60° C. for 4 hours. The mixture was cooled and stirred at room temperature for 72 hours The mixture was heated to 60° C. for a further 18 hours. Another aliquot 1-bromo-3-methyl-but-2-ene (100 μL, 0.866 mmol) was added, and the mixture was heated to 60° C. for 24 hours The mixture was cooled to room temperature and diisopropylethylamine (4 eq) and 1-bromo-3-methyl-but-2-ene (2 eq) were added and the mixture heated for 4 hours. Heating was dicontinued and the mixture was concentrated in vacuo. The residue was purified by flash chromatography (Silica, gradient elution with DCM to 30% ethyl acetate in DCM) and the title compound was isolated as a brown solid.

MS: 457[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.60 min.

### C 4-(7-Cyano-3-isoquinolin-1-ylmethyl-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic Acid tert-butyl Ester

1-(Bromomethyl)-isoquinoline hydrobromide (31.8 mg, 0.105 mmol) followed by potassium carbonate (36.3 mg, 0.263 mmol) were added to a solution of 4-(7-cyano-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic acid tert-butyl ester (24 mg, 0.053 mmol) in DMF (1 mL) and the mixture was stirred for 24 hours. The solvent was removed in vacuo and the residue was purified by flash chromatography (Silica, gradient elution with DCM to 20% ethyl acetate in DCM) to give the title compound as a pale yellow foam.

MS: 598[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.23 min.

### D 6-(1,4-Diazepan-1-yl)-3-(isoquinolin-1-ylmethyl)-1-methyl-5-(3-methylbut-2-en-1-yl)-2,4-dioxo-2,3,4,5-tetrahydro-1H-pyrrolo[3,2-d]pyrimidine-7-carbonitrile

A mixture of 4-(7-cyano-3-isoquinolin-1-ylmethyl-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic acid tert-butyl ester (20.4 mg, 0.034 mmol), TFA (1 mL) and DCM (1 mL) was stirred at room temperature for 1 hour. The solvent was removed in vacuo and the residue was purified by flash chromatography (SCX-2, washing with MeOH and eluting with 2M ammonia in MeOH/MeOH (1:15)), then purified further (Silica, gradient elution with DCM to 5% MeOH in DCM) to afford the title compound as a gum.

MS: 498[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.70 min.

### Example U2

### 6-(1,4-Diazepan-1-yl)-1-methyl-5-(3-methylbut-2-en-1-yl)-3-(2-(3-(methyloxy)phenyl)-2-oxoethyl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using 2-bromo-1-(3-methoxy-phenyl)-ethanone instead of 1-(bromomethyl)-isoquinoline hydrobromide.

MS: 505[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.58 min.

### Example U3

### 6-((S)-3-Aminopiperidin-1-yl)-3-(isoquinolin-1-ylmethyl)-1-methyl -5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester.

MS: 498[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.10 min.

### Example U4

### 6-((S)-3-Aminopiperidin-1-yl)-1-methyl -5-(3-methylbut-2-en-1-yl)-3-(2-(3-(methyloxy)phenyl)-2-oxoethyl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-bromo-1-(3-methoxy-phenyl)-ethanone instead of 1-(bromomethyl)-isoquinoline hydrobromide.

MS: 505[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.87 min.

### Example U5

### 1-Methyl-5-(3-methyl but-2-en-1-yl)-3-(2-(3-(methyloxy)phenyl)-2-oxoethyl)-2,4-dioxo-6-piperazin-1-yl-2,3,4,5-tetrahydro-1H-pyrrolo[3,2-d]pyrimidine-7-carbonitrile

This compound was prepared according to scheme 21.

The title compound was prepared analogously as described in Example U1 using piperazine-1-carboxylic acid tert-butyl ester instead of [1,4]diazepane-1-carboxylic acid tert-butyl ester and using 2-bromo-1-(3-methoxy-phenyl)-ethanone instead of 1-(bromomethyl)-isoquinoline hydrobromide.

MS: 491[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.64 min.

### Example U6

### 6-((S)-3-Aminopiperidin-1-yl)-1-methyl-5-(3-methylbut-2-en-1-yl)-3-((4-methylquinazolin-2-yl)methyl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-(chloromethyl)-4-methyl-quinazoline instead of 1-(bromomethyl)-isoquinoline hydrobromide.

MS: 513[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.83 min.

### Example U7

### 6-((S)-3-Aminopiperidin-1-yl)-1-methyl-5-(3-methylbut-2-en-1-yl)-3-((4-methyl-3-oxidoquinazolin-2-yl)methyl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-(chloromethyl)-4-methyl-quinazoline-3-oxide instead of 1-(bromomethyl)-isoquinoline hydrobromide.

MS: 529[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.12 min.

### Example U8

### 6-((S)-3-Aminopiperidin-1-yl)-3-(cyanomethyl)-1-methyl -5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using iodoacetonitrile instead of 1-(bromomethyl)-isoquinoline hydrobromide.

MS: 396[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.63 min.

### Example U9

### 6-((S)-3-Aminopiperidin-1-yl)-3-ethyl-1-methyl -5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using iodoethane instead of 1-(bromomethyl)-isoquinoline hydrobromide.

MS: 385[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.59 min.

### Example U10

### 6-((S)-3-Aminopiperidin-1-yl)-3-((2-cyanophenyl)methyl)-1-methyl-5-(3-methyl but-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-bromomethyl-benzonitrile instead of 1-(bromomethyl)-isoquinoline hydrobromide.

MS: 472[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.80 min.

### Example U11

### 6-((S)-3-Aminopiperidin-1-yl)-1-methyl-5-(3-methylbut-2-en-1-yl)-2,4-dioxo-3-(pyridazin-3-ylmethyl)-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 3-(chloromethyl)-pyridazine instead of 1-(bromomethyl)-isoquinoline hydrobromide.

MS: 449[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.38 min.

### Example U12

### 6-((S)-3-Aminopiperidin-1-yl)-1-methyl-5-(3-methylbut-2-en-1-yl)-2,4-dioxo-3-(pyrimidin-4-ylmethyl)-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 4-(chloromethyl)-pyrimidine instead of 1-(bromomethyl)-isoquinoline hydrobromide.

MS: 449[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.32 min.

### Example U13

### 6-((S)-3-Aminopiperidin-1-yl)-1-methyl-5-(3-methylbut-2-en-1-yl)-2,4-dioxo-3-(pyridin-2-ylmethyl)-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-picolyl chloride hydrochloride instead of 1-(bromomethyl)-isoquinoline hydrobromide.

MS: 448[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.15 min.

### Example U14

### 6-((S)-3-Aminopiperidin-1-yl)-1-methyl-5-(3-methylbut-2-en-1-yl)-2,4-dioxo-3-(pyridin-4-ylmethyl)-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 4-picolyl chloride hydrochloride instead of 1-(bromomethyl)-isoquinoline hydrobromide.

MS: 448[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.34 min.

### Example U15

### 6-((S)-3-Aminopiperidin-1-yl)-3-(2-(ethyloxy)ethyl)-1-methyl-5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 1-bromo-2-ethoxy-ethane instead of 1-(bromomethyl)-isoquinoline hydrobromide.

MS: 428[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.01 min.

### Example U16

### 6-((S)-3-Aminopiperidin-1-yl)-1-methyl-5-(3-methylbut-2-en-1-yl)-3-([1,3]oxazolo[4,5-b]pyridin-2-ylmethyl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-chloromethyl-oxazolo[4,5-b]pyridine instead of 1-(bromomethyl)-isoquinoline hydrobromide.

MS: 489[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.80 min.

### Example U17

### 6-((S)-3-Aminopiperidin-1-yl)-1-methyl-5-(3-methylbut-2-en-1-yl)-3-((5-methyl isoxazol-3-yl)methyl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 3-chloromethyl-5-methyl-isoxazole instead of 1-(bromomethyl)-isoquinoline hydrobromide.

MS: 452[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.06 min.

### Example U18

### 6-((S)-3-Aminopiperidin-1-yl)-3-((3-cyanopyridin-2-yl)methyl)-1-methyl-5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-(chloromethyl)-nicotinonitrile instead of 1-(bromomethyl)-isoquinoline hydrobromide.

MS: 473[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.09 min.

### Example U19

### 6-((S)-3-Aminopiperidin-1-yl)-1-methyl-5-(3-methylbut-2-en-1-yl)-2,4-dioxo-3-(quinoxalin-2-ylmethyl)-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-(chloromethyl)-quinoxaline instead of 1-(bromomethyl)-isoquinoline hydrobromide.

MS: 499[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.95 min.

### Example U20

### 6-((S)-3-Aminopiperidin-1-yl)-1-methyl-5-(3-methylbut-2-en-1-yl)-2,4-dioxo-3-(pyrazin-2-ylmethyl)-2,3,4,5-tetrahydro-1H-pyrrolo[3,2-d]pyrimidine-7-carbonitrile

This compound was prepared according to scheme 21.

The title compound was prepared analogously as described in Example U1 using (S)-piperidin-3-yl-carbamic acid tert-butyl ester instead of [1,4]diazepane-1-carboxylic acid tert-butyl ester and using 2-(chloromethyl)-pyrazine instead of 1-(bromomethyl)-isoquinoline hydrobromide.

MS: 449[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.43 min.

### Example U21

### 6-(1,4-Diazepan-1-yl)-1-methyl-5-(3-methylbut-2-en-1-yl)-3-[(2-oxidoisoquinolin-1-yl)methyl]-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using 1-(bromomethyl)-isoquinoline-2-oxide instead of 1-(bromomethyl)-isoquinoline hydrobromide.

MS: 514[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.86 min.

### Example U22

### 4-{[7-Cyano-6-(1,4-diazepan-1-yl)-1-methyl -5-(3-methylbut-2-en-1-yl)-2,4-dioxo-1,2,4,5-tetrahydro-3H-pyrrolo[3,2-d]pyrimidin-3-yl]methyl}quinoline-3-carbonitrile

This compound was prepared according to scheme 21.

The title compound was prepared analogously as described in Example U1 using 4-(chloromethyl)-quinoline-3-carbonitrile instead of 1-(bromomethyl)-isoquinoline hydrobromide.

MS: 523[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.18 min.

### Example U23

### 6-(1,4-Diazepan-1-yl)-1-methyl-5-(3-methylbut-2-en-1-yl)-3-[(4-methylquinazolin-2-yl)methyl]-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using 2-(chloromethyl)-4-methyl-quinazoline instead of 1-(bromomethyl)-isoquinoline hydrobromide.

MS: 513[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.38 min.

### Example U24

### 6-(1,4-Diazepan-1-yl)-1-methyl-5-(3-methylbut-2-en-1-yl)-3-[(4-methyl-3-oxidoquinazolin-2-yl)methyl]-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using 2-(chloromethyl)-4-methyl-quinazoline-3-oxide instead of 1-(bromomethyl)-isoquinoline hydrobromide.

MS: 529[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.12 min.

### Example U25

### 6-(1,4-Diazepan-1-yl)-3-(imidazo[1,2-a]pyridin-2-ylmethyl)-1-methyl-5-(3-methylbut-2-en-1-yl)-2,4-dioxo-2,3,4,5-tetrahydro-1H-pyrrolo[3,2-d]pyrimidine-7-carbonitrile

This compound was prepared according to scheme 21.

The title compound was prepared analogously as described in Example U1 using 2-(chloromethyl)-imidazo[1,2-a]pyridine hydrochloride instead of 1-(bromomethyl)-isoquinoline hydrobromide.

MS: 487[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.74 min.

### Example U26

### Methyl 2-{[7-cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}nicotinate

This compound was prepared according to scheme 21.

### A 4-[7-Cyano-3-(3-methoxycarbonyl-pyridin-2-ylmethyl)-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic Acid tert-butyl Ester

Potassium carbonate (90 mg, 0.652 mmol) then 2-(chloromethyl)-nicotinic acid methyl ester (61 mg, 0.329 mmol) were added to a solution of 4-(7-cyano-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic acid tert-butyl ester [from Example U1] (150 mg, 0.329 mmol) in dimethylformamide (5 mL) and the mixture was stirred at room temperature overnight. A further quantity of 2-(chloromethyl)-nicotinic acid methyl ester (30.5 mg, 0.165 mmol) was added and the mixture was stirred at 60° C. for 24 hours. The mixture was evaporated and the residue was partitioned between water and ethyl acetate. The organic layer was then evaporated and purified by flash chromatography (silica, eluting with 2% methanol in dichloromethane) to give the title compound as a foam.

MS: 606[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.23 min.

### B Methyl 2-{[7-cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}nicotinate

A solution of 4-[7-cyano-3-(3-methoxycarbonyl-pyridin-2-ylmethyl)-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic acid tert-butyl ester (20 mg, 0.033 mmol) in dichloromethane (0.5 mL) was treated with trifluoroacetic acid (0.5 mL) and the mixture was aged for 1 hour. The mixture was concentrated and the residue was partitioned between saturated aqueous sodium bicarbonate and ethyl acetate. The organic layer was concentrated and the residue was purified by flash chromatography (silica, eluting with 10% methanol in dichloromethane) to afford the title compound as a yellow solid.

MS: 506[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.68 min.

### Example U27

### 2-{[7-Cyano-6-(1,4-diazepan-1-yl)-1-methyl -5-(3-methylbut-2-en-1-yl)-2,4-dioxo-1,2,4,5-tetrahydro-3H-pyrrolo[3,2-d]pyrimidin-3-yl]methyl}-N-ethylnicotinamide

### A 4-[3-(3-Carboxy-pyridin-2-ylmethyl)-7-cyano-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic Acid tert-butyl Ester

A solution of methyl 2-{[7-cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}nicotinate (105 mg, 0.173 mmol) in dioxane (5 mL) was treated with 1M aqueous lithium hydroxide (0.5 mL) and the mixture was heated at 60° C. with stirring for 3 hours. The mixture was concentrated and the residue was partitioned between ethyl acetate and 1M aqueous ammonium chloride. The organic layer was then washed with water and evaporated to give the title compound as an off-white solid.

MS: 592 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.74 min.

### B 4-[7-Cyano-3-(3-ethylcarbamoyl-pyridin-2-ylmethyl)-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic Acid tert-butyl Ester

A solution of 4-[3-(3-carboxy-pyridin-2-ylmethyl)-7-cyano-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic acid tert-butyl ester (50 mg, 0.085 mmol) in dimethylformamide (10 mL) was treated sequentially with diisopropylethylamine (55 mg, 0.426 mmol), O-(7-azabenzotriazol-1-yl)-N,N,N′,N′-tetramethyluronium hexafluorophosphate (39 mg, 0.102 mmol) and a 2M solution of ethylamine in tetrahydrofuran (84 μL, 0.168 mmol) and the mixture was stirred at room temperature for 1 hour. The mixture was concentrated and the residue was partitioned between water and ethyl acetate. The organic layer was then evaporated and the residue was purified by flash chromatography (silica, eluting with 5% methanol in dichloromethane). Fractions containing the main UV spot were combined and evaporated to give the title compound as a gum.

MS: 619 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.78 min.

### C 2-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}-N-ethylnicotinamide

A solution of 4-[7-cyano-3-(3-ethylcarbamoyl-pyridin-2-ylmethyl)-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic acid tert-butyl ester (30 mg, 0.049 mmol) in dichloromethane (1 mL) was treated with trifluoroacetic acid (1 mL) and the mixture was stirred at room temperature for 1 hour. The mixture was concentrated and the residue was partitioned between saturated aqueous sodium bicarbonate and ethyl acetate. The organic layer was concentrated and the residue was purified by flash chromatography (silica, eluting with 10% methanol in dichloromethane) to afford the title compound as a yellow gum.

MS: 519[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.51 min.

### Example U28

### 2-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}nicotinamide

The title compound was prepared analogously as described in Example U27 using ammonium chloride instead of ethylamine.

MS: 491[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.16 min.

### Example U29

### 3-{[7-Cyano-6-(1,4-diazepan-1-yl)-1-methyl-5-(3-methylbut-2-en-1-yl)-2,4-dioxo-1,2,4,5-tetrahydro-3H-pyrrolo[3,2-d]pyrimidin-3-yl]methyl}isoquinoline-4-carbonitrile

This compound was prepared according to scheme 21.

The title compound was prepared analogously as described in Example U1 using 3-bromomethyl-isoquinoline-4-carbonitrile instead of 1-(bromomethyl)-isoquinoline hydrobromide.

MS: 523[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 7.30 min.

### Example U30

### 4-{[5-But-2-yn-1-yl-7-cyano-6-(1,4-diazepan-1-yl)-1-methyl-2,4-dioxo-1,2,4,5-tetrahydro-3H-pyrrolo[3,2-d]pyrimidin-3-yl]methyl}quinoline-3-carbonitrile

This compound was prepared according to scheme 21.

### A 4-(5-But-2-ynyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester

A mixture of 4-(7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester (500 mg, 1.287 mmol) [from example U1], 1-bromo-but-2-yne (233 μL, 2.57 mmol) and diisopropylethylamine (0.741 mL, 5.14 mmol) was stirred at RT for 48 hours. The mixture was concentrated in vacuo. The residue was purified by flash chromatography (Silica, gradient elution with 5% MeOH/DCM) and the title compound was isolated as a brown foam.

MS: 441 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.26 min.

### B 4-{[5-But-2-yn-1-yl-7-cyano-6-(1,4-diazepan-1-yl)-1-methyl-2,4-dioxo-1,2,4,5-tetrahydro-3H-pyrrolo[3,2-d]pyrimidin-3-yl]methyl}quinoline-3-carbonitrile

The title compound was prepared from 4-(5-but-2-ynyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester analogously as described in Example U1 using 4-(chloromethyl)-quinoline-3-carbonitrile instead of 1-(bromomethyl)-isoquinoline hydrobromide.

MS: 507[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.53 min.

### Example U31

### 3-{[5-But-2-yn-1-yl-7-cyano-6-(1,4-diazepan-1-yl)-1-methyl-2,4-dioxo-1,2,4,5-tetrahydro-3H-pyrrolo[3,2-d]pyrimidin-3-yl]methyl}isoquinoline-4-carbonitrile

This compound was prepared according to scheme 21.

The title compound was prepared from 4-(5-but-2-ynyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester [from example U30] analogously as described in Example U1 using 3-bromomethyl-isoquinoline-4-carbonitrile instead of 1-(bromomethyl)-isoquinoline hydrobromide.

MS: 507[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.62 min.

### Example V1

### 5-Benzyl-6-(1,4-diazepan-1-yl)-3-(isoquinolin-1-ylmethyl)-1-methyl-2,4-dioxo-2,3,4,5-tetrahydro-1H-pyrrolo[3,2-d]pyrimidine-7-carboxamide Hydrochloride

This compound was prepared according to scheme 22.

### A 1-Benzyl-3-(3-benzyl-ureido)-4-cyano-1H-pyrrole-2-carboxylic Acid Ethyl Ester

A mixture of 3-amino-1-benzyl-4-cyano-1H-pyrrole-2-carboxylic acid ethyl ester (3.67 g, 13.6 mmol.) [From Example S1] and benzyl isocyanate (2.53 mL, 20.5 mmol.) in pyridine (73 mL) was treated with microwave irradiation at 120° C. for 30 mins. The reaction mixture was partitioned between ethyl acetate (100 mL) and 1M aq. hydrochloric acid (4×100 mL). The organic extract was dried (Na₂SO₄), filtered, concentrated in vacuo and the residue was purified by trituration with diethyl ether (50 mL), filtration and drying in a vacuum at 40° for 24 hours to afford the title compound as an off-white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.45 min.

### B 3,6-Dibenzyl-4-imino-2-oxo-2,3,4,6-tetrahydro-1H-pyrrolo[3,4-d]pyrimidine-7-carboxylic Acid Ethyl Ester

A mixture of 1-benzyl-3-(3-benzyl-ureido)-4-cyano-1H-pyrrole-2-carboxylic acid ethyl ester (2 g, 5 mmol.) and sodium methoxide (0.27 g, 5 mmol.) in MeOH (60 mL) was treated with microwave irradiation (Emrys Optimizer) at 60° C. for 5 mins. The solid that was formed was collected by filtration, washed with MeOH (20 mL) and air-dried to afford the title compound as a white solid.

MS: 403 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.13 min.

### C 3,5-Dibenzyl-2,4-dioxo-2,3,4,5-tetrahydro-1H-pyrrolo[3,2-d]pyrimidine-7-carbonitrile

A suspension of 3,6-dibenzyl-4-imino-2-oxo-2,3,4,6-tetrahydro-1H-pyrrolo[3,4-d]pyrimidine-7-carboxylic acid ethyl ester (1.13 g, 2.8 mmol.) and sodium methoxide (0.46 g, 8.4 mmol.) in MeOH (30 mL) was treated with microwave irradiation (Emrys Optimizer) at 140° C. for 20 mins. The reaction mixture was concentrated in vacuo and the solid obtained was triturated with water (10 mL), filtered and dried under vacuum at 40° C. for 24 hours to afford the title compound as a white solid.

MS: 357 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.36 min.

### D 3,5-Dibenzyl-1-methyl-2,4-dioxo-2,3,4,5-tetrahydro-1H-pyrrolo[3,2-d]pyrimidine-7-carbonitrile

3,5-Dibenzyl-2,4-dioxo-2,3,4,5-tetrahydro-1H-pyrrolo[3,2-d]pyrimidine-7-carbonitrile (0.95 g, 2.7 mmol.) was dissolved in DMSO (10 mL). To this was added potassium carbonate (0.74 g, 5.3 mmol.) followed by methyl iodide (0.25 mL, 4.0 mmol.). The reaction mixture was stirred at room temperature for 3 hours. A dense white precipitate was formed and the reaction mixture was diluted with water (20 mL). The solid was collected by filtration, washed with water (10 mL) and dried under vacuum at 40° C. for 72 hours to afford the title compound as a white solid.

MS: no mass ion.

T_{R} [HPLC, Phenomenex Luna 3 micron C18; 5-95% CH₃CN+0.1% Formic acid/H₂O+0.1% Formic acid for 5 min, flow 2.0 ml/min]: 3.86 min.

### E 5-Benzyl-1-methyl-2,4-dioxo-2,3,4,5-tetrahydro-1H-pyrrolo[3,2-d]pyrimidine-7-carbonitrile

A mixture of 3,5-dibenzyl-1-methyl-2,4-dioxo-2,3,4,5-tetrahydro-1H-pyrrolo[3,2-d]pyrimidine-7-carbonitrile (0.90 g, 2.4 mmol.) and boron tribromide (12.16 mL, 12.2 mmol.) in xylene (50 mL) was stirred at 140° C. for 5 hours. The reaction mixture was cooled, MeOH (15 mL) was added and the mixture was stirred at room temperature for 30 mins. The solvents were evaporated in vacuo and the residue was partitioned between ethyl acetate (100 mL) and saturated aq. sodium hydrogen carbonate (200 mL). The ethyl acetate suspension was concentrated in vacuo and the residue was triturated with diethyl ether (100 mL), filtered and air-dried to afford the title compound as a beige solid.

MS: 281 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.74 min.

### F 5-Benzyl-6-bromo-1-methyl-2,4-dioxo-2,3,4,5-tetrahydro-1H-pyrrolo[3,2-d]pyrimidine-7-carbonitrile

5-Benzyl-1-methyl-2,4-dioxo-2,3,4,5-tetrahydro-1H-pyrrolo[3,2-d]pyrimidine-7-carbonitrile (0.37 g, 1.3 mmol.) was suspended in acetic acid (8 mL) and warmed to 45° C. To this was added bromine (0.10 mL, 2.0 mmol.) dropwise, in acetic acid (2 mL). Once the addition was complete, water (3 mL) was added and the reaction mixture was stirred at 45° C. for 18 hours. Another 1.5 equivalents of bromine, in 3 mL acetic acid was added and the reaction mixture was stirred at 45° C. for 4 hours. Another 2 equivalents of bromine and 10 mL acetic acid were added and the reaction mixture was stirred at 70° C. for 72 hours. The solvents were removed in vacuo and the residue was triturated with saturated aq. sodium thiosulphite solution (20 mL), followed by water (10 mL). The solid was collected by filtration and was dried under vacuum at 40° C. for 18 hours to obtain the title compound as a fawn coloured solid.

MS: 359 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.07 min.

### G 4-(5-Benzyl-7-cyano-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic Acid tert-butyl Ester

5-Benzyl-6-bromo-1-methyl-2,4-dioxo-2,3,4,5-tetrahydro-1H-pyrrolo[3,2-d]pyrimidine-7-carbonitrile (1.5 g, 4.18 mmol) in DMF (20 mL) was treated with potassium carbonate (1.73 g, 12.53 mmol) and 1-(bromomethyl)-isoquinoline hydrobromide (1.265 g, 4.18 mmol) and was stirred at room temperature for 18 hours. The solvent was removed in vacuo and the residue was triturated with water to give a solid. This solid was treated with [1,4]diazepane (4.19 g, 41.8 mmol) in DMA (15 mL) and the mixture was heated by microwave irradiation at 160° C. for 15 min. The solvent was removed in vacuo and the residue was triturated to afford a beige coloured solid. The solid was taken dissolved in 1,4-dioxane (10 mL) and di-tert-butyl dicarbonate (1.14 g, 5.22 mmol) followed by aqueous 1M sodium hydroxide solution (10 mL) were added and the suspension was stirred for 18 h at room temperature. Water (50 mL) and DCM (50 mL) was added and the mixture was separated. The aqueous layer was extracted with DCM (2×50 mL) and the organic phases were combined and washed with brine (50 mL), then dried (MgSO₄). Evaporation of the solvent gave the title compound as a pale yellow solid.

MS: 620 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.02 min.

### H 4-(5-Benzyl-7-carbamoyl-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic Acid tert-butyl Ester

Potassium carbonate (22.3 mg, 0.161 mmol) followed by hydrogen peroxide (80.5 μL of 35% in water) were added to a stirred solution of 4-(5-benzyl-7-cyano-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic acid tert-butyl ester in a mixture of DMSO (1.2 mL) and water (0.12 mL). The resulting mixture was stirred at room temperature for 18 hours Another aliquot of hydrogen peroxide (80.5 μL of 35% in water) was added and the mixture was stirred for 100 hours. Water (20 mL) was added and the solution was filtered to give a white solid. Purification by flash chromatography (Silica, with ethyl acetate as eluent) gave the title compound as a white solid.

MS: 638 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.15 min.

### I 5-Benzyl-6-(1,4-diazepan-1-yl)-3-(isoquinolin-1-ylmethyl)-1-methyl-2,4-dioxo-2,3,4,5-tetrahydro-1H-pyrrolo[3,2-d]pyrimidine-7-carboxamide Hydrochloride

4-(5-Benzyl-7-carbamoyl-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic acid tert-butyl ester (50 mg, 0.0784 mmol) was treated with TFA (2 mL) in DCM (2 mL) at room temperature for 30 min. The solvent was removed in vacuo and the residue was purified by flash chromatography [(SCX-2, washing with MeOH before eluting with a mixture of 2M ammonia in MeOH in MeOH (1:10)) and then (Silica, gradient elution with DCM to 20% MeOH in DCM)]. The free base of the title compound was isolated and this was converted to the hydrochloride salt by treatment with hydrogen chloride (1.25 M in MeOH). The title compound was isolated as a yellow solid.

MS: 538[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.52 min.

### Example V2

### 5-Benzyl-6-(1,4-diazepan-1-yl)-3-(isoquinolin-1-ylmethyl)-N,N,1-trimethyl-2,4-dioxo-2,3,4,5-tetrahydro-1H-pyrrolo[3,2-d]pyrimidine-7-carboxamide Hydrochloride

This compound was prepared according to scheme 22.

### A 4-(5-Benzyl-7-carbamoyl-3-isoquinolin-1-ylmethyl-N,N,1-trimethyl-2,4-dioxo-2,3,4,5-tetrahydro-1H-pyrrolo[3,2-d]pyrimidin-6-yl)-[1,4]diazepane-1-carboxylic Acid tert-butyl Ester

Sodium hydride (10.8 mg of a 60% dispersion in mineral oil, 0.27 mmol) was added to a solution of 4-(5-benzyl-7-carbamoyl-3-isoquinolin-1-ylmethyl-1-methyl-2,4-dioxo-2,3,4,5-tetrahydro-1H-pyrrolo[3,2-d]pyrimidin-6-yl)-[1,4]diazepane-1-carboxylic acid tert-butyl ester (43 mg, 0.0674 mmol) [From Example V1] in DMF (1 mL) and the mixture was stirred at room temperature for 30 min. Iodomethane (16.8 μL, 0.27 mmol) was added and the mixture was stirred at room temperature for 18 hours. The solvent was removed in vacuo and the residue was purified by flash chromatography (Si, ethyl acetate as eluent) to give the title compound as a beige coloured solid.

MS: 666 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.33 min.

### B 5-Benzyl-6-(1,4-diazepan-1-yl)-3-(isoquinolin-1-ylmethyl)-N,N,1-trimethyl-2,4-dioxo-2,3,4,5-tetrahydro-1H-pyrrolo[3,2-d]pyrimidine-7-carboxamide Hydrochloride

4-(5-Benzyl-7-carbamoyl-3-isoquinolin-1-ylmethyl-N,N,1-trimethyl-2,4-dioxo-2,3,4,5-tetrahydro-1H-pyrrolo[3,2-d]pyrimidin-6-yl)-[1,4]diazepane-1-carboxylic acid tert-butyl ester (4 mg, 0.0676 mmol) was treated with TFA (2 mL) in DCM (2 mL) at room temperature for 30 min. The solvent was removed in vacuo and the residue was purified by flash chromatography [(SCX-2, washing with MeOH before eluting with a mixture of 2M ammonia in MeOH in MeOH (1:10)) and then (Silica, gradient elution with DCM to 20% MeOH in DCM)]. The resulting product was further purified by flash chromatography (Silica, gradient elution with DCM to 20% MeOH in DCM) and the appropriate fractions were combined and concentrated to give the free base of the title compound as a colourless glass. The free base was treated with hydrogen chloride (1.25 M in MeOH). After evaporation of the volatiles and drying in vacuo at 40° C. for 72 hours, the title compound was obtained as a yellow solid.

MS: 566[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.77 min.

### Example V3

### 5-Benzyl-6-(1,4-diazepan-1-yl)-3-(isoquinolin-1-ylmethyl)-1-methyl-7-(morpholin-4-ylcarbonyl)-1H-pyrrolo[3,2-d]pyrimidine-2,4(3H,5H)-dione

This compound was prepared according to scheme 22.

### A 4-[5-Benzyl-3-isoquinolin-1-ylmethyl-1-methyl-7-(morpholine-4-carbonyl)-2,4-dioxo-2,3,4,5-tetrahydro-1H-pyrrolo[3,2-d]pyrimidin-6-yl]-[1,4]diazepane-1-carboxylic Acid tert-butyl Ester

Sodium hydride (9.4 mg, 0.235 mmol of a 60% dispersion in mineral oil) was added to a solution of 4-(5-benzyl-7-carbamoyl-3-isoquinolin-1-ylmethyl-N,N,1-trimethyl-2,4-dioxo-2,3,4,5-tetrahydro-1H-pyrrolo[3,2-d]pyrimidin-6-yl)-[1,4]diazepane-1-carboxylic acid tert-butyl ester (50 mg, 0.0784 mmol) in dimethylformamide (5 mL)_and the mixture was stirred for 10 min. 2,2-Dichlorodiethylether (11.2 mg, 0.0784 mmol) was then added and the mixture was stirred at room temperature for 1 hour and then at 50° C. for 22 hours. Additional quantities of sodium hydride (28.2 mg, 0.705 mmol of a 60% dispersion in mineral oil) and 2,2-dichlorodiethylether (11.2 mg, 0.0784 mmol) were added and heating was continued for 21 hours. The volatiles were removed in vacuo and the residue was triturated with water to give the title compound as a yellow solid.

MS: 708 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.38 min.

### B 5-Benzyl-6-(1,4-diazepan-1-yl)-3-(isoquinolin-1-ylmethyl)-1-methyl-7-(morpholin-4-ylcarbonyl)-1H-pyrrolo[3,2-d]pyrimidine-2,4(3H,5H)-dione

4-[5-Benzyl-3-isoquinolin-1-ylmethyl-1-methyl-7-(morpholine-4-carbonyl)-2,4-dioxo-2,3,4,5-tetrahydro-1H-pyrrolo[3,2-d]pyrimidin-6-yl]-[1,4]diazepane-1-carboxylic acid tert-butyl ester (40.4 mg, 0.0571 mmol) was dissolved in a mixture of dichloromethane (1 mL) and trifluoroacetic acid (1 mL) and allowed to react for 30 min. The solvents were removed in vacuo and the residue was purified by flash chromatography [(SCX-2 column, washing with methanol before eluting with a mixture of 2M ammonia in methanol/methanol (1:10)) and then (Silica column, gradient elution with dichloromethane to 10% methanol in dichloromethane)] to give the title compound as a gum.

MS: 608 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.90 min.

### Example V4

### 5-Benzyl-6-(1,4-diazepan-1-yl)-1,3-dimethyl-2,4-dioxo-2,3,4,5-tetrahydro-1H-pyrrolo[3,2-d]pyrimidine-7-carboxamide Hydrochloride

This compound was prepared according to scheme 22.

The title compound was prepared analogously as described in Example V1 using iodomethane instead of 1-(bromomethyl)-isoquinoline hydrobromide.

MS: 411[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 3.85 min.

### Example V5

### 5-Benzyl-6-(1,4-diazepan-1-yl)-N,N,1,3-tetramethyl-2,4-dioxo-2,3,4,5-tetrahydro-1H-pyrrolo[3,2-d]pyrimidine-7-carboxamide Hydrochloride

This compound was prepared according to scheme 22.

The title compound was prepared analogously as described in Example V2 from 4-(5-benzyl-7-carbamoyl-1,3-dimethyl-2,4-dioxo-2,3,4,5-tetrahydro-1H-pyrrolo[3,2-d]pyrimidin-6-yl)-[1,4]diazepane-1-carboxylic acid tert-butyl ester which is prepared analogously as described in Example V1 and using iodomethane instead of 1-(bromomethyl)-isoquinoline hydrobromide.

MS: 439[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.17 min.

### Example W1

### 6-(1,4-Diazepan-1-yl)-3-(isoquinolin-1-ylmethyl)-N,N,1-trimethyl-5-(3-methylbut-2-en-1-yl)-2,4-dioxo-2,3,4,5-tetrahydro-1H-pyrrolo[3,2-d]pyrimidine-7-carboxamide

This compound was prepared according to scheme 23.

A 4-(7-Cyano-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic Acid tert-butyl Ester

A mixture of 4-(7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester (168 mg, 0.433 mmol), 1-bromo-3-methyl-but-2-ene (100 μL, 0.866 mmol) and diisopropylethylamine (297 μL, 1.732 mmol) was stirred at 60° C. for 4 hours. The mixture was cooled and stirred at room temperature for 72 hours The mixture was heated to 60° C. for a further 18 hours. Another aliquot 1-bromo-3-methyl-but-2-ene (100 μL, 0.866 mmol) was added, and the mixture was heated to 60° C. for 24 hours The mixture was cooled to room temperature and diisopropylethylamine (4 eq) and 1-bromo-3-methyl-but-2-ene (2 eq) were added and the mixture heated for 4 hours. Heating was dicontinued and the mixture was concentrated in vacuo. The residue was purified by flash chromatography (Silica, gradient elution with DCM to 30% ethyl acetate in DCM) and the title compound was isolated as a brown solid.

MS: 457[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.60 min.

### B 4-(7-Cyano-3-isoquinolin-1-ylmethyl-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic Acid tert-butyl Ester

1-(Bromomethyl)-isoquinoline hydrobromide (31.8 mg, 0.105 mmol) followed by potassium carbonate (36.3 mg, 0.263 mmol) were added to a solution of 4-(7-cyano-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic acid tert-butyl ester (24 mg, 0.053 mmol) in DMF (1 mL) and the mixture was stirred for 24 hours The solvent was removed in vacuo and the residue was purified by flash chromatography (Silica, gradient elution with DCM to 20% ethyl acetate in DCM) to give the title compound as a pale yellow foam.

MS: 598[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.23 min.

### C 4-[7-Carbamoyl-3-isoquinolin-1-ylmethyl-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic Acid tert-butyl Ester

Potassium carbonate (273 mg, 1.98 mmol) followed by hydrogen peroxide (0.56 mL of a 27.5 wt. % in water) were added to a solution of 4-(7-cyano-3-isoquinolin-1-ylmethyl-1-methyl-2,4-dioxo-5-(3-methyl-but-2-enyl)-2,3,4,5-tetrahydro-1H-pyrrolo[3,2-d]pyrimidin-6-yl)-[1,4]diazepane-1-carboxylic acid tert-butyl ester (590 mg, 0.988 mmol) in a mixture of dimethyl sulphoxide (15 mL) and water (0.75 mL). The mixture was stirred at room temperature for 18 hours. Water (20 mL) was added and the precipitate was collected and dried at 70° C. under high vacuum for 2 hours to afford the title compound as a cream coloured solid.

MS: 616[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.22 min.

### D 4-[7-Dimethylcarbamoyl-3-isoquinolin-1-ylmethyl-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic Acid tert-butyl Ester

A solution of 4-[7-carbamoyl-3-isoquinolin-1-ylmethyl-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic acid tert-butyl ester (250 mg, 0.407 mmol) in dimethylformamide (8.5 mL) was treated with sodium hydride (65 mg, 1.63 mmol of a 60% mineral oil dispersion) and stirred at room temperature for 20 mins. Iodomethane (101 μL, 1.63 mmol) was added and stirring was continued for a further 30 minutes. The reaction mixture was triturated with water (25 mL) and the precipitate was collected by vacuum filtration. The pad was washed with water (20 mL) and the solid was dried in vacuo at 70° C. for 2 hours to give the title compound as a cream coloured solid.

MS: 644[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.37 min.

### E 6-(1,4-Diazepan-1-yl)-3-(isoquinolin-1-ylmethyl)-N,N,1-trimethyl-5-(3-methylbut-2-en-1-yl)-2,4-dioxo-2,3,4,5-tetrahydro-1H-pyrrolo[3,2-d]pyrimidine-7-carboxamide

A solution of the foregoing 4-[7-dimethylcarbamoyl-3-isoquinolin-1-ylmethyl-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic acid tert-butyl ester in dichloromethane (18 mL) was treated drop-wise with trifluoroacetic acid (2 mL) and was allowed to stir at room temperature for 30 minutes. With rapid stirring the reaction mixture was treated dropwise with saturated sodium bicarbonate (aq) until carbon dioxide evolution ceased. The organic phase was collected and the aqueous layer extracted with dichloromethane. The combined organic phases was washed successively with water (10 mL) and brine (10 mL), then dried (Na₂SO₄), filtered and concentrated in vacuo. The residue was purified by flash chromatography (silica, eluting with 5% methanol in dichloromethane containing 0.1% triethylamine) to give the title product as a cream coloured foam.

MS: 544[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.75 min.

### Example W2

### 6-(1,4-Diazepan-1-yl)-3-(isoquinolin-1-ylmethyl)-1-methyl-5-(3-methylbut-2-en-1-yl)-2,4-dioxo-2,3,4,5-tetrahydro-1H-pyrrolo[3,2-d]pyrimidine-7-carboxamide

This compound was prepared according to scheme 23.

A solution of 4-[7-carbamoyl-3-isoquinolin-1-ylmethyl-1-methyl-5-(3-methyl-but-2-enyl)-2,4-dioxo-2,3,4,5-tetrahydro-1H-pyrrolo[3,2-d]pyrimidin-6-yl]-[1,4]diazepane-1-carboxylic acid tert-butyl ester (50 mg, 0.0813 mmol) in dichloromethane (9 mL) was treated dropwise with trifluoroacetic acid (1 mL) and the mixture was stirred at room temperature for 30 minutes. With rapid stirring the reaction mixture was treated dropwise with saturated sodium bicarbonate (aq) until carbon dioxide evolution ceased. The organic phase was collected and the aqueous layer extracted with dichloromethane. The combined organic phases was washed successively with water (10 mL) and brine (10 mL), then dried (Na₂SO₄), filtered and concentrated in vacuo. The crude product was semi-purified by flash chromatography (silica, eluting with 10% methanol in dichloromethane containing 0.1% triethylamine) final purification by Reversed Phase HPLC (gradient elution from 10% to 30% acetonitrile/water (containing 0.1% formic acid). Appropriate fractions were combined and basified with solid potassium carbonate to pH12. The mixture was extracted with dichloromethane, dried (Na₂SO₄), filtered and concentrated in vacuo. The residue was dissolved in methanol and passed through an SCX-2 cartridge (2 g), eluting with 2M ammonia in methanol to give the title product as a cream coloured foam.

MS: 516[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.54 min.

### Example W3

### 6-(1,4-Diazepan-1-yl)-3-(isoquinolin-1-ylmethyl)-1-methyl -5-(3-methylbut-2-en-1-yl)-7-(morpholin-4-ylcarbonyl)-1H-pyrrolo[3,2-d]pyrimidine-2,4(3H,5H)-dione

This compound was prepared according to scheme 23.

The title compound was prepared analogously as described in Example W1 using 1-chloro-2-(2-chloroethoxy)-ethane instead of iodomethane.

MS: 586 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.98 min.

### Example W4

### 6-(1,4-Diazepan-1-yl)-3-(isoquinolin-1-ylmethyl)-1-methyl -5-(3-methylbut-2-en-1-yl)-7-(piperidin-1-ylcarbonyl)-1H-pyrrolo[3,2-d]pyrimidine-2,4(3H,5H)-dione

This compound was prepared according to scheme 23.

The title compound was prepared analogously as described in Example W1 using 1,5-dibromopentane instead of iodomethane.

MS: 584 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.37 min.

### Example W5

### 5-But-2-yn-1-yl-6-(1,4-diazepan-1-yl)-3-(isoquinolin-1-ylmethyl)-N,N,1-trimethyl-2,4-dioxo-2,3,4,5-tetrahydro-1H-pyrrolo[3,2-d]pyrimidine-7-carboxamide

This compound was prepared according to scheme 23.

The title compound was prepared analogously as described in Example W1 using 1-bromo-but-2-yne instead of 1-bromo-3-methyl-but-2-ene.

MS: 528 [M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 5.13 min.

### Example XI

### 6-(1,4-Diazepan-1-yl)-3-(isoquinolin-1-ylmethyl)-5-(3-methylbut-2-en-1-yl)-3,5-dihydro-4H-pyrrolo[3,2-d]pyrimidin-4-one

The title compound was prepared according to Scheme 24.

### A 6-Chloro-4-oxo-5-(2-trimethylsilanyl-ethoxymethyl)-4,5-dihydro-3H-pyrrolo[3,2-d]pyrimidine-7-carboxylic Acid Methyl Ester

Tetrabutylammonium fluoride (210 mL of a 1M solution in tetrahydrofuran) was added to a suspension of 6-chloro-4-oxo-3,5-bis-(2-trimethylsilanyl-ethoxymethyl)-4,5-dihydro-3H-pyrrolo[3,2-d]pyrimidine-7-carboxylic acid methyl ester [Example G1] (9.8 g, 20.0 mmol) and the mixture was heated at 60° C. for 2 hours. The mixture was evaporated and the residue was triturated with water. The solid was collected, washed well with water and dried to afford the title compound as a white solid.

MS: 358[M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.30 min.

### B 6-Chloro-3-isoquinolin-1-ylmethyl-4-oxo-5-(2-trimethylsilanyl-ethoxymethyl)-4,5-dihydro-3H-pyrrolo[3,2-d]pyrimidine-7-carboxylic Acid Methyl Ester

A mixture of 6-chloro-4-oxo-5-(2-trimethylsilanyl-ethoxymethyl)-4,5-dihydro-3H-pyrrolo[3,2-d]pyrimidine-7-carboxylic acid methyl ester (2.61 g, 7.29 mmol), 2-(bromomethyl)-isoquinoline hydrobromide (2.21, 7.29 mmol) and potassium carbonate (2.02 g, 14.62 mmol) in dimethylformamide (30 mL) was stirred at ambient temperature for 3 hours. The volatiles were removed in vacuo and the residue was dissolved in dichloromethane. The dichloromethane solution was washed with water and brine, dried (MgSO4) and evaporated to dryness to afford the title compound as a gum which was used directly in the next step.

MS: 499/501 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.10 min.

### C 6-Chloro-3-isoquinolin-1-ylmethyl-4-oxo-4,5-dihydro-3H-pyrrolo[3,2-d]pyrimidine-7-carboxylic Acid Methyl Ester

The product from step C above was dissolved in a mixture of dichloromethane (100 mL) and added trifluoroacetic acid (50 mL) and the mixture was stirred at ambient temperature for 3 hours. The volatiles were evaporated and the residue was purified by flash chromatography (Silica, gradient elution with 2% methanol in dichloromethane to 5% methanol in dichloromethane) to give the title compound as a tan coloured solid.

MS: 368/370 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.51 min.

### D 6-Chloro-3-isoquinolin-1-ylmethyl-5-(3-methyl-but-2-enyl)-4-oxo-4,5-dihydro-3H-pyrrolo[3,2-d]pyrimidine-7-carboxylic Acid Methyl Ester

Diisopropylethylamine (0.52 mL, 2.98 mmol) and 4-bromo-2-methyl-2-butene (0.18 mL 1.56 mmol) were added dropwise to a solution of 6-chloro-3-isoquinolin-1-ylmethyl-4-oxo-4,5-dihydro-3H-pyrrolo[3,2-d]pyrimidine-7-carboxylic acid methyl ester (0.55 g, 1.49 mmol) in dimethylformamide (10 mL) and the mixture was stirred at ambient temperature for 18 hours. The volatiles were removed and the residue was triturated with water. The solids were collected and dried to afford the title compound as a tan coloured solid.

MS: 437 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.33 min.

### E 6-(4-tert-Butoxycarbonyl-[1,4]diazepan-1-yl)-3-isoquinolin-1-ylmethyl-5-(3-methyl-but-2-enyl)-4-oxo-4,5-dihydro-3H-pyrrolo[3,2-d]pyrimidine-7-carboxylic Acid Methyl Ester

A mixture of 6-chloro-3-isoquinolin-1-ylmethyl-5-(3-methyl-but-2-enyl)-4-oxo-4,5-dihydro-3H-pyrrolo[3,2-d]pyrimidine-7-carboxylic acid methyl ester (0.51 g, 1.17 mmol) and [1,4]diazepane-1-carboxylic acid tert-butyl ester (1.2 mL, 6.09 mmol) in dimethylacetamide (6 mL) was irradiated with microwaves at 160° C. for 4 hour. A further quantity of [1,4]diazepane-1-carboxylic acid tert-butyl ester (0.5 mL, 2.54 mmol) was added and the mixture was heated at 120° C. for 65 hours. The reaction mixture was concentrated and added the residue was dissolved in dichloromethane. The dichloromethane solution was washed with aq. acetic acid (20% v/v), saturated aqueous sodium bicarbonate and brine, then dried (MgSO₄) and evaporated to dryness. The residue was purified by flash chromatography (Silica, gradient elution from neat dichloromethane to 2% methanol in dichloromethane) to give the title compound as a brown gel.

MS: 601 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.64 min.

### F A Mixture of 4-[3-Isoquinolin-1-ylmethyl-5-(3-methyl-but-2-enyl)-4-oxo-4,5-dihydro-3H-pyrrolo[3,2-d]pyrimidin-6-yl]-[1,4]diazepane-1-carboxylic Acid tert-butyl Ester and 6-(4-tert-Butoxycarbonyl-[1,4]diazepan-1-yl)-3-isoquinolin-1-ylmethyl-5-(3-methyl-but-2-enyl)-4-oxo-4,5-dihydro-3H-pyrrolo[3,2-d]pyrimidine-7-carboxylic Acid

Aqueous lithium hydroxide (10 mL of a 0.5M solution) was added to a solution of 6-(4-tert-butoxycarbonyl-[1,4]diazepan-1-yl)-3-isoquinolin-1-ylmethyl-5-(3-methyl-but-2-enyl)-4-oxo-4,5-dihydro-3H-pyrrolo[3,2-d]pyrimidine-7-carboxylic acid methyl ester (0.55 g, 0.916 mmol) in dioxane and the mixture was heated at 60° C. for 3 hours. The reaction mixture was concentrated to low volume, neutralised with ammonium chloride and extracted with ethyl acetate. The organic phase was washed with brine, dried (MgSO4) and evaporated to dryness to give a 2:1 mixture of 4-[3-isoquinolin-1-ylmethyl-5-(3-methyl-but-2-enyl)-4-oxo-4,5-dihydro-3H-pyrrolo[3,2-d]pyrimidin-6-yl]-[1,4]diazepane-1-carboxylic acid tert-butyl ester

MS: 543 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.61 min. and 6-(4-tert-butoxycarbonyl-[1,4]diazepan-1-yl)-3-isoquinolin-1-ylmethyl-5-(3-methyl-but-2-enyl)-4-oxo-4,5-dihydro-3H-pyrrolo[3,2-d]pyrimidine-7-carboxylic acid

MS: 587 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.78 min.

### G 6-(1,4-Diazepan-1-yl)-3-(isoquinolin-1-ylmethyl)-5-(3-methylbut-2-en-1-yl)-3,5-dihydro-4H-pyrrolo[3,2-d]pyrimidin-4-one

Trifluoroacetic acid (0.5 mL) was added to a solution of 4-[3-isoquinolin-1-ylmethyl-5-(3-methyl-but-2-enyl)-4-oxo-4,5-dihydro-3H-pyrrolo[3,2-d]pyrimidin-6-yl]-[1,4]diazepane-1-carboxylic acid tert-butyl ester (23 mg, 0.042 mmol) in dichloromethane (1 mL) and the mixture was stirred at ambient temperature for 2 hours. The reaction mixture was purified by loading onto an ion exchange column (SCX-2, and washing with dichloromethane then methanol, and eluting with a 2M solution of ammonia in methanol). Final purification was achieved using reversed phase HPLC (10-80% methanol/water (containing 0.1% TFA)) to afford the title compound as an oil.

MS: 443[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.39 min.

### Example Y1

### 5-But-2-yn-1-yl-6-(1,4-diazepan-1-yl)-N,N,1,3-tetramethyl-2,4-dioxo-2,3,4,5-tetrahydro-1H-pyrrolo[3,2-d]pyrimidine-7-carboxamide

The title compound was prepared according to Scheme 25.

### A 4-(5-Benzyl-7-carbamoyl-1,3-dimethyl-2,4-dioxo-2,3,4,5-tetrahydro-1H-pyrrolo[3,2-d]pyrimidin-6-yl)-[1,4]diazepane-1-carboxylic Acid tert-butyl Ester

Potassium carbonate (392 mg, 2.83 mmol) and then hydrogen peroxide (1.4 mL of 27.5 wt %) were added to a solution of 4-(5-benzyl-7-cyano-1-methyl-2,4-dioxo-2,3,4,5-tetrahydro-1H-pyrrolo[3,2-d]pyrimidin-6-yl)-[1,4]diazepane-1-carboxylic acid tert-butyl ester [Example S4] (541 mg, 1.10 mmol) in a mixture of dimethyl sulphoxide (10 mL) and water (1 mL). The mixture was stirred for 30 min then allowed to stand at ambient temperature for 120 hours. Further quantities of dimethyl sulphoxide (10 mL), potassium carbonate (392 mg, 2.83 mmol) and then hydrogen peroxide (1.4 mL of 27.5 wt %) were added and the mixture was stirred for 18 hours._Water (50 mL) was added and the mixture was extracted with chloroform (2×50 mL). The combined extracts were washed with water (100 mL), dried (MgSO₄) and concentrated in vacuo. The residue was triturated with water to afford the title compound as a white solid.

MS: 511 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.14 min.

### B 4-(5-Benzyl-7-dimethylcarbamoyl-1,3-dimethyl-2,4-dioxo-2,3,4,5-tetrahydro-1H-pyrrolo[3,2-d]pyrimidin-6-yl)-[1,4]diazepane-1-carboxylic Acid tert-butyl Ester

Sodium hydride (116 mg, 2.9 mmol of a 60% dispersion in oil) was added to a solution of 4-(5-benzyl-7-carbamoyl-1,3-dimethyl-2,4-dioxo-2,3,4,5-tetrahydro-1H-pyrrolo[3,2-d]pyrimidin-6-yl)-[1,4]diazepane-1-carboxylic acid tert-butyl ester (370 mg, 0.725 mmol) in dimethylformamide (10 mL) and the stirred mixture was aged for 30 min. Iodomethane (181 μL, 2.9 mmol) was then added and the mixture was stirred at room temperature for 18 hours. Water (2 mL) was added and the solvents were removed in vacuo. The residue was tritureated with water and extracted with dichloromethane (2×50 mL). The combined organic phases were dried (MgSO₄) and concentrated to afford a gum. The gum was purified by flash chromatography (Silica, gradient elution with 10% dichloromethane in ethyl acetate to ethyl acetate) to give the title compound as a pale yellow foam.

MS: 539 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.39 min.

### C 4-(7-Dimethylcarbamoyl-1,3-dimethyl-2,4-dioxo-2,3,4,5-tetrahydro-1H-pyrrolo[3,2-d]pyrimidin-6-yl)-[1,4]diazepane-1-carboxylic Acid tert-butyl Ester

10% Palladium on charcoal (133 mg) was added to a solution of 4-(5-benzyl-7-dimethylcarbamoyl-1,3-dimethyl-2,4-dioxo-2,3,4,5-tetrahydro-1H-pyrrolo[3,2-d]pyrimidin-6-yl)-[1,4]diazepane-1-carboxylic acid tert-butyl ester (260 mg, 0.483 mmol) under a nitrogen atmosphere. Ammonium formate (457 mg, 7.25 mmol) was added in one portion and the mixture was stirred at 75° C. for 19 h. After cooling to room temperature, additional ammonium formate (457 mg, 7.25 mmol) was added and the mixture was heated to 75° C. for 2 hours. A further quantity of ammonium formate (914 mg, 14.5 mmol) was added and the mixture was heated to 75° C. for 18 hours. After cooling, the mixture was filtered through a pad of diatomaceous earth and the filter cake was washed with dimethylformamide (2×50 mL). The filtrate was concentrated in vacuo and the residue was purified by flash chromatography (Silica, eluting with dichloromethane to 5% methanol in dichloromethane) to afford the title compound as a beige solid.

MS: 449 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.52 min.

### D 4-(5-But-2-ynyl-7-dimethylcarbamoyl-1,3-dimethyl-2,4-dioxo-2,3,4,5-tetrahydro-1H-pyrrolo[3,2-d]pyrimidin-6-yl)-[1,4]diazepane-1-carboxylic Acid tert-butyl Ester

Sodium hydride (12 mg, 0.299 mmol of a 60% dispersion in oil) was added to a solution of 4-(7-dimethylcarbamoyl-1,3-dimethyl-2,4-dioxo-2,3,4,5-tetrahydro-1H-pyrrolo[3,2-d]pyrimidin-6-yl)-[1,4]diazepane-1-carboxylic acid tert-butyl ester (67 mg, 0.149 mmol) in dimethylformamide (3 mL) and the mixture was aged at room temperature for 30 min. 1-Bromo-2-butyne (26.2 μL, 0.299 mmol) was added and the mixture was stirred at room temperature for 20 hours. The solvent was removed in vacuo and the residue was purified by flash chromatography (Silica, gradient elution with dichloromethane to 10% methanol in dichloromethane) and then by reversed phase HPLC (5% to 95% acetonitrile in water containing 0.1% formic acid) to give the title compound as a pale yellow foam.

MS: 501 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.14 min.

### E 5-But-2-yn-1-yl-6-(1,4-diazepan-1-yl)-N,N,1,3-tetramethyl-2,4-dioxo-2,3,4,5-tetrahydro-1H-pyrrolo[3,2-d]pyrimidine-7-carboxamide

A solution of 4-(5-but-2-ynyl-7-dimethylcarbamoyl-1,3-dimethyl-2,4-dioxo-2,3,4,5-tetrahydro-1H-pyrrolo[3,2-d]pyrimidin-6-yl)-[1,4]diazepane-1-carboxylic acid tert-butyl ester (12.3 mg, 0.0245 mmol) in a mixture of dichloromethane/trifluoroacetic acid 9/1 (3 mL) was aged at room temperature for 30 min. The mixture was added to saturated aqueous sodium bicarbonate (20 mL) and extracted with dichloromethane (20 mL). The extract was dried (Na₂SO₄) and concentrated in vacuo. The residue was purified by flash chromatography (Silica, eluting with ethyl acetate and then 10% methanol in dichloromethane) to give the title compound as a gum.

MS: 401[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 4.47 min.

### Example Z1

### 6-[(3S)-3-Aminopiperidin-1-yl]-5-benzyl -1,3-dimethyl-7-(methylthio)-1H-pyrrolo[3,2-d]pyrimidine-2,4(3H,5H)-dione

The title compound was prepared according to Scheme 26.

### A [(S)-1-(1,3-Dimethyl-2,4-dioxo-2,3,4,5-tetrahydro-1H-pyrrolo[3,2-d]pyrimidin-6-yl)-piperidin-3-yl]-carbamic Acid tert-butyl Ester

A suspension of [(S)-1-(5-benzyl-7-cyano-1,3-dimethyl-2,4-dioxo-2,3,4,5-tetrahydro-1H-pyrrolo[3,2-d]pyrimidin-6-yl)-piperidin-3-yl]-carbamic acid tert-butyl ester [Example R1] (2.85 g, 5.79 mmol) in concentrated sulphuric acid (20 mL) was heated at 100° C. for 30 min. The mixture was poured on to crushed ice (250 g) and, with ice bath cooling, the mixture was carefully adjusted to pH 12 with 20M aqueous potassium hydroxide. The mixture was then adjusted to pH 7-8 with concentrated hydrochloric acid. The solids were collected and the filter pad was washed with water and methanol. The washings were concentrated to remove most of the methanol and the residual aqueous phase was extracted with a mixture of chloroform and 2-propanol 3:1 (3×300 mL). The extracts were dried (Na₂SO₄) and concentrated in vacuo. The residue (1.21 g) was dissolved in a mixture of 1M aqueous sodium hydroxide and dioxane 1:1 (20 mL) and was treated with a solution of di-tert-butyl-dicarbonate (1.43 g, 6.54 mmol) in dioxane. After stirring at room temperature for 2 hours, the mixture was diluted with water (50 mL) and the solids were collected washed with water (50 mL) and a mixture of water and dioxane 1:1 (50 mL). The residue was dried at 60° C. in vacuo for 20 hours to give the title compound as a white solid.

MS: 378 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 2.85 min.

### B [(S)-1-(1,3-Dimethyl-2,4-dioxo-7-thiocyanato-2,3,4,5-tetrahydro-1H-pyrrolo[3,2-d]pyrimidin-6-yl)-piperidin-3-yl]-carbamic Acid tert-butyl Ester

A suspension of [(S)-1-(1,3-dimethyl-2,4-dioxo-2,3,4,5-tetrahydro-1H-pyrrolo[3,2-d]pyrimidin-6-yl)-piperidin-3-yl]-carbamic acid tert-butyl ester (1.19 g, 3.16 mmol) in warm (60° C.) dimethylformamide (85 mL) was treated with a solution of ammonium thiocyanate (720 mg, 9.46 mmol) and iodine (802 mg, 3.16 mmol) in methanol (10 mL) and the mixture was stirred at 60° C. for 1 hour. The reaction mixture was concentrated in vacuo and the residue partitioned between dichloromethane (2×50 mL) and water (50 mL). The combined organic extracts was washed with aqueous 15 wt % sodium thiosulphate and brine (50 mL). The organic extract was dried (Na₂SO₄), filtered, and concentrated in vacuo to give the title compound as a beige coloured solid.

MS: 435 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.17 min.

### C [(S)-1-(1,3-Dimethyl-7-methylsulfanyl-2,4-dioxo-2,3,4,5-tetrahydro-1H-pyrrolo[3,2-d]pyrimidin-6-yl)-piperidin-3-yl]-carbamic Acid tert-butyl Ester

A stirred solution of [(S)-1-(1,3-dimethyl-2,4-dioxo-7-thiocyanato-2,3,4,5-tetrahydro-1H-pyrrolo[3,2-d]pyrimidin-6-yl)-piperidin-3-yl]-carbamic acid tert-butyl ester (1.23 g, 2.83 mmol) in methanol (100 mL) was treated with sodium borohydride (322 mg, 8.49 mmol) followed by the addition of iodomethane (1.76 mL, 2.83 mmol). The mixture was stirred for 10 min and then acetone (10 mL) was added. After a further 10 minutes, the reaction was concentrated in vacuo and the residue was partitioned between dichloromethane (2×50 mL) and water (50 mL). The combined organic extracts was washed with brine (50 mL), dried (Na₂SO₄), filtered and concentrated in vacuo to give the crude product as a beige coloured solid. The crude product was purified by flash chromatography (silica, eluting initially with dichloromethane, and then 1% methanol in dichloromethane) to afford the title compound as a white solid.

MS: 424 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 3.35 min.

### D [(S)-1-(5-Benzyl-1,3-dimethyl-7-methylsulfanyl-2,4-dioxo-2,3,4,5-tetrahydro-1H-pyrrolo[3,2-d]pyrimidin-6-yl)-piperidin-3-yl]-carbamic Acid tert-butyl Ester

A mixture of [(S)-1-(1,3-dimethyl-7-methylsulfanyl-2,4-dioxo-2,3,4,5-tetrahydro-1H-pyrrolo[3,2-d]pyrimidin-6-yl)-piperidin-3-yl]-carbamic acid tert-butyl ester (200 mg, 0.472 mmol) and diisopropylethylamine (0.21 mL, 1.18 mmol) in dimethylformamide (5 mL) was treated with benzyl bromide (67 μL, 0.567 mmol) and the mixture was stirred at room temperature for 18 hours. The solution was warmed to 60° C. for 30 min. A further amount of benzyl bromide (56 μL) and diisopropylethylamine (85 μL) were added and heating was continued for 18 hours at 60° C. An additional 3 eq of benzyl bromide (168 μL) and diisopropylethylamine (255 μL) were added and heating continued for 18 hours at 60° C. The reaction was concentrated in vacuo and the residue purified by flash chromatography (silica, eluting with 10% ethyl acetate in petrol) to give the desired product as a cream foam.

MS: 514 [M+H]⁺

T_{R} [HPLC, Phenomenex Luna 3 micron C18; 5-95% CH₃CN+0.1% Formic acid/H₂O+0.1% Formic acid for 5 min, flow 2.0 ml/min]: 4.24 min.

### E 6-[(3S)-3-Aminopiperidin-1-yl]-5-benzyl-1,3-dimethyl-7-(methylthio)-1H-pyrrolo[3,2-d]pyrimidine-2,4(3H,5H)-dione

A solution of [(S)-1-(5-benzyl-1,3-dimethyl-7-methylsulfanyl-2,4-dioxo-2,3,4,5-tetrahydro-1H-pyrrolo[3,2-d]pyrimidin-6-yl)-piperidin-3-yl]-carbamic acid tert-butyl ester (25 mg, 0.049 mmol) in dichloromethane (2 mL) was treated with trifluoroacetic acid (1 mL) and the mixture was stirred at room temperature for 20 min. The reaction mixture was concentrated and toluene added to chase the last traces of trifluoroacetic acid. The crude product was purified by ion exchange chromatography (SCX-2 cartridge, eluting with 2M ammonia in methanol) to afford the title compound as a clear oil.

MS: 414[M+H]⁺

T_{R} [HPLC, Higgins Clipeus 5 micron C18; 5-95% CH₃CN+0.1% Formic acid/H₂O+0.1% Formic acid for 20 min, flow 2.0 ml/min]: 6.59 min.

### Example AA

### Activity Assay

The compounds of Examples A1, A2, B1 and B2 were tested for their inhibitory activity to human DPP-IV.

### Materials

Human DPP-IV consisting of amino acids 39 to 766 followed by a C-terminal Streptavidin-tag was expressed using the baculovirus system and purified to >80% purity. The enzyme was stored in 25 mM Tris buffer, pH 9.0, containing 300 mM NaCl at −80° C. The fluorogenic substrates H-Gly-Pro-AMC was purchased from Bachem AG (Bubendorf, Switzerland). The substrate was kept as a 5 mM stock solution in DMSO at −20° C. All other chemicals were purchased from Sigma (Buchs, Switzerland).

The assay buffer for the DPP-IV reaction was 25 mM Tris/HCl, pH 7.5, containing 140 mM NaCl, 10 mM KCl and 0.05% (w/v) CHAPS.

### Compound and Liquid Handling

The test compounds were dissolved in 90% DMSO/10% H2O (v/v). Serial dilutions of the compounds from 3 mM to 0.03 μM in 90% DMSO/10% H2O (v/v) followed by a 1:33.3 dilution in assay buffer was done in 96-well polypropylene plates using a CyBio Dilus 8-channel pipettor (CyBio AG, Jena, Germany) with tip change after each pipetting step. The compound solutions as well as the substrate and the enzyme solutions were transferred to the assay plates (384-well black Cliniplate; cat. no. 95040020 Labsystems Oy, Finland) by means of a CyBi-Well 96-channel pipettor (CyBio AG, Jena, Germany).

### Kinetic Measurements

Enzyme kinetics were measured by mixing 10 μl of a 3-fold concentrated substrate solution in assay buffer (final substrate concentration was 10 μM) with 10 μl of the corresponding compound solution. The reactions were initiated by addition of 10 μl of a 3-fold concentrated solution of the enzyme in assay buffer. Final enzyme (active site) concentrations in the assay was 10 pM for DPP-IV. Fluorescence product (AMC) formation was monitored for 1 hour at room temperature at 35 second intervals by measuring the fluorescence emission at 500 nm using an exitation wavelength of 350 nm in a TECAN Ultra fluorescence reader (TECAN, Maennedorf, Switzerland). The fluorescence in each well was excited by one flash per measurement. The Origin software package (Origin 7.5 Mircocal, Northampton, Mass., USA) was used to generate all graphs and to perform the IC50 calculations.

### Results

The inhibitory activities (IC₅₀ values) of the compounds to human DPP-IV were found to be less than 50 μm and in many cases less than 0.1 μm. Particular compounds were found to have IC₅₀ values of 50 nm or less, e.g. 10 nm or less.

1. A compound of formula (I): 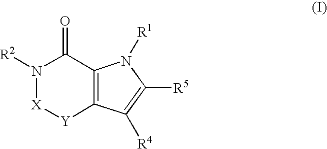
wherein
X is —CH═ and Y is ═N—; or X is —C(O)— and Y is —N(R³)—;
R¹, R² and R³ are independently each hydrogen, —W-hydrocarbyl or —W-heterocyclyl, any of which is optionally substituted, particularly on the hydrocarbyl or heterocyclyl part, with 1, 2, 3, 4 or 5 R¹²; wherein the or each W is independently a bond or a linker having from 1 to 8 in-chain atoms and selected from, for example, —CH₂—, —O—, —C(O)—, —S(O)ₘ—, —NR^{a}—, cyclopropylene; C₁, C₂, C₃, C₄, C₅ or C₆ alkyl; and chemically appropriate combinations thereof; and wherein the or each R^{a} is independently hydrogen, hydroxy or hydrocarbyl optionally interrupted by: an —O— or —NH— linkage;
R⁴ is hydrogen, or an electron withdrawing group, for example —CF₃, —CN, —C(O)OR⁸, —C(O)NR⁸R⁹, —S(O)ₘR⁸ or —CH₂OR¹⁰;
R⁵ is a group of formula (i): 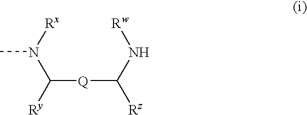
wherein
Q is a bond or alkylene comprising 1, 2 or 3 in-chain carbon atoms optionally substituted with 1, 2, 3, 4 or 5 R¹²; and
R^{w}, R^{x}, R^{y} and R^{z} are each independently hydrogen or C₁₋₆ alkyl optionally substituted with 1, 2, 3, 4 or 5 R¹²;
or two of R^{w}, R^{x}, R^{y} and R^{z} taken together form an alkylene bridge comprising 1, 2, 3, 4, 5 or 6 in-chain carbon atoms, the bridge optionally substituted with 1, 2, 3, 4 or 5 R¹²; and the other two are each hydrogen or C₁₋₆ alkyl optionally substituted with 1, 2, 3, 4 or 5 R¹²;
R⁸ and R⁹ are independently each hydrogen or C₁₋₆ alkyl optionally substituted with 1, 2, 3, 4 or 5 R¹²; or R⁸ and R⁹ taken together with the nitrogen atom to which they are attached form heterocyclyl optionally substituted with 1, 2, 3, 4 or 5 R¹²;
R¹⁰ is C₁₋₆ alkyl, C₂₋₆ alkenyl or C₂₋₆ alkynyl, any of which is optionally substituted with 1, 2, 3, 4 or 5 substituents selected from R¹¹ and R¹²;
R¹¹ is aryl or heteroaryl, either of which is optionally substituted with 1, 2, 3, 4 or 5 R¹²;
each R¹² is independently selected from:
(i) functional moieties selected from hydroxy, halogen, amino and —CN;
(ii) alkyl having 1, 2, 3, 4, 5 or 6 carbon atoms and optionally substituted with 1, 2, 3, 4 or 5 halogens;
(iii) alkyl having 1, 2, 3, 4, 5 or 6 carbon atoms and optionally substituted with one or two of said functional moieties (i);
(iv) alkoxy having 1, 2, 3, 4, 5 or 6 carbon atoms and optionally substituted with 1, 2, 3, 4 or 5 halogens; and
(v) alkoxy having 1, 2, 3, 4, 5 or 6 carbon atoms and optionally substituted with one or two of said functional moieties (i);
or two R¹² attached to the same carbon atom form oxo; and
m is 0, 1 or 2;
or a pharmaceutically acceptable salt or prodrug thereof.

2. The compound according to claim 1, wherein Q is a bond, R⁵ is a group of formula (ii):

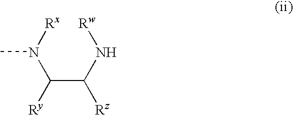

3. The compound according to claim 1, wherein Q is methylene optionally substituted with 1 or 2 R¹²; or ethylene optionally substituted with 1, 2, 3 or 4 R¹².

4. The compound according to claim 1, wherein:
R^{w} and R^{x} form —CH₂—, —(CH₂)₂— or —(CH₂)₃—; and R^{y} and R^{z} are each hydrogen;
R^{x} and R^{z} form —CH₂—, —(CH₂)₂— or —(CH₂)₃—; and R^{w} and R^{z} are each hydrogen; or
R^{y} and R^{z} form —(CH₂)₃—, —(CH₂)₄— or —(CH₂)₅—; and R^{x} and R^{w} are each hydrogen.

5. The compound according to claim 1, wherein R⁵ is 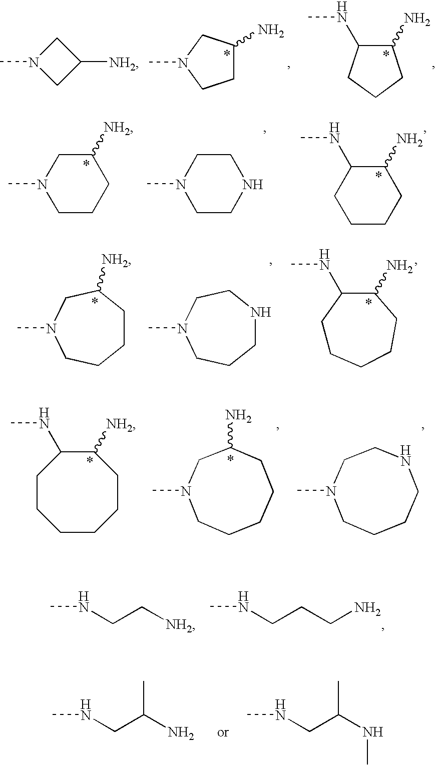
wherein the symbol * signifies a chiral centre of (S)- or (R)-configuration.

6. The compound according to claim 1, which is of formula (IV) or formula (V): 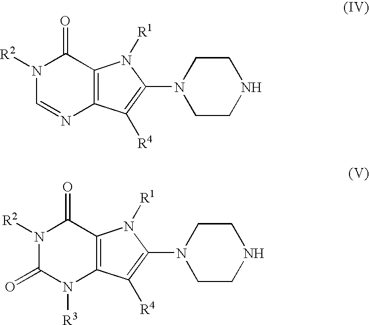
or a pharmaceutically acceptable salt of prodrug thereof.

7. (canceled)

8. The compound according to claim 1, wherein R⁵ is homopiperazinyl optionally substituted with 1, 2, 3, 4 or 5 R¹².

9. The compound according to claim 8, which is of the formula (VII) or (IX): 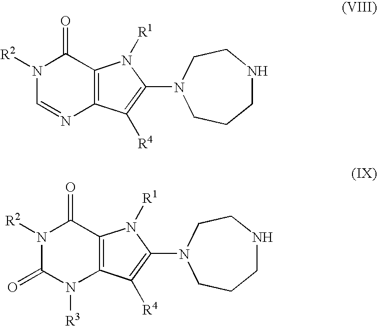
or a pharmaceutically acceptable salt or prodrug thereof.

10. The compound according to claim 1, wherein R¹ is C₁₋₆ alkyl, C₂₋₆ alkenyl or C₂₋₆ alkynyl, any of which is optionally substituted with 1, 3, 4 or 5 R¹².

11. The compound according to claim 10, wherein R¹ is selected from C₁, C₂, C₃ or C₄ alkyl; C₂, C₃, C₄, C₅ or C₆ alkenyl; and C₂, C₃, C₄, C₅ or C6 alkynyl; any of which is optionally substituted with 1, 2, 3, 4 or 5 R¹², wherein the or each R¹² is, for example, C₁₋₆ alkoxy, hydroxy or halogen.

12. The compound according to claim 11, wherein R¹ is methyl, butyl, 2-methoxyethyl, 3-methyl-buten-2-yl or but-2-ynyl.

13. The compound according to claim 1, wherein R¹ is —(CH₂)ₙ—R⁶, wherein n is 0, 1, 2, 3, 4, 5 or 6, and R⁶ is carbocyclyl or heterocyclyl, either of which is optionally substituted with 1, 2, 3, 4 or 5 R¹²; and wherein the or each R¹² is selected from, for example, cyano, trifluoromethyl, hydroxy; halogen; C₁, C₂, C₃ or C₄ alkyl optionally substituted with 1, 2 or 3 hydroxy or with 1, 2, 3 or more halogen; and C₁, C₂, C₃ or C₄ alkoxy, optionally substituted with 1, 2, 3 or more halogen atoms.

14. The compound according to claim 13, wherein R⁶ is aryl optionally substituted with 1, 2, 3, 4 or 5 R¹².

15. The compound according to claim 14, wherein R⁶ is phenyl optionally substituted with 1, 2 or 3 substituents selected from halogen, hydroxy, cyano, methoxy, ethoxy, trifluoromethyl, methyl and ethyl; and n is 0, 1 or 2.

16. The compound according to claim 15, wherein R¹ is 2-fluorobenzyl or unsubstituted benzyl.

17. The compound according to claim 13, wherein R⁶ is cycloalkyl optionally substituted with 1, 2, 3, 4 or 5 R¹².

18. The compound according to claim 17, wherein R⁶ is cyclopropyl, cyclobutyl, cyclopentyl or cyclohexyl, any of which is optionally substituted with 1, 2 or 3 substituents selected from halogen, hydroxy, cyano, methoxy, ethoxy, trifluoromethyl, methyl and ethyl; and n is 0, 1 or 2.

19. The compound according to claim 18, wherein R¹ is cyclopropylmethyl, 2-methylcyclopropylmethyl, cyclopropylethyl, or cyclobutylmethyl.

20. The compound according to claim 13, wherein R⁶ is heterocycloalkyl optionally substituted with 1, 2, 3, 4 or 5 R¹².

21. The compound according to claim 20, wherein R⁶ is tetrahydrofuranyl; and n is 0, 1 or 2.

22. The compound according to claim 21, wherein R¹ is tetrahydrofuranylmethyl, for example tetrahydrofuran-2-ylmethyl.

23. The compound according to claim 13, wherein R⁶ is heteroaryl optionally substituted with 1, 2, 3, 4 or 5 R¹².

24. The compound according to claim 23, wherein R⁶ is thiazolyl, furanyl or oxazolyl, any of which is optionally substituted with 1, 2 or 3 substituents selected from halogen, hydroxy, cyano, methoxy, ethoxy, trifluoromethyl, methyl and ethyl; and n is 0, 1 or 2.

25. The compound according to claim 24, wherein R¹ is thiazolylmethyl, furanylmethyl or oxazolylmethyl.

26. The compound according to claim 1, wherein R² is —(CH₂)ₙ—R⁷, —(CH₂)ₙ—OR⁷, —(CH₂)ₙ—C(O)R⁷ or —(CH₂)ₙ—S(O)ₘR⁷, wherein n is 0, 1, 2, 3, 4, 5 or 6; and R⁷ is aryl or heteroaryl; either of which is optionally substituted with 1, 2, 3, 4 or 5 R¹²; wherein the or each R¹² is selected from, for example, cyano, trifluoromethyl, hydroxy; halogen; C₁, C₂, C₃ or C₄ alkyl optionally substituted with 1, 2 or 3 hydroxy or with 1 or more halogen (e.g. chlorine or fluorine); and C₁, C₂, C₃ or C₄ alkoxy, optionally substituted with 1, 2 or 3 or more halogen atoms.

27. The compound according to claim 26, wherein n is 1 or 2, and R⁷ is phenyl, naphthyl, thiophen-1-yl, thiophen-2-yl, benzo[b]thiophenyl, pyridin-1-yl, pyridin-2-yl, pyridin-3-yl, pyrazin-2-yl or quinolinyl, any of which is optionally substituted with 1, 2 or 3 substituents selected from halogen, hydroxy, cyano, methoxy, ethoxy, trifluoromethyl, methyl and ethyl.

28. The compound according to claim 25, wherein R² is 2-oxo-2-phenyl-ethyl, 2-oxo-2-(3-methoxyphenyl)-ethyl or R² is isoquinolin-1-ylmethyl.

29-68. (canceled)

69. The compound according to claim 1, selected from: 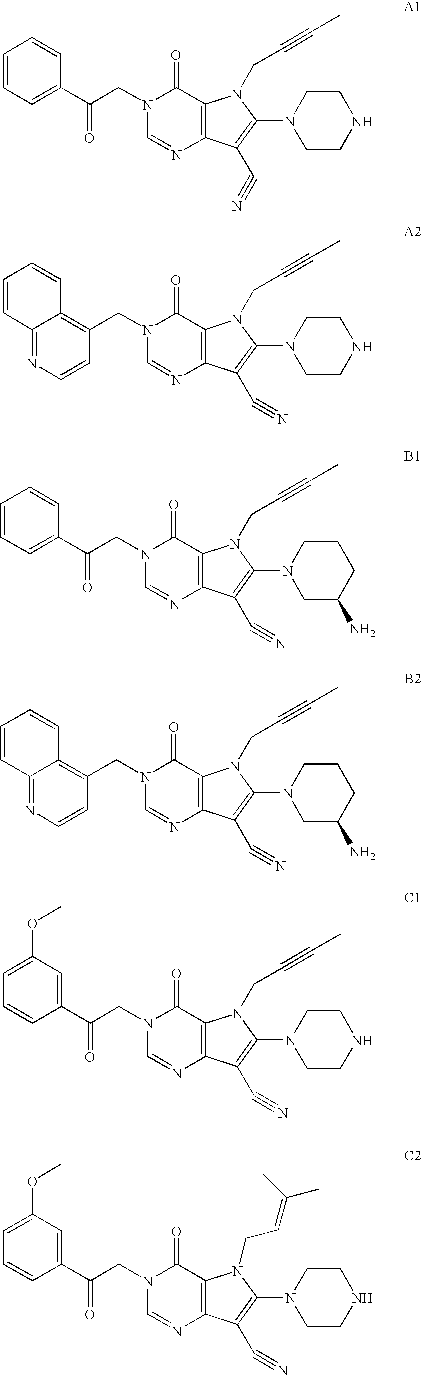 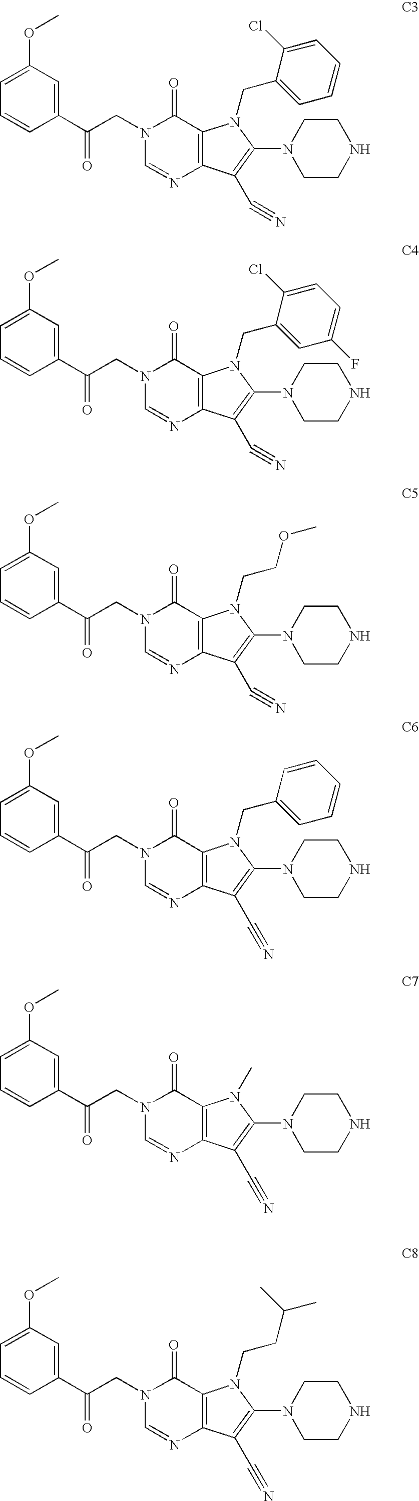 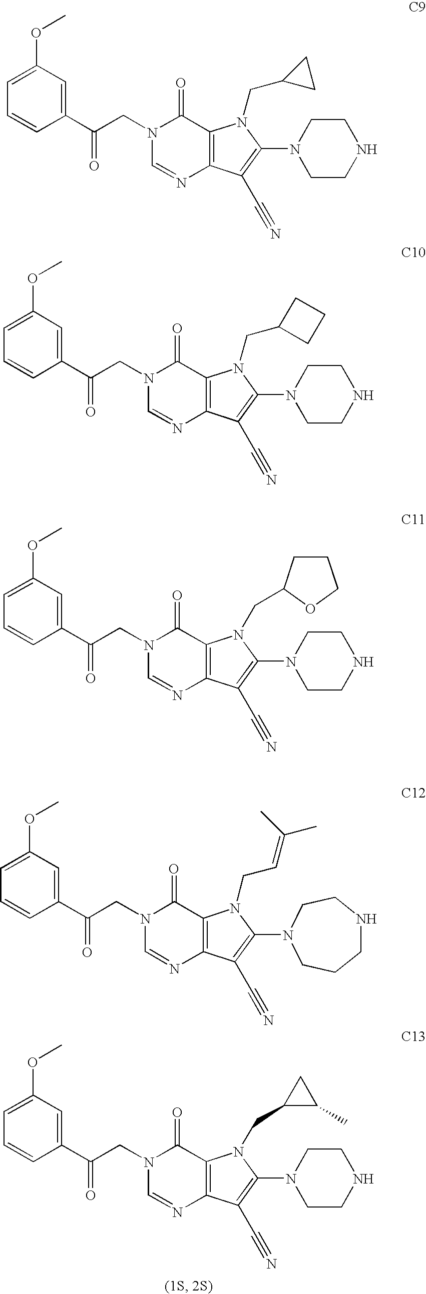 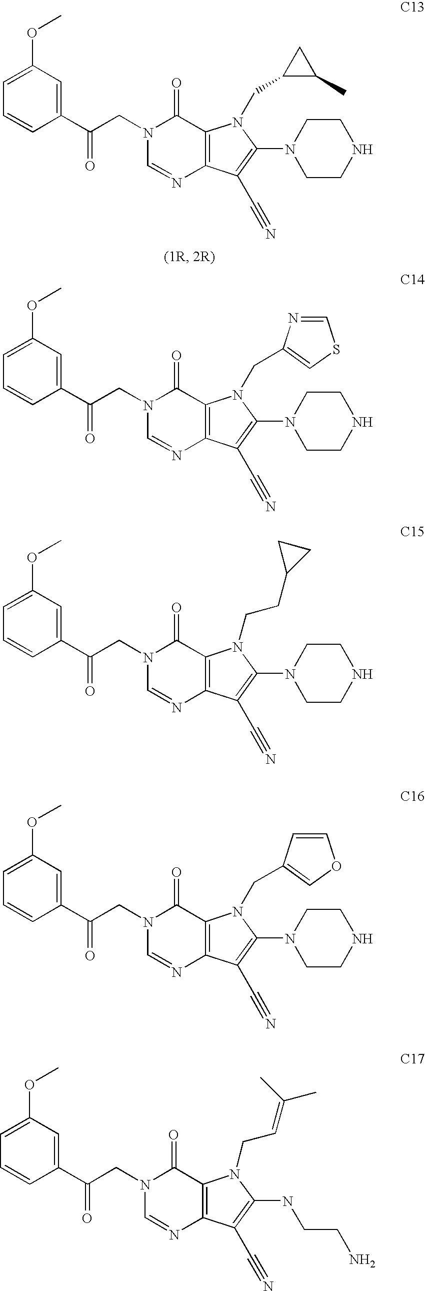 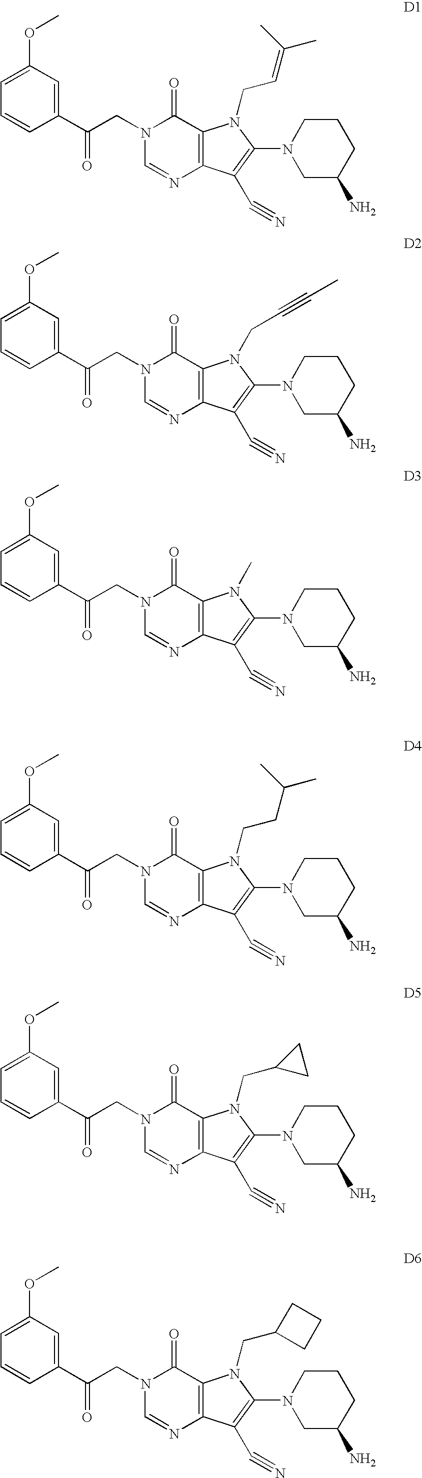 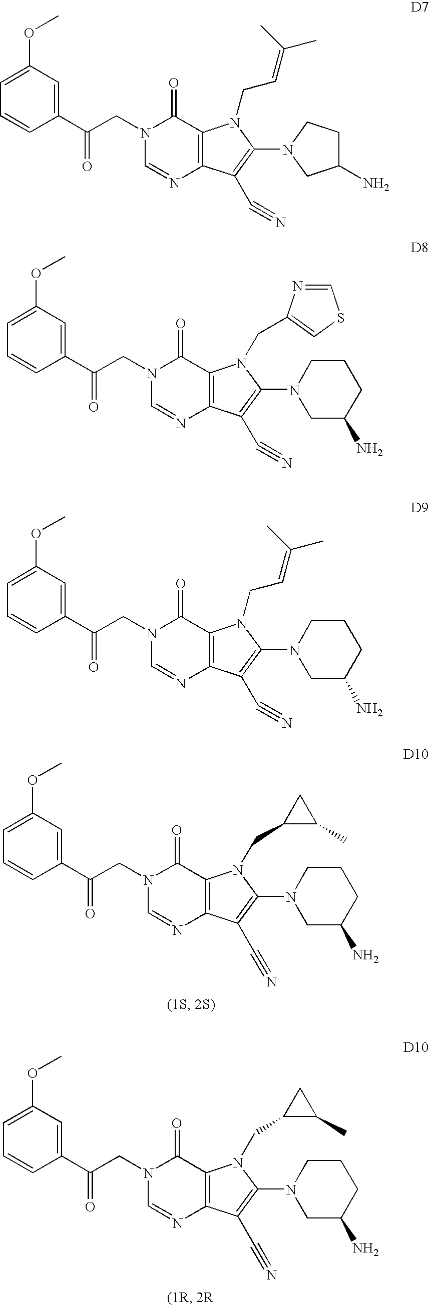 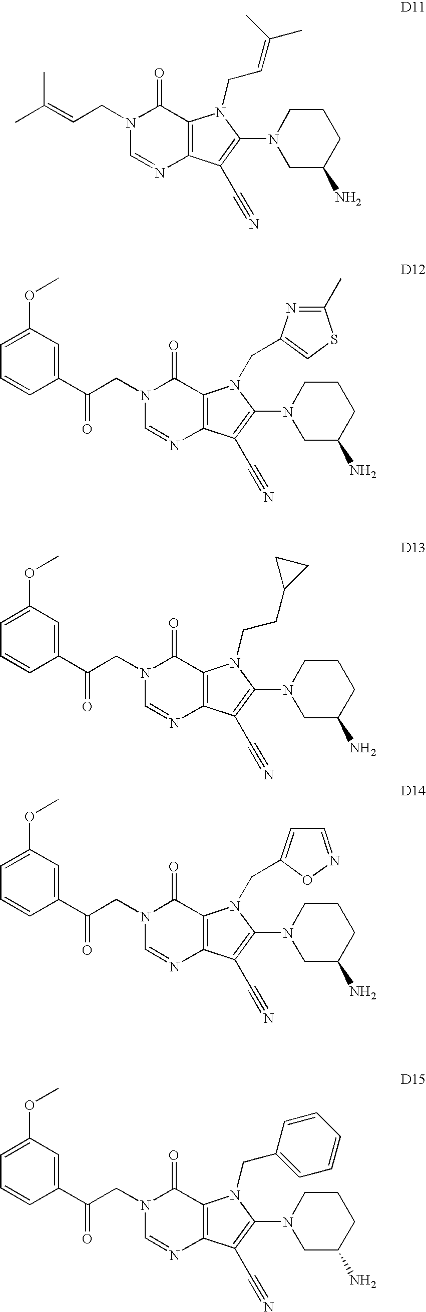 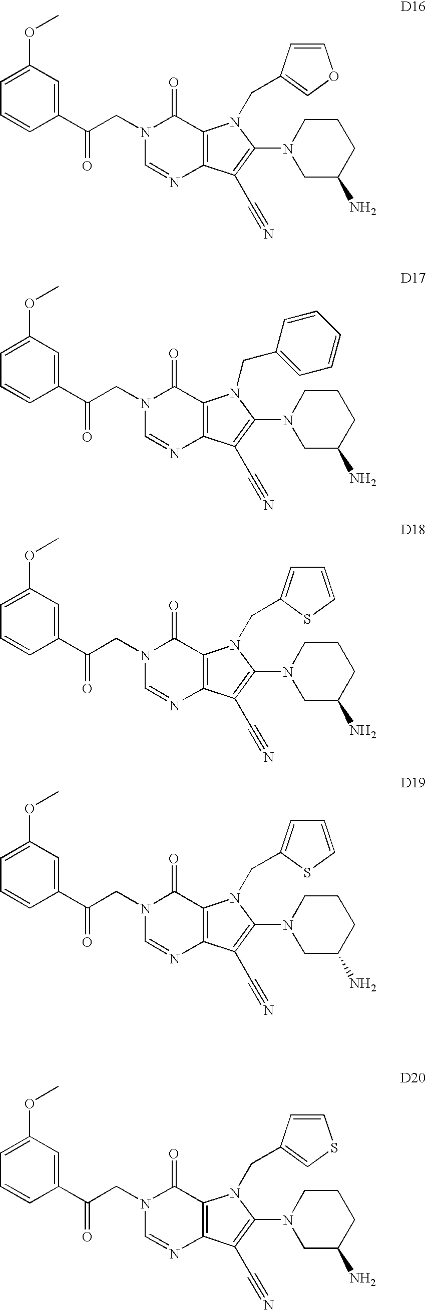 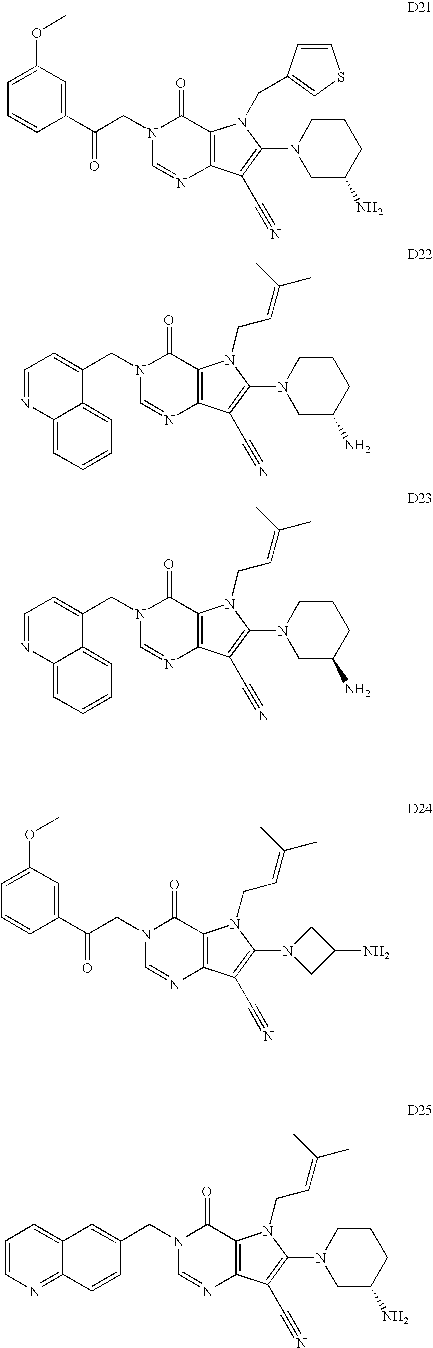 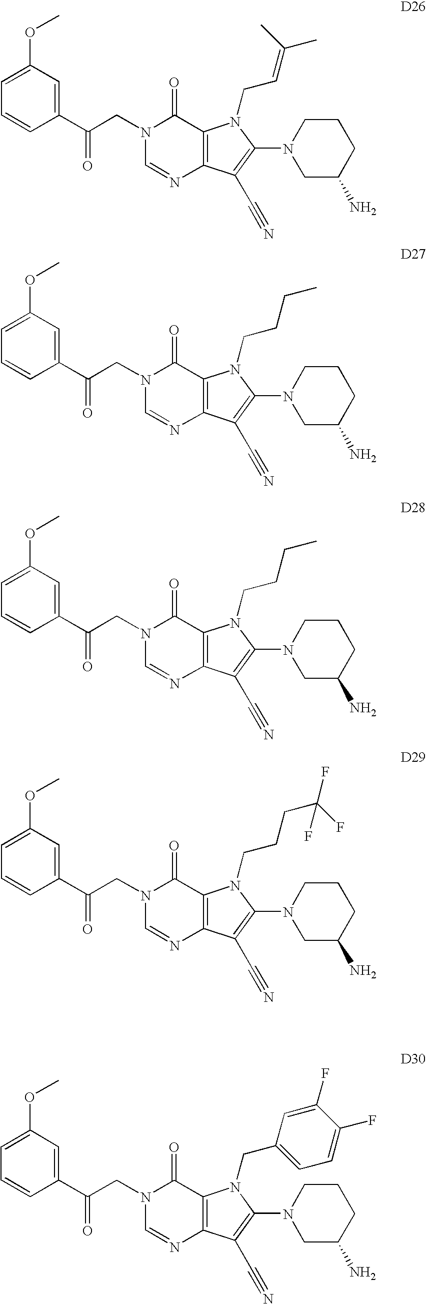 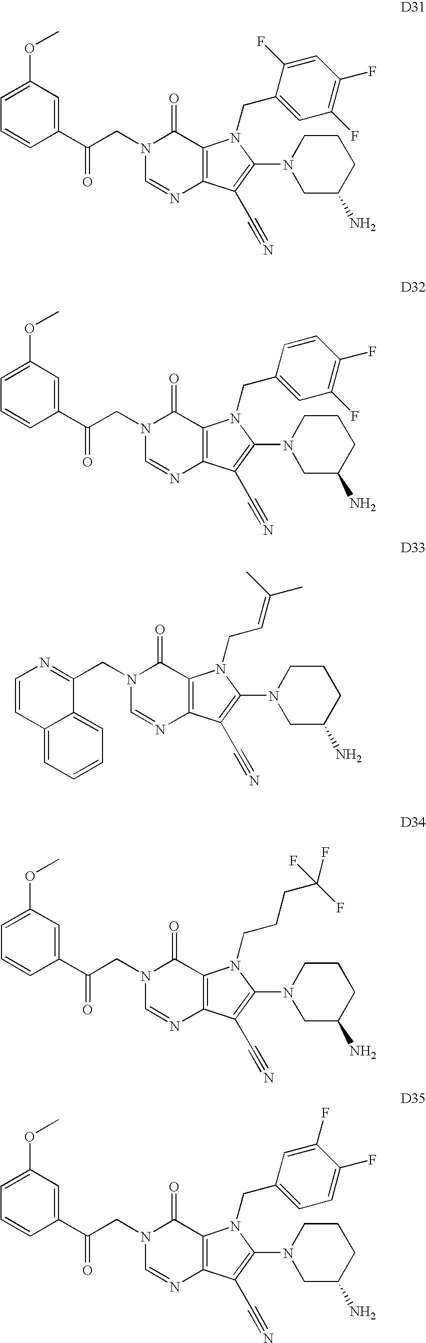 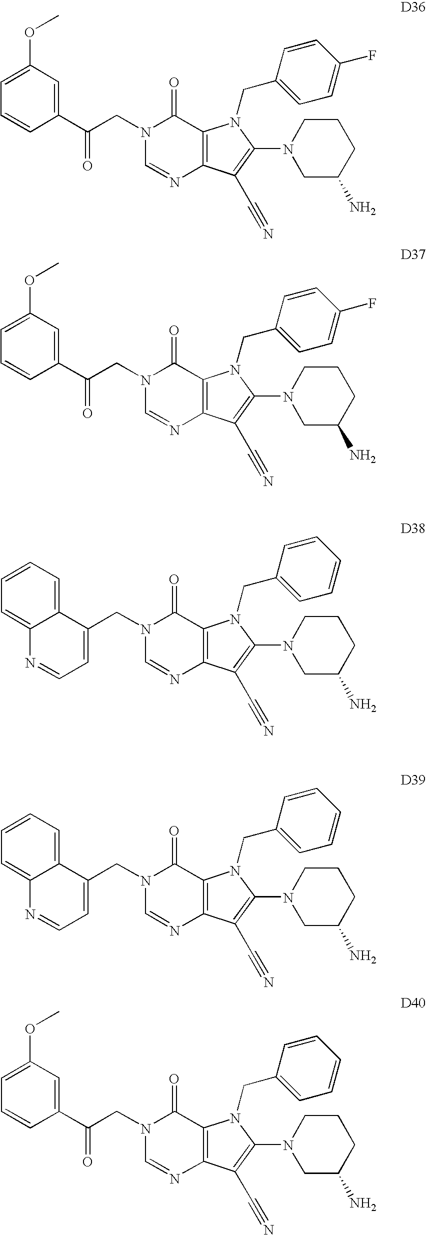 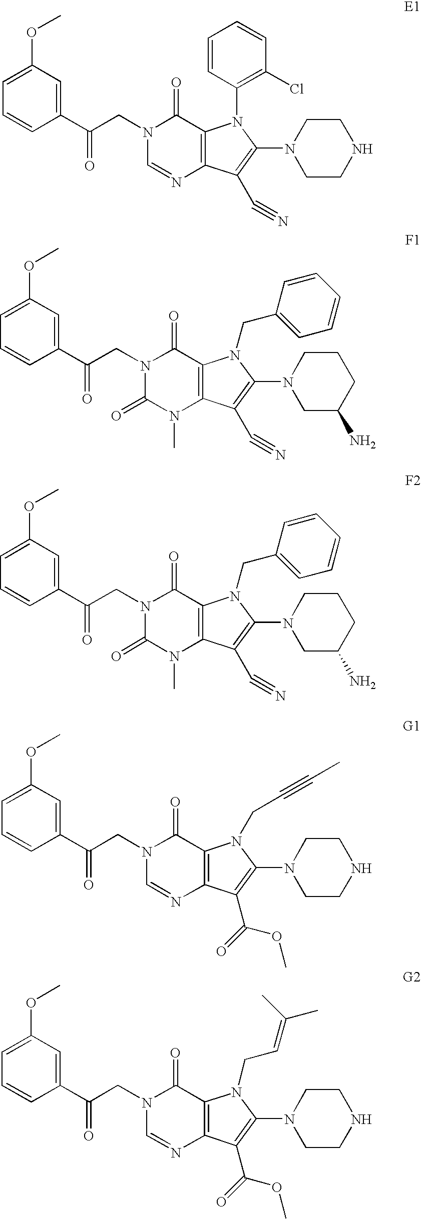 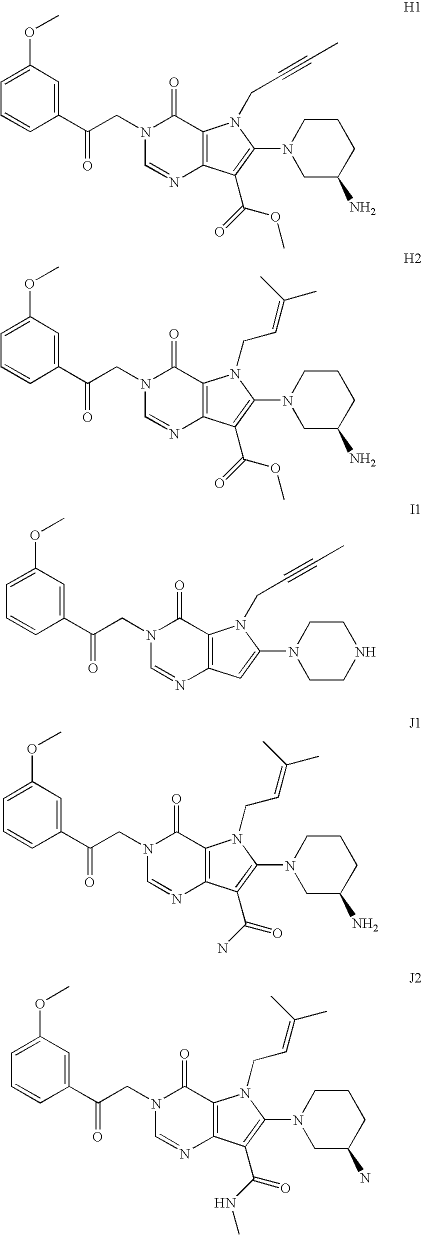 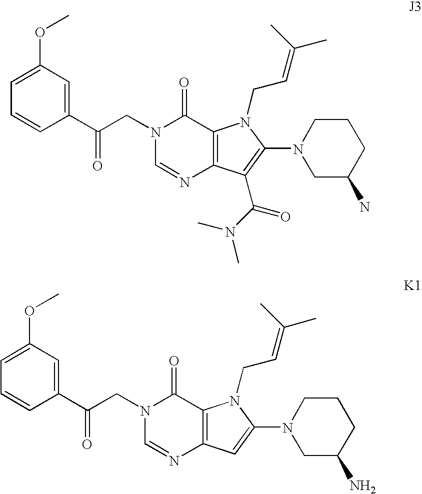
or, in each case, a pharmaceutically acceptable salt or prodrug thereof.

70. The compound according to claim 1, selected from: 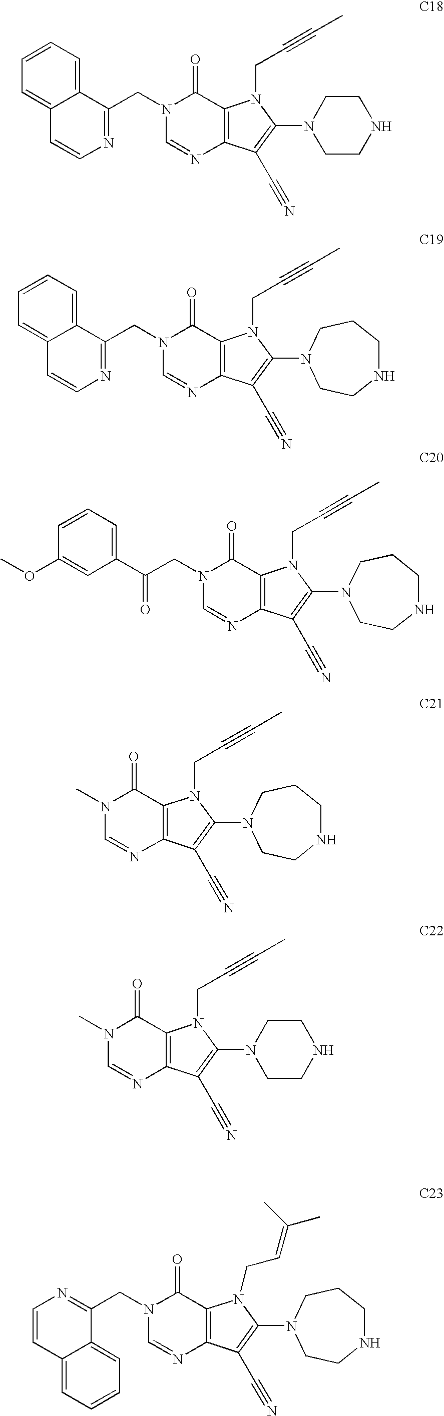 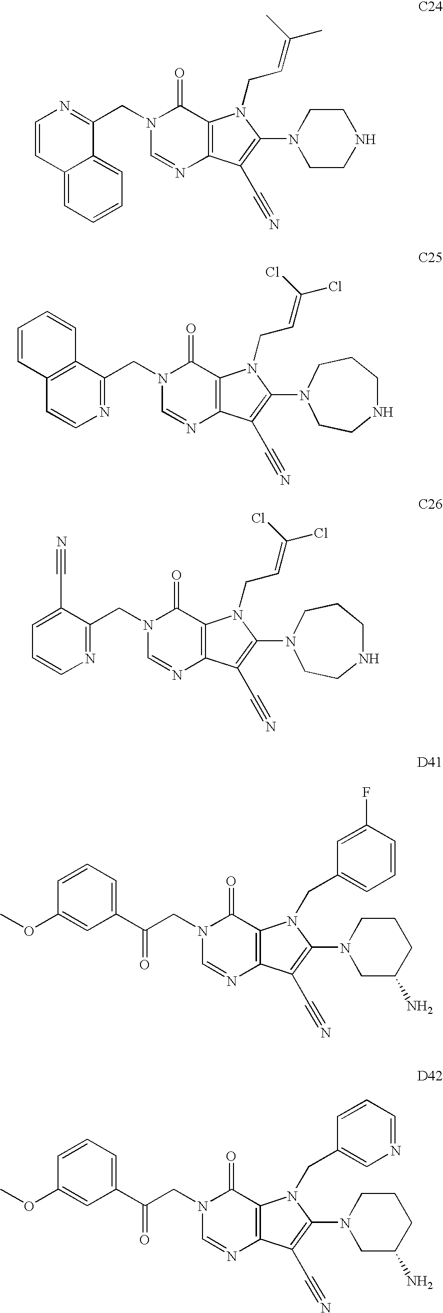 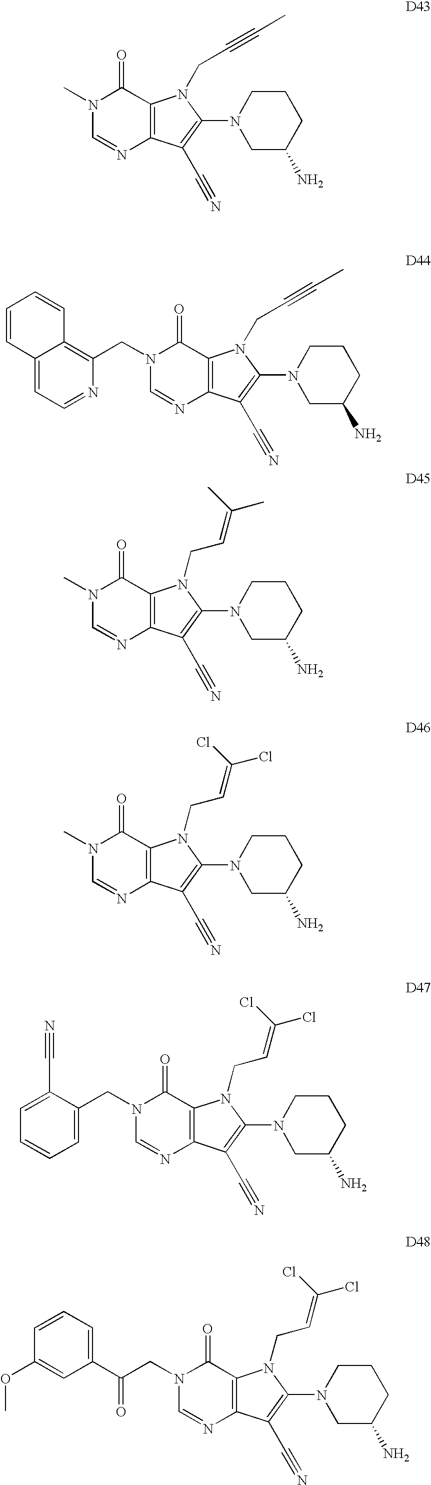 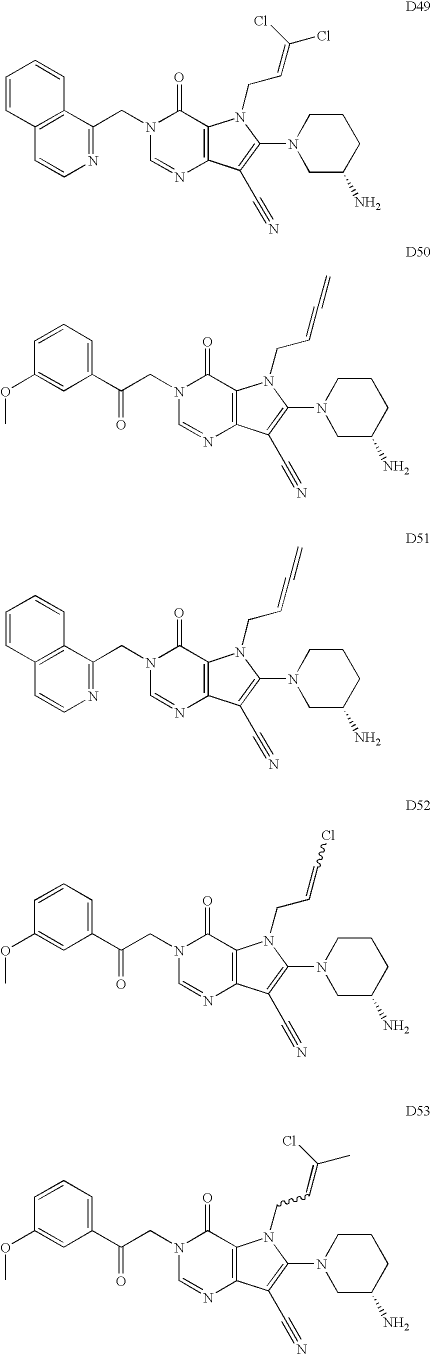 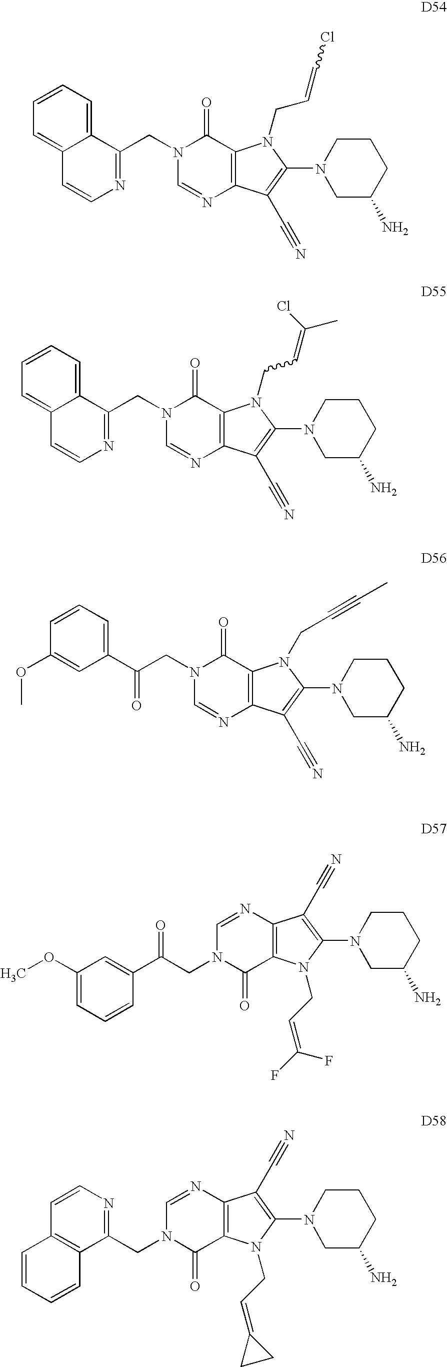 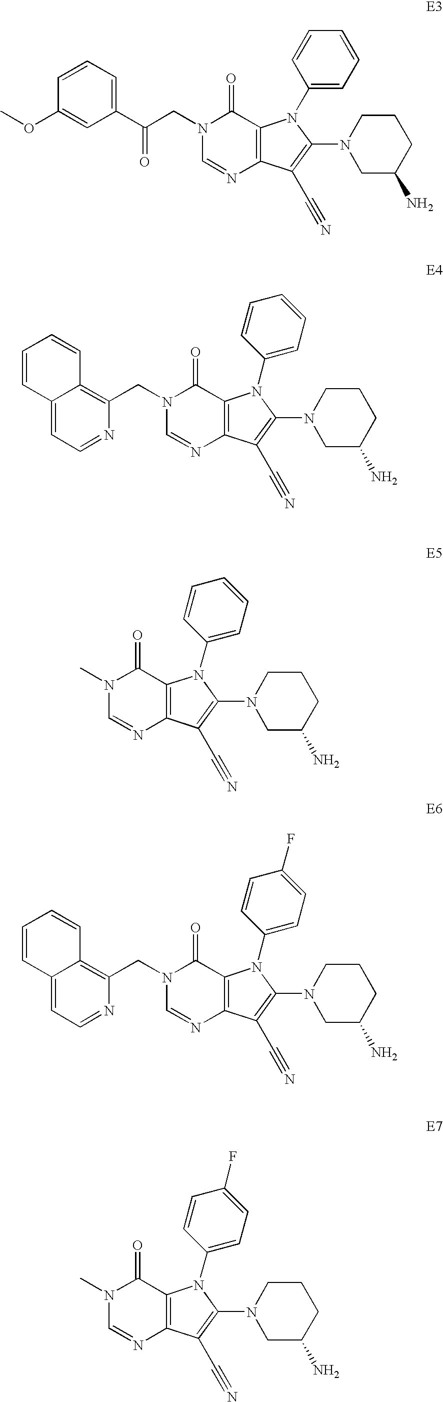 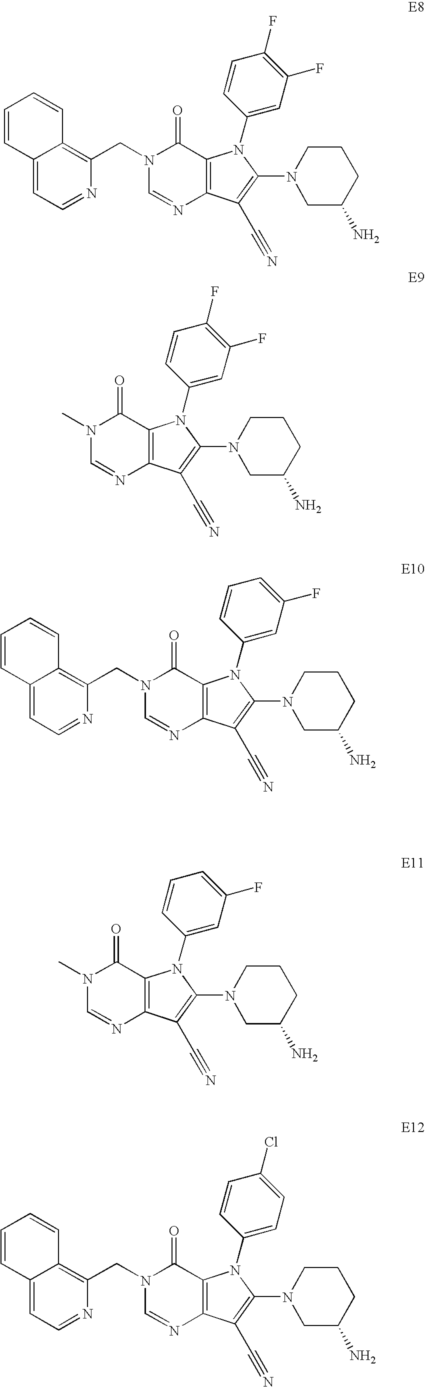 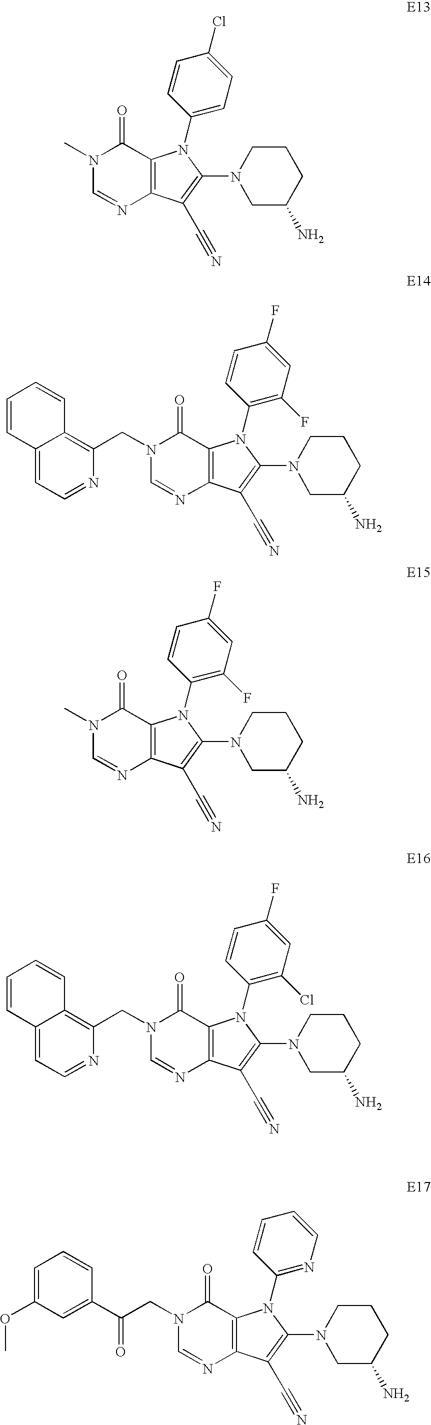 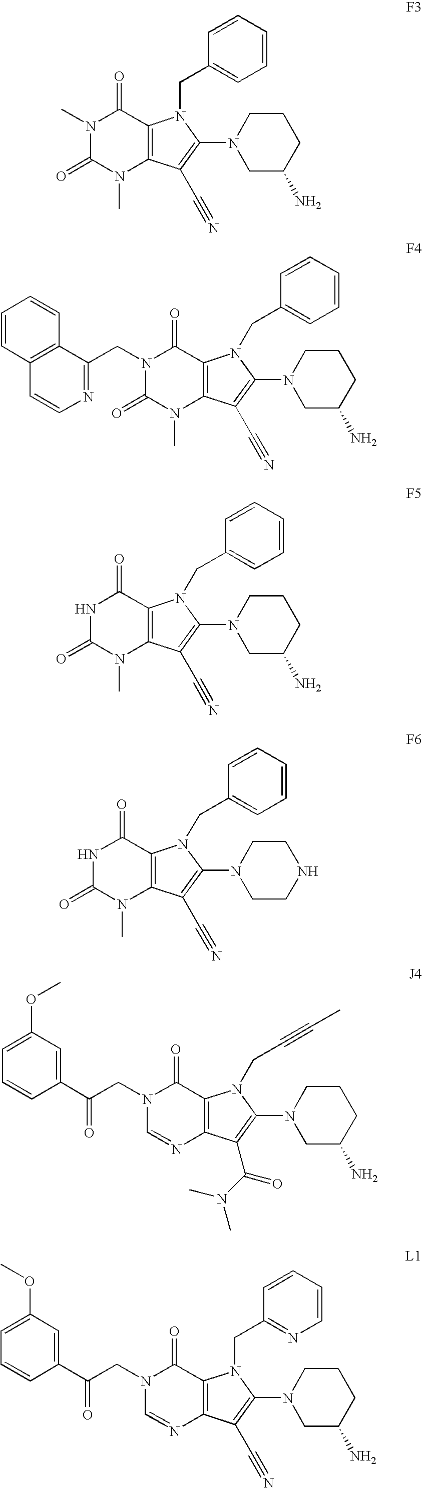 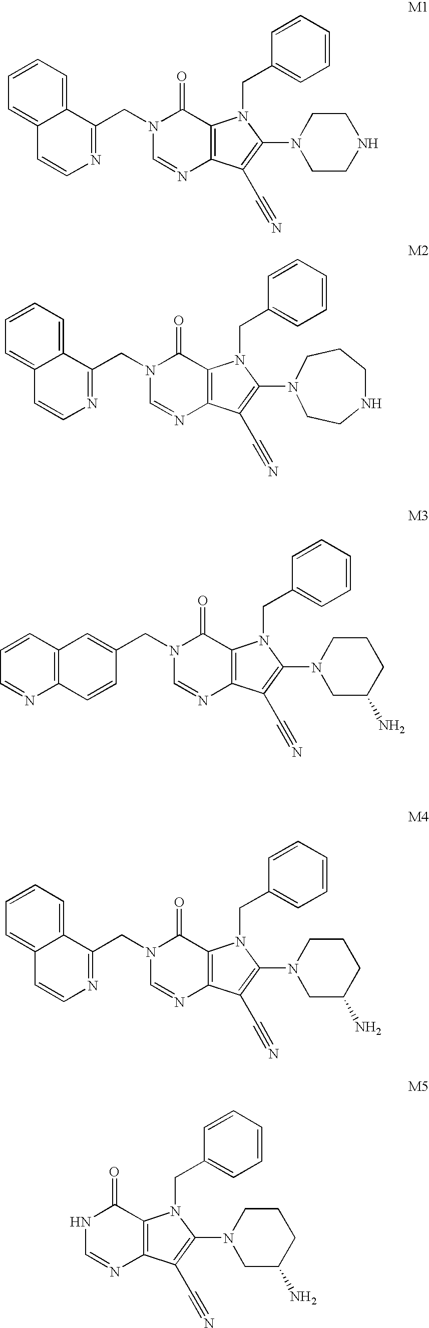 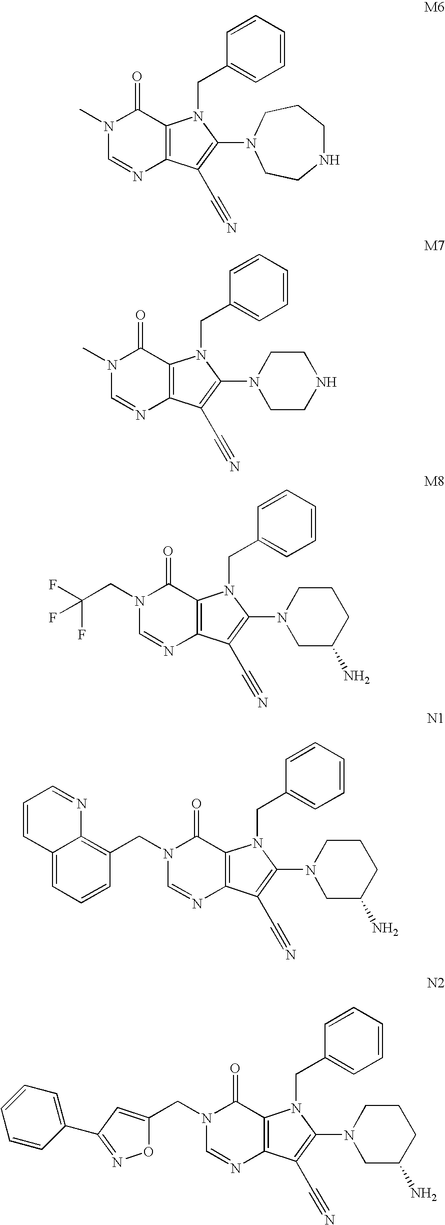 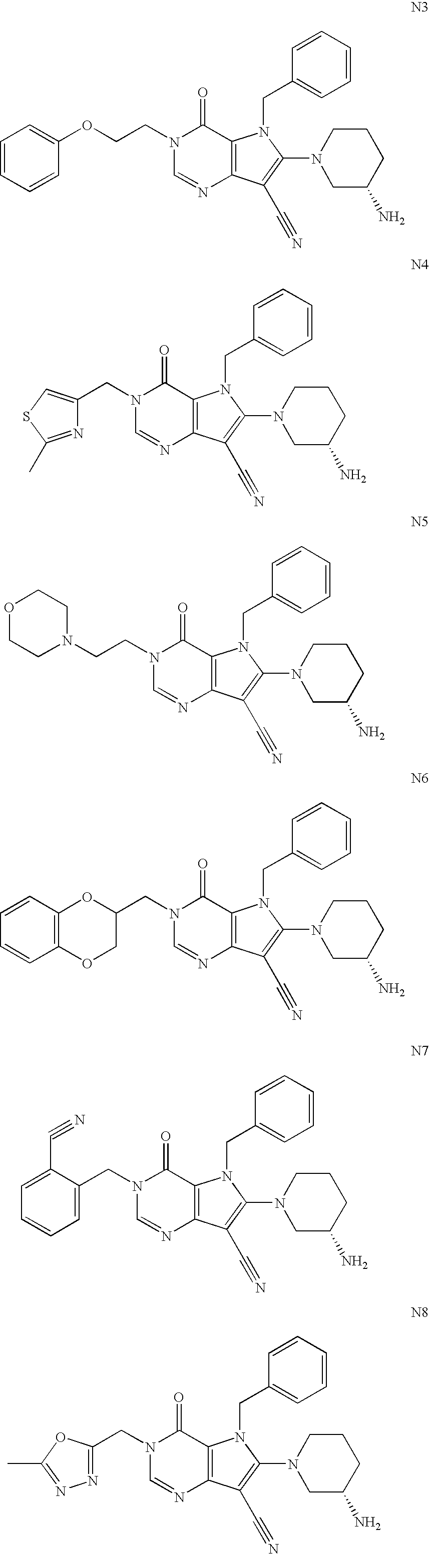 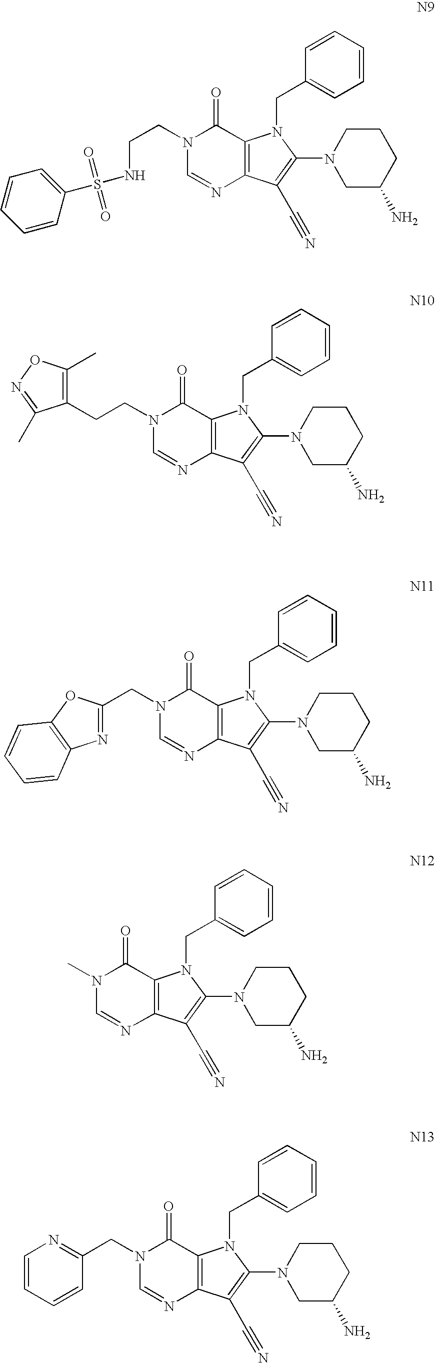 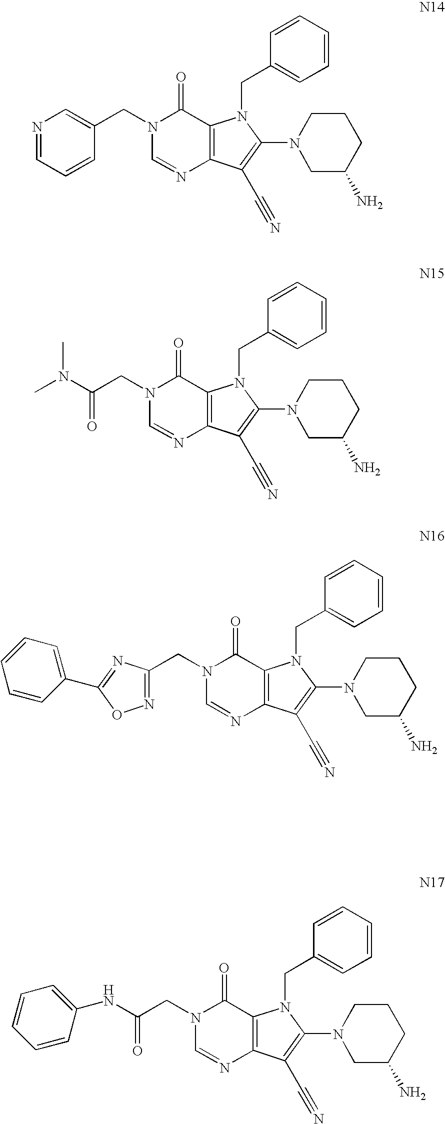 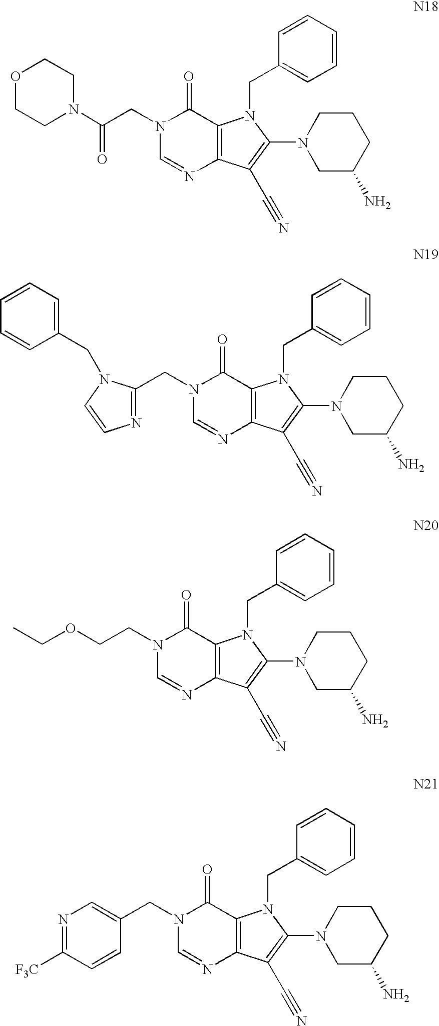 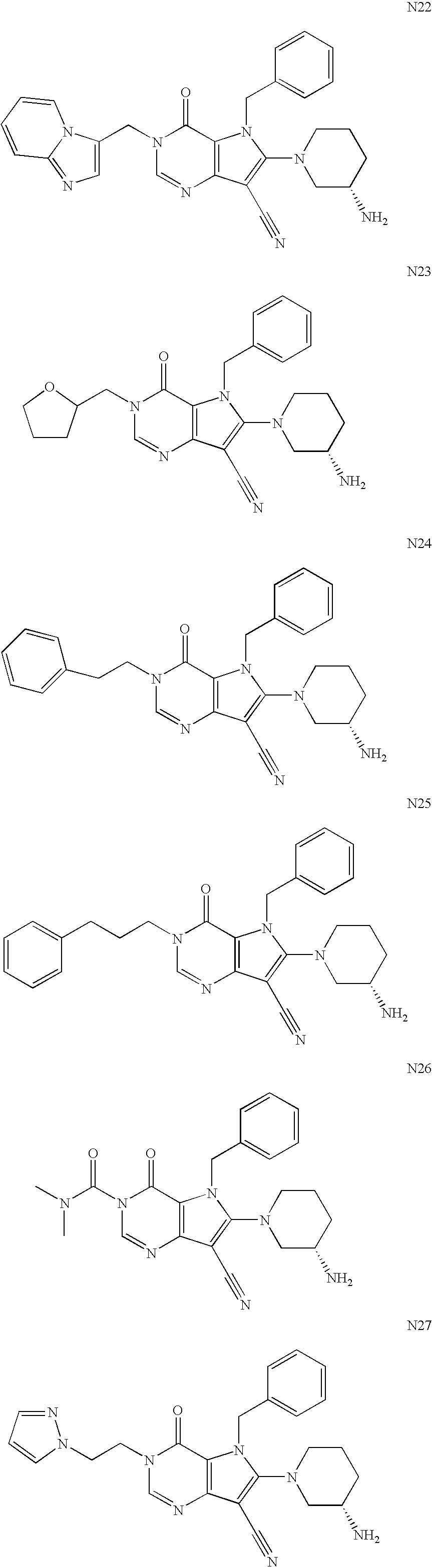 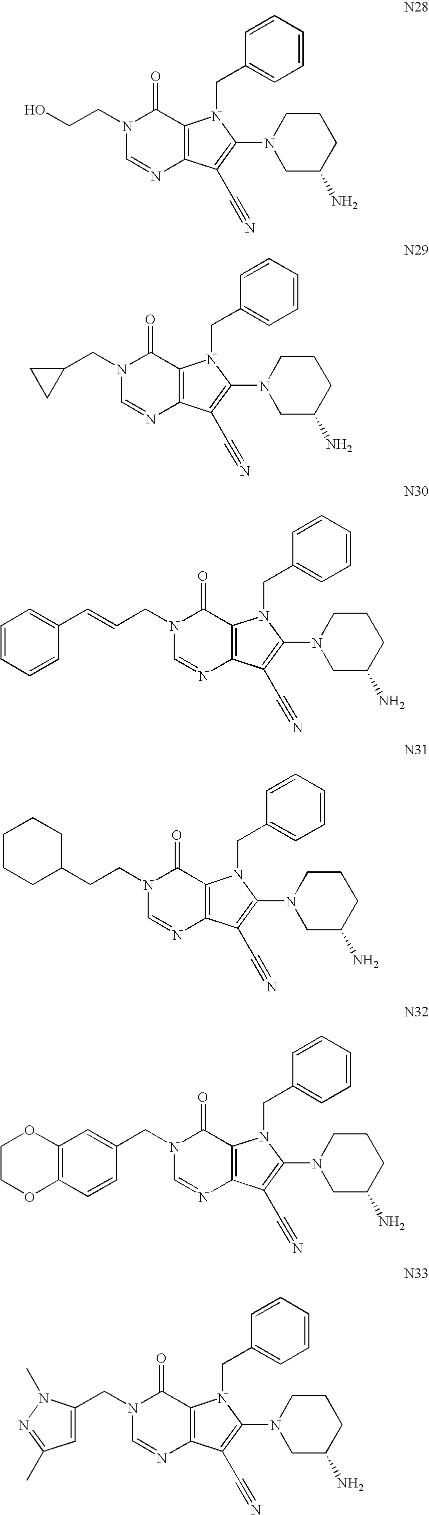 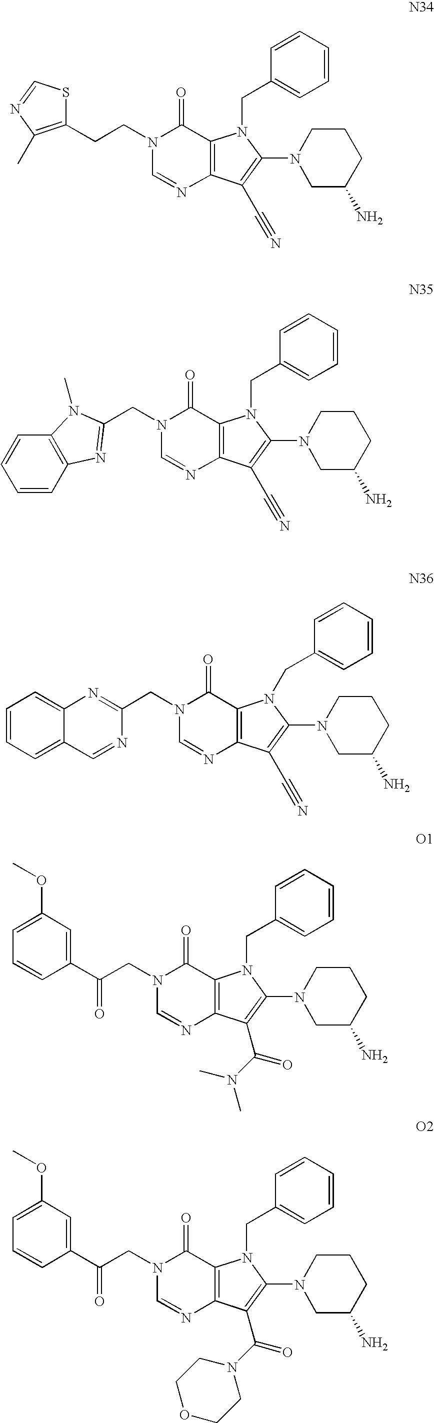 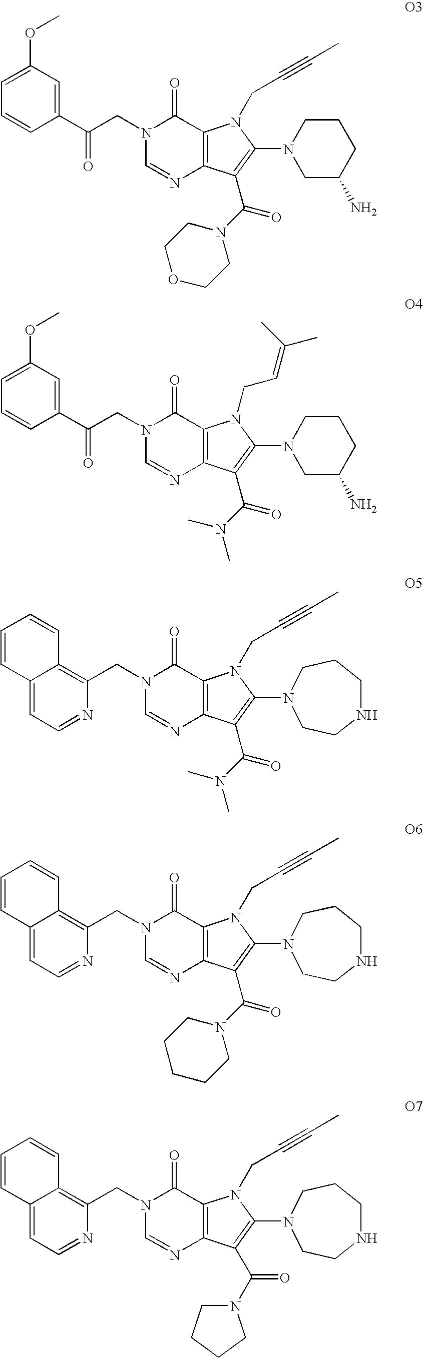 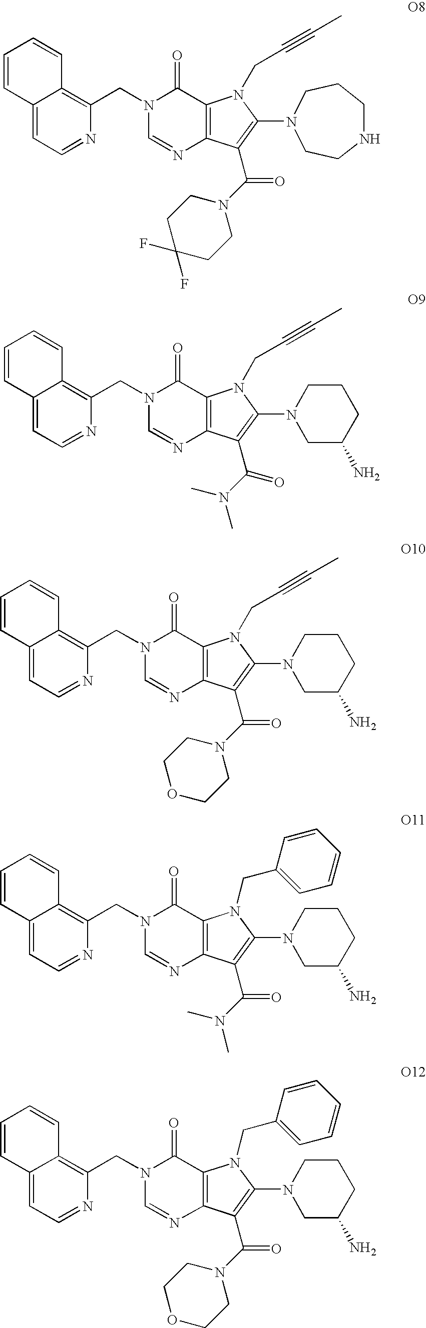 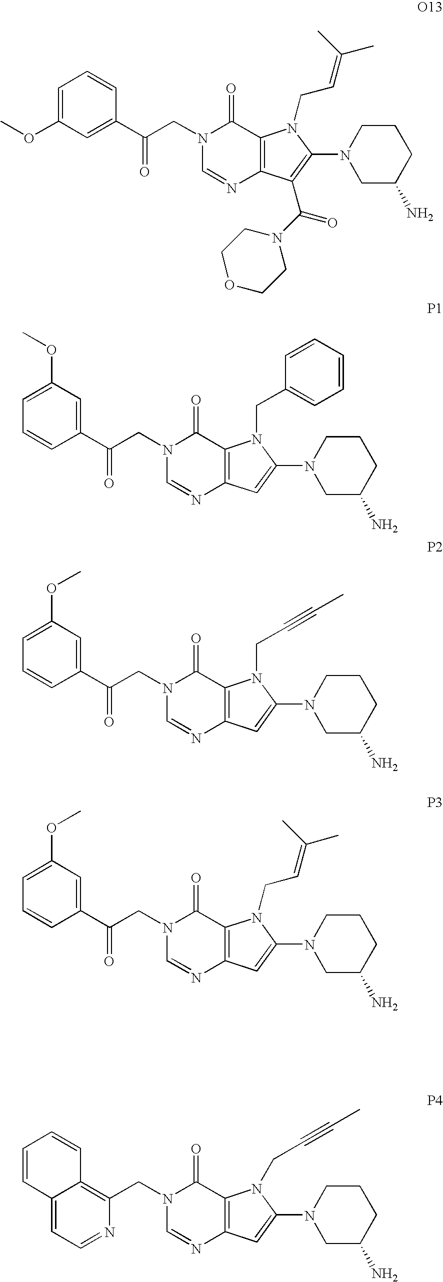 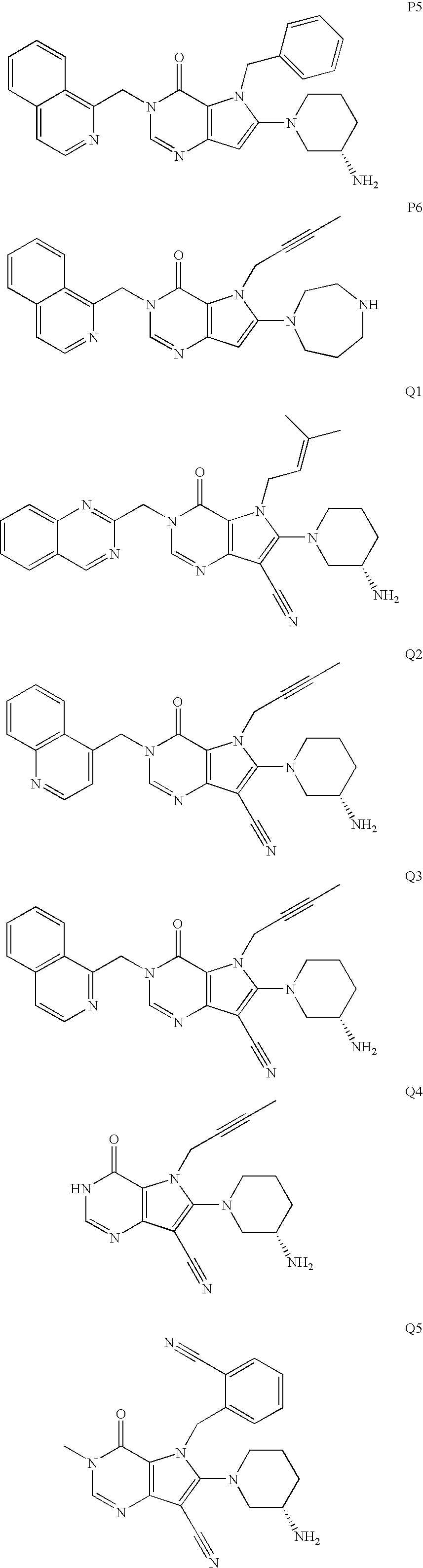 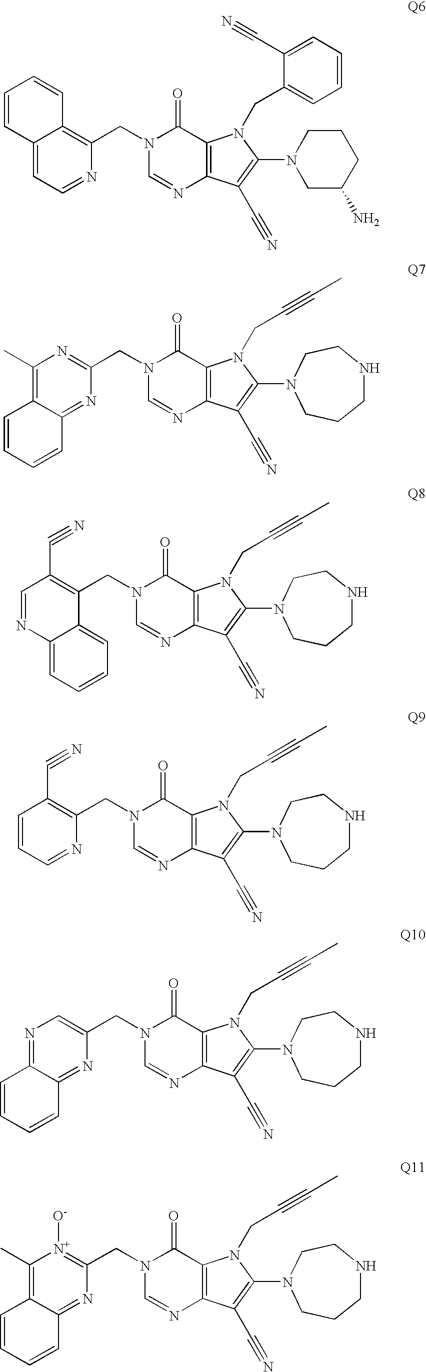 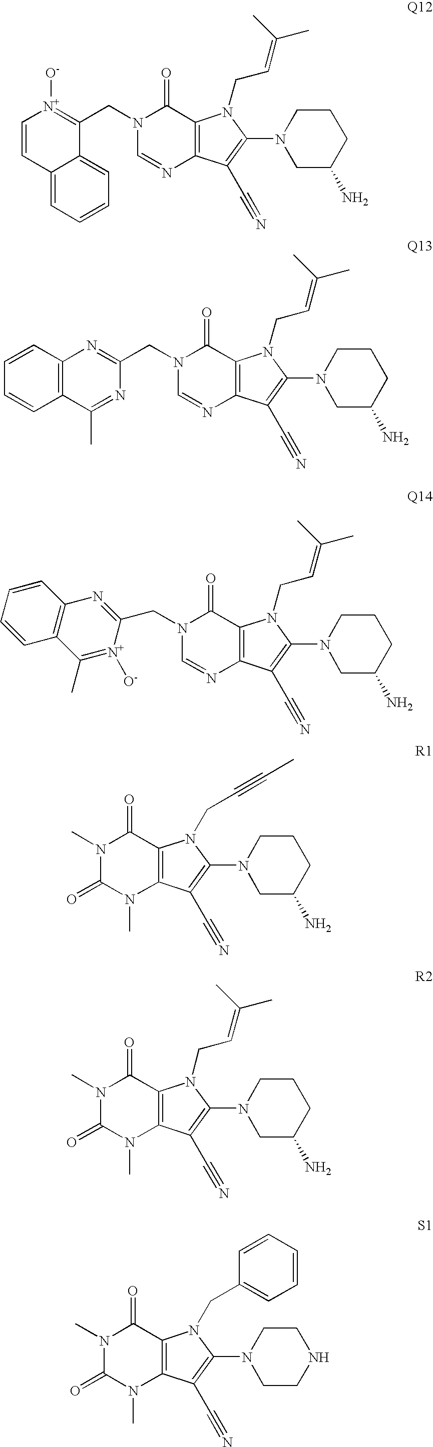 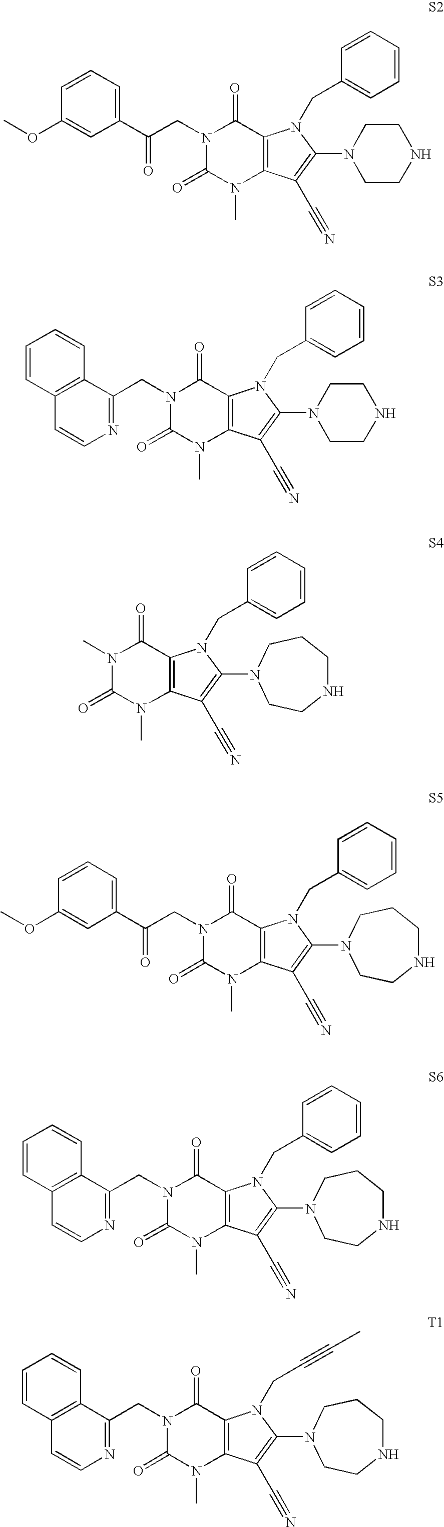 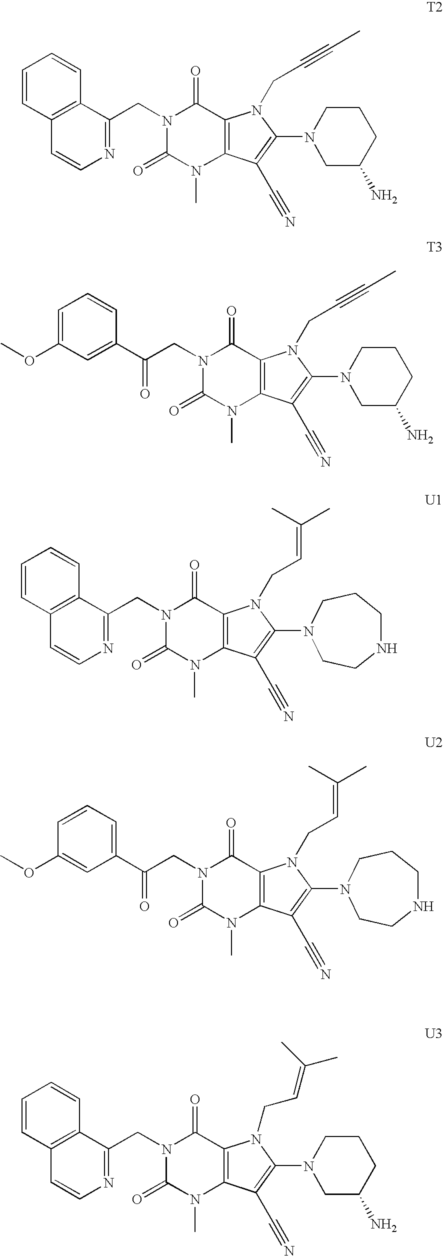 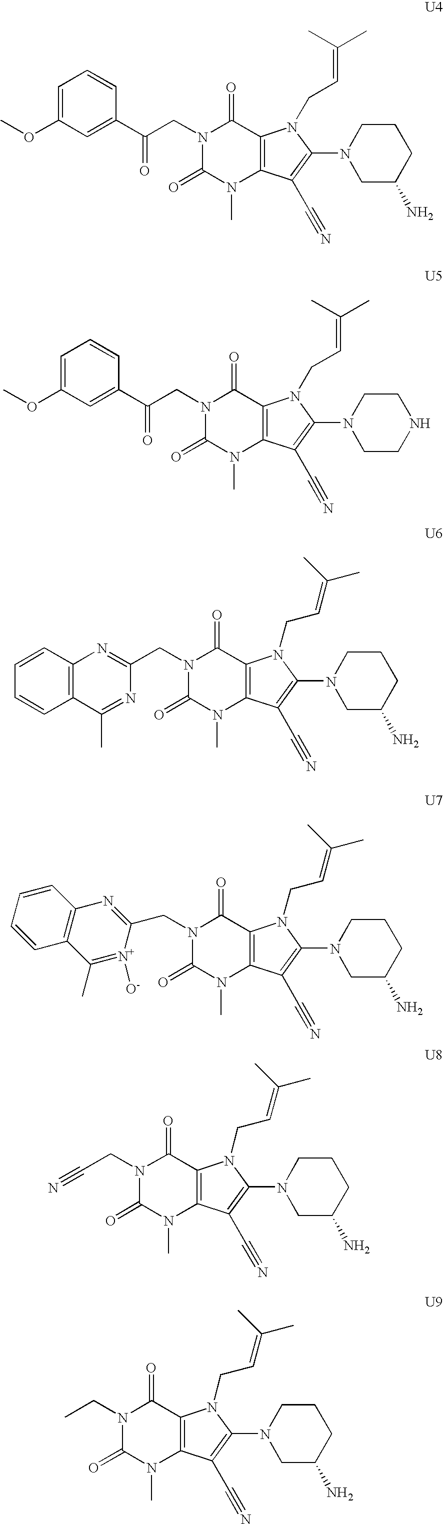 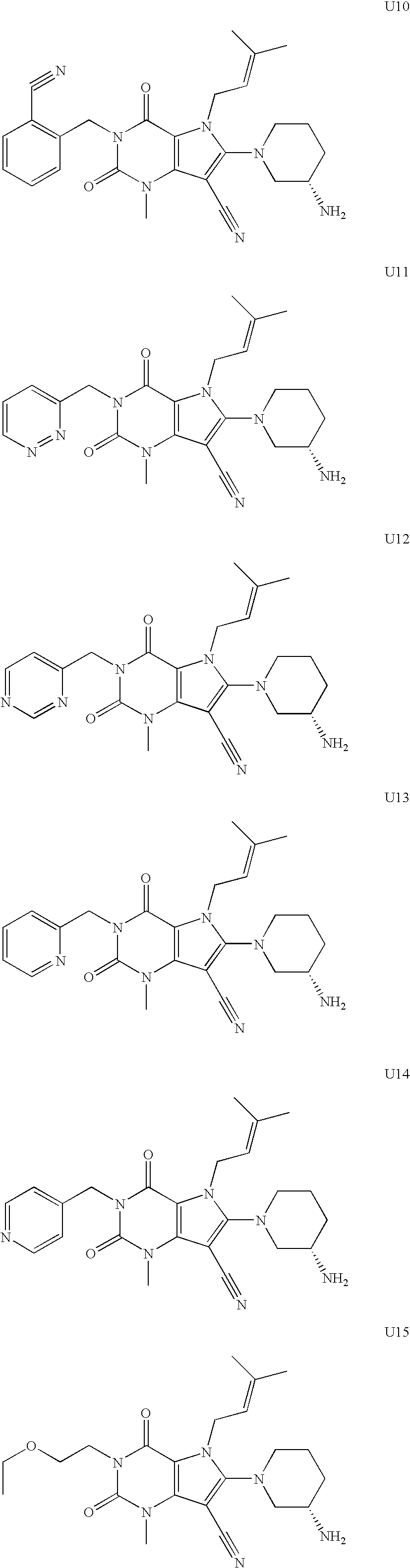 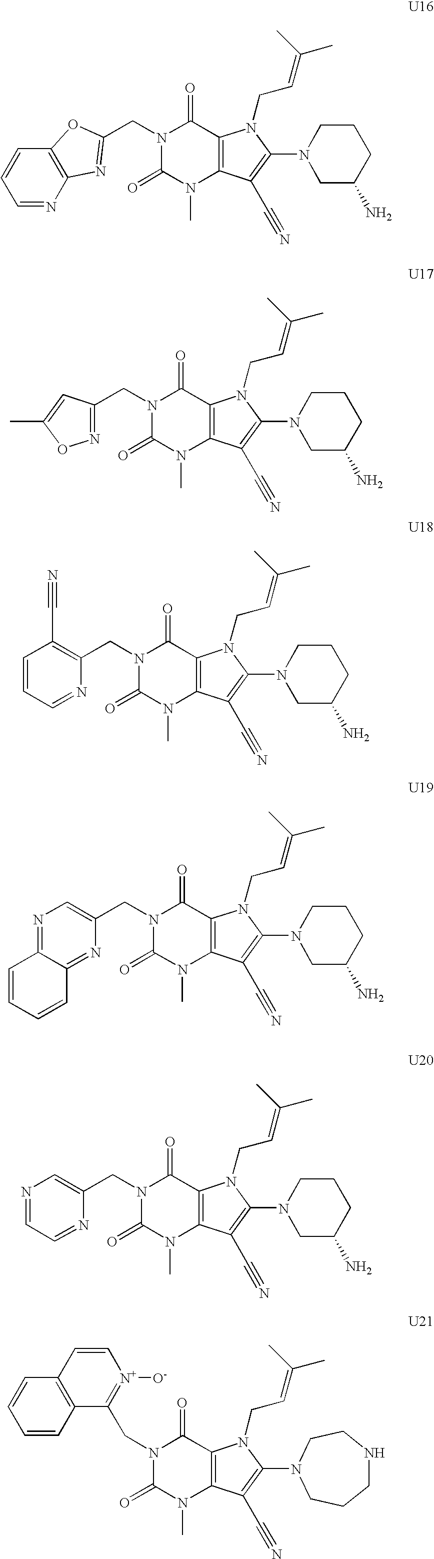 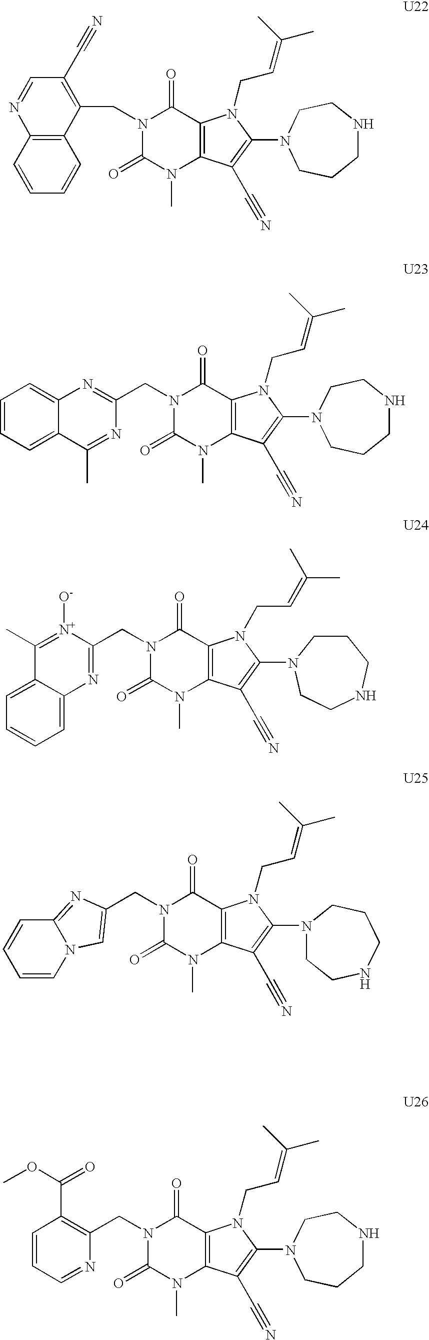 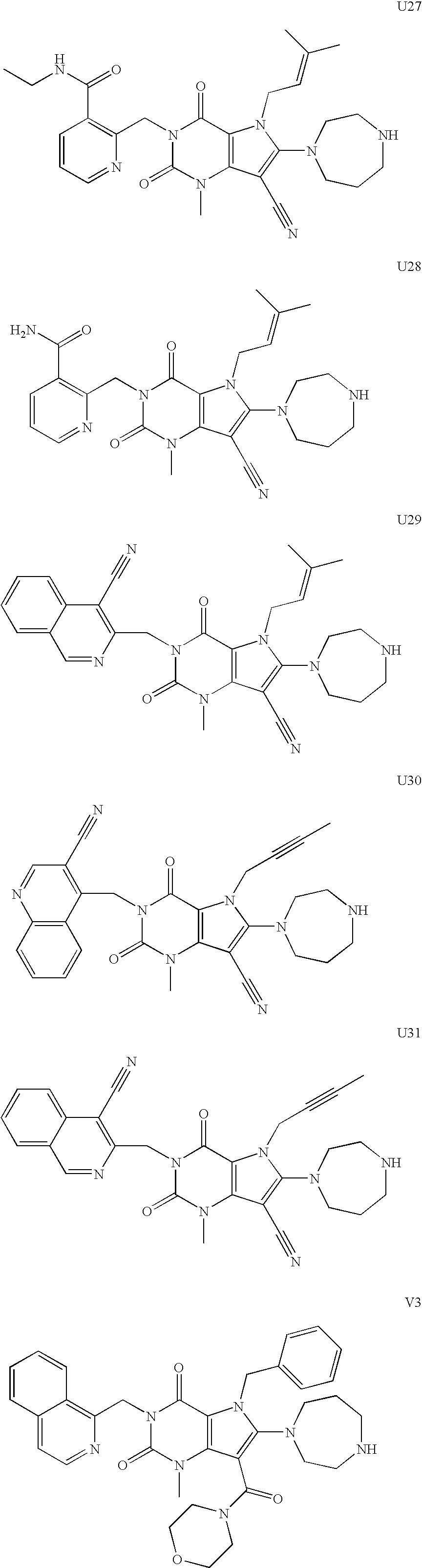 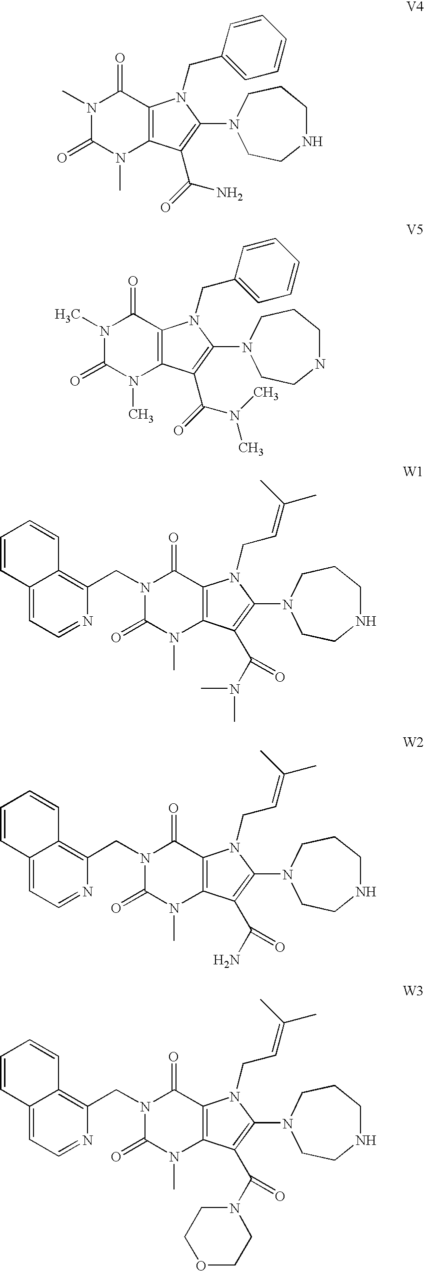 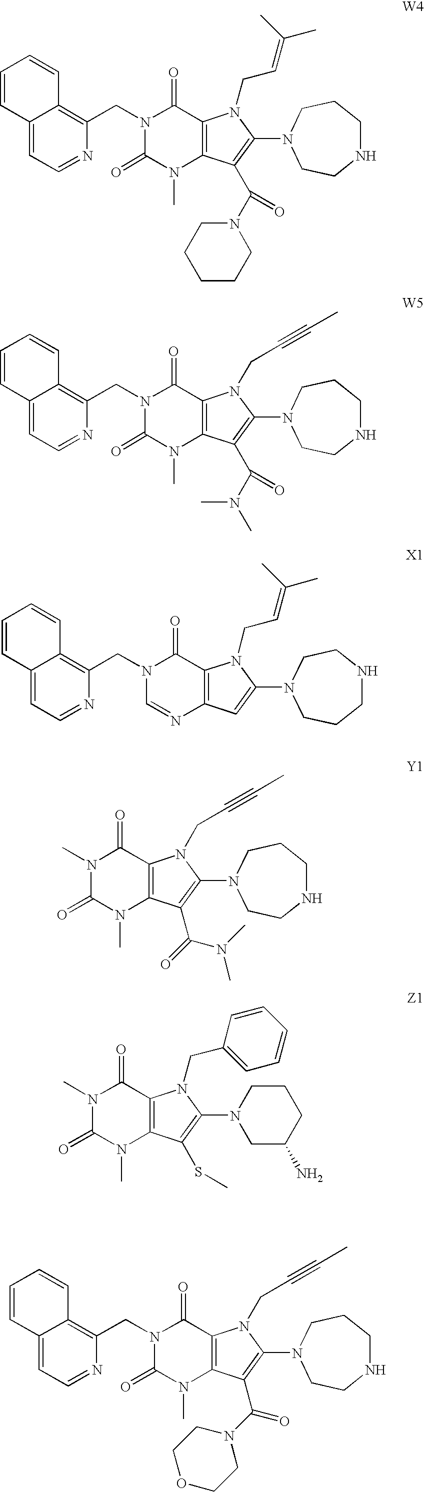 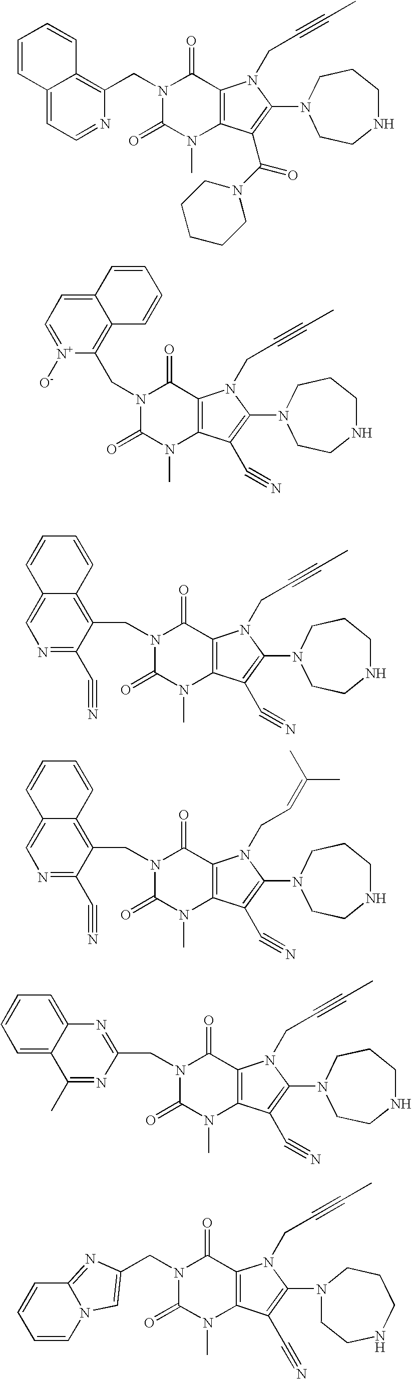
or, in each case, a pharmaceutically acceptable salt or prodrug thereof.

71. (canceled)

72. A pharmaceutical composition, comprising:
the compound of claim 1 and
a pharmaceutically acceptable carrier.

73-81. (canceled)

82. A method of treating a disease or condition in a patient, comprising:
administering a therapeutically effective amount of the compound of claim 1,
wherein the disease or condition selected from non-insulin-dependent diabetes mellitus, arthritis, obesity, allograft transplantation, calcitonin-osteoporosis, heart failure, impaired glucose metabolism or impaired glucose tolerance, neurodegenerative diseases, cardiovascular or renal diseases, and neurodegenerative or cognitive disorders, hyperglycemia, insulin resistance, lipid disorders, dyslipidemia, hyperlipidemia, hypertriglyceridemia, hypercholesterolemia, low HDL levels, high LDL levels, atherosclerosis, vascular restenosis, irritable bowel syndrome, inflammatory bowel disease, pancreatitis, retinopathy, nephropathy, neuropathy, syndrome X, ovarian hyperandrogenism (polycystic ovarian syndrome), type 2 diabetes, growth hormone deficiency, neutropenia, neuronal disorders, tumor metastasis, benign prostatic hypertrophy, gingivitis, hypertension and osteoporosis.

83. (canceled)
